SEQUENCE LISTING (1122 sequences)

SEQ ID NO: 1 - DNA, 290 bp, synthetic construct
  source 1..290: mol_type = other DNA
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 2 - DNA, 173 bp, synthetic construct
  source 1..173: mol_type = other DNA
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 3 - DNA, 305 bp, synthetic construct
  source 1..305: mol_type = other DNA
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 4 - DNA, 180 bp, synthetic construct
  source 1..180: mol_type = other DNA
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 5 - DNA, 199 bp, synthetic construct
  source 1..199: mol_type = other DNA
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 6 - protein, 490 aa, Ruminococcus gnavus
MSQVIKKKRNTFMIGTEYILNSTQLEEAIKSFVHDFCAEKHEIHDQPVVVEAKEHQEDKIKQIKIPEKGRPVNEVVSEMMNEVYRYRGDANHPRFFSFVPGPASSVSWLGDIMTSAYNIHAGGSKLAPMVNCIEQEVLKWLAKQVGFTENPGGVFVSGGSMANITALTAARDNKLTDINLHLGTAYISDQTHSSVAKGLRIIGITDSRIRRIPTNSHFQMDTTKLEEAIETDKKSGYIPFVVIGTAGTTNTGSIDPLTEISALCKKHDMWFHIDGAYGASVLLSPKYKSLLTGTGLADSISWDAHKWLFQTYGCAMVLVKDIRNLFHSFHVNPEYLKDLENDIDNVNTWDIGMELTRPARGLKLWLTLQVLGSDLIGSAIEHGFQLAVWAEEALNPKKDWEIVSPAQMAMINFRYAPKDLTKEEQDILNEKISHRILESGYAAIFTTVLNGKTVLRICAIHPEATQEDMQHTIDLLDQYGREIYTEMKKA

SEQ ID NO: 7 - DNA, 18 bp, synthetic construct
  source 1..18: mol_type = other DNA
ttgttgayryrtcaacwa

SEQ ID NO: 8 - DNA, 15 bp, synthetic construct
  source 1..15: mol_type = other DNA
  misc_difference 8: note = a, c, t, or g
ttataatnattataa

SEQ ID NO: 9 - DNA, 24 bp, synthetic construct
  source 1..24: mol_type = other DNA
ggaaaatttttttaaaaaaaaaac

SEQ ID NO: 10 - DNA, 1251 bp, synthetic construct
  source 1..1251: mol_type = other DNA
atgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaa

SEQ ID NO: 11 - DNA, 1354 bp, synthetic construct
  source 1..1354: mol_type = other DNA
taattcctaatttttgttgacactctatcattgatagagttattttaccactccctatcagtgatagagaaaagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaa

SEQ ID NO: 12 - DNA, 1985 bp, synthetic construct
  source 1..1985: mol_type = other DNA
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaaggag

SEQ ID NO: 13 - DNA, 1398 bp, synthetic construct
  source 1..1398: mol_type = other DNA
atggagccttcatctttagaactgccagcggacacggtgcagcgcatcgcggcggaactgaagtgccatccgactgatgagcgtgtggcgctgcatctggacgaagaagataaactgcgccactttcgtgaatgtttttatattcctaaaattcaagacttgccgccggtagatttgagtctcgttaacaaagatgaaaacgcgatctactttctgggcaactctctgggtctgcaaccaaaaatggttaaaacgtacctggaggaagaactggataaatgggcaaaaatcgcggcttatggtcacgaagtgggcaagcgtccttggattactggcgacgagtctattgtgggtttgatgaaagatattgtgggcgcgaatgaaaaggaaattgcactgatgaatgctctgaccgttaatctgcacctgctgatgctgtctttttttaaaccgaccccgaaacgctacaaaatactgctggaagcgaaagcgtttccgtcggatcactatgctatagaaagtcaactgcagttgcatggtctgaatatcgaggaatctatgcgcatgattaaaccgcgtgagggtgaagaaacgctgcgtattgaagacattctggaagttattgaaaaagaaggtgattctatcgcagttatactgttttctggcgtgcacttttatacaggtcagcacttcaatatcccggcaatcactaaagcggggcaggcaaaaggctgctatgttggttttgacctggcgcatgcagtggggaatgttgaactgtatctgcacgattggggcgttgatttcgcgtgttggtgtagctacaaatatctgaacgctggcgcgggtggcattgctggcgcttttattcacgaaaaacacgcgcacaccattaaaccggctctggttggctggttcggtcatgagctgagtactcgctttaaaatggataacaaactgcaattgattccgggtgtttgcggcttccgtatcagcaatccgccgattctgctggtttgcagcctgcacgctagtctggaaatctttaagcaggcgactatgaaagcgctgcgcaaaaaatctgtgctgctgaccggctatctggagtatctgatcaaacacaattatggcaaagataaagctgcaactaaaaaaccggtagtgaacattatcaccccctcacacgtggaggagcgcggttgtcagctgactattactttcagtgtacctaataaagatgtgttccaggaactggaaaaacgcggcgttgtttgtgataaacgtaacccgaatggtattcgcgtggctcctgtgccgctgtacaattcattccacgatgtttataaattcaccaacctgctgacttctattctcgacagtgctgagactaaaaattaa

SEQ ID NO: 14 - DNA, 1501 bp, synthetic construct
  source 1..1501: mol_type = other DNA
taattcctaatttttgttgacactctatcattgatagagttattttaccactccctatcagtgatagagaaaagtgaatatcaagacacgaggaggtaagattatggagccttcatctttagaactgccagcggacacggtgcagcgcatcgcggcggaactgaagtgccatccgactgatgagcgtgtggcgctgcatctggacgaagaagataaactgcgccactttcgtgaatgtttttatattcctaaaattcaagacttgccgccggtagatttgagtctcgttaacaaagatgaaaacgcgatctactttctgggcaactctctgggtctgcaaccaaaaatggttaaaacgtacctggaggaagaactggataaatgggcaaaaatcgcggcttatggtcacgaagtgggcaagcgtccttggattactggcgacgagtctattgtgggtttgatgaaagatattgtgggcgcgaatgaaaaggaaattgcactgatgaatgctctgaccgttaatctgcacctgctgatgctgtctttttttaaaccgaccccgaaacgctacaaaatactgctggaagcgaaagcgtttccgtcggatcactatgctatagaaagtcaactgcagttgcatggtctgaatatcgaggaatctatgcgcatgattaaaccgcgtgagggtgaagaaacgctgcgtattgaagacattctggaagttattgaaaaagaaggtgattctatcgcagttatactgttttctggcgtgcacttttatacaggtcagcacttcaatatcccggcaatcactaaagcggggcaggcaaaaggctgctatgttggttttgacctggcgcatgcagtggggaatgttgaactgtatctgcacgattggggcgttgatttcgcgtgttggtgtagctacaaatatctgaacgctggcgcgggtggcattgctggcgcttttattcacgaaaaacacgcgcacaccattaaaccggctctggttggctggttcggtcatgagctgagtactcgctttaaaatggataacaaactgcaattgattccgggtgtttgcggcttccgtatcagcaatccgccgattctgctggtttgcagcctgcacgctagtctggaaatctttaagcaggcgactatgaaagcgctgcgcaaaaaatctgtgctgctgaccggctatctggagtatctgatcaaacacaattatggcaaagataaagctgcaactaaaaaaccggtagtgaacattatcaccccctcacacgtggaggagcgcggttgtcagctgactattactttcagtgtacctaataaagatgtgttccaggaactggaaaaacgcggcgttgtttgtgataaacgtaacccgaatggtattcgcgtggctcctgtgccgctgtacaattcattccacgatgtttataaattcaccaacctgctgacttctattctcgacagtgctgagactaaaaattaa

SEQ ID NO: 15 - DNA, 2127 bp, synthetic construct
  source 1..2127: mol_type = other DNA
taagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaatatcaagacacgaggaggtaagattatggagccttcatctttagaactgccagcggacacggtgcagcgcatcgcggcggaactgaagtgccatccgactgatgagcgtgtggcgctgcatctggacgaagaagataaactgcgccactttcgtgaatgtttttatattcctaaaattcaagacttgccgccggtagatttgagtctcgttaacaaagatgaaaacgcgatctactttctgggcaactctctgggtctgcaaccaaaaatggttaaaacgtacctggaggaagaactggataaatgggcaaaaatcgcggcttatggtcacgaagtgggcaagcgtccttggattactggcgacgagtctattgtgggtttgatgaaagatattgtgggcgcgaatgaaaaggaaattgcactgatgaatgctctgaccgttaatctgcacctgctgatgctgtctttttttaaaccgaccccgaaacgctacaaaatactgctggaagcgaaagcgtttccgtcggatcactatgctatagaaagtcaactgcagttgcatggtctgaatatcgaggaatctatgcgcatgattaaaccgcgtgagggtgaagaaacgctgcgtattgaagacattctggaagttattgaaaaagaaggtgattctatcgcagttatactgttttctggcgtgcacttttatacaggtcagcacttcaatatcccggcaatcactaaagcggggcaggcaaaaggctgctatgttggttttgacctggcgcatgcagtggggaatgttgaactgtatctgcacgattggggcgttgatttcgcgtgttggtgtagctacaaatatctgaacgctggcgcgggtggcattgctggcgcttttattcacgaaaaacacgcgcacaccattaaaccggctctggttggctggttcggtcatgagctgagtactcgctttaaaatggataacaaactgcaattgattccgggtgtttgcggcttccgtatcagcaatccgccgattctgctggtttgcagcctgcacgctagtctggaaatctttaagcaggcgactatgaaagcgctgcgcaaaaaatctgtgctgctgaccggctatctggagtatctgatcaaacacaattatggcaaagataaagctgcaactaaaaaaccggtagtgaacattatcaccccctcacacgtggaggagcgcggttgtcagctgactattactttcagtgtacctaataaagatgtgttccaggaactggaaaaacgcggcgttgtttgtgataaacgtaacccgaatggtattcgcgtggctcctgtgccgctgtacaattcattccacgatgtttataaattcaccaacctgctgacttctattctcgacagtgctgagactaaaaattaa

SEQ ID NO: 16 - DNA, 64 bp, synthetic construct
  source 1..64: mol_type = other DNA
ggaaaatttttttaaaaaaaaaacttgacagctagctcagtccttggtataatgctagcacgaa

SEQ ID NO: 17 - DNA, 25 bp, synthetic construct
  source 1..25: mol_type = other DNA
ttatataaaagtgggaggtgcccga

SEQ ID NO: 18 - DNA, 94 bp, synthetic construct
  source 1..94: mol_type = other DNA
ggaaaatttttttaaaaaaaaaacttgacagctagctcagtccttggtataatgctagcacgaagtgaattatataaaagtgggaggtgcccga

SEQ ID NO: 19 - DNA, 951 bp, Escherichia coli
  source 1..951: mol_type = genomic DNA
caaatatcacataatcttaacatatcaataaacacagtaaagtttcatgtgaaaaacatcaaacataaaatacaagctcggaatacgaatcacgctatacacattgctaacaggaatgagattatctaaatgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagatttttttacctttccatactatagatacagtagccgtgctattagttctcactgcgagcttggtgtcgatattgaacaaatagagatttagacaactctatctgaatatcagtcagcattttttactccacaggaagctactacatagtttcacttcctcgttatgaaggtcaattactttttggaaatgtggacgctcaaagagcttacatcaatatcgaggtaaggcctatctttaggactggattgtattgaatttcatttaacaaataaaaactaactcaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattgaatcgccacggataatctagacacttctgagccgtcgataatattgattttcatattccgtcggtggtgtaagtatcccgcataatcgtgccattcacatttag

SEQ ID NO: 20 - DNA, 424 bp, synthetic construct
  source 1..424: mol_type = other DNA
ggatggggggaaacatggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaaatgaggattgatgtgtaggctggagctgcttcgaagttcctatactttctagagaataggaacttcggaataggaacttcggaataggaactaaggaggatattcatatgtcgtcaaatgggcagaattgaatcgccacggataatctagacacttctgagccgtcgataatattgattttcatattccgtcggtgg

SEQ ID NO: 21 - DNA, 1225 bp, synthetic construct
  source 1..1225: mol_type = other DNA
ggaaaatttttttaaaaaaaaaacttgacagctagctcagtccttggtataatgctagcacgaagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggt

SEQ ID NO: 22 - DNA, 66 bp, synthetic construct
  source 1..66: mol_type = other DNA
ataagtgccttcccatcaaaaaaatattctcaacataaaaaactttgtgtaatacttgtaacgcta

SEQ ID NO: 23 - DNA, 25 bp, synthetic construct
  source 1..25: mol_type = other DNA
ttatataaaagtgggaggtgcccga

SEQ ID NO: 24 - DNA, 96 bp, synthetic construct
  source 1..96: mol_type = other DNA
ataagtgccttcccatcaaaaaaatattctcaacataaaaaactttgtgtaatacttgtaacgctagtgaattatataaaagtgggaggtgcccga

SEQ ID NO: 25 - DNA, 1347 bp, synthetic construct
  source 1..1347: mol_type = other DNA
ataagtgccttcccatcaaaaaaatattctcaacataaaaaactttgtgtaatacttgtaacgctagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaa

SEQ ID NO: 26 - DNA, 2372 bp, Clostridium sporogenes
  source 1..2372: mol_type = genomic DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgaaattttggcgcaagtatacgcaacaggagatggatgagaaaatcacagaatcgcttgagaagacattaaattacgataacacgaaaaccatcggcatcccaggtactaagctggatgatactgtattttatgacgatcactccttcgttaagcactctccctatttacgtacgttcatccaaaaccctaatcacattggttgtcacacgtacgataaagcagacatcttgtttggcggcacgtttgacatcgaacgcgaactgattcagcttttggccatcgatgtcttaaacggaaatgatgaggaattcgatggatatgtgacacaggggggaaccgaggcgaatattcaggcaatgtgggtttatcgtaactatttcaaaaaagaacgtaaagcaaaacatgaggaaatcgcaatcatcacgagcgcggatacccattacagtgcatataaggggagcgacttgctgaacattgatattatcaaggtcccagtagacttctattcgcgtaagatccaggagaacacgttagactcgattgtcaaggaggcgaaggaaattggaaagaagtacttcattgtcatctcaaacatgggtacgactatgtttggcagtgtagacgaccctgatctttatgctaacatttttgataagtataacttagaatacaaaatccacgtcgatggagcttttgggggtttcatttatcctatcgataataaggagtgcaaaacagatttctcgaacaagaacgtctcatccatcacgcttgacggtcacaaaatgcttcaagccccctatgggactggtatcttcgtgtcacgtaagaacttgatccataacaccctgacaaaggaagcaacgtatattgaaaacctggacgttaccctgagtgggtcccgctccggatccaacgccgttgcgatctggatggttttagcctcttatggcccctacgggtggatggagaagattaacaagttgcgcaatcgcactaagtggctttgcaagcagcttaacgacatgcgcatcaaatactataaggaggatagcatgaatatcgtcacgattgaagagcaatacgtaaataaagagattgcagagaaatacttccttgtgcctgaagtacacaatcctaccaacaattggtacaagattgtagtcatggaacatgttgaacttgacatcttgaactcccttgtttatgatttacgtaaattcaacaaggagcacctgaaggcaatgtga

SEQ ID NO: 27 - DNA, 1254 bp, Clostridium sporogenes
  source 1..1254: mol_type = genomic DNA
atgaaattttggcgcaagtatacgcaacaggagatggatgagaaaatcacagaatcgcttgagaagacattaaattacgataacacgaaaaccatcggcatcccaggtactaagctggatgatactgtattttatgacgatcactccttcgttaagcactctccctatttacgtacgttcatccaaaaccctaatcacattggttgtcacacgtacgataaagcagacatcttgtttggcggcacgtttgacatcgaacgcgaactgattcagcttttggccatcgatgtcttaaacggaaatgatgaggaattcgatggatatgtgacacaggggggaaccgaggcgaatattcaggcaatgtgggtttatcgtaactatttcaaaaaagaacgtaaagcaaaacatgaggaaatcgcaatcatcacgagcgcggatacccattacagtgcatataaggggagcgacttgctgaacattgatattatcaaggtcccagtagacttctattcgcgtaagatccaggagaacacgttagactcgattgtcaaggaggcgaaggaaattggaaagaagtacttcattgtcatctcaaacatgggtacgactatgtttggcagtgtagacgaccctgatctttatgctaacatttttgataagtataacttagaatacaaaatccacgtcgatggagcttttgggggtttcatttatcctatcgataataaggagtgcaaaacagatttctcgaacaagaacgtctcatccatcacgcttgacggtcacaaaatgcttcaagccccctatgggactggtatcttcgtgtcacgtaagaacttgatccataacaccctgacaaaggaagcaacgtatattgaaaacctggacgttaccctgagtgggtcccgctccggatccaacgccgttgcgatctggatggttttagcctcttatggcccctacgggtggatggagaagattaacaagttgcgcaatcgcactaagtggctttgcaagcagcttaacgacatgcgcatcaaatactataaggaggatagcatgaatatcgtcacgattgaagagcaatacgtaaataaagagattgcagagaaatacttccttgtgcctgaagtacacaatcctaccaacaattggtacaagattgtagtcatggaacatgttgaacttgacatcttgaactcccttgtttatgatttacgtaaattcaacaaggagcacctgaaggcaatgtga

SEQ ID NO: 28 - protein, 417 aa, Clostridium sporogenes
MKFWRKYTQQEMDEKITESLEKTLNYDNTKTIGIPGTKLDDTVFYDDHSFVKHSPYLRTFIQNPNHIGCHTYDKADILFGGTFDIERELIQLLAIDVLNGNDEEFDGYVTQGGTEANIQAMWVYRNYFKKERKAKHEEIAIITSADTHYSAYKGSDLLNIDIIKVPVDFYSRKIQENTLDSIVKEAKEIGKKYFIVISNMGTTMFGSVDDPDLYANIFDKYNLEYKIHVDGAFGGFIYPIDNKECKTDFSNKNVSSITLDGHKMLQAPYGTGIFVSRKNLIHNTLTKEATYIENLDVTLSGSRSGSNAVAIWMVLASYGPYGWMEKINKLRNRTKWLCKQLNDMRIKYYKEDSMNIVTIEEQYVNKEIAEKYFLVPEVHNPTNNWYKIVVMEHVELDILNSLVYDLRKFNKEHLKAM

SEQ ID NO: 29 - DNA, 60 bp, synthetic construct
  source 1..60: mol_type = other DNA
tagaactgatgcaaaaagtgctcgacgaaggcacacagatgtgtaggctggagctgcttc

SEQ ID NO: 30 - DNA, 60 bp, synthetic construct
  source 1..60: mol_type = other DNA
gtttcgtaattagatagccaccggcgctttaatgcccggacatatgaatatcctccttag

SEQ ID NO: 31 - DNA, 52 bp, synthetic construct
  source 1..52: mol_type = other DNA
caacacgtttcctgaggaaccatgaaacagtatttagaactgatgcaaaaag

SEQ ID NO: 32 - DNA, 46 bp, synthetic construct
  source 1..46: mol_type = other DNA
cgcacactggcgtcggctctggcaggatgtttcgtaattagatagc

SEQ ID NO: 33 - DNA, 1096 bp, synthetic construct
  source 1..1096: mol_type = other DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaa

SEQ ID NO: 34 - DNA, 1053 bp, synthetic construct
  source 1..1053: mol_type = other DNA
atgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaa

SEQ ID NO: 35 - DNA, 2351 bp, synthetic construct
  source 1..2351: mol_type = other DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatggcaaaggtatcgctggagaaagacaagattaagtttctgctggtagaaggcgtgcaccaaaaggcgctggaaagccttcgtgcagctggttacaccaacatcgaatttcacaaaggcgcgctggatgatgaacaattaaaagaatccatccgcgatgcccacttcatcggcctgcgatcccgtacccatctgactgaagacgtgatcaacgccgcagaaaaactggtcgctattggctgtttctgtatcggaacaaatcaggttgatctggatgcggcggcaaagcgcgggatcccggtatttaacgcaccgttctcaaatacgcgctctgttgcggagctggtgattggcgaactgctgctgctattgcgcggcgtgccagaagccaatgctaaagcgcatcgtggcgtgtggaacaaactggcggcgggttcttttgaagcgcgcggcaaaaagctgggtatcatcggctacggtcatattggtacgcaattgggcattctggctgaatcgctgggaatgtatgtttacttttatgatattgaaaacaaactgccgctgggcaacgccactcaggtacagcatctttctgacctgctgaatatgagcgatgtggtgagtctgcatgtaccagagaatccgtccaccaaaaatatgatgggcgcgaaagagatttcgctaatgaagcccggctcgctgctgattaatgcttcgcgcggtactgtggtggatattccagcgctgtgtgacgcgctggcgagcaaacatctggcgggggcggcaatcgacgtattcccgacggaaccggcgaccaatagcgatccatttacctctccgctgtgtgaattcgacaatgtccttctgacgccacacattggcggttcgactcaggaagcgcaggagaatatcggcttggaagttgcgggtaaattgatcaagtattctgacaatggctcaacgctctctgcggtgaacttcccggaagtctcgctgccactgcacggtgggcgtcgtctgatgcacatccacgaaaaccgtccgggcgtgctaactgcgctcaacaaaatttttgccgagcagggcgtcaacatcgccgcgcaatatctacaaacttccgcccagatgggttatgtagttattgatattgaagccgacgaagacgttgccgaaaaagcgctgcaggcaatgaaagctattccgggtaccattcgcgcccgtctgctgtactaa

SEQ ID NO: 36 - DNA, 1233 bp, synthetic construct
  source 1..1233: mol_type = other DNA
atggcaaaggtatcgctggagaaagacaagattaagtttctgctggtagaaggcgtgcaccaaaaggcgctggaaagccttcgtgcagctggttacaccaacatcgaatttcacaaaggcgcgctggatgatgaacaattaaaagaatccatccgcgatgcccacttcatcggcctgcgatcccgtacccatctgactgaagacgtgatcaacgccgcagaaaaactggtcgctattggctgtttctgtatcggaacaaatcaggttgatctggatgcggcggcaaagcgcgggatcccggtatttaacgcaccgttctcaaatacgcgctctgttgcggagctggtgattggcgaactgctgctgctattgcgcggcgtgccagaagccaatgctaaagcgcatcgtggcgtgtggaacaaactggcggcgggttcttttgaagcgcgcggcaaaaagctgggtatcatcggctacggtcatattggtacgcaattgggcattctggctgaatcgctgggaatgtatgtttacttttatgatattgaaaacaaactgccgctgggcaacgccactcaggtacagcatctttctgacctgctgaatatgagcgatgtggtgagtctgcatgtaccagagaatccgtccaccaaaaatatgatgggcgcgaaagagatttcgctaatgaagcccggctcgctgctgattaatgcttcgcgcggtactgtggtggatattccagcgctgtgtgacgcgctggcgagcaaacatctggcgggggcggcaatcgacgtattcccgacggaaccggcgaccaatagcgatccatttacctctccgctgtgtgaattcgacaatgtccttctgacgccacacattggcggttcgactcaggaagcgcaggagaatatcggcttggaagttgcgggtaaattgatcaagtattctgacaatggctcaacgctctctgcggtgaacttcccggaagtctcgctgccactgcacggtgggcgtcgtctgatgcacatccacgaaaaccgtccgggcgtgctaactgcgctcaacaaaatttttgccgagcagggcgtcaacatcgccgcgcaatatctacaaacttccgcccagatgggttatgtagttattgatattgaagccgacgaagacgttgccgaaaaagcgctgcaggcaatgaaagctattccgggtaccattcgcgcccgtctgctgtactaa

SEQ ID NO: 37 - DNA, 2621 bp, Catharanthus roseus
  source 1..2621: mol_type = genomic DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgggttctattgactcgacgaatgtggccatgtctaattctcctgttggcgagtttaagccccttgaagcagaagagttccgtaaacaggcacaccgcatggtggattttattgcggattattacaagaacgtagaaacatacccggtcctttccgaggttgaacccggctatctgcgcaaacgtattcccgaaaccgcaccatacctgccggagccacttgatgatattatgaaggatattcaaaaggacattatccccggaatgacgaactggatgtccccgaacttttacgccttcttcccggccacagttagctcagcagctttcttgggggaaatgctttcaacggcccttaacagcgtaggatttacctgggtcagttccccggcagcgactgaattagagatgatcgttatggattggcttgcgcaaattttgaaacttccaaaaagctttatgttctccggaaccgggggtggtgtcatccaaaacactacgtcagagtcgatcttgtgcactattatcgcggcccgtgaacgcgccttggaaaaattgggccctgattcaattggtaagcttgtctgctatgggtccgatcaaacgcacacaatgtttccgaaaacctgtaagttagcaggaatttatccgaataatatccgccttatccctaccacggtagaaaccgactttggcatctcaccgcaggtacttcgcaagatggtcgaagacgacgtcgctgcggggtacgttcccttatttttgtgtgccaccttgggaacgacatcaactacggcaacagatcctgtagattcgctgtccgaaatcgcaaacgagtttggtatctggattcatgtcgacgccgcatatgctggatcggcttgcatctgcccagaatttcgtcactaccttgatggcatcgaacgtgtggattccttatcgctgtctccccacaaatggcttttagcatatctggattgcacgtgcttgtgggtaaaacaacctcacctgctgcttcgcgctttaacgactaatcccgaatacttgaagaataaacagagtgatttagataaggtcgtggattttaagaactggcagatcgcaacaggacgtaagttccgctctttaaaactttggttaattctgcgttcctacggggtagttaacctgcaaagtcatatccgtagtgatgtagcgatggggaagatgtttgaggaatgggtccgttccgatagccgctttgaaatcgtcgtgccacgtaatttttcgcttgtatgctttcgcttgaaaccggatgtatctagtttacatgtcgaggaggtcaacaagaagttgttggatatgcttaactccaccggtcgcgtatatatgacgcatacaattgttggcggaatctatatgttacgtttggctgtaggtagcagcttgacagaggaacatcacgtgcgccgcgtttgggacttgatccagaagcttacggacgacctgcttaaagaggcgtga

SEQ ID NO: 38 - DNA, 1503 bp, Catharanthus roseus
  source 1..1503: mol_type = genomic DNA
atgggttctattgactcgacgaatgtggccatgtctaattctcctgttggcgagtttaagccccttgaagcagaagagttccgtaaacaggcacaccgcatggtggattttattgcggattattacaagaacgtagaaacatacccggtcctttccgaggttgaacccggctatctgcgcaaacgtattcccgaaaccgcaccatacctgccggagccacttgatgatattatgaaggatattcaaaaggacattatccccggaatgacgaactggatgtccccgaacttttacgccttcttcccggccacagttagctcagcagctttcttgggggaaatgctttcaacggcccttaacagcgtaggatttacctgggtcagttccccggcagcgactgaattagagatgatcgttatggattggcttgcgcaaattttgaaacttccaaaaagctttatgttctccggaaccgggggtggtgtcatccaaaacactacgtcagagtcgatcttgtgcactattatcgcggcccgtgaacgcgccttggaaaaattgggccctgattcaattggtaagcttgtctgctatgggtccgatcaaacgcacacaatgtttccgaaaacctgtaagttagcaggaatttatccgaataatatccgccttatccctaccacggtagaaaccgactttggcatctcaccgcaggtacttcgcaagatggtcgaagacgacgtcgctgcggggtacgttcccttatttttgtgtgccaccttgggaacgacatcaactacggcaacagatcctgtagattcgctgtccgaaatcgcaaacgagtttggtatctggattcatgtcgacgccgcatatgctggatcggcttgcatctgcccagaatttcgtcactaccttgatggcatcgaacgtgtggattccttatcgctgtctccccacaaatggcttttagcatatctggattgcacgtgcttgtgggtaaaacaacctcacctgctgcttcgcgctttaacgactaatcccgaatacttgaagaataaacagagtgatttagataaggtcgtggattttaagaactggcagatcgcaacaggacgtaagttccgctctttaaaactttggttaattctgcgttcctacggggtagttaacctgcaaagtcatatccgtagtgatgtagcgatggggaagatgtttgaggaatgggtccgttccgatagccgctttgaaatcgtcgtgccacgtaatttttcgcttgtatgctttcgcttgaaaccggatgtatctagtttacatgtcgaggaggtcaacaagaagttgttggatatgcttaactccaccggtcgcgtatatatgacgcatacaattgttggcggaatctatatgttacgtttggctgtaggtagcagcttgacagaggaacatcacgtgcgccgcgtttgggacttgatccagaagcttacggacgacctgcttaaagaggcgtga

SEQ ID NO: 39 - DNA, 5377 bp, synthetic construct
  source 1..5377: mol_type = other DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtgaaatgtaagaaggagatatacatatgcgtacaccctactgtgtcgccgattatcttttagatcgtctgacggactgcggggccgatcacctgtttggcgtaccgggcgattacaacttgcagtttctggaccacgtcattgactcaccagatatctgctgggtagggtgtgcgaacgagcttaacgcgagctacgctgctgacggatatgcgcgttgtaaaggctttgctgcacttcttactaccttcggggtcggtgagttatcggcgatgaacggtatcgcaggctcgtacgctgagcacgtcccggtattacacattgtgggagctccgggtaccgcagctcaacagcgcggagaactgttacaccacacgctgggcgacggagaattccgccacttttaccatatgtccgagccaattactgtagcccaggctgtacttacagagcaaaatgcctgttacgagatcgaccgtgttttgaccacgatgcttcgcgagcgccgtcccgggtatttgatgctgccagccgatgttgccaaaaaagctgcgacgcccccagtgaatgccctgacgcataaacaagctcatgccgattccgcctgtttaaaggcttttcgcgatgcagctgaaaataaattagccatgtcgaaacgcaccgccttgttggcggactttctggtcctgcgccatggccttaaacacgcccttcagaaatgggtcaaagaagtcccgatggcccacgctacgatgcttatgggtaaggggatttttgatgaacgtcaagcgggattttatggaacttattccggttcggcgagtacgggggcggtaaaggaagcgattgagggagccgacacagttctttgcgtggggacacgtttcaccgatacactgaccgctggattcacacaccaacttactccggcacaaacgattgaggtgcaaccccatgcggctcgcgtgggggatgtatggtttacgggcattccaatgaatcaagccattgagactcttgtcgagctgtgcaaacagcacgtccacgcaggactgatgagttcgagctctggggcgattccttttccacaaccagatggtagtttaactcaagaaaacttctggcgcacattgcaaacctttatccgcccaggtgatatcatcttagcagaccagggtacttcagcctttggagcaattgacctgcgcttaccagcagacgtgaactttattgtgcagccgctgtgggggtctattggttatactttagctgcggccttcggagcgcagacagcgtgtccaaaccgtcgtgtgatcgtattgacaggagatggagcagcgcagttgaccattcaggagttaggctcgatgttacgcgataagcagcaccccattatcctggtcctgaacaatgaggggtatacagttgaacgcgccattcatggtgcggaacaacgctacaatgacatcgctttatggaattggacgcacatcccccaagccttatcgttagatccccaatcggaatgttggcgtgtgtctgaagcagagcaactggctgatgttctggaaaaagttgctcatcatgaacgcctgtcgttgatcgaggtaatgttgcccaaggccgatatccctccgttactgggagccttgaccaaggctttagaagcctgcaacaacgcttaaaggttaagaaggagatatacatatgcccaccttgaacttggacttacccaacggtattaagagcacgattcaggcagaccttttcatcaataataagtttgtgccggcgcttgatgggaaaacgttcgcaactattaatccgtctacggggaaagagatcggacaggtggcagaggcttcggcgaaggatgtggatcttgcagttaaggccgcgcgtgaggcgtttgaaactacttggggggaaaacacgccaggtgatgctcgtggccgtttactgattaagcttgctgagttggtggaagcgaatattgatgagttagcggcaattgaatcactggacaatgggaaagcgttctctattgctaagtcattcgacgtagctgctgtggccgcaaacttacgttactacggcggttgggctgataaaaaccacggtaaagtcatggaggtagacacaaagcgcctgaactatacccgccacgagccgatcggggtttgcggacaaatcattccgtggaatttcccgcttttgatgtttgcatggaagctgggtcccgctttagccacagggaacacaattgtgttaaagactgccgagcagactcccttaagtgctatcaagatgtgtgaattaatcgtagaagccggctttccgcccggagtagttaatgtgatctcgggattcggaccggtggcgggggccgcgatctcgcaacacatggacatcgataagattgcctttacaggatcgacattggttggccgcaacattatgaaggcagctgcgtcgactaacttaaaaaaggttacacttgagttaggaggaaaatccccgaatatcattttcaaagatgccgaccttgaccaagctgttcgctggagcgccttcggtatcatgtttaaccacggacaatgctgctgcgctggatcgcgcgtatatgtggaagaatccatctatgacgccttcatggaaaaaatgactgcgcattgtaaggcgcttcaagttggagatcctttcagcgcgaacaccttccaaggaccacaagtctcgcagttacaatacgaccgtatcatggaatacatcgaatcagggaaaaaagatgcaaatcttgctttaggcggcgttcgcaaagggaatgaggggtatttcattgagccaactatttttacagacgtgccgcacgacgcgaagattgccaaagaggagatcttcggtccagtggttgttgtgtcgaaatttaaggacgaaaaagatctgatccgtatcgcaaatgattctatttatggtttagctgcggcagtcttttcccgcgacatcagccgcgcgatcgagacagcacacaaactgaaagcaggcacggtctgggtcaactgctataatcagcttattccgcaggtgccattcggagggtataaggcttccggtatcggccgtgagttgggggaatatgccttgtctaattacacaaatatcaaggccgtccacgttaacctttctcaaccggcgcccatttga

SEQ ID NO: 40 - DNA, 1080 bp, synthetic construct
  source 1..1080: mol_type = other DNA
taagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtga

SEQ ID NO: 41 - DNA, 1674 bp, synthetic construct
  source 1..1674: mol_type = other DNA
gaaggagatatacatatgcgtacaccctactgtgtcgccgattatcttttagatcgtctgacggactgcggggccgatcacctgtttggcgtaccgggcgattacaacttgcagtttctggaccacgtcattgactcaccagatatctgctgggtagggtgtgcgaacgagcttaacgcgagctacgctgctgacggatatgcgcgttgtaaaggctttgctgcacttcttactaccttcggggtcggtgagttatcggcgatgaacggtatcgcaggctcgtacgctgagcacgtcccggtattacacattgtgggagctccgggtaccgcagctcaacagcgcggagaactgttacaccacacgctgggcgacggagaattccgccacttttaccatatgtccgagccaattactgtagcccaggctgtacttacagagcaaaatgcctgttacgagatcgaccgtgttttgaccacgatgcttcgcgagcgccgtcccgggtatttgatgctgccagccgatgttgccaaaaaagctgcgacgcccccagtgaatgccctgacgcataaacaagctcatgccgattccgcctgtttaaaggcttttcgcgatgcagctgaaaataaattagccatgtcgaaacgcaccgccttgttggcggactttctggtcctgcgccatggccttaaacacgcccttcagaaatgggtcaaagaagtcccgatggcccacgctacgatgcttatgggtaaggggatttttgatgaacgtcaagcgggattttatggaacttattccggttcggcgagtacgggggcggtaaaggaagcgattgagggagccgacacagttctttgcgtggggacacgtttcaccgatacactgaccgctggattcacacaccaacttactccggcacaaacgattgaggtgcaaccccatgcggctcgcgtgggggatgtatggtttacgggcattccaatgaatcaagccattgagactcttgtcgagctgtgcaaacagcacgtccacgcaggactgatgagttcgagctctggggcgattccttttccacaaccagatggtagtttaactcaagaaaacttctggcgcacattgcaaacctttatccgcccaggtgatatcatcttagcagaccagggtacttcagcctttggagcaattgacctgcgcttaccagcagacgtgaactttattgtgcagccgctgtgggggtctattggttatactttagctgcggccttcggagcgcagacagcgtgtccaaaccgtcgtgtgatcgtattgacaggagatggagcagcgcagttgaccattcaggagttaggctcgatgttacgcgataagcagcaccccattatcctggtcctgaacaatgaggggtatacagttgaacgcgccattcatggtgcggaacaacgctacaatgacatcgctttatggaattggacgcacatcccccaagccttatcgttagatccccaatcggaatgttggcgtgtgtctgaagcagagcaactggctgatgttctggaaaaagttgctcatcatgaacgcctgtcgttgatcgaggtaatgttgcccaaggccgatatccctccgttactgggagccttgaccaaggctttagaagcctgcaacaacgcttaa

SEQ ID NO: 42 - DNA, 1509 bp, synthetic construct
  source 1..1509: mol_type = other DNA
gaaggagatatacatatgcccaccttgaacttggacttacccaacggtattaagagcacgattcaggcagaccttttcatcaataataagtttgtgccggcgcttgatgggaaaacgttcgcaactattaatccgtctacggggaaagagatcggacaggtggcagaggcttcggcgaaggatgtggatcttgcagttaaggccgcgcgtgaggcgtttgaaactacttggggggaaaacacgccaggtgatgctcgtggccgtttactgattaagcttgctgagttggtggaagcgaatattgatgagttagcggcaattgaatcactggacaatgggaaagcgttctctattgctaagtcattcgacgtagctgctgtggccgcaaacttacgttactacggcggttgggctgataaaaaccacggtaaagtcatggaggtagacacaaagcgcctgaactatacccgccacgagccgatcggggtttgcggacaaatcattccgtggaatttcccgcttttgatgtttgcatggaagctgggtcccgctttagccacagggaacacaattgtgttaaagactgccgagcagactcccttaagtgctatcaagatgtgtgaattaatcgtagaagccggctttccgcccggagtagttaatgtgatctcgggattcggaccggtggcgggggccgcgatctcgcaacacatggacatcgataagattgcctttacaggatcgacattggttggccgcaacattatgaaggcagctgcgtcgactaacttaaaaaaggttacacttgagttaggaggaaaatccccgaatatcattttcaaagatgccgaccttgaccaagctgttcgctggagcgccttcggtatcatgtttaaccacggacaatgctgctgcgctggatcgcgcgtatatgtggaagaatccatctatgacgccttcatggaaaaaatgactgcgcattgtaaggcgcttcaagttggagatcctttcagcgcgaacaccttccaaggaccacaagtctcgcagttacaatacgaccgtatcatggaatacatcgaatcagggaaaaaagatgcaaatcttgctttaggcggcgttcgcaaagggaatgaggggtatttcattgagccaactatttttacagacgtgccgcacgacgcgaagattgccaaagaggagatcttcggtccagtggttgttgtgtcgaaatttaaggacgaaaaagatctgatccgtatcgcaaatgattctatttatggtttagctgcggcagtcttttcccgcgacatcagccgcgcgatcgagacagcacacaaactgaaagcaggcacggtctgggtcaactgctataatcagcttattccgcaggtgccattcggagggtataaggcttccggtatcggccgtgagttgggggaatatgccttgtctaattacacaaatatcaaggccgtccacgttaacctttctcaaccggcgcccatttga

SEQ ID NO: 43 - DNA, 6573 bp, synthetic construct
  source 1..6573: mol_type = other DNA
ctctagaaataattttgtttaactttaagaaggagatatacatatgcaaacacaaaaaccgactctcgaactgctaacctgcgaaggcgcttatcgcgacaacccgactgcgctttttcaccagttgtgtggggatcgtccggcaacgctgctgctggaatccgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttctaatggctgacattctgctgctcgataatatcgactcttttacgtacaacctggcagatcagttgcgcagcaatggtcataacgtggtgatttaccgcaaccatattccggcgcagaccttaattgaacgcctggcgacgatgagcaatccggtgctgatgctttctcctggccccggtgtgccgagcgaagccggttgtatgccggaactcctcacccgcttgcgtggcaagctgccaattattggcatttgcctcggacatcaggcgattgtcgaagcttacgggggctatgtcggtcaggcgggcgaaattcttcacggtaaagcgtcgagcattgaacatgacggtcaggcgatgtttgccggattaacaaacccgctgccagtggcgcgttatcactcgctggttggcagtaacattccggccggtttaaccatcaacgcccattttaatggcatggtgatggcggtgcgtcacgatgcagatcgcgtttgtggattccagttccatccggaatccattcttactacccagggcgctcgcctgctggaacaaacgctggcctgggcgcagcagaaactagagccaaccaacacgctgcaaccgattctggaaaaactgtatcaggcacagacgcttagccaacaagaaagccaccagctgttttcagcggtggtacgtggcgagctgaagccggaacaactggcggcggcgctggtgagcatgaaaattcgcggtgaacacccgaacgagatcgccggggcagcaaccgcgctactggaaaacgccgcgccattcccgcgcccggattatctgtttgccgatatcgtcggtactggcggtgacggcagcaacagcatcaatatttctaccgccagtgcgtttgtcgccgcggcctgcgggctgaaagtggcgaaacacggcaaccgtagcgtctccagtaaatccggctcgtcggatctgctggcggcgttcggtattaatcttgatatgaacgccgataaatcgcgccaggcgctggatgagttaggcgtctgtttcctctttgcgccgaagtatcacaccggattccgccatgcgatgccggttcgccagcaactgaaaacccgcactctgttcaacgtgctgggaccattgattaacccggcgcatccgccgctggcgctaattggtgtttatagtccggaactggtgctgccgattgccgaaaccttgcgcgtgctggggtatcaacgcgcggcagtggtgcacagcggcgggatggatgaagtttcattacacgcgccgacaatcgttgccgaactacatgacggcgaaattaagagctatcaattgaccgctgaagattttggcctgacaccctaccaccaggagcaattggcaggcggaacaccggaagaaaaccgtgacattttaacacgcttgttacaaggtaaaggcgacgccgcccatgaagcagccgtcgcggcgaatgtcgccatgttaatgcgcctgcatggccatgaagatctgcaagccaatgcgcaaaccgttcttgaggtactgcgcagtggttccgcttacgacagagtcaccgcactggcggcacgagggtaaatgatgcaaaccgttttagcgaaaatcgtcgcagacaaggcgatttgggtagaaacccgcaaagagcagcaaccgctggccagttttcagaatgaggttcagccgagcacgcgacatttttatgatgcacttcagggcgcacgcacggcgtttattctggagtgtaaaaaagcgtcgccgtcaaaaggcgtgatccgtgatgatttcgatccggcacgcattgccgccatttataaacattacgcttcggcaatttcagtgctgactgatgagaaatattttcaggggagctttgatttcctccccatcgtcagccaaatcgccccgcagccgattttatgtaaagacttcattatcgatccttaccagatctatctggcgcgctattaccaggccgatgcctgcttattaatgctttcagtactggatgacgaacaatatcgccagcttgcagccgtcgcccacagtctggagatgggtgtgctgaccgaagtcagtaatgaagaggaactggagcgcgccattgcattgggggcaaaggtcgttggcatcaacaaccgcgatctgcgcgatttgtcgattgatctcaaccgtacccgcgagcttgcgccgaaactggggcacaacgtgacggtaatcagcgaatccggcatcaatacttacgctcaggtgcgcgagttaagccacttcgctaacggctttctgattggttcggcgttgatggcccatgacgatttgaacgccgccgtgcgtcgggtgttgctgggtgagaataaagtatgtggcctgacacgtgggcaagatgctaaagcagcttatgacgcgggcgcgatttacggtgggttgatttttgttgcgacatcaccgcgttgcgtcaacgttgaacaggcgcaggaagtgatggctgcagcaccgttgcagtatgttggcgtgttccgcaatcacgatattgccgatgtggcggacaaagctaaggtgttatcgctggcggcagtgcaactgcatggtaatgaagatcagctgtatatcgacaatctgcgtgaggctctgccagcacacgtcgccatctggaaggctttaagtgtcggtgaaactcttcccgcgcgcgattttcagcacatcgataaatatgtattcgacaacggtcagggcgggagcggacaacgtttcgactggtcactattaaatggtcaatcgcttggcaacgttctgctggcggggggcttaggcgcagataactgcgtggaagcggcacaaaccggctgcgccgggcttgattttaattctgctgtagagtcgcaaccgggtatcaaagacgcacgtcttttggcctcggttttccagacgctgcgcgcatattaaggaaaggaacaatgacaacattacttaacccctattttggtgagtttggcggcatgtacgtgccacaaatcctgatgcctgctctgcgccagctggaagaagcttttgtcagcgcgcaaaaagatcctgaatttcaggctcagttcaacgacctgctgaaaaactatgccgggcgtccaaccgcgctgaccaaatgccagaacattacagccgggacgaacaccacgctgtatctgaagcgcgaagatttgctgcacggcggcgcgcataaaactaaccaggtgctcggtcaggctttactggcgaagcggatgggtaaaactgaaattattgccgaaaccggtgccggtcagcatggcgtggcgtcggcccttgccagcgccctgctcggcctgaaatgccgaatttatatgggtgccaaagacgttgaacgccagtcgcccaacgttttccggatgcgcttaatgggtgcggaagtgatcccggtacatagcggttccgcgaccctgaaagatgcctgtaatgaggcgctacgcgactggtccggcagttatgaaaccgcgcactatatgctgggtaccgcagctggcccgcatccttacccgaccattgtgcgtgagtttcagcggatgattggcgaagaaacgaaagcgcagattctggaaagagaaggtcgcctgccggatgccgttatcgcctgtgttggcggtggttcgaatgccatcggtatgtttgcagatttcatcaacgaaaccgacgtcggcctgattggtgtggagcctggcggccacggtatcgaaactggcgagcacggcgcaccgttaaaacatggtcgcgtgggcatctatttcggtatgaaagcgccgatgatgcaaaccgaagacgggcaaattgaagagtcttactccatttctgccgggctggatttcccgtccgtcggcccgcaacatgcgtatctcaacagcactggacgcgctgattacgtgtctattaccgacgatgaagccctggaagcctttaaaacgctttgcctgcatgaagggatcatcccggcgctggaatcctcccacgccctggcccatgcgctgaaaatgatgcgcgaaaatccggaaaaagagcagctactggtggttaacctttccggtcgcggcgataaagacatcttcaccgttcacgatattttgaaagcacgaggggaaatctgatggaacgctacgaatctctgtttgcccagttgaaggagcgcaaagaaggcgcattcgttcctttcgtcaccctcggtgatccgggcattgagcagtcgttgaaaattatcgatacgctaattgaagccggtgctgacgcgctggagttaggcatccccttctccgacccactggcggatggcccgacgattcaaaacgccacactgcgtgcttttgcggcgggagtaaccccggcgcagtgctttgagatgctggcactcattcgccagaagcacccgaccattcccatcggccttttgatgtatgccaacctggtgtttaacaaaggcattgatgagttttatgccgagtgcgagaaagtcggcgtcgattcggtgctggttgccgatgtgcccgtggaagagtccgcgcccttccgccaggccgcgttgcgtcataatgtcgcacctatctttatttgcccgccgaatgccgacgatgatttgctgcgccagatagcctcttacggtcgtggttacacctatttgctgtcgcgagcgggcgtgaccggcgcagaaaaccgcgccgcgttacccctcaatcatctggttgcgaagctgaaagagtacaacgctgcgcctccattgcagggatttggtatttccgccccggatcaggtaaaagccgcgattgatgcaggagctgcgggcgcgatttctggttcggccatcgttaaaatcatcgagcaacatattaatgagccagagaaaatgctggcggcactgaaagcttttgtacaaccgatgaaagcggcgacgcgcagtta

SEQ ID NO: 44 - DNA, 1562 bp, synthetic construct
  source 1..1562: mol_type = other DNA
atgcaaacacaaaaaccgactctcgaactgctaacctgcgaaggcgcttatcgcgacaacccgactgcgctttttcaccagttgtgtggggatcgtccggcaacgctgctgctggaatccgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttcta

SEQ ID NO: 45 - DNA, 1596 bp, synthetic construct
  source 1..1596: mol_type = other DNA
atggctgacattctgctgctcgataatatcgactcttttacgtacaacctggcagatcagttgcgcagcaatggtcataacgtggtgatttaccgcaaccatattccggcgcagaccttaattgaacgcctggcgacgatgagcaatccggtgctgatgctttctcctggccccggtgtgccgagcgaagccggttgtatgccggaactcctcacccgcttgcgtggcaagctgccaattattggcatttgcctcggacatcaggcgattgtcgaagcttacgggggctatgtcggtcaggcgggcgaaattcttcacggtaaagcgtcgagcattgaacatgacggtcaggcgatgtttgccggattaacaaacccgctgccagtggcgcgttatcactcgctggttggcagtaacattccggccggtttaaccatcaacgcccattttaatggcatggtgatggcggtgcgtcacgatgcagatcgcgtttgtggattccagttccatccggaatccattcttactacccagggcgctcgcctgctggaacaaacgctggcctgggcgcagcagaaactagagccaaccaacacgctgcaaccgattctggaaaaactgtatcaggcacagacgcttagccaacaagaaagccaccagctgttttcagcggtggtacgtggcgagctgaagccggaacaactggcggcggcgctggtgagcatgaaaattcgcggtgaacacccgaacgagatcgccggggcagcaaccgcgctactggaaaacgccgcgccattcccgcgcccggattatctgtttgccgatatcgtcggtactggcggtgacggcagcaacagcatcaatatttctaccgccagtgcgtttgtcgccgcggcctgcgggctgaaagtggcgaaacacggcaaccgtagcgtctccagtaaatccggctcgtcggatctgctggcggcgttcggtattaatcttgatatgaacgccgataaatcgcgccaggcgctggatgagttaggcgtctgtttcctctttgcgccgaagtatcacaccggattccgccatgcgatgccggttcgccagcaactgaaaacccgcactctgttcaacgtgctgggaccattgattaacccggcgcatccgccgctggcgctaattggtgtttatagtccggaactggtgctgccgattgccgaaaccttgcgcgtgctggggtatcaacgcgcggcagtggtgcacagcggcgggatggatgaagtttcattacacgcgccgacaatcgttgccgaactacatgacggcgaaattaagagctatcaattgaccgctgaagattttggcctgacaccctaccaccaggagcaattggcaggcggaacaccggaagaaaaccgtgacattttaacacgcttgttacaaggtaaaggcgacgccgcccatgaagcagccgtcgcggcgaatgtcgccatgttaatgcgcctgcatggccatgaagatctgcaagccaatgcgcaaaccgttcttgaggtactgcgcagtggttccgcttacgacagagtcaccgcactggcggcacgagggtaa

SEQ ID NO: 46 - DNA, 355 bp, synthetic construct
  source 1..355: mol_type = other DNA
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 47 - DNA, 228 bp, synthetic construct
  source 1..228: mol_type = other DNA
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 48 - DNA, 334 bp, synthetic construct
  source 1..334: mol_type = other DNA
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 49 - DNA, 134 bp, synthetic construct
  source 1..134: mol_type = other DNA
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 50 - DNA, 36 bp, synthetic construct
  source 1..36: mol_type = other DNA
atatcgtcgcagcccacagcaacacgtttcctgagg

SEQ ID NO: 51 - DNA, 47 bp, synthetic construct
  source 1..47: mol_type = other DNA
aagaatttaacggagggcaaaaaaaaccgacgcacactggcgtcggc

SEQ ID NO: 52 - DNA, 1359 bp, synthetic construct
  source 1..1359: mol_type = other DNA
atgcaaaccgttttagcgaaaatcgtcgcagacaaggcgatttgggtagaaacccgcaaagagcagcaaccgctggccagttttcagaatgaggttcagccgagcacgcgacatttttatgatgcacttcagggcgcacgcacggcgtttattctggagtgtaaaaaagcgtcgccgtcaaaaggcgtgatccgtgatgatttcgatccggcacgcattgccgccatttataaacattacgcttcggcaatttcagtgctgactgatgagaaatattttcaggggagctttgatttcctccccatcgtcagccaaatcgccccgcagccgattttatgtaaagacttcattatcgatccttaccagatctatctggcgcgctattaccaggccgatgcctgcttattaatgctttcagtactggatgacgaacaatatcgccagcttgcagccgtcgcccacagtctggagatgggtgtgctgaccgaagtcagtaatgaagaggaactggagcgcgccattgcattgggggcaaaggtcgttggcatcaacaaccgcgatctgcgcgatttgtcgattgatctcaaccgtacccgcgagcttgcgccgaaactggggcacaacgtgacggtaatcagcgaatccggcatcaatacttacgctcaggtgcgcgagttaagccacttcgctaacggctttctgattggttcggcgttgatggcccatgacgatttgaacgccgccgtgcgtcgggtgttgctgggtgagaataaagtatgtggcctgacacgtgggcaagatgctaaagcagcttatgacgcgggcgcgatttacggtgggttgatttttgttgcgacatcaccgcgttgcgtcaacgttgaacaggcgcaggaagtgatggctgcagcaccgttgcagtatgttggcgtgttccgcaatcacgatattgccgatgtggcggacaaagctaaggtgttatcgctggcggcagtgcaactgcatggtaatgaagatcagctgtatatcgacaatctgcgtgaggctctgccagcacacgtcgccatctggaaggctttaagtgtcggtgaaactcttcccgcgcgcgattttcagcacatcgataaatatgtattcgacaacggtcagggcgggagcggacaacgtttcgactggtcactattaaatggtcaatcgcttggcaacgttctgctggcggggggcttaggcgcagataactgcgtggaagcggcacaaaccggctgcgccgggcttgattttaattctgctgtagagtcgcaaccgggtatcaaagacgcacgtcttttggcctcggttttccagacgctgcgcgcatattaa

SEQ ID NO: 53 - DNA, 1193 bp, synthetic construct
  source 1..1193: mol_type = other DNA
atgacaacattacttaacccctattttggtgagtttggcggcatgtacgtgccacaaatcctgatgcctgctctgcgccagctggaagaagcttttgtcagcgcgcaaaaagatcctgaatttcaggctcagttcaacgacctgctgaaaaactatgccgggcgtccaaccgcgctgaccaaatgccagaacattacagccgggacgaacaccacgctgtatctgaagcgcgaagatttgctgcacggcggcgcgcataaaactaaccaggtgctcggtcaggctttactggcgaagcggatgggtaaaactgaaattattgccgaaaccggtgccggtcagcatggcgtggcgtcggcccttgccagcgccctgctcggcctgaaatgccgaatttatatgggtgccaaagacgttgaacgccagtcgcccaacgttttccggatgcgcttaatgggtgcggaagtgatcccggtacatagcggttccgcgaccctgaaagatgcctgtaatgaggcgctacgcgactggtccggcagttatgaaaccgcgcactatatgctgggtaccgcagctggcccgcatccttacccgaccattgtgcgtgagtttcagcggatgattggcgaagaaacgaaagcgcagattctggaaagagaaggtcgcctgccggatgccgttatcgcctgtgttggcggtggttcgaatgccatcggtatgtttgcagatttcatcaacgaaaccgacgtcggcctgattggtgtggagcctggcggccacggtatcgaaactggcgagcacggcgcaccgttaaaacatggtcgcgtgggcatctatttcggtatgaaagcgccgatgatgcaaaccgaagacgggcaaattgaagagtcttactccatttctgccgggctggatttcccgtccgtcggcccgcaacatgcgtatctcaacagcactggacgcgctgattacgtgtctattaccgacgatgaagccctggaagcctttaaaacgctttgcctgcatgaagggatcatcccggcgctggaatcctcccacgccctggcccatgcgctgaaaatgatgcgcgaaaatccggaaaaagagcagctactggtggttaacctttccggtcgcggcgataaagacatcttcaccgttcacgatattttgaaagcacgaggggaaatctg

SEQ ID NO: 54 - DNA, 807 bp, synthetic construct
  source 1..807: mol_type = other DNA
atggaacgctacgaatctctgtttgcccagttgaaggagcgcaaagaaggcgcattcgttcctttcgtcaccctcggtgatccgggcattgagcagtcgttgaaaattatcgatacgctaattgaagccggtgctgacgcgctggagttaggcatccccttctccgacccactggcggatggcccgacgattcaaaacgccacactgcgtgcttttgcggcgggagtaaccccggcgcagtgctttgagatgctggcactcattcgccagaagcacccgaccattcccatcggccttttgatgtatgccaacctggtgtttaacaaaggcattgatgagttttatgccgagtgcgagaaagtcggcgtcgattcggtgctggttgccgatgtgcccgtggaagagtccgcgcccttccgccaggccgcgttgcgtcataatgtcgcacctatctttatttgcccgccgaatgccgacgatgatttgctgcgccagatagcctcttacggtcgtggttacacctatttgctgtcgcgagcgggcgtgaccggcgcagaaaaccgcgccgcgttacccctcaatcatctggttgcgaagctgaaagagtacaacgctgcgcctccattgcagggatttggtatttccgccccggatcaggtaaaagccgcgattgatgcaggagctgcgggcgcgatttctggttcggccatcgttaaaatcatcgagcaacatattaatgagccagagaaaatgctggcggcactgaaagcttttgtacaaccgatgaaagcggcgacgcgcagttaa

SEQ ID NO: 55 - DNA, 6574 bp, synthetic construct
  source 1..6574: mol_type = other DNA
ctctagaaataattttgtttaactttaagaaggagatatacatatgcaaacacaaaaaccgactctcgaactgctaacctgcgaaggcgcttatcgcgacaacccgactgcgctttttcaccagttgtgtggggatcgtccggcaacgctgctgctggaattcgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttctaatggctgacattctgctgctcgataatatcgactcttttacgtacaacctggcagatcagttgcgcagcaatggtcataacgtggtgatttaccgcaaccatattccggcgcagaccttaattgaacgcctggcgacgatgagcaatccggtgctgatgctttctcctggccccggtgtgccgagcgaagccggttgtatgccggaactcctcacccgcttgcgtggcaagctgccaattattggcatttgcctcggacatcaggcgattgtcgaagcttacgggggctatgtcggtcaggcgggcgaaattcttcacggtaaagcgtcgagcattgaacatgacggtcaggcgatgtttgccggattaacaaacccgctgccagtggcgcgttatcactcgctggttggcagtaacattccggccggtttaaccatcaacgcccattttaatggcatggtgatggcggtgcgtcacgatgcagatcgcgtttgtggattccagttccatccggaatccattcttactacccagggcgctcgcctgctggaacaaacgctggcctgggcgcagcagaaactagagccaaccaacacgctgcaaccgattctggaaaaactgtatcaggcacagacgcttagccaacaagaaagccaccagctgttttcagcggtggtacgtggcgagctgaagccggaacaactggcggcggcgctggtgagcatgaaaattcgcggtgaacacccgaacgagatcgccggggcagcaaccgcgctactggaaaacgccgcgccattcccgcgcccggattatctgtttgccgatatcgtcggtactggcggtgacggcagcaacagcatcaatatttctaccgccagtgcgtttgtcgccgcggcctgcgggctgaaagtggcgaaacacggcaaccgtagcgtctccagtaaatccggctcgtcggatctgctggcggcgttcggtattaatcttgatatgaacgccgataaatcgcgccaggcgctggatgagttaggcgtctgtttcctctttgcgccgaagtatcacaccggattccgccatgcgatgccggttcgccagcaactgaaaacccgcactctgttcaacgtgctgggaccattgattaacccggcgcatccgccgctggcgctaattggtgtttatagtccggaactggtgctgccgattgccgaaaccttgcgcgtgctggggtatcaacgcgcggcagtggtgcacagcggcgggatggatgaagtttcattacacgcgccgacaatcgttgccgaactacatgacggcgaaattaagagctatcaattgaccgctgaagattttggcctgacaccctaccaccaggagcaattggcaggcggaacaccggaagaaaaccgtgacattttaacacgcttgttacaaggtaaaggcgacgccgcccatgaagcagccgtcgcggcgaatgtcgccatgttaatgcgcctgcatggccatgaagatctgcaagccaatgcgcaaaccgttcttgaggtactgcgcagtggttccgcttacgacagagtcaccgcactggcggcacgagggtaaatgatgcaaaccgttttagcgaaaatcgtcgcagacaaggcgatttgggtagaaacccgcaaagagcagcaaccgctggccagttttcagaatgaggttcagccgagcacgcgacatttttatgatgcacttcagggcgcacgcacggcgtttattctggagtgtaaaaaagcgtcgccgtcaaaaggcgtgatccgtgatgatttcgatccggcacgcattgccgccatttataaacattacgcttcggcaatttcagtgctgactgatgagaaatattttcaggggagctttgatttcctccccatcgtcagccaaatcgccccgcagccgattttatgtaaagacttcattatcgatccttaccagatctatctggcgcgctattaccaggccgatgcctgcttattaatgctttcagtactggatgacgaacaatatcgccagcttgcagccgtcgcccacagtctggagatgggtgtgctgaccgaagtcagtaatgaagaggaactggagcgcgccattgcattgggggcaaaggtcgttggcatcaacaaccgcgatctgcgcgatttgtcgattgatctcaaccgtacccgcgagcttgcgccgaaactggggcacaacgtgacggtaatcagcgaatccggcatcaatacttacgctcaggtgcgcgagttaagccacttcgctaacggctttctgattggttcggcgttgatggcccatgacgatttgaacgccgccgtgcgtcgggtgttgctgggtgagaataaagtatgtggcctgacacgtgggcaagatgctaaagcagcttatgacgcgggcgcgatttacggtgggttgatttttgttgcgacatcaccgcgttgcgtcaacgttgaacaggcgcaggaagtgatggctgcagcaccgttgcagtatgttggcgtgttccgcaatcacgatattgccgatgtggcggacaaagctaaggtgttatcgctggcggcagtgcaactgcatggtaatgaagatcagctgtatatcgacaatctgcgtgaggctctgccagcacacgtcgccatctggaaggctttaagtgtcggtgaaactcttcccgcgcgcgattttcagcacatcgataaatatgtattcgacaacggtcagggcgggagcggacaacgtttcgactggtcactattaaatggtcaatcgcttggcaacgttctgctggcggggggcttaggcgcagataactgcgtggaagcggcacaaaccggctgcgccgggcttgattttaattctgctgtagagtcgcaaccgggtatcaaagacgcacgtcttttggcctcggttttccagacgctgcgcgcatattaaggaaaggaacaatgacaacattacttaacccctattttggtgagtttggcggcatgtacgtgccacaaatcctgatgcctgctctgcgccagctggaagaagcttttgtcagcgcgcaaaaagatcctgaatttcaggctcagttcaacgacctgctgaaaaactatgccgggcgtccaaccgcgctgaccaaatgccagaacattacagccgggacgaacaccacgctgtatctgaagcgcgaagatttgctgcacggcggcgcgcataaaactaaccaggtgctcggtcaggctttactggcgaagcggatgggtaaaactgaaattattgccgaaaccggtgccggtcagcatggcgtggcgtcggcccttgccagcgccctgctcggcctgaaatgccgaatttatatgggtgccaaagacgttgaacgccagtcgcccaacgttttccggatgcgcttaatgggtgcggaagtgatcccggtacatagcggttccgcgaccctgaaagatgcctgtaatgaggcgctacgcgactggtccggcagttatgaaaccgcgcactatatgctgggtaccgcagctggcccgcatccttacccgaccattgtgcgtgagtttcagcggatgattggcgaagaaacgaaagcgcagattctggaaagagaaggtcgcctgccggatgccgttatcgcctgtgttggcggtggttcgaatgccatcggtatgtttgcagatttcatcaacgaaaccgacgtcggcctgattggtgtggagcctggcggccacggtatcgaaactggcgagcacggcgcaccgttaaaacatggtcgcgtgggcatctatttcggtatgaaagcgccgatgatgcaaaccgaagacgggcaaattgaagagtcttactccatttctgccgggctggatttcccgtccgtcggcccgcaacatgcgtatctcaacagcactggacgcgctgattacgtgtctattaccgacgatgaagccctggaagcctttaaaacgctttgcctgcatgaagggatcatcccggcgctggaatcctcccacgccctggcccatgcgctgaaaatgatgcgcgaaaatccggaaaaagagcagctactggtggttaacctttccggtcgcggcgataaagacatcttcaccgttcacgatattttgaaagcacgaggggaaatctgatggaacgctacgaatctctgtttgcccagttgaaggagcgcaaagaaggcgcattcgttcctttcgtcaccctcggtgatccgggcattgagcagtcgttgaaaattatcgatacgctaattgaagccggtgctgacgcgctggagttaggcatccccttctccgacccactggcggatggcccgacgattcaaaacgccacactgcgtgcttttgcggcgggagtaaccccggcgcagtgctttgagatgctggcactcattcgccagaagcacccgaccattcccatcggccttttgatgtatgccaacctggtgtttaacaaaggcattgatgagttttatgccgagtgcgagaaagtcggcgtcgattcggtgctggttgccgatgtgcccgtggaagagtccgcgcccttccgccaggccgcgttgcgtcataatgtcgcacctatctttatttgcccgccgaatgccgacgatgatttgctgcgccagatagcctcttacggtcgtggttacacctatttgctgtcgcgagcgggcgtgaccggcgcagaaaaccgcgccgcgttacccctcaatcatctggttgcgaagctgaaagagtacaacgctgcgcctccattgcagggatttggtatttccgccccggatcaggtaaaagccgcgattgatgcaggagctgcgggcgcgatttctggttcggccatcgttaaaatcatcgagcaacatattaatgagccagagaaaatgctggcggcactgaaagcttttgtacaaccgatgaaagcggcgacgcgcagttaa

SEQ ID NO: 56 - DNA, 1562 bp, synthetic construct
  source 1..1562: mol_type = other DNA
atgcaaacacaaaaaccgactctcgaactgctaacctgcgaaggcgcttatcgcgacaacccgactgcgctttttcaccagttgtgtggggatcgtccggcaacgctgctgctggaattcgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttcta

SEQ ID NO: 57 - DNA, 7615 bp, synthetic construct
  source 1..7615: mol_type = other DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtgaaatgtaagaaggagatatacatatggaaaacaacaccaatatgttctctggagtgaaggtgatcgaactggccaactttatcgctgctccggcggcaggtcgcttctttgctgatgggggagcagaagtaattaagatcgaatctccagcaggcgacccgctgcgctacacggccccatcagaaggacgcccgctttctcaagaggaaaacacaacgtatgatttggaaaacgcgaataagaaagcaattgttctgaacttaaaatcggaaaaaggaaagaaaattcttcacgagatgcttgctgaggcagacatcttgttaacaaattggcgcacgaaagcgttagtcaaacaggggttagattacgaaacactgaaagagaagtatccaaaattggtatttgcacagattacaggatacggggagaaaggacccgacaaagacctgcctggtttcgactacacggcgtttttcgcccgcggaggagtctccggtacattatatgaaaaaggaactgtccctcctaatgtggtaccgggtctgggtgaccaccaggcaggaatgttcttagctgccggtatggctggtgcgttgtataaggccaaaaccaccggacaaggcgacaaagtcaccgttagtctgatgcatagcgcaatgtacggcctgggaatcatgattcaggcagcccagtacaaggaccatgggctggtgtacccgatcaaccgtaatgaaacgcctaatcctttcatcgtttcatacaagtccaaagatgattactttgtccaagtttgcatgcctccctatgatgtgttttatgatcgctttatgacggccttaggacgtgaagacttggtaggtgacgaacgctacaataagatcgagaacttgaaggatggtcgcgcaaaagaagtctattccatcatcgaacaacaaatggtaacgaagacgaaggacgaatgggacaagatttttcgtgatgcagacattccattcgctattgcccaaacgtgggaagatcttttagaagacgagcaggcatgggccaacgactacctgtataaaatgaagtatcccacaggcaacgaacgtgccctggtacgtttacctgtgttcttcaaagaagctggacttcctgaatacaaccagtcgccacagattgctgagaataccgtggaagtgttaaaggagatgggatataccgagcaagaaattgaggagcttgagaaagacaaagacatcatggtacgtaaagagaaatgaaggttaagaaggagatatacatatgtcagaccgcaacaaagaagtgaaagaaaagaaggctaaacactatctgcgcgagatcacagctaaacactacaaggaagcgttagaggctaaagagcgtggggagaaagtgggttggtgtgcctctaacttcccccaagagattgcaaccacgttgggtgtaaaggttgtttatcccgaaaaccacgccgccgccgtagcggcacgtggcaatgggcaaaatatgtgcgaacacgcggaggctatgggattcagtaatgatgtgtgtggatatgcacgtgtaaatttagccgtaatggacatcggccatagtgaagatcaacctattccaatgcctgatttcgttctgtgctgtaataatatctgcaatcagatgattaaatggtatgaacacattgcaaaaacgttggatattcctatgatccttatcgatattccatataatactgagaacacggtgtctcaggaccgcattaagtacatccgcgcccagttcgatgacgctatcaagcaactggaagaaatcactggcaaaaagtgggacgagaataaattcgaagaagtgatgaagatttcgcaagaatcggccaagcaatggttacgcgccgcgagctacgcgaaatacaaaccatcaccgttttcgggctttgacctttttaatcacatggctgtagccgtttgtgctcgcggcacccaggaagccgccgatgcattcaaaatgttagcagatgaatatgaagagaacgttaagacaggaaagtctacttatcgcggcgaggagaagcagcgtatcttgttcgagggcatcgcttgttggccttatctgcgccacaagttgacgaaactgagtgaatatggaatgaacgtcacagctacggtgtacgccgaagcttttggggttatttacgaaaacatggatgaactgatggccgcttacaataaagtgcctaactcaatctccttcgagaacgcgctgaagatgcgtcttaatgccgttacaagcaccaatacagaaggggctgttatccacattaatcgcagttgtaagctgtggtcaggattcttatacgaactggcccgtcgtttggaaaaggagacggggatccctgttgtttcgttcgacggagatcaagcggatccccgtaacttctccgaggctcaatatgacactcgcatccaaggtttaaatgaggtgatggtcgcgaaaaaagaagcagagtgagctttaagaaggagatatacatatgtcgaatagtgacaagttttttaacgacttcaaggacattgtggaaaacccaaagaagtatatcatgaagcatatggaacaaacgggacaaaaagccatcggttgcatgcctttatacaccccagaagagcttgtcttagcggcgggtatgtttcctgttggagtatggggctcgaatactgagttgtcaaaagccaagacctactttccggcttttatctgttctatcttgcaaactactttagaaaacgcattgaatggggagtatgacatgctgtctggtatgatgatcacaaactattgcgattcgctgaaatgtatgggacaaaacttcaaacttacagtggaaaatatcgaattcatcccggttacggttccacaaaaccgcaagatggaggcgggtaaagaatttctgaaatcccagtataaaatgaatatcgaacaactggaaaaaatctcagggaataagatcactgacgagagcttggagaaggctattgaaatttacgatgagcaccgtaaagtcatgaacgatttctctatgcttgcgtccaagtaccctggtatcattacgccaacgaaacgtaactacgtgatgaagtcagcgtattatatggacaagaaagaacatacagagaaggtacgtcagttgatggatgaaatcaaggccattgagcctaaaccattcgaaggaaaacgcgtgattaccactgggatcattgcagattcggaggaccttttgaaaatcttggaggagaataacattgctatcgtgggagatgatattgcacacgagtctcgccaataccgcactttgaccccggaggccaacacacctatggaccgtcttgctgaacaatttgcgaaccgcgagtgttcgacgttgtatgaccctgaaaaaaaacgtggacagtatattgtcgagatggcaaaagagcgtaaggccgacggaatcatcttcttcatgacaaaattctgcgatcccgaagaatacgattaccctcagatgaaaaaagacttcgaagaagccggtattccccacgttctgattgagacagacatgcaaatgaagaactacgaacaagctcgcaccgctattcaagcattttcagaaaccctttgacgcttaagaaggagatatacatatgcgtgctgtcttaatcgagaagtcagatgacacccagagtgtttcagttacggagttggctgaagaccaattacccgaaggtgacgtccttgtggatgtcgcgtacagcacattgaattacaaggatgctcttgcgattactggaaaagcacccgttgtacgccgttttcctatggtccccggaattgactttactgggactgtcgcacagagttcccatgctgatttcaagccaggcgaccgcgtaattctgaacggatggggagttggtgagaaacactggggcggtcttgcagaacgcgcacgcgtacgtggggactggcttgtcccgttgccagcccccttagacttgcgccaggctgcaatgattggcactgcggggtacacagctatgctgtgcgtgcttgcccttgagcgccatggagtcgtacctgggaacggcgagattgtcgtctcaggcgcagcaggaggggtaggttctgtagcaaccacactgttagcagccaaaggctacgaagtggccgccgtgaccgggcgcgcaagcgaggccgaatatttacgcggattaggcgccgcgtcggtcattgatcgcaatgaattaacggggaaggtgcgtccattagggcaggaacgctgggcaggaggaatcgatgtagcaggatcaaccgtacttgctaatatgttgagcatgatgaaataccgtggcgtggtggcggcctgtggcctggcggctggaatggacttgcccgcgtctgtcgccccttttattctgcgtggtatgactttggcaggggtagattcagtcatgtgccccaaaactgatcgtctggctgcttgggcacgcctggcatccgacctggaccctgcaaagctggaagagatgacaactgaattaccgttctctgaggtgattgaaacggctccgaagttcttggatggaacagtgcgtgggcgtattgtcattccggtaacaccttgatacttaagaaggagatatacatatgaaaatcttggcatactgcgtccgcccagacgaggtagactcctttaagaaatttagtgaaaagtacgggcatacagttgatcttattccagactcttttggacctaatgtcgctcatttggcgaagggttacgatgggatttctattctgggcaacgacacgtgtaaccgtgaggcactggagaagatcaaggattgcgggatcaaatatctggcaacccgtacagccggagtgaacaacattgacttcgatgcagcaaaggagttcggtattaacgtggctaatgttcccgcatattcccccaactcggtcagcgaatttaccattggattggcattaagtctgacgcgtaagattccatttgccctgaaacgcgtggaactgaacaattttgcgcttggcggccttattggtgtggaattgcgtaacttaactttaggagtcatcggtactggtcgcatcggattgaaagtgattgagggcttctctgggtttggaatgaaaaaaatgatcggttatgacatttttgaaaatgaagaagcaaagaagtacatcgaatacaaatcattagacgaagtttttaaagaggctgatattatcactctgcatgcgcctctgacagacgacaactatcatatgattggtaaagaatccattgctaaaatgaaggatggggtatttattatcaacgcagcgcgtggagccttaatcgatagtgaggccctgattgaagggttaaaatcggggaagatt

SEQ ID NO: 58 - DNA, 1239 bp, synthetic construct
  source 1..1239: mol_type = other DNA
atggaaaacaacaccaatatgttctctggagtgaaggtgatcgaactggccaactttatcgctgctccggcggcaggtcgcttctttgctgatgggggagcagaagtaattaagatcgaatctccagcaggcgacccgctgcgctacacggccccatcagaaggacgcccgctttctcaagaggaaaacacaacgtatgatttggaaaacgcgaataagaaagcaattgttctgaacttaaaatcggaaaaaggaaagaaaattcttcacgagatgcttgctgaggcagacatcttgttaacaaattggcgcacgaaagcgttagtcaaacaggggttagattacgaaacactgaaagagaagtatccaaaattggtatttgcacagattacaggatacggggagaaaggacccgacaaagacctgcctggtttcgactacacggcgtttttcgcccgcggaggagtctccggtacattatatgaaaaaggaactgtccctcctaatgtggtaccgggtctgggtgaccaccaggcaggaatgttcttagctgccggtatggctggtgcgttgtataaggccaaaaccaccggacaaggcgacaaagtcaccgttagtctgatgcatagcgcaatgtacggcctgggaatcatgattcaggcagcccagtacaaggaccatgggctggtgtacccgatcaaccgtaatgaaacgcctaatcctttcatcgtttcatacaagtccaaagatgattactttgtccaagtttgcatgcctccctatgatgtgttttatgatcgctttatgacggccttaggacgtgaagacttggtaggtgacgaacgctacaataagatcgagaacttgaaggatggtcgcgcaaaagaagtctattccatcatcgaacaacaaatggtaacgaagacgaaggacgaatgggacaagatttttcgtgatgcagacattccattcgctattgcccaaacgtgggaagatcttttagaagacgagcaggcatgggccaacgactacctgtataaaatgaagtatcccacaggcaacgaacgtgccctggtacgtttacctgtgttcttcaaagaagctggacttcctgaatacaaccagtcgccacagattgctgagaataccgtggaagtgttaaaggagatgggatataccgagcaagaaattgaggagcttgagaaagacaaagacatcatggtacgtaaagagaaatga

SEQ ID NO: 59 - DNA, 1224 bp, synthetic construct
  source 1..1224: mol_type = other DNA
atgtcagaccgcaacaaagaagtgaaagaaaagaaggctaaacactatctgcgcgagatcacagctaaacactacaaggaagcgttagaggctaaagagcgtggggagaaagtgggttggtgtgcctctaacttcccccaagagattgcaaccacgttgggtgtaaaggttgtttatcccgaaaaccacgccgccgccgtagcggcacgtggcaatgggcaaaatatgtgcgaacacgcggaggctatgggattcagtaatgatgtgtgtggatatgcacgtgtaaatttagccgtaatggacatcggccatagtgaagatcaacctattccaatgcctgatttcgttctgtgctgtaataatatctgcaatcagatgattaaatggtatgaacacattgcaaaaacgttggatattcctatgatccttatcgatattccatataatactgagaacacggtgtctcaggaccgcattaagtacatccgcgcccagttcgatgacgctatcaagcaactggaagaaatcactggcaaaaagtgggacgagaataaattcgaagaagtgatgaagatttcgcaagaatcggccaagcaatggttacgcgccgcgagctacgcgaaatacaaaccatcaccgttttcgggctttgacctttttaatcacatggctgtagccgtttgtgctcgcggcacccaggaagccgccgatgcattcaaaatgttagcagatgaatatgaagagaacgttaagacaggaaagtctacttatcgcggcgaggagaagcagcgtatcttgttcgagggcatcgcttgttggccttatctgcgccacaagttgacgaaactgagtgaatatggaatgaacgtcacagctacggtgtacgccgaagcttttggggttatttacgaaaacatggatgaactgatggccgcttacaataaagtgcctaactcaatctccttcgagaacgcgctgaagatgcgtcttaatgccgttacaagcaccaatacagaaggggctgttatccacattaatcgcagttgtaagctgtggtcaggattcttatacgaactggcccgtcgtttggaaaaggagacggggatccctgttgtttcgttcgacggagatcaagcggatccccgtaacttctccgaggctcaatatgacactcgcatccaaggtttaaatgaggtgatggtcgcgaaaaaagaagcagagtga

SEQ ID NO: 60 - DNA, 1124 bp, synthetic construct
  source 1..1124: mol_type = other DNA
atgtcgaatagtgacaagttttttaacgacttcaaggacattgtggaaaacccaaagaagtatatcatgaagcatatggaacaaacgggacaaaaagccatcggttgcatgcctttatacaccccagaagagcttgtcttagcggcgggtatgtttcctgttggagtatggggctcgaatactgagttgtcaaaagccaagacctactttccggcttttatctgttctatcttgcaaactactttagaaaacgcattgaatggggagtatgacatgctgtctggtatgatgatcacaaactattgcgattcgctgaaatgtatgggacaaaacttcaaacttacagtggaaaatatcgaattcatcccggttacggttccacaaaaccgcaagatggaggcgggtaaagaatttctgaaatcccagtataaaatgaatatcgaacaactggaaaaaatctcagggaataagatcactgacgagagcttggagaaggctattgaaatttacgatgagcaccgtaaagtcatgaacgatttctctatgcttgcgtccaagtaccctggtatcattacgccaacgaaacgtaactacgtgatgaagtcagcgtattatatggacaagaaagaacatacagagaaggtacgtcagttgatggatgaaatcaaggccattgagcctaaaccattcgaaggaaaacgcgtgattaccactgggatcattgcagattcggaggaccttttgaaaatcttggaggagaataacattgctatcgtgggagatgatattgcacacgagtctcgccaataccgcactttgaccccggaggccaacacacctatggaccgtcttgctgaacaatttgcgaaccgcgagtgttcgacgttgtatgaccctgaaaaaaaacgtggacagtatattgtcgagatggcaaaagagcgtaaggccgacggaatcatcttcttcatgacaaaattctgcgatcccgaagaatacgattaccctcagatgaaaaaagacttcgaagaagccggtattccccacgttctgattgagacagacatgcaaatgaagaactacgaacaagctcgcaccgctattcaagcattttcagaaaccctttg

SEQ ID NO: 61 - DNA, 981 bp, synthetic construct
  source 1..981: mol_type = other DNA
atgcgtgctgtcttaatcgagaagtcagatgacacccagagtgtttcagttacggagttggctgaagaccaattacccgaaggtgacgtccttgtggatgtcgcgtacagcacattgaattacaaggatgctcttgcgattactggaaaagcacccgttgtacgccgttttcctatggtccccggaattgactttactgggactgtcgcacagagttcccatgctgatttcaagccaggcgaccgcgtaattctgaacggatggggagttggtgagaaacactggggcggtcttgcagaacgcgcacgcgtacgtggggactggcttgtcccgttgccagcccccttagacttgcgccaggctgcaatgattggcactgcggggtacacagctatgctgtgcgtgcttgcccttgagcgccatggagtcgtacctgggaacggcgagattgtcgtctcaggcgcagcaggaggggtaggttctgtagcaaccacactgttagcagccaaaggctacgaagtggccgccgtgaccgggcgcgcaagcgaggccgaatatttacgcggattaggcgccgcgtcggtcattgatcgcaatgaattaacggggaaggtgcgtccattagggcaggaacgctgggcaggaggaatcgatgtagcaggatcaaccgtacttgctaatatgttgagcatgatgaaataccgtggcgtggtggcggcctgtggcctggcggctggaatggacttgcccgcgtctgtcgccccttttattctgcgtggtatgactttggcaggggtagattcagtcatgtgccccaaaactgatcgtctggctgcttgggcacgcctggcatccgacctggaccctgcaaagctggaagagatgacaactgaattaccgttctctgaggtgattgaaacggctccgaagttcttggatggaacagtgcgtgggcgtattgtcattccggtaacaccttga

SEQ ID NO: 62 - DNA, 996 bp, synthetic construct
  source 1..996: mol_type = other DNA
atgaaaatcttggcatactgcgtccgcccagacgaggtagactcctttaagaaatttagtgaaaagtacgggcatacagttgatcttattccagactcttttggacctaatgtcgctcatttggcgaagggttacgatgggatttctattctgggcaacgacacgtgtaaccgtgaggcactggagaagatcaaggattgcgggatcaaatatctggcaacccgtacagccggagtgaacaacattgacttcgatgcagcaaaggagttcggtattaacgtggctaatgttcccgcatattcccccaactcggtcagcgaatttaccattggattggcattaagtctgacgcgtaagattccatttgccctgaaacgcgtggaactgaacaattttgcgcttggcggccttattggtgtggaattgcgtaacttaactttaggagtcatcggtactggtcgcatcggattgaaagtgattgagggcttctctgggtttggaatgaaaaaaatgatcggttatgacatttttgaaaatgaagaagcaaagaagtacatcgaatacaaatcattagacgaagtttttaaagaggctgatattatcactctgcatgcgcctctgacagacgacaactatcatatgattggtaaagaatccattgctaaaatgaaggatggggtatttattatcaacgcagcgcgtggagccttaatcgatagtgaggccctgattgaagggttaaaatcggggaagattgcgggcgcggctctggatagctatgagtatgagcaaggtgtctttcacaacaataagatgaatgaaattatgcaggatgataccttggaacgtctgaaatcttttcccaacgtcgtgatcacgccgcatttgggtttttatactgatgaggcggtttccaatatggtagagatcacactgatgaaccttcaggaattcgagttgaaaggaacctgtaagaaccagcgtgtttgtaaatga

SEQ ID NO: 63 - DNA, 8008 bp, synthetic construct
  source 1..8008: mol_type = other DNA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtgaaatgtaagaaggagatatacatatggaaaacaacaccaatatgttctctggagtgaaggtgatcgaactggccaactttatcgctgctccggcggcaggtcgcttctttgctgatgggggagcagaagtaattaagatcgaatctccagcaggcgacccgctgcgctacacggccccatcagaaggacgcccgctttctcaagaggaaaacacaacgtatgatttggaaaacgcgaataagaaagcaattgttctgaacttaaaatcggaaaaaggaaagaaaattcttcacgagatgcttgctgaggcagacatcttgttaacaaattggcgcacgaaagcgttagtcaaacaggggttagattacgaaacactgaaagagaagtatccaaaattggtatttgcacagattacaggatacggggagaaaggacccgacaaagacctgcctggtttcgactacacggcgtttttcgcccgcggaggagtctccggtacattatatgaaaaaggaactgtccctcctaatgtggtaccgggtctgggtgaccaccaggcaggaatgttcttagctgccggtatggctggtgcgttgtataaggccaaaaccaccggacaaggcgacaaagtcaccgttagtctgatgcatagcgcaatgtacggcctgggaatcatgattcaggcagcccagtacaaggaccatgggctggtgtacccgatcaaccgtaatgaaacgcctaatcctttcatcgtttcatacaagtccaaagatgattactttgtccaagtttgcatgcctccctatgatgtgttttatgatcgctttatgacggccttaggacgtgaagacttggtaggtgacgaacgctacaataagatcgagaacttgaaggatggtcgcgcaaaagaagtctattccatcatcgaacaacaaatggtaacgaagacgaaggacgaatgggacaagatttttcgtgatgcagacattccattcgctattgcccaaacgtgggaagatcttttagaagacgagcaggcatgggccaacgactacctgtataaaatgaagtatcccacaggcaacgaacgtgccctggtacgtttacctgtgttcttcaaagaagctggacttcctgaatacaaccagtcgccacagattgctgagaataccgtggaagtgttaaaggagatgggatataccgagcaagaaattgaggagcttgagaaagacaaagacatcatggtacgtaaagagaaatgaaggttaagaaggagatatacatatgtcagaccgcaacaaagaagtgaaagaaaagaaggctaaacactatctgcgcgagatcacagctaaacactacaaggaagcgttagaggctaaagagcgtggggagaaagtgggttggtgtgcctctaacttcccccaagagattgcaaccacgttgggtgtaaaggttgtttatcccgaaaaccacgccgccgccgtagcggcacgtggcaatgggcaaaatatgtgcgaacacgcggaggctatgggattcagtaatgatgtgtgtggatatgcacgtgtaaatttagccgtaatggacatcggccatagtgaagatcaacctattccaatgcctgatttcgttctgtgctgtaataatatctgcaatcagatgattaaatggtatgaacacattgcaaaaacgttggatattcctatgatccttatcgatattccatataatactgagaacacggtgtctcaggaccgcattaagtacatccgcgcccagttcgatgacgctatcaagcaactggaagaaatcactggcaaaaagtgggacgagaataaattcgaagaagtgatgaagatttcgcaagaatcggccaagcaatggttacgcgccgcgagctacgcgaaatacaaaccatcaccgttttcgggctttgacctttttaatcacatggctgtagccgtttgtgctcgcggcacccaggaagccgccgatgcattcaaaatgttagcagatgaatatgaagagaacgttaagacaggaaagtctacttatcgcggcgaggagaagcagcgtatcttgttcgagggcatcgcttgttggccttatctgcgccacaagttgacgaaactgagtgaatatggaatgaacgtcacagctacggtgtacgccgaagcttttggggttatttacgaaaacatggatgaactgatggccgcttacaataaagtgcctaactcaatctccttcgagaacgcgctgaagatgcgtcttaatgccgttacaagcaccaatacagaaggggctgttatccacattaatcgcagttgtaagctgtggtcaggattcttatacgaactggcccgtcgtttggaaaaggagacggggatccctgttgtttcgttcgacggagatcaagcggatccccgtaacttctccgaggctcaatatgacactcgcatccaaggtttaaatgaggtgatggtcgcgaaaaaagaagcagagtgagctttaagaaggagatatacatatgtcgaatagtgacaagttttttaacgacttcaaggacattgtggaaaacccaaagaagtatatcatgaagcatatggaacaaacgggacaaaaagccatcggttgcatgcctttatacaccccagaagagcttgtcttagcggcgggtatgtttcctgttggagtatggggctcgaatactgagttgtcaaaagccaagacctactttccggcttttatctgttctatcttgcaaactactttagaaaacgcattgaatggggagtatgacatgctgtctggtatgatgatcacaaactattgcgattcgctgaaatgtatgggacaaaacttcaaacttacagtggaaaatatcgaattcatcccggttacggttccacaaaaccgcaagatggaggcgggtaaagaatttctgaaatcccagtataaaatgaatatcgaacaactggaaaaaatctcagggaataagatcactgacgagagcttggagaaggctattgaaatttacgatgagcaccgtaaagtcatgaacgatttctctatgcttgcgtccaagtaccctggtatcattacgccaacgaaacgtaactacgtgatgaagtcagcgtattatatggacaagaaagaacatacagagaaggtacgtcagttgatggatgaaatcaaggccattgagcctaaaccattcgaaggaaaacgcgtgattaccactgggatcattgcagattcggaggaccttttgaaaatcttggaggagaataacattgctatcgtgggagatgatattgcacacgagtctcgccaataccgcactttgaccccggaggccaacacacctatggaccgtcttgctgaacaatttgcgaaccgcgagtgttcgacgttgtatgaccctgaaaaaaaacgtggacagtatattgtcgagatggcaaaagagcgtaaggccgacggaatcatcttcttcatgacaaaattctgcgatcccgaagaatacgattaccctcagatgaaaaaagacttcgaagaagccggtattccccacgttctgattgagacagacatgcaaatgaagaactacgaacaagctcgcaccgctattcaagcattttcagaaaccctttgacgcttaagaaggagatatacatatgttctttacggagcaacacgaacttattcgcaaactggcgcgtgactttgccgaacaggaaatcgagcctatcgcagacgaagtagataaaaccgcagagttcccaaaagaaatcgtgaagaagatggctcaaaatggatttttcggcattaaaatgcctaaagaatacggaggggcgggtgcggataaccgcgcttatgtcactattatggaggaaatttcacgtgcttccggggtagcgggtatctacctgagctcgccgaacagtttgttaggaactcccttcttattggtcggaaccgatgagcaaaaagaaaagtaccttaagcctatgatccgcggcgagaagactctggcgttcgccctgacagagcctggtgctggctctgatgcgggtgcgttggctactactgcccgtgaagagggcgactattatatcttaaatggccgcaagacgtttattacaggggctcctattagcgacaatattattgtgttcgcaaaaaccgatatgagcaaagggaccaaaggtatcaccactttcattgtggactcaaagcaggaaggggtaagttttggtaagccagaggacaaaatgggaatgattggttgtccgacaagcgacatcatcttggaaaacgttaaagttcataagtccgacatcttgggagaagtcaataaggggtttattaccgcgatgaaaacactttccgttggtcgtatcggagtggcgtcacaggcgcttggaattgcacaggccgccgtagatgaggcggtaaagtacgccaagcaacgtaaacaattcaatcgcccaatcgcgaaatttcaggccattcaatttaaacttgccaatatggagactaaattaaatgccgctaaacttcttgtttataacgcagcgtacaaaatggattgtggagaaaaagccgacaaggaagcctctatggctaaatactttgctgctgaatcagcgatccaaatcgttaacgacgcgctgcaaatccatggcgggtatggctatatcaaagactacaagattgaacgtttgtaccgcgatgtgcgtgtgatcgctatttatgagggcacttccgaggtccaacagatggttatcgcgtccaatctgctgaagtaatacttaagaaggagatatacatatgaaaatcttggcatactgcgtccgcccagacgaggtagactcctttaagaaatttagtgaaaagtacgggcatacagttgatcttattccagactcttttggacctaatgtcgctcatttggcgaagggttacgatgggatttctattctgggcaacgacacgtgtaaccgtgaggcactggagaagatcaaggattgcgggatcaaatatctggcaacccgtacagccggagtgaacaacattgacttcgatgcagcaaaggagttcggtattaacgtggctaatgttcccgcatattcccccaactcggtcagcgaatttaccattggattggcattaagtctgacgcgtaagattccatttgccctgaaacgcgtggaactgaacaattttgcgcttggcggccttattggtgtggaattgcgtaacttaactttaggagtcatcggtactggtcgcatcggattgaaagtgattgagggcttctctgggtttggaatgaaaaaaatgatcggttatgacatttttgaaaatgaagaagcaaagaagtacatcgaatacaaatcattagacgaagtttttaaagaggctgatattatcactctgcatgcgcctctgacagacgacaactatcatatgattggtaaagaatccattgctaaaatgaaggatggggtatttattatcaacgcagcgcgtggagccttaatcgatagtgaggccctgattgaagggttaaaatcggggaagattgcgggcgcggctctggatagctatgagtatgagcaaggtgtctttcacaacaataagatgaatgaaattatgcaggatgataccttggaacgtctgaaatcttttcccaacgtcgtgatcacgccgcatttgggtttttatactgatgaggcggtttccaatatggtagagatcacactgatgaaccttcaggaattcgagttgaaaggaacctgtaagaaccagcgtgtttgtaaatga

SEQ ID NO: 64 - DNA, 1134 bp, synthetic construct
  source 1..1134: mol_type = other DNA
atgttctttacggagcaacacgaacttattcgcaaactggcgcgtgactttgccgaacaggaaatcgagcctatcgcagacgaagtagataaaaccgcagagttcccaaaagaaatcgtgaagaagatggctcaaaatggatttttcggcattaaaatgcctaaagaatacggaggggcgggtgcggataaccgcgcttatgtcactattatggaggaaatttcacgtgcttccggggtagcgggtatctacctgagctcgccgaacagtttgttaggaactcccttcttattggtcggaaccgatgagcaaaaagaaaagtaccttaagcctatgatccgcggcgagaagactctggcgttcgccctgacagagcctggtgctggctctgatgcgggtgcgttggctactactgcccgtgaagagggcgactattatatcttaaatggccgcaagacgtttattacaggggctcctattagcgacaatattattgtgttcgcaaaaaccgatatgagcaaagggaccaaaggtatcaccactttcattgtggactcaaagcaggaaggggtaagttttggtaagccagaggacaaaatgggaatgattggttgtccgacaagcgacatcatcttggaaaacgttaaagttcataagtccgacatcttgggagaagtcaataaggggtttattaccgcgatgaaaacactttccgttggtcgtatcggagtggcgtcacaggcgcttggaattgcacaggccgccgtagatgaggcggtaaagtacgccaagcaacgtaaacaattcaatcgcccaatcgcgaaatttcaggccattcaatttaaacttgccaatatggagactaaattaaatgccgctaaacttcttgtttataacgcagcgtacaaaatggattgtggagaaaaagccgacaaggaagcctctatggctaaatactttgctgctgaatcagcgatccaaatcgttaacgacgcgctgcaaatccatggcgggtatggctatatcaaagactacaagattgaacgtttgtaccgcgatgtgcgtgtgatcgctatttatgagggcacttccgaggtccaacagatggttatcgcgtccaatctgctgaagtaa

SEQ ID NO: 65 - DNA, 117 bp, synthetic construct
  source 1..117: mol_type = other DNA
atccccatcactcttgatggagatcaattccccaagctgctagagcgttaccttgcccttaaacattagcaatgtcgatttatcagagggccgacaggctcccacaggagaaaaccg

SEQ ID NO: 66 - DNA, 108 bp, synthetic construct
  source 1..108: mol_type = other DNA
ctcttgatcgttatcaattcccacgctgtttcagagcgttaccttgcccttaaacattagcaatgtcgatttatcagagggccgacaggctcccacaggagaaaaccg

SEQ ID NO: 67 - DNA, 433 bp, synthetic construct
  source 1..433: mol_type = other DNA
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 68 - DNA, 290 bp, synthetic construct
  source 1..290: mol_type = other DNA
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 69 - DNA, 207 bp, synthetic construct
  source 1..207: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 70 - DNA, 390 bp, synthetic construct
  source 1..390: mol_type = other DNA
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 71 - DNA, 200 bp, synthetic construct
  source 1..200: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 72-95 - skipped (24 entries)

SEQ ID NO: 96 - DNA, 1470 bp, synthetic construct
  source 1..1470: mol_type = other DNA
atgagtcaagtgattaagaagaaacgtaacacctttatgatcggaacggagtacattcttaacagtacacaattggaggaagcgattaaatcattcgtacatgatttctgcgcagagaagcatgagatccatgatcaacctgtggtagtagaagctaaagaacatcaggaggacaaaatcaaacaaatcaaaatcccggaaaagggacgtcctgtaaatgaagtcgtttctgagatgatgaatgaagtgtatcgctaccgcggagacgccaaccatcctcgctttttttcttttgtgcccggacctgcaagcagtgtgtcgtggttgggggatattatgacgtccgcctacaatattcatgctggaggctcaaagctggcaccgatggttaactgcattgagcaggaagttctgaagtggttagcaaagcaagtggggttcacagaaaatccaggtggcgtatttgtgtcgggcggttcaatggcgaatattacggcacttactgcggctcgtgacaataaactgaccgacattaaccttcatttgggaactgcttatattagtgaccagactcatagttcagttgcgaaaggattacgcattattggaatcactgacagtcgcatccgtcgcattcccactaactcccacttccagatggataccaccaagctggaggaagccatcgagaccgacaagaagtctggctacattccgttcgtcgttatcggaacagcaggtaccaccaacactggttcgattgaccccctgacagaaatctctgcgttatgtaagaagcatgacatgtggtttcatatcgacggagcgtatggagctagtgttctgctgtcacctaagtacaagagccttcttaccggaaccggcttggctgacagtatttcgtgggatgctcataaatggttgttccaaacgtacggctgtgcaatggtacttgtcaaagatatccgtaatttattccactcttttcatgtgaatcccgagtatcttaaggatctggaaaacgacatcgataacgttaatacatgggacatcggcatggagctgacgcgccctgcacgcggtcttaaattgtggcttactttacaggtccttggatctgacttgattgggagtgccattgaacacggtttccagctggcagtttgggctgaggaagcattgaatccaaagaaagactgggagatcgtttctccagctcagatggctatgattaatttccgttatgcccctaaggatttaaccaaagaggaacaggatattctgaatgaaaagatctcccaccgcattttagagagcggatacgctgcaattttcactactgtattaaacggcaagaccgttttacgcatctgtgcaattcacccggaggcaactcaagaggatatgcaacacacaatcgacttattagaccaatacggtcgtgaaatctataccgagatgaagaaagcg

SEQ ID NO: 97 - protein, 444 aa, Cryptococcus deuterogattii
MTATTISIETVPQAPAAGTKTNGTSGKYNPRTYLSDRAKVTEIDGSDAGRPNPDTFPFNSITLNLKPPLGLPESSNNMPVSITIEDPDLATALQYAPSAGIPKLREWLADLQAHVHERPRGDYAISVGSGSQDLMFKGFQAVLNPGDPVLLETPMYSGVLPALRILKADYAEVDVDDQGLSAKNLEKVLSEWPADKKRPRVLYTSPIGSNPSGCSASKERKLEVLKVCKKYDVLIFEDDPYYYLAQELIPSYFALEKQVYPEGGHVVRFDSFSKLLSAGMRLGFATGPKEILHAIDVSTAGANLHTSAVSQGVALRLMQYWGIEGFLAHGRAVAKLYTERRAQFEATAHKYLDGLATWVSPVAGMFLWIDLRPAGIEDSYELIRHEALAKGVLGVPGMAFYPTGRKSSHVRVSFSIVDLEDESDLGFQRLAEAIKDKRKALGLA

SEQ ID NO: 98 - DNA, 1332 bp, synthetic construct
  source 1..1332: mol_type = other DNA
atgacggcaactacaatttctattgagaccgtacctcaggccccggcggcggggaccaaaactaatgggacttcaggaaaatacaacccccgcacttacctgtccgaccgcgccaaagtcactgagattgatggatctgacgccggtcgccccaatcccgatactttcccatttaactcgattaccttaaatttgaaaccacctttaggcttgcccgagagttcaaataacatgccggtctctatcacgattgaagaccccgatttagcgacggccttacaatatgcacctagcgccggtattcctaagctgcgcgaatggctggctgacttacaagctcacgttcatgagcgcccccgtggcgattatgccatctcggtcgggtcggggtcacaggatttgatgtttaagggcttccaagctgtcttgaatccaggtgatccagtccttctggaaaccccaatgtattcaggtgttctgccagcgctgcgcattctgaaggcggattatgcagaagttgatgtagacgaccaggggttatctgctaaaaaccttgaaaaagttttatcagagtggcccgcagataagaagcgtcctcgtgtcctgtatacgtcgccaatcggctccaatccttccggatgttcagcatccaaggaacgcaagttagaggtactgaaagtctgtaagaagtacgatgtgctgatcttcgaagacgatccgtattattaccttgctcaagagcttattccatcctattttgcgttggaaaaacaagtttatccggagggtgggcacgttgtacgctttgactcatttagtaaattgctttctgctgggatgcgcttgggatttgctacagggccgaaggaaattcttcatgcgattgacgtcagtacagcaggcgcaaatttacatacttcagcggtctctcaaggtgtcgctcttcgcctgatgcagtattgggggatcgagggattccttgcacatggccgcgcggtggccaaactttacacggagcgccgcgctcagttcgaggcaaccgcacataagtacctggacgggctggccacttgggtatctcccgtagcgggaatgtttttatggatcgatcttcgtccagcaggaatcgaagattcttacgaattaattcgccatgaagcattagccaaaggcgttttaggcgttccagggatggcgttttatccgacaggccgtaagtcttcccatgttcgtgtcagtttcagtatcgtcgacctggaagacgaatctgaccttggttttcaacgcctggctgaagctattaaggataaacgcaaggctttagggctggct

SEQ ID NO: 99 - protein, 500 aa, Catharanthus roseus
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMGKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 100 - protein, 757 aa, Escherichia coli
MGSPSLYSARKTTLALAVALSFAWQAPVFAHGGEAHMVPMDKTLKEFGADVQWDDYAQLFTLIKDGAYVKVKPGAQTAIVNGQPLALQVPVVMKDNKAWVSDTFINDVFQSGLDQTFQVEKRPHPLNALTADEIKQAVEIVKASADFKPNTRFTEISLLPPDKEAVWAFALENKPVDQPRKADVIMLDGKHIIEAVVDLQNNKLLSWQPIKDAHGMVLLDDFASVQNIINNSEEFAAAVKKRGITDAKKVITTPLTVGYFDGKDGLKQDARLLKVISYLDVGDGNYWAHPIENLVAVVDLEQKKIVKIEEGPVVPVPMTARPFDGRDRVAPAVKPMQIIEPEGKNYTITGDMIHWRNWDFHLSMNSRVGPMISTVTYNDNGTKRKVMYEGSLGGMIVPYGDPDIGWYFKAYLDSGDYGMGTLTSPIARGKDAPSNAVLLNETIADYTGVPMEIPRAIAVFERYAGPEYKHQEMGQPNVSTERRELVVRWISTVGNYDYIFDWIFHENGTIGIDAGATGIEAVKGVKAKTMHDETAKDDTRYGTLIDHNIVGTTHQHIYNFRLDLDVDGENNSLVAMDPVVKPNTAGGPRTSTMQVNQYNIGNEQDAAQKFDPGTIRLLSNPNKENRMGNPVSYQIIPYAGGTHPVAKGAQFAPDEWIYHRLSFMDKQLWVTRYHPGERFPEGKYPNRSTHDTGLGQYSKDNESLDNTDAVVWMTTGTTHVARAEEWPIMPTEWVHTLLKPWNFFDETPTLGALKKDK

SEQ ID NO: 101 - protein, 1368 aa, Arabidopsis thaliana
MGEKAIDEDKVEAMKSSKTSLVFAINGQRFELELSSIDPSTTLVDFLRNKTPFKSVKLGCGEGGCGACVVLLSKYDPLLEKVDEFTISSCLTLLCSIDGCSITTSDGLGNSRVGFHAVHERIAGFHATQCGFCTPGMSVSMFSALLNADKSHPPPRSGFSNLTAVEAEKAVSGNLCRCTGYRPLVDACKSFAADVDIEDLGFNAFCKKGENRDEVLRRLPCYDHTSSHVCTFPEFLKKEIKNDMSLHSRKYRWSSPVSVSELQGLLEVENGLSVKLVAGNTSTGYYKEEKERKYERFIDIRKIPEFTMVRSDEKGVELGACVTISKAIEVLREEKNVSVLAKIATHMEKIANRFVRNTGTIGGNIMMAQRKQFPSDLATILVAAQATVKIMTSSSSQEQFTLEEFLQQPPLDAKSLLLSLEIPSWHSAKKNGSSEDSILLFETYRAAPRPLGNALAFLNAAFSAEVTEALDGIVVNDCQLVFGAYGTKHAHRAKKVEEFLTGKVISDEVLMEAISLLKDEIVPDKGTSNPGYRSSLAVTFLFEFFGSLTKKNAKTTNGWLNGGCKEIGFDQNVESLKPEAMLSSAQQIVENQEHSPVGKGITKAGACLQASGEAVYVDDIPAPENCLYGAFIYSTMPLARIKGIRFKQNRVPEGVLGIITYKDIPKGGQNIGTNGFFTSDLLFAEEVTHCAGQIIAFLVADSQKHADIAANLVVIDYDTKDLKPPILSLEEAVENFSLFEVPPPLRGYPVGDITKGMDEAEHKILGSKISFGSQYFFYMETQTALAVPDEDNCMVVYSSTQTPEFVHQTIAGCLGVPENNVRVITRRVGGGFGGKAVKSMPVAAACALAASKMQRPVRTYVNRKTDMITTGGRHPMKVTYSVGFKSNGKITALDVEVLLDAGLTEDISPLMPKGIQGALMKYDWGALSFNVKVCKTNTVSRTALRAPGDVQGSYIGEAIIEKVASYLSVDVDEIRKVNLHTYESLRLFHSAKAGEFSEYTLPLLWDRIDEFSGFNKRRKVVEEFNASNKWRKRGISRVPAVYAVNMRSTPGRVSVLGDGSIVVEVQGIEIGQGLWTKVKQMAAYSLGLIQCGTTSDELLKKIRVIQSDTLSMVQGSMTAGSTTSEASSEAVRICCDGLVERLLPVKTALVEQTGGPVTWDSLISQAYQQSINMSVSSKYMPDSTGEYLNYGIAASEVEVNVLTGETTILRTDIIYDCGKSLNPAVDLGQIEGAFVQGLGFFMLEEFLMNSDGLVVTDSTWTYKIPTVDTIPRQFNVEILNSGQHKNRVLSSKASGEPPLLLAASVHCAVRAAVKEARKQILSWNSNKQGTDMYFELPVPATMPIVKEFCGLDVVEKYLEWKIQQRKNV

SEQ ID NO: 102 - protein, 513 aa, Saccharomyces cerevisiae
MTAGSAPPVDYTSLKKNFQPFLSRRVENRSLKSFWDASDISDDVIELAGGMPNERFFPIESMDLKISKVPFNDNPKWHNSFTTAHLDLGSPSELPIARSFQYAETKGLPPLLHFVKDFVSRINRPAFSDETESNWDVILSGGSNDSMFKVFETICDESTTVMIEEFTFTPAMSNVEATGAKVIPIKMNLTFDRESQGIDVEYLTQLLDNWSTGPYKDLNKPRVLYTIATGQNPTGMSVPQWKREKIYQLAQRHDFLIVEDDPYGYLYFPSYNPQEPLENPYHSSDLTTERYLNDFLMKSFLTLDTDARVIRLETFSKIFAPGLRLSFIVANKFLLQKILDLADITTRAPSGTSQAIVYSTIKAMAESNLSSSLSMKEAMFEGWIRWIMQIASKYNHRKNLTLKALYETESYQAGQFTVMEPSAGMFIIIKINWGNFDRPDDLPQQMDILDKFLLKNGVKVVLGYKMAVCPNYSKQNSDFLRLTIAYARDDDQLIEASKRIGSGIKEFFDNYKS

SEQ ID NO: 103 - protein, 396 aa, Escherichia coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 104 - protein, 391 aa, Arabidopsis thaliana
MVKLENSRKPEKISNKNIPMSDFVVNLDHGDPTAYEEYWRKMGDRCTVTIRGCDLMSYFSDMTNLCWFLEPELEDAIKDLHGVVGNAATEDRYIVVGTGSTQLCQAAVHALSSLARSQPVSVVAAAPFYSTYVEETTYVRSGMYKWEGDAWGFDKKGPYIELVTSPNNPDGTIRETVVNRPDDDEAKVIHDFAYYWPHYTPITRRQDHDIMLFTFSKITGHAGSRIGWALVKDKEVAKKMVEYIIVNSIGVSKESQVRTAKILNVLKETCKSESESENFFKYGREMMKNRWEKLREVVKESDAFTLPKYPEAFCNYFGKSLESYPAFAWLGTKEETDLVSELRRHKVMSRAGERCGSDKKHVRVSMLSREDVFNVFLERLANMKLIKSIDL

SEQ ID NO: 105 - protein, 504 aa, Streptomyces sp.
MTAPLQDSDGPDDAIGGPKQVTVIGAGIAGLVTAYELERLGHHVQIIEGSDDIGGRIHTHRFSGAGGPGPFAEMGAMRIPAGHRLTMHYIAELGLQNQVREFRTLFSDDAAYLPSSAGYLRVREAHDTLVDEFATGLPSAHYRQDTLLFGAWLDASIRAIAPRQFYDGLHNDIGVELLNLVDDIDLTPYRCGTARNRIDLHALFADHPRVRASCPPRLERFLDDVLDETSSSIVRLKDGMDELPRRLASRIRGKISLGQEVTGIDVHDDTVTLTVRQGLRTVTRTCDYVVCTIPFTVLRTLRLTGFDQDKLDIVHETKYWPATKIAFHCREPFWEKDGISGGASFTGGHVRQTYYPPAEGDPALGAVLLASYTIGPDAEALARMDEAERDALVAKELSVMHPELRRPGMVLAVAGRDWGARRWSRGAATVRWGQEAALREAERRECARPQKGLFFAGEHCSSKPAWIEGAIESAIDAAHEIEWYEPRASRVFAASRLSRSDRSA

SEQ ID NO: 106 - protein, 552 aa, Enterobacter cloacae
MRTPYCVADYLLDRLTDCGADHLFGVPGDYNLQFLDHVIDSPDICWVGCANELNASYAADGYARCKGFAALLTTFGVGELSAMNGIAGSYAEHVPVLHIVGAPGTAAQQRGELLHHTLGDGEFRHFYHMSEPITVAQAVLTEQNACYEIDRVLTTMLRERRPGYLMLPADVAKKAATPPVNALTHKQAHADSACLKAFRDAAENKLAMSKRTALLADFLVLRHGLKHALQKWVKEVPMAHATMLMGKGIFDERQAGFYGTYSGSASTGAVKEAIEGADTVLCVGTRFTDTLTAGFTHQLTPAQTIEVQPHAARVGDVWFTGIPMNQAIETLVELCKQHVHAGLMSSSSGAIPFPQPDGSLTQENFWRTLQTFIRPGDIILADQGTSAFGAIDLRLPADVNFIVQPLWGSIGYTLAAAFGAQTACPNRRVIVLTGDGAAQLTIQELGSMLRDKQHPIILVLNNEGYTVERAIHGAEQRYNDIALWNWTHIPQALSLDPQSECWRVSEAEQLADVLEKVAHHERLSLIEVMLPKADIPPLLGALTKALEACNNA

SEQ ID NO: 107 - protein, 497 aa, Ustilago maydis
MPTLNLDLPNGIKSTIQADLFINNKFVPALDGKTFATINPSTGKEIGQVAEASAKDVDLAVKAAREAFETTWGENTPGDARGRLLIKLAELVEANIDELAAIESLDNGKAFSIAKSFDVAAVAANLRYYGGWADKNHGKVMEVDTKRLNYTRHEPIGVCGQIIPWNFPLLMFAWKLGPALATGNTIVLKTAEQTPLSAIKMCELIVEAGFPPGVVNVISGFGPVAGAAISQHMDIDKIAFTGSTLVGRNIMKAAASTNLKKVTLELGGKSPNIIFKDADLDQAVRWSAFGIMFNHGQCCCAGSRVYVEESIYDAFMEKMTAHCKALQVGDPFSANTFQGPQVSQLQYDRIMEYIESGKKDANLALGGVRKGNEGYFIEPTIFTDVPHDAKIAKEEIFGPVVVVSKFKDEKDLIRIANDSIYGLAAAVFSRDISRAIETAHKLKAGTVWVNCYNQLIPQVPFGGYKASGIGRELGEYALSNYTNIKAVHVNLSQPAPI

SEQ ID NO: 108 - protein, 415 aa, Arabidopsis thaliana
MEFVTETLGKRIHDPYVEETRCLMIPGPIIVGSGPSGLATAACLKSRDIPSLILERSTCIASLWQHKTYDRLRLHLPKDFCELPLMPFPSSYPTYPTKQQFVQYLESYAEHFDLKPVFNQTVEEAKFDRRCGLWRVRTTGGKKDETMEYVSRWLVVATGENAEEVMPEIDGIPDFGGPILHTSSYKSGEIFSEKKILVVGCGNSGMEVCLDLCNFNALPSLVVRDSVHVLPQEMLGISTFGISTSLLKWFPVHVVDRFLLRMSRLVLGDTDRLGLVRPKLGPLERKIKCGKTPVLDVGTLAKIRSGHIKVYPELKRVMHYSAEFVDGRVDNFDAIILATGYKSNVPMWLKGVNMFSEKDGFPHKPFPNGWKGESGLYAVGFTKLGLLGAAIDAKKIAEDIEVQRHFLPLARPQHC

SEQ ID NO: 109 - protein, 557 aa, Pseudomonas savastanoi
MYDHFNSPSIDILYDYGPFLKKCEMTGGIGSYSAGTPTPRVAIVGAGISGLVAATELLRAGVKDVVLYESRDRIGGRVWSQVFDQTRPRYIAEMGAMRFPPSATGLFHYLKKFGISTSTTFPDPGVVDTELHYRGKRYHWPAGKKPPELFRRVYEGWQSLLSEGYLLEGGSLVAPLDITAMLKSGRLEEAAIAWQGWLNVFRDCSFYNAIVCIFTGRHPPGGDRWARPEDFELFGSLGIGSGGFLPVFQAGFTEILRMVINGYQSDQRLIPDGISSLAARLADQSFDGKALRDRVCFSRVGRISREAEKIIIQTEAGEQRVFDRVIVTSSNRAMQMIHCLTDSESFLSRDVARAVRETHLTGSSKLFILTRTKFWIKNKLPTTIQSDGLVRGVYCLDYQPDEPEGHGVVLLSYTWEDDAQKMLAMPDKKTRCQVLVDDLAAIHPTFASYLLPVDGDYERYVLHHDWLTDPHSAGAFKLNYPGEDVYSQRLFFQPMTANSPNKDTGLYLAGCSCSFAGGWIEGAVQTALNSACAVLRSTGGQLSKGNPLDCINASYRY

SEQ ID NO: 110 - protein, 455 aa, Pseudomonas savastanoi
MHEIITLESLCQALADGEIAAAELRERALDTEARLARLNCFIREGDAVSQFGEADHAMKGTPLWGMPVSFKDNICVRGLPLTAGTRGMSGFVSDQDAAIVSQLRALGAVVAGKNNMHELSFGVTSINPHWGTVGNPVAPGYCAGGSSGGSAAAVASGIVPLSVGTDTGGSIRIPAAFCGITGFRPTTGRWSTAGIIPVSHTKDCVGLLTRTAGDAGFLYGLLSGKQQSFPLSRTAPCRIGLPVSMWSDLDGEVERACVNALSLLRKTGFEFIEIDDADIVELNQTLTFTVPLYEFFADLAQSLLSLGWKHGIHHIFAQVDDANVKGIINHHLGEGAIKPAHYLSSLQNGELLKRKMDELFARHNIELLGYPTVPCRVPHLDHADRPEFFSQAIRNTDLASNAMLPSITIPVGPEGRLPVGLSFDALRGRDALLLSRVSAIEQVLGFVRKVLPHTT

SEQ ID NO: 111 - protein, 353 aa, Nostoc punctiforme
MLLFETVREMGHEQVLFCHSKNPEIKAIIAIHDTTLGPAMGATRILPYINEEAALKDALRLSRGMTYKAACANIPAGGGKAVIIANPENKTDDLLRAYGRFVDSLNGRFITGQDVNITPDDVRTISQETKYVVGVSEKSGGPAPITSLGVFLGIKAAVESRWQSKRLDGMKVAVQGLGNVGKNLCRHLHEHDVQLFVSDVDPIKAEEVKRLFGATVVEPTEIYSLDVDIFAPCALGGILNSHTIPFLQASIIAGAANNQLENEQLHSQMLAKKGILYSPDYVINAGGLINVYNEMIGYDEEKAFKQVHNIYDTLLAIFEIAKEQGVTTNDAARRLAEDRINNSKRSKSKAIAA

SEQ ID NO: 112 - protein, 541 aa, Arabidopsis thaliana
MNTFTSNSSDLTTTATETSSFSTLYLLSTLQAFVAITLVMLLKKLMTDPNKKKPYLPPGPTGWPIIGMIPTMLKSRPVFRWLHSIMKQLNTEIACVKLGNTHVITVTCPKIAREILKQQDALFASRPLTYAQKILSNGYKTCVITPFGDQFKKMRKVVMTELVCPARHRWLHQKRSEENDHLTAWVYNMVKNSGSVDFRFMTRHYCGNAIKKLMFGTRTFSKNTAPDGGPTVEDVEHMEAMFEALGFTFAFCISDYLPMLTGLDLNGHEKIMRESSAIMDKYHDPIIDERIKMWREGKRTQIEDFLDIFISIKDEQGNPLLTADEIKPTIKELVMAAPDNPSNAVEWAMAEMVNKPEILRKAMEEIDRVVGKERLVQESDIPKLNYVKAILREAFRLHPVAAFNLPHVALSDTTVAGYHIPKGSQVLLSRYGLGRNPKVWADPLCFKPERHLNECSEVTLTENDLRFISFSTGKRGCAAPALGTALTTMMLARLLQGFTWKLPENETRVELMESSHDMFLAKPLVMVGDLRLPEHLYPTVK

SEQ ID NO: 113 - protein, 543 aa, Arabidopsis thaliana
MDTLASNSSDLTTKSSLGMSSFTNMYLLTTLQALAALCFLMILNKIKSSSRNKKLHPLPPGPTGFPIVGMIPAMLKNRPVFRWLHSLMKELNTEIACVRLGNTHVIPVTCPKIAREIFKQQDALFASRPLTYAQKILSNGYKTCVITPFGEQFKKMRKVIMTEIVCPARHRWLHDNRAEETDHLTAWLYNMVKNSEPVDLRFVTRHYCGNAIKRLMFGTRTFSEKTEADGGPTLEDIEHMDAMFEGLGFTFAFCISDYLPMLTGLDLNGHEKIMRESSAIMDKYHDPIIDERIKMWREGKRTQIEDFLDIFISIKDEAGQPLLTADEIKPTIKELVMAAPDNPSNAVEWAIAEMINKPEILHKAMEEIDRVVGKERFVQESDIPKLNYVKAIIREAFRLHPVAAFNLPHVALSDTTVAGYHIPKGSQVLLSRYGLGRNPKVWSDPLSFKPERHLNECSEVTLTENDLRFISFSTGKRGCAAPALGTAITTMMLARLLQGFKWKLAGSETRVELMESSHDMFLSKPLVLVGELRLSEDLYPMVK

SEQ ID NO: 114 - protein, 503 aa, Arabidopsis thaliana
MSNIQEMEMILSISLCLTTLITLLLLRRFLKRTATKVNLPPSPWRLPVIGNLHQLSLHPHRSLRSLSLRYGPLMLLHFGRVPILVVSSGEAAQEVLKTHDHKFANRPRSKAVHGLMNGGRDVVFAPYGEYWRQMKSVCILNLLTNKMVESFEKVREDEVNAMIEKLEKASSSSSSENLSELFITLPSDVTSRVALGRKHSEDETARDLKKRVRQIMELLGEFPIGEYVPILAWIDGIRGFNNKIKEVSRGFSDLMDKVVQEHLEASNDKADFVDILLSIEKDKNSGFQVQRNDIKFMILDMFIGGTSTTSTLLEWTMTELIRSPKSMKKLQDEIRSTIRPHGSYIKEKEVENMKYLKAVIKEVLRLHPSLPMILPRLLSEDVKVKGYNIAAGTEVIINAWAIQRDTAIWGPDAEEFKPERHLDSGLDYHGKNLNYIPFGSGRRICPGINLALGLAEVTVANLVGRFDWRVEAGPNGDQPDLTEAIGIDVCRKFPLIAFPSSVV

SEQ ID NO: 115 - protein, 560 aa, Arabidopsis thaliana
MAHLQRTFPTEMSKGRASFPKGFLFGTASSSYQYEGAVNEGARGQSVWDHFSNRFPHRISDSSDGNVAVDFYHRYKEDIKRMKDINMDSFRLSIAWPRVLPYGKRDRGVSEEGIKFYNDVIDELLANEITPLVTIFHWDIPQDLEDEYGGFLSEQIIDDFRDYASLCFERFGDRVSLWCTMNEPWVYSVAGYDTGRKAPGRCSKYVNGASVAGMSGYEAYIVSHNMLLAHAEAVEVFRKCDHIKNGQIGIAHNPLWYEPYDPSDPDDVEGCNRAMDFMLGWHQHPTACGDYPETMKKSVGDRLPSFTPEQSKKLIGSCDYVGINYYSSLFVKSIKHVDPTQPTWRTDQGVDWMKTNIDGKQIAKQGGSEWSFTYPTGLRNILKYVKKTYGNPPILITENGYGEVAEQSQSLYMYNPSIDTERLEYIEGHIHAIHQAIHEDGVRVEGYYVWSLLDNFEWNSGYGVRYGLYYIDYKDGLRRYPKMSALWLKEFLRFDQEDDSSTSKKEEKKESYGKQLLHSVQDSQFVHSIKDSGALPAVLGSLFVVSATVGTSLFFKGANN

SEQ ID NO: 116 - protein, 346 aa, Arabidopsis thaliana
MSSTKDMSTVQNATPFNGVAPSTTVRVTIVQSSTVYNDTPATIDKAEKYIVEAASKGAELVLFPEGFIGGYPRGFRFGLAVGVHNEEGRDEFRKYHASAIHVPGPEVARLADVARKNHVYLVMGAIEKEGYTLYCTVLFFSPQGQFLGKHRKLMPTSLERCIWGQGDGSTIPVYDTPIGKLGAAICWENRMPLYRTALYAKGIELYCAPTADGSKEWQSSMLHIAIEGGCFVLSACQFCQRKHFPDHPDYLFTDWYDDKEHDSIVSQGGSVIISPLGQVLAGPNFESEGLVTADIDLGDIARAKLYFDSVGHYSRPDVLHLTVNEHPRKSVTFVTKVEKAEDDSNK

SEQ ID NO: 117 - protein, 403 aa, Homo sapiens [sequence omitted]

SEQ ID NO: 118 - protein, 406 aa, Homo sapiens
MSGCPFLGNNFGYTFKKLPVEGSEEDKSQTGVNRASKGGLIYGNYLHLEKVLNAQELQSETKGNKIHDEHLFIITHQAYELWFKQILWELDSVREIFQNGHVRDERNMLKVVSRMHRVSVILKLLVQQFSILETMTALDFNDFREYLSPASGFQSLQFRLLENKIGVLQNMRVPYNRRHYRDNFKGEENELLLKSEQEKTLLELVEAWLERTPGLEPHGFNFWGKLEKNITRGLEEEFIRIQAKEESEEKEEQVAEFQKQKEVLLSLFDEKRHEHLLSKGERRLSYRALQGALMIYFYREEPRFQVPFQLLTSLMDIDSLMTKWRYNHVCMVHRMLGSKAGTGGSSGYHYLRSTVSDRYKVFVDLFNLSTYLIPRHWIPKMNPTIHKFLYTAEYCDSSYFSSDESD

SEQ ID NO: 119 - protein, 453 aa, Saccharomyces cerevisiae
MNNTSITGPQVLHRTKMRPLPVLEKYCISPHHGFLDDRLPLTRLSSKKYMKWEEIVADLPSLLQEDNKVRSVIDGLDVLDLDETILGDVRELRRAYSILGFMAHAYIWASGTPRDVLPECIARPLLETAHILGVPPLATYSSLVLWNFKVTDECKKTETGCLDLENITTINTFTGTVDESWFYLVSVRFEKIGSACLNHGLQILRAIRSGDKGDANVIDGLEGLAATIERLSKALMEMELKCEPNVFYFKIRPFLAGWTNMSHMGLPQGVRYGAEGQYRIFSGGSNAQSSLIQTLDILLGVKHTANAAHSSQGDSKINYLDEMKKYMPREHREFLYHLESVCNIREYVSRNASNRALQEAYGRCISMLKIFRDNHIQIVTKYIILPSNSKQHGSNKPNVLSPIEPNTKASGCLGHKVASSKTIGTGGTRLMPFLKQCRDETVATADIKNEDKN

SEQ ID NO: 120 - protein, 305 aa, Mus musculus
MAFPSLSAGQNPWRNLSSEELEKQYSPSRWVIHTKPEEVVGNFVQIGSQATQKARATRRNQLDVPYGDGEGEKLDIYFPDEDSKAFPLFLFLHGGYWQSGSKDDSAFMVNPLTAQGIVVVIVAYDIAPKGTLDQMVDQVTRSVVFLQRRYPSNEGIYLCGHSAGAHLAAMVLLARWTKHGVTPNLQGFLLVSGIYDLEPLIATSQNDPLRMTLEDAQRNSPQRHLDVVPAQPVAPACPVLVLVGQHDSPEFHRQSKEFYETLLRVGWKASFQQLRGVDHFDIIENLTREDDVLTQIILKTVFQKL

SEQ ID NO: 121 - protein, 444 aa, Saccharomyces cerevisiae
MKQRFIRQFTNLMSTSRPKVVANKYFTSNTAKDVWSLTNEAAAKAANNSKNQGRELINLGQGFFSYSPPQFAIKEAQKALDIPMVNQYSPTRGRPSLINSLIKLYSPIYNTELKAENVTVTTGANEGILSCLMGLLNAGDEVIVFEPFFDQYIPNIELCGGKVVYVPINPPKELDQRNTRGEEWTIDFEQFEKAITSKTKAVIINTPHNPIGKVFTREELTTLGNICVKHNVVIISDEVYEHLYFTDSFTRIATLSPEIGQLTLTVGSAGKSFAATGWRIGWVLSLNAELLSYAAKAHTRICFASPSPLQEACANSINDALKIGYFEKMRQEYINKFKIFTSIFDELGLPYTAPEGTYFVLVDFSKVKIPEDYPYPEEILNKGKDFRISHWLINELGVVAIPPTEFYIKEHEKAAENLLRFAVCKDDAYLENAVERLKLLKDYL

SEQ ID NO: 122 - protein, 430 aa, Homo sapiens
MALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 123 - protein, 425 aa, unidentified
MNYARFITAASAARNPSPIRTMTDILSRGPKSMISLAGGLPNPNMFPFKTAVITVENGKTIQFGEEMMKRALQYSPSAGIPELLSWLKQLQIKLHNPPTIHYPPSQGQMDLCVTSGSQQGLCKVFEMIINPGDNVLLDEPAYSGTLQSLHPLGCNIINVASDESGIVPDSLRDILSRWKPEDAKNPQKNTPKFLYTVPNGNNPTGNSLTSERKKEIYELARKYDFLIIEDDPYYFLQFNKFRVPTFLSMDVDGRVIRADSFSKIISSGLRIGFLTGPKPLIERVILHIQVSTLHPSTFNQLMISQLLHEWGEEGFMAHVDRVIDFYSNQKDAILAAADKWLTGLAEWHVPAAGMFLWIKVKGINDVKELIEEKAVKMGVLMLPGNAFYVDSSAPSPYLRASFSSASPEQMDVAFQVLAQLIKESL

SEQ ID NO: 124 - protein, 422 aa, Homo sapiens
MAKQLQARRLDGIDYNPWVEFVKLASEHDVVNLGQGFPDFPPPDFAVEAFQHAVSGDFMLNQYTKTFGYPPLTKILASFFGELLGQEIDPLRNVLVTVGGYGALFTAFQALVDEGDEVIIIEPFFDCYEPMTMMAGGRPVFVSLKPGPIQNGELGSSSNWQLDPMELAGKFTSRTKALVLNTPNNPLGKVFSREELELVASLCQQHDVVCITDEVYQWMVYDGHQHISIASLPGMWERTLTIGSAGKTFSATGWKVGWVLGPDHIMKHLRTVHQNSVFHCPTQSQAAVAESFEREQLLFRQPSSYFVQFPQAMQRCRDHMIRSLQSVGLKPIIPQGSYFLITDISDFKRKMPDLPGAVDEPYDRRFVKWMIKNKGLVAIPVSIFYSVPHQKHFDHYIRFCFVKDEATLQAMDEKLRKWKVEL

SEQ ID NO: 125 - protein, 454 aa, Homo sapiens
MFLAQRSLCSLSGRAKFLKTISSSKILGFSTSAKMSLKFTNAKRIEGLDSNVWIEFTKLAADPSVVNLGQGFPDISPPTYVKEELSKIAAIDSLNQYTRGFGHPSLVKALSYLYEKLYQKQIDSNKEILVTVGAYGSLFNTIQALIDEGDEVILIVPFYDCYEPMVRMAGATPVFIPLRSKPVYGKRWSSSDWTLDPQELESKFNSKTKAIILNTPHNPLGKVYNREELQVIADLCIKYDTLCISDEVYEWLVYSGNKHLKIATFPGMWERTITIGSAGKTFSVTGWKLGWSIGPNHLIKHLQTVQQNTIYTCATPLQEALAQAFWIDIKRMDDPECYFNSLPKELEVKRDRMVRLLESVGLKPIVPDGGYFIIADVSLLDPDLSDMKNNEPYDYKFVKWMTKHKKLSAIPVSAFCNSETKSQFEKFVRFCFIKKDSTLDAAEEIIKAWSVQKS

SEQ ID NO: 126 - protein, 471 aa, Escherichia coli
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEV

SEQ ID NO: 127 - protein, 412 aa, Clostridium sporogenes
MENNTNMFSGVKVIELANFIAAPAAGRFFADGGAEVIKIESPAGDPLRYTAPSEGRPLSQEENTTYDLENANKKAIVLNLKSEKGKKILHEMLAEADILLTNWRTKALVKQGLDYETLKEKYPKLVFAQITGYGEKGPDKDLPGFDYTAFFARGGVSGTLYEKGTVPPNVVPGLGDHQAGMFLAAGMAGALYKAKTTGQGDKVTVSLMHSAMYGLGIMIQAAQYKDHGLVYPINRNETPNPFIVSYKSKDDYFVQVCMPPYDVFYDRFMTALGREDLVGDERYNKIENLKDGRAKEVYSIIEQQMVTKTKDEWDKIFRDADIPFAIAQTWEDLLEDEQAWANDYLYKMKYPTGNERALVRLPVFFKEAGLPEYNQSPQIAENTVEVLKEMGYTEQEIEELEKDKDIMVRKEK

SEQ ID NO: 128 - protein, 407 aa, Clostridium sporogenes
MSDRNKEVKEKKAKHYLREITAKHYKEALEAKERGEKVGWCASNFPQEIATTLGVKVVYPENHAAAVAARGNGQNMCEHAEAMGFSNDVCGYARVNLAVMDIGHSEDQPIPMPDFVLCCNNICNQMIKWYEHIAKTLDIPMILIDIPYNTENTVSQDRIKYIRAQFDDAIKQLEEITGKKWDENKFEEVMKISQESAKQWLRAASYAKYKPSPFSGFDLFNHMAVAVCARGTQEAADAFKMLADEYEENVKTGKSTYRGEEKQRILFEGIACWPYLRHKLTKLSEYGMNVTATVYAEAFGVIYENMDELMAAYNKVPNSISFENALKMRLNAVTSTNTEGAVIHINRSCKLWSGFLYELARRLEKETGIPVVSFDGDQADPRNFSEAQYDTRIQGLNEVMVAKKEAE

SEQ ID NO: 129 - protein, 374 aa, Clostridium sporogenes
MSNSDKFFNDFKDIVENPKKYIMKHMEQTGQKAIGCMPLYTPEELVLAAGMFPVGVWGSNTELSKAKTYFPAFICSILQTTLENALNGEYDMLSGMMITNYCDSLKCMGQNFKLTVENIEFIPVTVPQNRKMEAGKEFLKSQYKMNIEQLEKISGNKITDESLEKAIEIYDEHRKVMNDFSMLASKYPGIITPTKRNYVMKSAYYMDKKEHTEKVRQLMDEIKAIEPKPFEGKRVITTGIIADSEDLLKILEENNIAIVGDDIAHESRQYRTLTPEANTPMDRLAEQFANRECSTLYDPEKKRGQYIVEMAKERKADGIIFFMTKFCDPEEYDYPQMKKDFEEAGIPHVLIETDMQMKNYEQARTAIQAFSETL

SEQ ID NO: 130 - protein, 377 aa, Clostridium sporogenes
MFFTEQHELIRKLARDFAEQEIEPIADEVDKTAEFPKEIVKKMAQNGFFGIKMPKEYGGAGADNRAYVTIMEEISRASGVAGIYLSSPNSLLGTPFLLVGTDEQKEKYLKPMIRGEKTLAFALTEPGAGSDAGALATTAREEGDYYILNGRKTFITGAPISDNIIVFAKTDMSKGTKGITTFIVDSKQEGVSFGKPEDKMGMIGCPTSDIILENVKVHKSDILGEVNKGFITAMKTLSVGRIGVASQALGIAQAAVDEAVKYAKQRKQFNRPIAKFQAIQFKLANMETKLNAAKLLVYNAAYKMDCGEKADKEASMAKYFAAESAIQIVNDALQIHGGYGYIKDYKIERLYRDVRVIAIYEGTSEVQQMVIASNLLK

SEQ ID NO: 131 - protein, 331 aa, Clostridium sporogenes
MKILAYCVRPDEVDSFKKFSEKYGHTVDLIPDSFGPNVAHLAKGYDGISILGNDTCNREALEKIKDCGIKYLATRTAGVNNIDFDAAKEFGINVANVPAYSPNSVSEFTIGLALSLTRKIPFALKRVELNNFALGGLIGVELRNLTLGVIGTGRIGLKVIEGFSGFGMKKMIGYDIFENEEAKKYIEYKSLDEVFKEADIITLHAPLTDDNYHMIGKESIAKMKDGVFIINAARGALIDSEALIEGLKSGKIAGAALDSYEYEQGVFHNNKMNEIMQDDTLERLKSFPNVVITPHLGFYTDEAVSNMVEITLMNLQEFELKGTCKNQRVCK

SEQ ID NO: 132 - protein, 334 aa, Clostridium sporogenes
MKILMYSVREHEKPAIKKWLEANPGVQIDLCNNALSEDTVCKAKEYDGIAIQQTNSIGGKAVYSTLKEYGIKQIASRTAGVDMIDLKMASDSNILVTNVPAYSPNAIAELAVTHTMNLLRNIKTLNKRIAYGDYRWSADLIAREVRSVTVGVVGTGKIGRTSAKLFKGLGANVIGYDAYPDKKLEENNLLTYKESLEDLLREADVVTLHTPLLESTKYMINKNNLKYMKPDAFIVNTGRGGIINTEDLIEALEQNKIAGAALDTFENEGLFLNKVVDPTKLPDSQLDKLLKMDQVLITHHVGFFTTTAVQNIVDTSLDSVVEVLKTNNSVNKVN

SEQ ID NO: 133 - protein, 326 aa, Rhodobacter sphaeroides
MRAVLIEKSDDTQSVSVTELAEDQLPEGDVLVDVAYSTLNYKDALAITGKAPVVRRFPMVPGIDFTGTVAQSSHADFKPGDRVILNGWGVGEKHWGGLAERARVRGDWLVPLPAPLDLRQAAMIGTAGYTAMLCVLALERHGVVPGNGEIVVSGAAGGVGSVATTLLAAKGYEVAAVTGRASEAEYLRGLGAASVIDRNELTGKVRPLGQERWAGGIDVAGSTVLANMLSMMKYRGVVAACGLAAGMDLPASVAPFILRGMTLAGVDSVMCPKTDRLAAWARLASDLDPAKLEEMTTELPFSEVIETAPKFLDGTVRGRIVIPVTP

SEQ ID NO: 134-151 - skipped (18 entries)

SEQ ID NO: 152 - DNA, 30 bp, synthetic construct
  source 1..30: mol_type = other DNA
gtttatacataggcgagtactctgttatgg

SEQ ID NO: 153 - DNA, 30 bp, synthetic construct
  source 1..30: mol_type = other DNA
agaggttccaactttcaccataatgaaaca

SEQ ID NO: 154 - DNA, 30 bp, synthetic construct
  source 1..30: mol_type = other DNA
taaacaactaacggacaattctacctaaca

SEQ ID NO: 155 - DNA, 30 bp, synthetic construct
  source 1..30: mol_type = other DNA
acatcaagccaaattaaacaggattaacac

SEQ ID NO: 156 - DNA, 30 bp, synthetic construct
  source 1..30: mol_type = other DNA
gaggtaaaatagtcaacacgcacggtgtta

SEQ ID NO: 157 - DNA, 30 bp, synthetic construct
  source 1..30: mol_type = other DNA
caggccggaataactccctataatgcgcca

SEQ ID NO: 158 - DNA, 30 bp, synthetic construct
  source 1..30: mol_type = other DNA
ggctagctcagtcctaggtacagtgctagc

SEQ ID NO: 159 - DNA, 30 bp, synthetic construct
  source 1..30: mol_type = other DNA
agctagctcagtcctaggtattatgctagc

SEQ ID NO: 160 - DNA, 30 bp, synthetic construct
  source 1..30: mol_type = other DNA
agctagctcagtcctaggtactgtgctagc

SEQ ID NO: 161 - DNA, 30 bp, synthetic construct
  source 1..30: mol_type = other DNA
agctagctcagtcctagggattatgctagc

SEQ ID NO: 162 - DNA, 30 bp, synthetic construct
  source 1..30: mol_type = other DNA
agctagctcagtcctaggtattgtgctagc

SEQ ID NO: 163 - DNA, 30 bp, synthetic construct
  source 1..30: mol_type = other DNA
ggctagctcagtcctaggtactatgctagc

SEQ ID NO: 164 - DNA, 30 bp, synthetic construct
  source 1..30: mol_type = other DNA
ggctagctcagtcctaggtatagtgctagc

SEQ ID NO: 165 - DNA, 30 bp, synthetic construct
  source 1..30: mol_type = other DNA
ggctagctcagccctaggtattatgctagc

SEQ ID NO: 166 - DNA, 30 bp, synthetic construct
  source 1..30: mol_type = other DNA
agctagctcagtcctaggtataatgctagc

SEQ ID NO: 167 - DNA, 30 bp, synthetic construct
  source 1..30: mol_type = other DNA
agctagctcagtcctagggactgtgctagc

SEQ ID NO: 168 - DNA, 30 bp, synthetic construct
  source 1..30: mol_type = other DNA
ggctagctcagtcctaggtacaatgctagc

SEQ ID NO: 169 - DNA, 30 bp, synthetic construct; identical to SEQ ID NO: 164
  source 1..30: mol_type = other DNA

SEQ ID NO: 170 - DNA, 30 bp, synthetic construct; identical to SEQ ID NO: 161
  source 1..30: mol_type = other DNA

SEQ ID NO: 171 - DNA, 30 bp, synthetic construct
  source 1..30: mol_type = other DNA
ggctagctcagtcctagggattatgctagc

SEQ ID NO: 172 - DNA, 30 bp, synthetic construct; identical to SEQ ID NO: 168
  source 1..30: mol_type = other DNA

SEQ ID NO: 173 - DNA, 30 bp, synthetic construct
  source 1..30: mol_type = other DNA
agctagctcagcccttggtacaatgctagc

SEQ ID NO: 174 - DNA, 30 bp, synthetic construct
  source 1..30: mol_type = other DNA
agctagctcagtcctagggactatgctagc

SEQ ID NO: 175 - DNA, 30 bp, synthetic construct
  source 1..30: mol_type = other DNA
agctagctcagtcctagggattgtgctagc

SEQ ID NO: 176 - DNA, 30 bp, synthetic construct
  source 1..30: mol_type = other DNA
ggctagctcagtcctaggtattgtgctagc

SEQ ID NO: 177 - DNA, 30 bp, synthetic construct; identical to SEQ ID NO: 166
  source 1..30: mol_type = other DNA

SEQ ID NO: 178 - DNA, 30 bp, synthetic construct
  source 1..30: mol_type = other DNA
ggctagctcagtcctaggtattatgctagc

SEQ ID NO: 179 - DNA, 30 bp, synthetic construct; identical to SEQ ID NO: 168
  source 1..30: mol_type = other DNA

SEQ ID NO: 180 - DNA, 30 bp, synthetic construct
  source 1..30: mol_type = other DNA
aaagtgtgacgccgtgcaaataatcaatgt

SEQ ID NO: 181 - DNA, 30 bp, synthetic construct
  source 1..30: mol_type = other DNA
gacgaatacttaaaatcgtcatacttattt

SEQ ID NO: 182 - DNA, 30 bp, synthetic construct
  source 1..30: mol_type = other DNA
aaacctttcgcggtatggcatgatagcgcc

SEQ ID NO: 183 - DNA, 30 bp, synthetic construct
  source 1..30: mol_type = other DNA
tgatagcgcccggaagagagtcaattcagg

SEQ ID NO: 184 - DNA, 30 bp, synthetic construct
  source 1..30: mol_type = other DNA
ttatttaccgtgacgaactaattgctcgtg

SEQ ID NO: 185 - DNA, 30 bp, synthetic construct
  source 1..30: mol_type = other DNA
catacgccgttatacgttgtttacgctttg

SEQ ID NO: 186 - DNA, 30 bp, synthetic construct
  source 1..30: mol_type = other DNA
ttatgcttccggctcgtatgttgtgtggac

SEQ ID NO: 187 - DNA, 30 bp, synthetic construct
  source 1..30: mol_type = other DNA
ttatgcttccggctcgtatggtgtgtggac

SEQ ID NO: 188 - DNA, 30 bp, synthetic construct; identical to SEQ ID NO: 163
  source 1..30: mol_type = other DNA

SEQ ID NO: 189 - DNA, 30 bp, synthetic construct
  source 1..30: mol_type = other DNA
atatatatatatatataatggaagcgtttt

SEQ ID NO: 190 - DNA, 30 bp, synthetic construct; identical to SEQ ID NO: 189
  source 1..30: mol_type = other DNA

SEQ ID NO: 191 - DNA, 30 bp, synthetic construct
  source 1..30: mol_type = other DNA
ccccgaaagcttaagaatataattgtaagc

SEQ ID NO: 192 - DNA, 30 bp, synthetic construct; identical to SEQ ID NO: 191
  source 1..30: mol_type = other DNA

SEQ ID NO: 193 - DNA, 30 bp, synthetic construct
  source 1..30: mol_type = other DNA
tgacaatatatatatatatataatgctagc

SEQ ID NO: 194 - DNA, 30 bp, synthetic construct
  source 1..30: mol_type = other DNA
acaatatatatatatatatataatgctagc

SEQ ID NO: 195 - DNA, 30 bp, synthetic construct
  source 1..30: mol_type = other DNA
aatatatatatatatatatataatgctagc

SEQ ID NO: 196 - DNA, 30 bp, synthetic construct
  source 1..30: mol_type = other DNA
tatatatatatatatatatataatgctagc

SEQ ID NO: 197 - DNA, 30 bp, synthetic construct; identical to SEQ ID NO: 196
  source 1..30: mol_type = other DNA

SEQ ID NO: 198 - DNA, 30 bp, synthetic construct
  source 1..30: mol_type = other DNA
aaaaaaaaaaaaaaaaaatataatgctagc

SEQ ID NO: 199 - DNA, 30 bp, synthetic construct; identical to SEQ ID NO: 198
  source 1..30: mol_type = other DNA

SEQ ID NO: 200 - DNA, 30 bp, synthetic construct
  source 1..30: mol_type = other DNA
ggaattgtgagcggataacaatttcacaca

SEQ ID NO: 201 - DNA, 30 bp, synthetic construct; identical to SEQ ID NO: 200
  source 1..30: mol_type = other DNA

SEQ ID NO: 202 - DNA, 30 bp, synthetic construct; identical to SEQ ID NO: 200
  source 1..30: mol_type = other DNA

SEQ ID NO: 203 - DNA, 30 bp, synthetic construct; identical to SEQ ID NO: 200
  source 1..30: mol_type = other DNA

SEQ ID NO: 204 - DNA, 30 bp, synthetic construct; identical to SEQ ID NO: 200
  source 1..30: mol_type = other DNA

SEQ ID NO: 205 - DNA, 30 bp, synthetic construct; identical to SEQ ID NO: 200
  source 1..30: mol_type = other DNA

SEQ ID NO: 206 - DNA, 30 bp, synthetic construct; identical to SEQ ID NO: 200
  source 1..30: mol_type = other DNA

SEQ ID NO: 207 - DNA, 30 bp, synthetic construct; identical to SEQ ID NO: 200
  source 1..30: mol_type = other DNA

SEQ ID NO: 208 - DNA, 30 bp, synthetic construct; identical to SEQ ID NO: 200
  source 1..30: mol_type = other DNA

SEQ ID NO: 209 - DNA, 30 bp, synthetic construct; identical to SEQ ID NO: 200
  source 1..30: mol_type = other DNA

SEQ ID NO: 210 - DNA, 30 bp, synthetic construct; identical to SEQ ID NO: 200
  source 1..30: mol_type = other DNA

SEQ ID NO: 211 - DNA, 30 bp, synthetic construct; identical to SEQ ID NO: 200
  source 1..30: mol_type = other DNA

SEQ ID NO: 212 - DNA, 30 bp, synthetic construct; identical to SEQ ID NO: 200
  source 1..30: mol_type = other DNA

SEQ ID NO: 213 - DNA, 30 bp, synthetic construct; identical to SEQ ID NO: 200
  source 1..30: mol_type = other DNA

SEQ ID NO: 214 - DNA, 30 bp, synthetic construct
  source 1..30: mol_type = other DNA
gattaaagaggagaaatactagagtactag

SEQ ID NO: 215 - DNA, 30 bp, synthetic construct
  source 1..30: mol_type = other DNA
caccttcgggtgggcctttctgcgtttata

SEQ ID NO: 216 - DNA, 30 bp, synthetic construct; identical to SEQ ID NO: 215
  source 1..30: mol_type = other DNA

SEQ ID NO: 217 - DNA, 30 bp, synthetic construct; identical to SEQ ID NO: 215
  source 1..30: mol_type = other DNA

SEQ ID NO: 218 - DNA, 30 bp, synthetic construct; identical to SEQ ID NO: 215
  source 1..30: mol_type = other DNA

SEQ ID NO: 219 - DNA, 30 bp, synthetic construct; identical to SEQ ID NO: 158
  source 1..30: mol_type = other DNA

SEQ ID NO: 220 - DNA, 30 bp, synthetic construct
  source 1..30: mol_type = other DNA
tgctagctactagagattaaagaggagaaa

SEQ ID NO: 221 - DNA, 30 bp, synthetic construct
  source 1..30: mol_type = other DNA
ttgtgagcggataacaagatactgagcaca

SEQ ID NO: 222 - DNA, 30 bp, synthetic construct; identical to SEQ ID NO: 221
  source 1..30: mol_type = other DNA

SEQ ID NO: 223 - DNA, 30 bp, synthetic construct; identical to SEQ ID NO: 221
  source 1..30: mol_type = other DNA

SEQ ID NO: 224 - DNA, 30 bp, synthetic construct; identical to SEQ ID NO: 158
  source 1..30: mol_type = other DNA

SEQ ID NO: 225 - DNA, 30 bp, synthetic construct; identical to SEQ ID NO: 159
  source 1..30: mol_type = other DNA

SEQ ID NO: 226 - DNA, 30 bp, synthetic construct; identical to SEQ ID NO: 160
  source 1..30: mol_type = other DNA

SEQ ID NO: 227 - DNA, 30 bp, synthetic construct; identical to SEQ ID NO: 161
  source 1..30: mol_type = other DNA

SEQ ID NO: 228 - DNA, 30 bp, synthetic construct; identical to SEQ ID NO: 164
  source 1..30: mol_type = other DNA

SEQ ID NO: 229 - DNA, 30 bp, synthetic construct; identical to SEQ ID NO: 171
  source 1..30: mol_type = other DNA

SEQ ID NO: 230 - DNA, 30 bp, synthetic construct; identical to SEQ ID NO: 168
  source 1..30: mol_type = other DNA

SEQ ID NO: 231 - DNA, 30 bp, synthetic construct; identical to SEQ ID NO: 175
  source 1..30: mol_type = other DNA

SEQ ID NO: 232 - DNA, 30 bp, synthetic construct; identical to SEQ ID NO: 176
  source 1..30: mol_type = other DNA

SEQ ID NO: 233 - DNA, 30 bp, synthetic construct
  source 1..30: mol_type = other DNA
cctgtttttatgttattctctctgtaaagg

SEQ ID NO: 234 - DNA, 30 bp, synthetic construct
  source 1..30: mol_type = other DNA
aaatatttgcttatacaatcttcctgtttt

SEQ ID NO: 235 - DNA, 30 bp, synthetic construct
  source 1..30: mol_type = other DNA
gctgataaaccgatacaattaaaggctcct

SEQ ID NO: 236 - DNA, 30 bp, synthetic construct
  source 1..30: mol_type = other DNA
ctcttctcagcgtcttaatctaagctatcg

SEQ ID NO: 237 - DNA, 30 bp, synthetic construct
  source 1..30: mol_type = other DNA
atgagccagttcttaaaatcgcataaggta

SEQ ID NO: 238 - DNA, 30 bp, synthetic construct
  source 1..30: mol_type = other DNA
ctattgattgtgacaaaataaacttattcc

SEQ ID NO: 239 - DNA, 30 bp, synthetic construct
  source 1..30: mol_type = other DNA
gtttcgcgcttggtataatcgctgggggtc

SEQ ID NO: 240 - DNA, 30 bp, synthetic construct
  source 1..30: mol_type = other DNA
ctttgcttctgactataatagtcagggtaa

SEQ ID NO: 241 - DNA, 30 bp, synthetic construct
  source 1..30: mol_type = other DNA
aaaccgatacaattaaaggctcctgctagc

SEQ ID NO: 242 - DNA, 30 bp, synthetic construct
  source 1..30: mol_type = other DNA
caccacactgatagtgctagtgtagatcac

SEQ ID NO: 243 - DNA, 30 bp, synthetic construct
  source 1..30: mol_type = other DNA
gccggaataactccctataatgcgccacca

SEQ ID NO: 244 - DNA, 30 bp, synthetic construct
  source 1..30: mol_type = other DNA
ttgacaagcttttcctcagctccgtaaact

SEQ ID NO: 245 - DNA, 30 bp, synthetic construct
  source 1..30: mol_type = other DNA
ggtttcaaaattgtgatctatatttaacaa

SEQ ID NO: 246 - DNA, 30 bp, synthetic construct; identical to SEQ ID NO: 245
  source 1..30: mol_type = other DNA

SEQ ID NO: 247 - DNA, 30 bp, synthetic construct
  source 1..30: mol_type = other DNA
tctattccaataaagaaatcttcctgcgtg

SEQ ID NO: 248 - DNA, 30 bp, synthetic construct
  source 1..30: mol_type = other DNA
gaccgaatatatagtggaaacgtttagatg

SEQ ID NO: 249 - DNA, 30 bp, synthetic construct
  source 1..30: mol_type = other DNA
ccacatcctgtttttaaccttaaaatggca

SEQ ID NO: 250 - DNA, 30 bp, synthetic construct
  source 1..30: mol_type = other DNA
aaaaatgggctcgtgttgtacaataaatgt

SEQ ID NO: 251 - DNA, 30 bp, synthetic construct
  source 1..30: mol_type = other DNA
aaaaaaagcgcgcgattatgtaaaatataa

SEQ ID NO: 252 - DNA, 30 bp, synthetic construct
  source 1..30: mol_type = other DNA
aattgcagtaggcatgacaaaatggactca

SEQ ID NO: 253 - DNA, 30 bp, synthetic construct
  source 1..30: mol_type = other DNA
caagcttttcctttataatagaatgaatga

SEQ ID NO: 254 - DNA, 30 bp, synthetic construct
  source 1..30: mol_type = other DNA
tctaagctagtgtattttgcgtttaatagt

SEQ ID NO: 255 - DNA, 30 bp, synthetic construct
  source 1..30: mol_type = other DNA
aatgggctcgtgttgtacaataaatgtagt

SEQ ID NO: 256 - DNA, 30 bp, synthetic construct
  source 1..30: mol_type = other DNA
atccttatcgttatgggtattgtttgtaat

SEQ ID NO: 257 - DNA, 30 bp, synthetic construct
  source 1..30: mol_type = other DNA
taaaagaattgtgagcgggaatacaacaac

SEQ ID NO: 258 - DNA, 30 bp, synthetic construct; identical to SEQ ID NO: 251
  source 1..30: mol_type = other DNA

SEQ ID NO: 259 - DNA, 30 bp, synthetic construct
  source 1..30: mol_type = other DNA
tacaaaataattcccctgcaaacattatca

SEQ ID NO: 260 - DNA, 30 bp, synthetic construct
  source 1..30: mol_type = other DNA
tacaaaataattcccctgcaaacattatcg

SEQ ID NO: 261 - DNA, 30 bp, synthetic construct
  source 1..30: mol_type = other DNA
agggaatacaagctacttgttctttttgca

SEQ ID NO: 262 - DNA, 23 bp, synthetic construct
  source 1..23: mol_type = other DNA
taatacgactcactatagggaga

SEQ ID NO: 263 - DNA, 28 bp, synthetic construct
  source 1..28: mol_type = other DNA
gaatttaatacgactcactatagggaga

SEQ ID NO: 264 - DNA, 19 bp, synthetic construct
  source 1..19: mol_type = other DNA
taatacgactcactatagg

SEQ ID NO: 265 - DNA, 30 bp, synthetic construct
  source 1..30: mol_type = other DNA
gagtcgtattaatacgactcactatagggg

SEQ ID NO: 266 - DNA, 30 bp, synthetic construct
  source 1..30: mol_type = other DNA
agtgagtcgtactacgactcactatagggg

SEQ ID NO: 267 - DNA, 30 bp, synthetic construct
  source 1..30: mol_type = other DNA
gagtcgtattaatacgactctctatagggg

SEQ ID NO: 268 - DNA, 18 bp, synthetic construct
  source 1..18: mol_type = other DNA
taatacgactcactatag

SEQ ID NO: 269 - DNA, 23 bp, synthetic construct
  source 1..23: mol_type = other DNA
taatacgactcactatagggaga

SEQ ID NO: 270 - DNA, 23 bp, synthetic construct
  source 1..23: mol_type = other DNA
ttatacgactcactatagggaga

SEQ ID NO: 271 - DNA, 23 bp, synthetic construct
  source 1..23: mol_type = other DNA
gaatacgactcactatagggaga

SEQ ID NO: 272 - DNA, 23 bp, synthetic construct
  source 1..23: mol_type = other DNA
taatacgtctcactatagggaga

SEQ ID NO: 273 - DNA, 23 bp, synthetic construct
  source 1..23: mol_type = other DNA
tcatacgactcactatagggaga

SEQ ID NO: 274 - DNA, 30 bp, synthetic construct
  source 1..30: mol_type = other DNA
taatacgactcactatagggagaccacaac

SEQ ID NO: 275 - DNA, 30 bp, synthetic construct
  source 1..30: mol_type = other DNA
taattgaactcactaaagggagaccacagc

SEQ ID NO: 276 - DNA, 30 bp, synthetic construct
  source 1..30: mol_type = other DNA
cgaagtaatacgactcactattagggaaga

SEQ ID NO: 277 - DNA, 19 bp, synthetic construct
  source 1..19: mol_type = other DNA
atttaggtgacactataga

SEQ ID NO: 278 - DNA, 30 bp, synthetic construct
  source 1..30: mol_type = other DNA
acaaacacaaatacacacactaaattaata

SEQ ID NO: 279 - DNA, 30 bp, synthetic construct
  source 1..30: mol_type = other DNA
ccaagcatacaatcaactatctcatataca

SEQ ID NO: 280 - DNA, 30 bp, synthetic construct
  source 1..30: mol_type = other DNA
gatacaggatacagcggaaacaacttttaa

SEQ ID NO: 281 - DNA, 30 bp, synthetic construct
  source 1..30: mol_type = other DNA
tttcaagctataccaagcatacaatcaact

SEQ ID NO: 282 - DNA, 30 bp, synthetic construct
  source 1..30: mol_type = other DNA
cctttgcagcataaattactatacttctat

SEQ ID NO: 283 - DNA, 30 bp, synthetic construct; identical to SEQ ID NO: 282
  source 1..30: mol_type = other DNA

SEQ ID NO: 284 - DNA, 30 bp, synthetic construct; identical to SEQ ID NO: 282
  source 1..30: mol_type = other DNA

SEQ ID NO: 285 - DNA, 30 bp, synthetic construct; identical to SEQ ID NO: 282
  source 1..30: mol_type = other DNA

SEQ ID NO: 286 - DNA, 30 bp, synthetic construct; identical to SEQ ID NO: 282
  source 1..30: mol_type = other DNA

SEQ ID NO: 287 - DNA, 30 bp, synthetic construct
  source 1..30: mol_type = other DNA
ttatctactttttacaacaaatataaaaca

SEQ ID NO: 288 - DNA, 30 bp, synthetic construct; identical to SEQ ID NO: 278
  source 1..30: mol_type = other DNA

SEQ ID NO: 289 - DNA, 30 bp, synthetic construct
  source 1..30: mol_type = other DNA
gtttcgaataaacacacataaacaaacaaa

SEQ ID NO: 290 - DNA, 30 bp, synthetic construct; identical to SEQ ID NO: 279
  source 1..30: mol_type = other DNA

SEQ ID NO: 291 - DNA, 30 bp, synthetic construct
  source 1..30: mol_type = other DNA
accatcaaaggaagctttaatcttctcata

SEQ ID NO: 292 - DNA, 30 bp, synthetic construct
  source 1..30: mol_type = other DNA
agaacccactgcttactggcttatcgaaat

SEQ ID NO: 293 - DNA, 30 bp, synthetic construct
  source 1..30: mol_type = other DNA
ggccgtttttggcttttttgttagacgaag

SEQ ID NO: 294 - DNA, 66 bp, synthetic construct; identical to SEQ ID NO: 22
  source 1..66: mol_type = other DNA

SEQ ID NO: 295 - DNA, 52 bp, synthetic construct
  source 1..52: mol_type = other DNA
aaaaagagtattgacttcgcatctttttgtacctataatagattcattgcta

SEQ ID NO: 296 - DNA, 59 bp, synthetic construct
  source 1..59: mol_type = other DNA
ggaaaatttttttaaaaaaaaaactttacagctagctcagtcctaggtattatgctagc

SEQ ID NO: 297 - DNA, 59 bp, synthetic construct
  source 1..59: mol_type = other DNA
ggaaaatttttttaaaaaaaaaactttacggctagctcagccctaggtattatgctagc

SEQ ID NO: 298 - DNA, 64 bp, synthetic construct; identical to SEQ ID NO: 16
  source 1..64: mol_type = other DNA

SEQ ID NO: 299-308 - skipped (10 entries)

SEQ ID NO: 309 - DNA, 222 bp, synthetic construct
  source 1..222: mol_type = other DNA
acgttaaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 310 - DNA, 714 bp, synthetic construct
  source 1..714: mol_type = other DNA
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcat

SEQ ID NO: 311 - skipped

SEQ ID NO: 312 - protein, 237 aa, synthetic construct
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 313 - DNA, 84 bp, synthetic construct
  source 1..84: mol_type = other DNA
acgttaaatctatcaccgcaagggataaatatctaacaccgtgcgtgttgactattttacctctggcggtgataatggttgcat

SEQ ID NO: 314 - DNA, 714 bp, synthetic construct
  source 1..714: mol_type = other DNA
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgccttatttaatggcatcaatgcattaaatgcttataacgccgcatcgcttacaagaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgctctcacctgagcttagaacctttaccaaaggtggtgcggaaaggtgggtaagcacaaccaaaaaagccagtgattctgcattctggcttgaggttgaaggtaattccatgacagcaccaacaggctccaagccaagctttcctgacggaatgttaattctcgttgaccctgagcaggctgttgagccaggtgatttctgcatagccagactcgggggtggtgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctga

SEQ ID NO: 315 - protein, 237 aa, synthetic construct
MSTKKKPLTQEQLEDARRLKAIYEKKKNELGLSQESVADKMGMGQSGVGALFNGINALNAYNAASLTRILKVSVEEFSPSIAREIYEMYEAVSMQPSLRSEYEYPVFSHVQAGMLSPELRTFTKGGAERWVSTTKKASDSAFWLEVEGNSMTAPTGSKPSFPDGMLILVDPEQAVEPGDFCIARLGGGEFTFKKLIRDSGQVFLQPLNPQYPMIPCNESCSVVGKVIASQWPEETFG

SEQ ID NO: 316 - DNA, 183 bp, synthetic construct
  source 1..183: mol_type = other DNA
aaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataaggtagaaac

SEQ ID NO: 317-999 - skipped (683 entries)

SEQ ID NO: 1000 - DNA, 1182 bp, Brevibacterium aurantiacum
  source 1..1182: mol_type = genomic DNA
atgacgagccttcaccccgaaactttaatggtgcacgggggtatgaagggattgacagaggcgggtgttcacgtcccggcgatcgacctgtcgacaacgaatcctgttaatgacgtagcgacagggggtgattcttacgaatggcttgcaacgggtcacacgttgaaggatggtgattctgctgtttaccagcgtttatggcagcccggtgtagctcgtttcgagacggctctggctgggctggagcacgcggaggaggctgtagctttcgccacgggtatggccgccatgaccgcggccttattggcagcagtatcagctggcacgcctcatatcgtcgcagtccgcccattatacggcggttcagaccatcttttagagactggtttactggggacaactgtcacgtgggcgaaagaagctgacatcgccagtgcaatccaagacgatacagggttagtgatcgtggagacgccagccaatcctagtcttgatctggtagacctggattctgtcgtgtcagcggcagggaacgtccctgtccttgtagataatacgttttgtactcctgtccttcagcaacctatctcgcacggcgccgcacttgtattacattcggcaacaaagtatttaggggggcatggggacgccatgggcgggatcatcgcgactaatgctgattgggctatgcgcctgcgtcaggtccgtgcgatcactggtgcattactgcatccgatgggcgcgtacttgttacaccgcggtttgcgtactctggctgttcgcatgcgtgccgctcagacaactgctggtgagcttgctgagcgcttagacgctcatcccgctatttcagttgtgcactacccaggattgaaggggcaagatcctcgtggattgttaggccgtcagatgagtggaggtggggcaatgattgctatggagctggcagggggattcgatgcggcacgctcctttgttgagcattgtaacttggtcgtgcatgccgtatcacttgggggagcagacacattaattcaacatccggcaagtttgacacaccgcccggtcgcagcaaccgccaagcctggtgatggtttgatccgcttgagcgtggggcttgagcacgttgacgacttggccgatgacttgatcgctgctttggacgcctctcgcgctgctgcatga

SEQ ID NO: 1001 - DNA, 1197 bp, Citrobacter freundii
  source 1..1197: mol_type = genomic DNA
atgtctgattgtcgtgcctacggctttaacacacagatcgtccacgccggacaacaaccggacccaagcactggtgcattgtcgacgccaatctttcagacttctactttcgttttcgattcggcagaacagggggccgcacgtttcgcgttggaggaaccgggatacatctatacgcgcttgggcaatccaactactgatgcgcttgaaaagaaacttgcagtgctggaacgtggggacgcggcgttagccacagcgagcggcatctccgctattaccacaagtcttcttactctttgccagcaaggggaccatatcgtatcagctagtgcaatctacggttgcactcacgcattcttaagtcattcgcttcctaaatttggcattaacgtttcattcgtcgacgcagcgaagcccgaagaaatccgcgccgccatgcgtccagagacaaaagtggtatatatcgagacgcccgcgaaccctactttatccttagtggatattgagactgttgctggaatcgcgcaccagcaaggagccttgttagtcgtggacaacacttttatgtctccgtattgtcaacaaccattactgctgggtgctgacatcgtggtccatagtgttaccaagtacattaacgggcatggggatgttattggcggtgtcatcgtaggtaaacaggagttcatcgaccaagcacgctttgtgggattaaaggacattacgggcggatgcatgagcccctttaatgcatggctgacacttcgcggggtaaagacgttagggatccgtatggagcgtcattgcgaaaacgcattgaaaatcgcgcgttttcttgagggccatcctgctattacacgtgtctattatcctggattgccgtcacacccccaatacgaacttgggaagcgccaaatgtcgcttcctgggggaatcatttcgtttgaaattgctggtggccttgaagcaggtcgccgtatgattaacagcgtggagttatgtttgctggcagttagcttaggtgatacggagacgttgattcagcatccggcgagcatgacacattcccctgttgctcccgaggagcgtctgaaggctggcatcactgatggactgatccgtttgtccgtcggccttgaagatccggaagacatcattaacgacctggagcacgccattcgcaaggcaacgttttaa

SEQ ID NO: 1002 - DNA, 1200 bp, Porphyromonas gingivalis
  source 1..1200: mol_type = genomic DNA
atgaagaaagaggacttaatgcgttcggggttcgcaactcgtgctattcatggtggagccatcgaaaatgcttttgggtgcctggccacccccatctaccaaacttcaacgtttgttttcgatacggctgaacagggtggacgtcgctttgcgggtgaagaagatgggtacatttacactcgcttaggaaacccgaactgtacccaggttgaggagaagctggcgatgttagaaggaggggaagcagcggcctccgcaagtagtggtatcggagcgatctcttcagctatctgggtgtgcgtcaaggcgggagatcatattgtagcgggaaaaactttgtacggttgcacgtttgcgttcttgacccacggactttctcgctatggagttgaggtcaccttggtggacacgcgccatccagaagaggttgaggccgctattcgtccaaacactaagttggtctatttggaaactcccgcaaaccctaacatgtaccttactgatatcaaagctgtgtgtgatatcgcgcacaagcatgagggtgttcgcgtcatggtggataatacctactgtacaccttacatctgtcgcccactggagttaggtgctgatattgttgtccattccgccacaaaatatctgaacggacatggagatgttatcgctgggtttgtagtagggaaagaggattacatcaaggaggtcaaattggttggtgttaaggacttgacaggagccaatatgtcacccttcgacgcataccttattagtcgtggtatgaagacgttacaaatccgtatggaacaacattgccgtaacgcacaaacagttgctgagtttctggagaagcatcctgcggttgaggcggtgtacttcccgggcttgccttcattcccgcaatatgagttggccaagaagcagatggcacttccgggagcgatgattgcgtttgaagttaagggaggctgcgaagccggtaagaaacttatgaacaatcttcatctgtgttccttagccgtctccttaggcgataccgagactttaatccaacacccggcctcaatgacgcactcgccctatacgcccgaggaacgcgccgcaagcgacatctcggaggggcttgtacgcctttctgttggattggaaaatgttgaggatattatcgcggacctgaagcatggcctggatagtcttatctaa

SEQ ID NO: 1003 - DNA, 1674 bp, Streptomyces sp.
  source 1..1674: mol_type = genomic DNA
atgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatgcagggaacggccttggatttagcccgtttcatgcccaacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 1004 - DNA, 1032 bp, Escherichia coli
  source 1..1032: mol_type = genomic DNA
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccccactgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaggaacaccatgtaaaagtagaggtactgggttatgtctga

SEQ ID NO: 1005 - DNA, 654 bp, Escherichia coli
  source 1..654: mol_type = genomic DNA
atgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaa

SEQ ID NO: 1006 - DNA, 816 bp, Escherichia coli
  source 1..816: mol_type = genomic DNA
atggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaggatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 1007 - protein, 393 aa, Brevibacterium aurantiacum
MTSLHPETLMVHGGMKGLTEAGVHVPAIDLSTTNPVNDVATGGDSYEWLATGHTLKDGDSAVYQRLWQPGVARFETALAGLEHAEEAVAFATGMAAMTAALLAAVSAGTPHIVAVRPLYGGSDHLLETGLLGTTVTWAKEADIASAIQDDTGLVIVETPANPSLDLVDLDSVVSAAGNVPVLVDNTFCTPVLQQPISHGAALVLHSATKYLGGHGDAMGGIIATNADWAMRLRQVRAITGALLHPMGAYLLHRGLRTLAVRMRAAQTTAGELAERLDAHPAISVVHYPGLKGQDPRGLLGRQMSGGGAMIAMELAGGFDAARSFVEHCNLVVHAVSLGGADTLIQHPASLTHRPVAATAKPGDGLIRLSVGLEHVDDLADDLIAALDASRAAA

SEQ ID NO: 1008 - protein, 398 aa, Citrobacter freundii
MSDCRAYGFNTQIVHAGQQPDPSTGALSTPIFQTSTFVFDSAEQGAARFALEEPGYIYTRLGNPTTDALEKKLAVLERGDAALATASGISAITTSLLTLCQQGDHIVSASAIYGCTHAFLSHSLPKFGINVSFVDAAKPEEIRAAMRPETKVVYIETPANPTLSLVDIETVAGIAHQQGALLVVDNTFMSPYCQQPLLLGADIVVHSVTKYINGHGDVIGGVIVGKQEFIDQARFVGLKDITGGCMSPFNAWLTLRGVKTLGIRMERHCENALKIARFLEGHPAITRVYYPGLPSHPQYELGKRQMSLPGGIISFEIAGGLEAGRRMINSVELCLLAVSLGDTETLIQHPASMTHSPVAPEERLKAGITDGLIRLSVGLEDPEDIINDLEHAIRKATF

SEQ ID NO: 1009 - protein, 399 aa, Porphyromonas gingivalis
MKKEDLMRSGFATRAIHGGAIENAFGCLATPIYQTSTFVFDTAEQGGRRFAGEEDGYIYTRLGNPNCTQVEEKLAMLEGGEAAASASSGIGAISSAIWVCVKAGDHIVAGKTLYGCTFAFLTHGLSRYGVEVTLVDTRHPEEVEAAIRPNTKLVYLETPANPNMYLTDIKAVCDIAHKHEGVRVMVDNTYCTPYICRPLELGADIVVHSATKYLNGHGDVIAGFVVGKEDYIKEVKLVGVKDLTGANMSPFDAYLISRGMKTLQIRMEQHCRNAQTVAEFLEKHPAVEAVYFPGLPSFPQYELAKKQMALPGAMIAFEVKGGCEAGKKLMNNLHLCSLAVSLGDTETLIQHPASMTHSPYTPEERAASDISEGLVRLSVGLENVEDIIADLKHGLDSLI

SEQ ID NO: 1010 - protein, 557 aa, Streptomyces sp.
MSPTAFPAAETATAPATAVDPGPELDGGDFALPEGGLDDDRRLRALDAVDEYLTRKRKHLVGYQATQDMQGTALDLARFMPNNINNLGDPFQSGGYKPNTKVVERAVLDYYAKLWHAERPHDPADPESYWGYMLSMGSTEGNMYALWNARDYLSGKALIQPPTAPFDAVRYVKADPDRRNPNAHHPVAFYSEDTHYSFAKAVAVLGVETFHAVGLEKYADECPLVDPVTGLRTWPTEVPSRPGPSGLSWDGPGEIDVDALAVLVEFFAAKGHPVFVNLNLGSTFKGAHDDVRAVCERLLPIFERHGLVQREVVYGSCPQTGRPLVDVRRGFWIHVDGALGAGYAPFLRLAAEDPEGYGWTPEAELPEFDFGLRLPTAGHGEVDMVSSIAMSGHKWAGAPWPCGIYMTKVKYQISPPSQPDYIGAPDTTFAGSRNGFSPLILWDHLSRYSYRDQVERIREAQELAAYLERRLTAMERELGVELWPARTPGAVTVRFRKPSAELVAKWSLSSQDVLMVPGDETTRRSYVHVFVMPSVDRAKLDALLAELAEDPVILGAP

SEQ ID NO: 1011 - protein, 343 aa, Escherichia coli
MIKLSNITKVFHQGTRTIQALNNVSLHVPAGQIYGVIGASGAGKSTLIRCVNLLERPTEGSVLVDGQELTTLSESELTKARRQIGMIFQHFNLLSSRTVFGNVALPLELDNTPKDEIKRRVTELLSLVGLGDKHDSYPSNLSGGQKQRVAIARALASNPKVLLCDEATSALDPATTRSILELLKDINRRLGLTILLITHEMDVVKRICDCVAVISNGELIEQDTVSEVFSHPKTPLAQKFIQSTLHLDIPEDYQERLQAEPFTDCVPMLRLEFTGQSVDAPLLSETARRFNVNNNIISAQMDYAGGVKFGIMLTEMHGTQQDTQAAIAWLQEHHVKVEVLGYV

SEQ ID NO: 1012 - protein, 217 aa, Escherichia coli
MSEPMMWLLVRGVWETLAMTFVSGFFGFVIGLPVGVLLYVTRPGQIIANAKLYRTISAIVNIFRSIPFIILLVWMIPFTRVIVGTSIGLQAAIVPLTVGAAPFIARMVENALLEIPTGLIEASRAMGATPMQIVRKVLLPEALPGLVNAATITLITLVGYSAMGGAVGAGGLGQIGYQYGYIGYNATVMNTVLVLLVILVYLIQFAGDRIVRAVTRK

SEQ ID NO: 1013 - protein, 271 aa, Escherichia coli
MAFKFKTFAAVGALIGSLALVGCGQDEKDPNHIKVGVIVGAEQQVAEVAQKVAKDKYGLDVELVTFNDYVLPNEALSKGDIDANAFQHKPYLDQQLKDRGYKLVAVGNTFVYPIAGYSKKIKSLDELQDGSQVAVPNDPTNLGRSLLLLQKVGLIKLKDGVGLLPTVLDVVENPKNLKIVELEAPQLPRSLDDAQIALAVINTTYASQIGLTPAKDGIFVEDKESPYVNLIVTREDNKDAENVKKFVQAYQSDEVYEAANKVFNGGAVKGW

SEQ ID NO: 1014 - DNA, 1257 bp, Escherichia coli
  source 1..1257: mol_type = genomic DNA
atgagtggactcaaacaagaactggggctggcccagggcatcggcctactatcgacgtcattattaggcactggcgtgtttgccgttcctgcgttagctgcgctagtagcaggcaataacagcctgtgggcgtggcccgttttgattatcttagtgttcccgattgcgattgtgtttgcgattctgggtcgccactatcccagcgcaggcggcgtcgcacacttcgtcggtatggcgtttggttcgcggcttgagcgagtcaccggctggttgtttttatcggtcattcccgtgggtttgcctgccgcgctacaaattgctgccggattcggccaggcaatgtttggctggcatagcgggcaactgttgttggcagaactcggtacgctggcgctggtgtggtatatcggtactcgaggtgccagttccagtgctaatctacaaacagttattgccgggcttatcgtcgcactgattgtcgctatctggtgggcgggcgatatcaaacctgcgaatatccccttccctgcgccaggaaatatcgaacttaccgggttattcgctgcgttatcagtgatgttctggtgttttgtcggtctggaagcatttgcccatcttgcctcggaatttaaaaatccagagcgtgattttcctcgtgctttgatgattggcctgctgctggcaggattagtctattggggctgtacggtagtcgtcttacacttcgacgcctatggtgaacaaatggcggcggcagcatcgcttcccaaaattgtagtgcagttattcggtgtaggagcgttatggattgcctgcgtaattggctatctggcctgctttgccagtctcaacatttatatacagagcttcgcccgcctggtctggtcgcaggcgcaacataatcctgaccattacctggcacgcctctcttctcgccatattccgaataatgccctcaatgcggtgctcggctgctgcgtggtgagcacgttggtgattcatgctttagagatcaatctggacgctcttattatttatgccaatggcatctttattatgatttatctgttatgcatgctggcaggctgtaaattattgcaaggacgttatcgactactggcagtggttggcgggctattatgcgttctgttactggcaatggtcggctggaaaagtctctacgcgctgatcatgctggcggggttatggctgtttctgccaaaacgaaaaacgccggaaaatggcataaccacataa

SEQ ID NO: 1015 - DNA, 1197 bp, Citrobacter freundii
  source 1..1197: mol_type = genomic DNA
atgtctgactgtcgtgcttacggattcaatacccagatcgttcatgcgggccaacaacccgacccttccactggcgcgctcagtacgcctattttccagacgtcaaccttcgtttttgacagtgccgaacagggcgccgcccgctttgcgcttgaagaacccggctacatctatacgcgccttggaaaccccaccaccgacgcgctggagaaaaagctggctgtgctggaaagaggcgatgccgcgctggcaactgcatccggtatttcagccatcaccacctcgttgctgaccctttgccagcagggcgaccatatcgtttccgccagtgccatttacggctgcactcacgcttttctgtcacacagcctgccgaagttcggcattaacgtcagcttcgtcgacgccgccaagccggaagaaatccgcgcggccatgcgcccggaaaccaaagtggtgtatatcgaaacaccagcaaacccaacgctctcgctggttgatattgaaacggtcgccgggatcgcccatcagcaaggcgcattgctggtcgtggataacacctttatgtcaccctactgccagcaacctctgctgttaggtgccgacatcgtggtgcacagcgtgaccaagtacatcaacggccacggggacgtgattggcggtgtgattgttggcaagcaggaatttatcgaccaggcacgattcgtcgggctgaaagatatcaccggcggctgcatgagtccgttcaacgcctggctgacgctgcgcggcgtgaaaacgctgggcatccgaatggagcgccattgtgaaaacgcgttaaaaattgcccgcttcctggaagggcatccggccattacccgcgtgtattaccctggcttgccttcacatccgcagtatgagctgggtaaacgtcagatgagcctgccgggaggaattatcagcttcgaaatcgccggcggcctcgaggctggcagacggatgatcaattctgtagaattgtgcctcctcgcggtcagtctcggtgacaccgaaaccctcattcagcacccagcgtctatgacacattcgcccgttgcgccagaggaacggcttaaagcaggtattaccgacggacttatccgtctttctgttggtcttgaagatccagaagatattattaacgaccttgaacacgccatcagaaaggcaacattctga

SEQ ID NO: 1016 - DNA, 1200 bp, Porphyromonas gingivalis
  source 1..1200: mol_type = genomic DNA
atgaaaaaagaagaccttatgcgtagtggctttgccacacgtgccatccatggaggcgctatcgagaacgccttcggctgcttagccactcccatctaccaaacatcgactttcgtttttgacactgccgaacagggaggccgccgctttgccggagaggaagacggatacatctatacccgtctgggcaaccccaactgcacccaagtggaagagaaactggccatgctcgagggcggagaagccgccgcatcggcctcatccggtatcggagccatcagctctgccatctgggtatgcgtgaaggccggcgaccatatcgtagccggcaagacgctctacggctgcaccttcgccttcctcacccacggactgagccgctacggtgtggaagtcaccctcgtggatacccgccatccggaagaggtggaggctgccattcgcccgaatacgaagctcgtctatctggagactccggccaaccccaatatgtacctgaccgacatcaaggcagtctgcgacatcgctcataagcacgaaggcgtacgcgtcatggtggacaatacctactgcacgccctatatctgccgtccgctggagctgggtgccgacatcgtggtacacagcgcgaccaagtacctgaacggacatggcgacgtcatcgccggattcgtcgttggtaaagaggactacatcaaggaggtgaagctcgtcggcgtcaaggacctcacgggggccaatatgagtccgttcgatgcttatctgatcagccgcggcatgaagacgctgcagatacgtatggagcagcactgccgcaatgctcagaccgtagccgaattcctcgaaaagcatccggccgtagaagcagtttatttccccggacttccgagcttcccccaatacgaattggccaagaagcagatggcactgcccggagccatgatcgccttcgaagtgaagggcggttgcgaagccggtaagaagctgatgaacaacctgcacctctgctccctcgccgtgagcttgggcgatacggaaaccctcatccagcatccggccagcatgacccactcgccctacacacccgaagagcgtgctgccagcgacatatccgaaggactggtacgcctgtccgtgggtctggagaacgtggaggacatcatcgccgacctcaaacacggtctggacagcctgatctaa

SEQ ID NO: 1017 - DNA, 1182 bp, Brevibacterium aurantiacum
  source 1..1182: mol_type = genomic DNA
atgacctcactgcacccagaaacgctcatggtccacggcggaatgaaaggcctcaccgaggcaggagtccacgtaccggccatcgacctctcgaccaccaacccagtcaacgatgtcgccaccggcggtgactcctacgaatggctcgccaccggccatacgctcaaggacggcgactcggccgtctaccagcgcctctggcagcccggtgtcgcacgcttcgagaccgcgctggccgggctcgaacacgctgaggaagcagtcgccttcgccacgggcatggccgcaatgactgccgcacttctcgcggccgtcagcgcaggaacaccccacatcgtcgcagtgcgtcccctctatggcggaagcgaccacctcctcgaaaccggactgctggggacaacagtcacatgggcaaaggaagccgacatcgcctcggcgatccaagatgacaccggactcgtcattgtcgagaccccggcaaaccccagcctggaccttgttgatctcgacagtgtcgtctcagccgccggcaacgtgcctgtgctggtggacaacacattctgcacacctgttctccagcagcccatctcccacggagcggccctcgtactgcacagcgcgacaaaatacctcggcggtcatggcgatgccatgggcggcatcatcgccaccaacgccgactgggcgatgcgcctgcgacaggtccgagccatcacaggagccctgctccaccccatgggcgcgtatctccttcatcggggcttgcgcactctggccgtgcgcatgcgcgcggctcagaccaccgccggtgagctcgctgagcgcctggacgcgcaccctgccatctccgtcgtccactacccgggactgaaaggccaggacccacgcggactgctcggacgccaaatgtccggtggtggtgcgatgatcgcgatggagctcgccggtggattcgacgccgcccgcagcttcgtcgaacactgcaacctcgtcgtccacgcggtgtccctgggcggcgctgacactctcatccagcatccggcgtcactgactcacaggccagttgcggccacggcgaagcccggcgatggtctcatccgactctctgtgggactcgaacacgtcgatgacctggcagacgatctcatcgctgccctcgacgcgagtcgggccgctgcctga

SEQ ID NO: 1018 - DNA, 1674 bp, Streptomyces sp.
  source 1..1674: mol_type = genomic DNA
atgagcccgaccgccttccccgccgccgagaccgcgaccgcgcccgcgaccgccgtcgatcccggtccggagctggacggcggtgacttcgccctccccgagggcggcctggacgacgaccggcggctgcgcgcgctcgacgccgtggacgagtacctgacccgcaagcgcaagcacctggtcggctaccaggccacccaggacatgcagggcaccgcactggacctcgcccggttcatgccgaacaacatcaacaacctcggcgacccgttccagagcggcggatacaagcccaacaccaaggtcgtcgagcgggccgtgctcgactactacgcgaagctctggcacgccgagcgcccgcacgacccggccgacccggagtcgtactggggctacatgctgtccatgggctcgaccgagggcaacatgtacgccctctggaacgccagggactacctgagcggcaaggcgctgatccagccgccgaccgcccccttcgacgcggtgcgctacgtcaaggccgaccccgaccgacggaacccgaacgcccaccacccggtggccttctactccgaggacacccactactccttcgccaaggccgtggccgtcctcggcgtggagaccttccacgccgtcggcctggagaagtacgccgacgagtgcccgctggtcgacccggtgaccgggctgcgcacctggcccaccgaggtgccctcccgcccgggtccgtccggcctgtcctgggacggccccggcgagatagacgtcgacgccctcgccgtactcgtcgagttcttcgccgccaagggtcacccggtcttcgtcaacctcaacctcggcagcaccttcaagggcgcccacgacgacgtccgcgccgtctgcgagcgcttgctgccgatcttcgagcggcacgggctcgtccagcgcgaggtggtctacggcagctgcccgcagaccggccggccgctggtggacgtgcgccgcggcttctggatccacgtggacggcgcgctcggcgccggctacgcgccgttcctgcggctggccgccgaggacccggaagggtacggctggacgcccgaggcggagctgcccgagttcgacttcggcctgcggctgcccaccgccgggcacggcgaggtggacatggtctcctcgatcgcgatgagcggccacaagtgggccggcgcgccgtggccgtgcggcatctacatgaccaaggtgaagtaccagatctcgccgccgtcccagccggactacatcggcgccccggacaccaccttcgccggctcccgcaacggcttctccccgctgatcctctgggaccacctgtcccggtactcctaccgggaccaggtggagcggatccgcgaggcccaggagctggccgcctacctggagcggcggctgaccgccatggagcgcgaactcggcgtcgagctctggccggcccgtaccccgggcgccgtcaccgtacggttccgcaagccgagcgccgagctggtggccaagtggtcgctgtcctcccaggacgtgctgatggtcccgggcgacgagaccacccggcgcagctacgtgcacgtcttcgtgatgccctcggtcgaccgggccaagctggacgcgctgctcgccgaactcgccgaggacccggtgatcctgggcgcaccgtag

SEQ ID NO: 1019 - DNA, 4141 bp, synthetic construct
  source 1..4141: mol_type = other DNA
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaatctacgactcttgacgacgaggaggaaataaatgacgagccttcaccccgaaactttaatggtgcacgggggtatgaagggattgacagaggcgggtgttcacgtcccggcgatcgacctgtcgacaacgaatcctgttaatgacgtagcgacagggggtgattcttacgaatggcttgcaacgggtcacacgttgaaggatggtgattctgctgtttaccagcgtttatggcagcccggtgtagctcgtttcgagacggctctggctgggctggagcacgcggaggaggctgtagctttcgccacgggtatggccgccatgaccgcggccttattggcagcagtatcagctggcacgcctcatatcgtcgcagtccgcccattatacggcggttcagaccatcttttagagactggtttactggggacaactgtcacgtgggcgaaagaagctgacatcgccagtgcaatccaagacgatacagggttagtgatcgtggagacgccagccaatcctagtcttgatctggtagacctggattctgtcgtgtcagcggcagggaacgtccctgtccttgtagataatacgttttgtactcctgtccttcagcaacctatctcgcacggcgccgcacttgtattacattcggcaacaaagtatttaggggggcatggggacgccatgggcgggatcatcgcgactaatgctgattgggctatgcgcctgcgtcaggtccgtgcgatcactggtgcattactgcatccgatgggcgcgtacttgttacaccgcggtttgcgtactctggctgttcgcatgcgtgccgctcagacaactgctggtgagcttgctgagcgcttagacgctcatcccgctatttcagttgtgcactacccaggattgaaggggcaagatcctcgtggattgttaggccgtcagatgagtggaggtggggcaatgattgctatggagctggcagggggattcgatgcggcacgctcctttgttgagcattgtaacttggtcgtgcatgccgtatcacttgggggagcagacacattaattcaacatccggcaagtttgacacaccgcccggtcgcagcaaccgccaagcctggtgatggtttgatccgcttgagcgtggggcttgagcacgttgacgacttggccgatgacttgatcgctgctttggacgcctctcgcgctgctgcatgaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 1020 - DNA, 4156 bp, synthetic construct
  source 1..4156: mol_type = other DNA
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaacggtctttcaacaaccacaggaggtattaatatgtctgattgtcgtgcctacggctttaacacacagatcgtccacgccggacaacaaccggacccaagcactggtgcattgtcgacgccaatctttcagacttctactttcgttttcgattcggcagaacagggggccgcacgtttcgcgttggaggaaccgggatacatctatacgcgcttgggcaatccaactactgatgcgcttgaaaagaaacttgcagtgctggaacgtggggacgcggcgttagccacagcgagcggcatctccgctattaccacaagtcttcttactctttgccagcaaggggaccatatcgtatcagctagtgcaatctacggttgcactcacgcattcttaagtcattcgcttcctaaatttggcattaacgtttcattcgtcgacgcagcgaagcccgaagaaatccgcgccgccatgcgtccagagacaaaagtggtatatatcgagacgcccgcgaaccctactttatccttagtggatattgagactgttgctggaatcgcgcaccagcaaggagccttgttagtcgtggacaacacttttatgtctccgtattgtcaacaaccattactgctgggtgctgacatcgtggtccatagtgttaccaagtacattaacgggcatggggatgttattggcggtgtcatcgtaggtaaacaggagttcatcgaccaagcacgctttgtgggattaaaggacattacgggcggatgcatgagcccctttaatgcatggctgacacttcgcggggtaaagacgttagggatccgtatggagcgtcattgcgaaaacgcattgaaaatcgcgcgttttcttgagggccatcctgctattacacgtgtctattatcctggattgccgtcacacccccaatacgaacttgggaagcgccaaatgtcgcttcctgggggaatcatttcgtttgaaattgctggtggccttgaagcaggtcgccgtatgattaacagcgtggagttatgtttgctggcagttagcttaggtgatacggagacgttgattcagcatccggcgagcatgacacattcccctgttgctcccgaggagcgtctgaaggctggcatcactgatggactgatccgtttgtccgtcggccttgaagatccggaagacatcattaacgacctggagcacgccattcgcaaggcaacgttttaaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 1021 - DNA, 4156 bp, synthetic construct
  source 1..4156: mol_type = other DNA
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaagggtattattgtcgtaaggaggaatctatgaagaaagaggacttaatgcgttcggggttcgcaactcgtgctattcatggtggagccatcgaaaatgcttttgggtgcctggccacccccatctaccaaacttcaacgtttgttttcgatacggctgaacagggtggacgtcgctttgcgggtgaagaagatgggtacatttacactcgcttaggaaacccgaactgtacccaggttgaggagaagctggcgatgttagaaggaggggaagcagcggcctccgcaagtagtggtatcggagcgatctcttcagctatctgggtgtgcgtcaaggcgggagatcatattgtagcgggaaaaactttgtacggttgcacgtttgcgttcttgacccacggactttctcgctatggagttgaggtcaccttggtggacacgcgccatccagaagaggttgaggccgctattcgtccaaacactaagttggtctatttggaaactcccgcaaaccctaacatgtaccttactgatatcaaagctgtgtgtgatatcgcgcacaagcatgagggtgttcgcgtcatggtggataatacctactgtacaccttacatctgtcgcccactggagttaggtgctgatattgttgtccattccgccacaaaatatctgaacggacatggagatgttatcgctgggtttgtagtagggaaagaggattacatcaaggaggtcaaattggttggtgttaaggacttgacaggagccaatatgtcacccttcgacgcataccttattagtcgtggtatgaagacgttacaaatccgtatggaacaacattgccgtaacgcacaaacagttgctgagtttctggagaagcatcctgcggttgaggcggtgtacttcccgggcttgccttcattcccgcaatatgagttggccaagaagcagatggcacttccgggagcgatgattgcgtttgaagttaagggaggctgcgaagccggtaagaaacttatgaacaatcttcatctgtgttccttagccgtctccttaggcgataccgagactttaatccaacacccggcctcaatgacgcactcgccctatacgcccgaggaacgcgccgcaagcgacatctcggaggggcttgtacgcctttctgttggattggaaaatgttgaggatattatcgcggacctgaagcatggcctggatagtcttatctaaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 1022 - DNA, 4630 bp, synthetic construct
  source 1..4630: mol_type = other DNA
cgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaacaccagtagttcaaaaggaggtagaacagatgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatgcagggaacggccttggatttagcccgtttcatgcccaacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcac

SEQ ID NO: 1023 - DNA, 7482 bp, synthetic construct
  source 1..7482: mol_type = other DNA
gtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctaggttccaggcattcgaaatgccacgactaacttaatgacgataataaataatcaatgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccccactgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaggaacaccatgtaaaagtagaggtactgggttatgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaacgttcaacacaacataaataattgaagaaggaataaggtatggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaggatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaatgaagatataatccgcattgcggatgcaattattaatgatcaacttcctctgccagaaatcatgctggaagaataagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtagtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcg

SEQ ID NO: 1024 - protein, 393 aa, synthetic construct; identical to SEQ ID NO: 1007

SEQ ID NO: 1025 - protein, 398 aa, synthetic construct; identical to SEQ ID NO: 1008

SEQ ID NO: 1026 - protein, 399 aa, synthetic construct; identical to SEQ ID NO: 1009

SEQ ID NO: 1027 - protein, 557 aa, synthetic construct; identical to SEQ ID NO: 1010

SEQ ID NO: 1028 - protein, 207 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 1029 - protein, 343 aa, synthetic construct; identical to SEQ ID NO: 1011

SEQ ID NO: 1030 - protein, 217 aa, synthetic construct; identical to SEQ ID NO: 1012

SEQ ID NO: 1031 - protein, 271 aa, synthetic construct; identical to SEQ ID NO: 1013

SEQ ID NO: 1032 - protein, 316 aa, synthetic construct
MSELVVFKANELAISRYDLTEHETKLILCCVALLNPTIENPTRKERTVSFTYNQYAQMMNISRENAYGVLAKATRELMTRTVEIRNPLVKGFEIFQWTNYAKFSSEKLELVFSEEILPYLFQLKKFIKYNLEHVKSFENKYSMRIYEWLLKELTQKKTHKANIEISLDEFKFMLMLENNYHEFKRLNQWVLKPISKDLNTYSNMKLVVDKRGRPTDTLIFQVELDRQMDLVTELENNQIKMNGDKIPTTITSDSYLRNGLRKTLHDALTAKIQLTSFEAKFLSDMQSKHDLNGSFSWLTQKQRTTLENILAKYGRI

SEQ ID NO: 1033 - protein, 286 aa, synthetic construct
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 1034 - DNA, 1674 bp, synthetic construct
  source 1..1674: mol_type = other DNA
atgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatggacggaacggccttggatttagcccgtttcatgccccacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 1035 - DNA, 1674 bp, synthetic construct
  source 1..1674: mol_type = other DNA
atgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatgcagggaacggccttggatttagcccgtttcatgcccaacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctctgaccgtacgttttcgcaaaccctctccggagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 1036 - DNA, 1674 bp, synthetic construct
  source 1..1674: mol_type = other DNA
atgtccccgacggcgtttccagcggccgaaacagctactgcccctgcaactgccgtcgatcctgggccagaactggacggcggagatttcgcccttccagagggcgggctggatgacgatcagcgcttacgtgcattggacgcagttgacgagtatttgacccgcaagcgcaagcatttggttgggtaccaagctacccaggatatggacggaacggccttggatttagcccgtttcatgcccaacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 1037 - DNA, 1674 bp, unidentified
  source 1..1674: mol_type = unassigned DNA
atggggttccagttactgtctaaacataagctgtcagccgaggatcaacagaaacttgaccgcttttatcgtgatattcagacagaagcagaacgattcctgggttacccatgtaacgaactctttgactactcccccttgttccggttcctgcaatatccgctgaataacgtcggcgacccgtacctgccgagtaactaccacctgaacacgcacaactttgagtgcgaagtactggaaatcttccgtaccctgaccgaggctactgaaggttcgacttggggctacgtgaccaacggcggtacggaaggtaatcattatggtcttttcctggcgagagagctgctgcctgaaggccttgtttactattctcaggatgcgcactactcgatcgataaaatcctgaggtgcctcaacctccgtagcataatgattcgcagccacgacgacggacgcatggacctggatgatctgcgtgaaactctgcgtatccatcgcgacttgccgccgatcgtttgcgctaccattggtactacaatgaagggcgctgtagatgacatcgcaggcattaaaaagatcttcaaagatctggcaatacaccgtcactatatccatgctgacgcggccctaggtggcatgattttaccgttcctggataactccccaccgtggaattttaaagctggaatcgactctatcgctatctccggtcacaaaatggtgggcagtcctatcccgtgtggggttgtcctggctaaaaagtcgaacgttgaacgtattgcacagagcgtggaatacattggtactctggataccaccctgtctggctcccgtaacgccttgactccgttatttctgtggtacgcgttccacaccgttggtatcgaaggtttcaaacgtatcatcccggcatgcttaaaaatggcggactatgccatcgctcagctgaacaaaattaaccgcaatgcgtggcgctacccttacagcaacacggtagtcttcgatcgcccaagccccgaagtgactcgttattggcagctggcttgtcagggcaacctgagccacctaatcaccatgccacacgttacatctactcaaattgatcatctggttgctgacatcatcgcttctgagccgataccgccgctgccgaccctgtcagttactccggcatgcgaactgctgacttctaccccggaccaggatattacgctgatcggcaccgctaatcataatctgctctccgaagtatctaccgccctggctgccgagggtctgtcaattgaaaacctggctgctgtggcggtagaaagcgaggacgttgaagttgtaaggctccgcgttaacaaccgtgagcgtgcactgcaaatcctgaaccagaacctggatatcggtcgttgctacggtcaggctcgaccctttggcaacgaagaagcgacgcaggtactgtcccagctggaatatcaaagcgtgggggaggatgcactactggtccagcttgacgattgccctggcagcctggcggagctgttgaaggattgccgcaacgaagcggtaaaaatccgtaatatccgactgctttggcgtgggcacggtaagggcgtcgtagcaattgctaccacttctccagatgcgctgaaaacgctgctgaaagaccgtattcttttgagctaa

SEQ ID NO: 1038 - DNA, 1278 bp, Mus musculus
  source 1..1278: mol_type = genomic DNA
atgtccacacctagtgaagtaaagaaggatttgctgggtgcagcagggtcattatggccgtcggagcccattacgctgggtccaggtgaaagtgcttggcagctggtattgaagaagatccaagagttgagtgacagcggtcatcaagacccgttcatggttgcagaccttgatgtccttgtgtctcgtcatcagacgttctgtcaagcactgcctagagtacaacccttctatgcagtaaagtgcaatagtaacccatgggtgttacgggtgttggcagctcttggcacgggatttgattgtgcttctcagggagaattggagcaagttttgggcttgggtgtagcgccgtcacggataatcttcgcaaatccctgtaaagcagtcagccacattcagtttgcagctcggtgcggtgtgcaattgttgacattcgacagcgaagaggagttaatcaaggttgcgcagtaccatccaggcgcacggttggtgcttcggattcaaacccaggactcacaatcaacgttcccactttccaccaagttcggtgcttctttagaagcatgtggacaccttctgcaggttgccagagagctgggtcttgccgtggtaggtgctagctttcatgtaggaagcgactgccacacacctcagagttttcgtcaggccatcgcagattgtcatcgtgtgttcgagatgggccgtaaggcaggtcatgatatgtcgcttcttgatttgggtggagggttcccaggtgtggaaggttccgaggcgaagtttgaggagatggcaagagtaatcaatgccgctcttgctcagtactttccggaagagactggcatcgaggtgatcgcggaacctggtcgtttctacgctgggtcggtgtgcactgcagctgtgaacatcatcgcgaagaagtctgtcttggaaccaggtggtcatcgtaagcttatgtactaccttaatgaaggacattacggttctttcagattgttcttgcgtgatccagtgcctcgtattcccatcgtggtgaaagagttcccatccgaaccaccactgtttccttgcactttgtacggtcccacatgtgacgcctatgatcggttgttttccgaagaggtacaattgccagagctggatgttggagattggttgatcttcccagatatgggtgcctatacctcctcaatgtcctcgaccttcaacggatttccaccggccaccgtgtattgcgcaatgtcaccgcagttacgctccctgttggagactgtaccataa

SEQ ID NO: 1039 - DNA, 1278 bp, Mus musculus
  source 1..1278: mol_type = genomic DNA
atgaacacacctagtgaagtaaagaaggatttgctgggtgttgcagaacatttacgtccgtcggagcccattacgctgggtccaggtgcgagtgcttggcagctggtattgaagaagatcaaggagttgagtattagcggtcgtcaagacgctttcatggttgcagaccttgatgtccttgtgtctcgtcatcggacgttcttacaagcactgcctagagtacaacccttctatgcagtaaagtgcaatagtaacccatgggtgttacttgtgttggcagctcttggcacgggatttgattgtgcttctcagggagaattggagcaagttttgggcttgggtgtagcgccgtcacggataatcttcgcaaatccctgtaaagcagtcagccacattcagtttgcagctcggtgcggtgtgcaattgttgacattcgacaatgaagaggagttaatcaagttagcgcgttaccatccacgtgcacggttggtgcttcggattcaaaccctggactcacaatcaacgttcccacttagcaccaagttcggtgctcacttagaagcatgtggacaccttctgcaggttgccagagagctgggtcttgccgtggtaggtgctagctttcatgtaggaagcgactgccacacacctgagagttaccgtcaggccatcgcagattgtcatcgtgtgttcgagatgggctgtaaggcaggtcatcacatgtcgcttcttgatttgggtggagggttcccaggtgtgaaaggttccgaggcgaagtttgaggaggttgcaagagtaatcaataccgctcttgctcagtactttccggaagagactggcatcgaggtgatcgcggaacctggtcgtttctacgctgggtcggtgtgcactgcagctgtgaacatcatcgccaagaagtctagtttggacccaggtggtcatcgtaagcttgcttactaccttaatgaaggacattacggtgtattcagattgttcttgcgtgatccagtgcctcgtattcccatcgtggtgaaagagttcccatccgaaccaccactgtttccttgcactttgtacggtcccacatgtgacgcctatgatcggttgttttccaccgaggtacaattgccagagctggatgttggagattggttgatcttcccagatatgggtgcctattcgtcctcaatgtcctcgaccttcaacggatttccaatagccaccgtgtatgatgcaatgtcaccgcagttacgctccctgttggagactgtaccataa

SEQ ID NO: 1040 - DNA, 1242 bp, Entamoeba histolytica
  source 1..1242: mol_type = genomic DNA
atgaaacaaacgtcccttgaggtgaaggaatttgccttgaatctcatttctcagttcgaaccagaaaaccagcctctgggtttctggatattcgacaccgaaggcgttgagaaagcggtagaacgctggaaaaagaacatgccgactgtccgtccctgttttgcagttaaatgcaacccggagccgcacctggtgaaattactgggggaactgggttgcggcttcgattgcgctagcctgaacgaaatcaaagaggtactggacttgggttttaatccggaagatatcacttatagtcagaccttcaaaccgtacaaccagttaattgaagcttcgcatctgggcatcaaccacacgatcgttgattcaatcgacgaagttcaaaaaattgctaaatacgcgcctaagatgggtatcatgattcggatcatggaaaatgacacaagcgcaggccacgtctttggagagaaattcggtctgcatgatgatgaagttgagatcgtactgaaggaaattaaagacaaaggtctgaacctggacggcgttcatttccacgttggctctgattcccacaacagcgaagtgtttactaaggcactgaccaaagctcgtaacactgtaaccctggccgaacagttcggcatgaaaccgtacctgatcgacattggtggcgggttctctcaggttgcgccgttcgaagaatttgctgctaccatcgaaaaaactataaaggaactggaatttccagagcgaactcgtttcattgcagagccgggtcgctatatggcatcaaatgcctttcaccttgtctcttcgctgcatggtaaaagggtgcgcatccagaacggtaagaaacagatcgaatacaccagcggcgatgggctgcacggctccttcggctgttgcatctggttcgaaaaacagaagtcttgcgaatgtataacacaaaaagtaaacgagaacaccaaaatgtatgaaagcatcatctacggcccatcttgcaacggttcggacaaagtggccacgcaggagttgccggaaatggagccgggtaaagattggctgctgttccccaatatgggtgcttacactatttccatggcgaccaactttaacggcttcgaagaacgtaaccatgtaatctatacgttaccactcaaaagtactaaaataattcagatccctaaaagcattgaatgcaactccgttccgtctttaaacggaatcccacactacgcgtaa

SEQ ID NO: 1041 - DNA, 1509 bp, Flavobacterium segetis
  source 1..1509: mol_type = genomic DNA
atggggaccattaacacgaagatctataaatacatgagcatctggaaaacaaaacctctgtccgtgctcttgtctgaagcaactgaggatgaaaaaggcctgaagcgcactctgtcggcccgttcacttgttgcgctgggtgtcggtgctattatcggcgctggtttattctctctgaccggcatagctgcggcagacaatgctggaccggcagtaaccctgagctttatcctggcctccgttggttgcgcgttcgctggcctgtgttacgcagaatttgcttctatgattccagttgcgggtagcgcctacacttatagttatgctaccatgggcgagttcgtggcgtggatcatcggttgggatctggtactcgaatacgcattgggcgcagctactgttgccgttagctggtcccagtacgtggacaaattcttgcaaaactacggcatccatattccgaactctatcctccacgggccgtgggataccacccccggtattatcaatttaccgtcgatatttatcatctgcctgctgagcgtgctgctgattcgtggtactaaagaatctgctctgatcaacaacattctggtaatcctgaaagtcacggttgtcatcgtgttcattggcctgggctgggggttcatgaactccgcaaaccacacgccctttatcccggttaacgaaggtgaggctctactgtcttctggtgaaatgagtttcctcaactttttcagcagtgactactttggacactacggatggtccggtattcttcgcggcgctggtgtagtattcttcgcatttatcggcttcgacgcggtgagcactgcggcacaggaggccaaggatccgcagaaaggcatgccaatcggtattctgggctcactgatcatttgcaccgttctgtacgtgcttttcgctttcgttctgaccggtctggaaaactatctaaacttcaaaggtgacgcttctcctgtcaccactgcatttgccaaaacaggctatactttcctgaatagcggtctgacgatcgctatcatagcgggctacacatccgttatgctggtaatgttgatgggtcagtcccgtgtcttttatagtatgtctgtggatggcctgcttccgaagtttttctcgaccctgcataccaaaaacaggactccgtacaaaactaatttgctgttcatggttttcgtaagcctgttcgctggctttgttccggtcagcgacctgggccatatggtatccatcggtaccctcttcgctttctgcctggtgtgtatcggcgttatcgttatgcgaaaaaccaacccagacgccgttcgcggttttcgtgttccttttgtaccggttttcccgattatcggtgtagttatttgtctggttctaatggcgggcctgccgattgaatcttgggaacgtctggcgatctggatgattctgggtgtcgtgatctacttcttctactctaaaaagaactctaaactgaataaccccgaataa

SEQ ID NO: 1042 - DNA, 1481 bp, Flavobacterium frigoris
  source 1..1481: mol_type = genomic DNA
atggggacgatcaatactaagaccaacaaatatatgagcatttggaaaaccaaaccgttgtctgtactgttaaacgaggcctcagaagatgaaaagggcctgaaaaggactctgtcctctcgttccctcgtggctctgggtgtcggtgcgatcattggcgcaggtctgtttagcctaacaggcatcgcagctgcggaacatgctggtccagcggttactctgagtttcatactggccgctgttggttgtgctttcgcaggcctgtgctacgcggagtttgcgtcgatgatccctgtggctgggtctgcttacacctatagctacgcaaccatgggcgaatttatggcgtggatcattggctgggaccttgtactggaatacgctctgggtgcagcgactgttggtgtatcctggtcccgttacttactggaattgctgaacaaatatggtgttcacctgaacccgaaattcatctgctctccgtgggagacacttaccctgggcgacggcactattatcgatggcgggtacatcaatctgccggcaattctgatcgtgagcgccctcagcttgctgctgattagaggtacccaggaatctgcttctattaacaacatcctggttgtgctgaaagtaatagtcgtgatcatgttcatcgttttaggatgggactatatcgatcccgcaaattactcaccttacatcccggaaaacaccggcgtaaagggccaattcggttggtcgggtatcgctgcgggtgctggtacggttttctttgccttcattggtttcgacgccgtttccactgcggctcaggaggctaaaaacccgcagaaaggcatgccaattggcatcctggggtctttggtaatttgtacgatcctgtacgtcctttttgcccacgttatgacgggcctggtgccgtattataagttcgctggagatgctaaacccgctgcgacagcattcgcagtcaccggttacagttttctgcaaactggactgattgttgcgatcctggctggctatactagcgttatgctggtcatgctgatggggcagagtcgtgttttctacaccatgagcaaagacggtctgctaccaccgctgttcggtcagatccattcgaaatttcgcactccgtacaagactaacctgttctttatggtattcgtttctttattcgcgggtttcgtgccggttagcgacctcggccacatggtcagcatcggtaccctcctggcgtttgttcttgtgtgcataggtgtgctggtgatgcgaaaaaagatgccagatgctccgcgttctttcaaaaccccgttcgttccgtatgtacccatcgcaggcgtcctggtgtgcacttacctgatgtactccctcccttacgaatcctggattcgcttagtgctttggatggctatcggcgtagccctgtacttcgtgtatggaaaaaagcactcaaaactgaacaatccggataa

SEQ ID NO: 1043 - DNA, 2533 bp, Escherichia coli
  source 1..2533: mol_type = genomic DNA
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccccactgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaggaacaccatgtaaaagtagaggtactgggttatgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaacgttcaacacaacataaataattgaagaaggaataaggtatggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaggatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 1044 - DNA, 666 bp, Bacillus subtilis
  source 1..666: mol_type = genomic DNA
atgtttgagaagtattttccaaatgttgacttgaccgagttatggaatgccacatatgaaactctgtatatgacattgatttccttactgtttgccttcgtaatcggcgtcatcctgggattgctgttattcttaacatctaaggggtctctttggcaaaataaagcagtaaattccgttatcgcagccgttgtcaacatctttcgttcaattcccttccttattttaatcatcctgcttcttggtttcactaaattcttagtgggaacaattttgggaccaaatgcggctcttcccgcgttagtcatcggtagtgctcccttttatgctcgtctggtcgaaatcgcacttcgtgaagtggacaaaggagtgattgaggcggcgaaatcgatgggggctaagacgagcactattatttttaaggttcttatccccgagtccatgcccgcgctgatttccggaattacagtgactgcgattgcattgatcgggtcaaccgccatcgcaggagctattggttctggtggattgggaaacttagcatacgttgaaggctatcaatcgaataatgcggatgtgaccttcgtggccacagttttcatcctgattattgttttcatcattcagatcattggtgaccttattaccaacatcatcgataaacgc

SEQ ID NO: 1045 - DNA, 2533 bp, synthetic construct
  source 1..2533: mol_type = other DNA
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccggcctgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaagaacaccatgtaaaagtagaggtactgggttatgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaacgttcaacacaacataaataattgaagaaggaataaggtatggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaagatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 1046 - DNA, 2533 bp, synthetic construct
  source 1..2533: mol_type = other DNA
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgccccactgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaagaacaccatgtaaaagtagaggtactgggttatgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaacgttcaacacaacataaataattgaagaaggaataaggtatggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaagatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 1047 - DNA, 2533 bp, synthetic construct
  source 1..2533: mol_type = other DNA
atgataaaactttcgaatatcaccaaagtgttccaccagggcacccgcaccatccaggcgttgaacaacgtcagcctgcatgtgccagctggacaaatttatggcgttatcggtgcctcaggcgcgggtaagagtacgcttatacgttgtgtaaacctgctggagcgcccaaccgagggtagcgtgctggtcgatggccaggaactgaccacgctgtcagaatccgagttgaccaaagctcgtcgccagattggtatgattttccagcattttaacctgctctcttcgcgtactgtttttggcaacgtggctctgccgctggagctggacaacacaccgaaagacgagatcaaacgtcgcgtgacggaattgctgtcattggttggtcttggcgataagcatgatagctacccgtcgaatctttccggtgggcagaaacaacgtgtggcgattgcccgtgcattagccagcaatcccaaagtattgctgtgtgatgaagccaccagcgcgctggacccggcaacgacacgttctattctcgaactgctgaaagacatcaaccgccgtctgggtttgacgattctgttgatcactcacgaaatggacgttgtgaagcgcatttgtgattgcgtggcggtcatcagcaatggcgaactgatcgagcaggacacggtaagtgaagtgttctcgcatccgaaaacgccgctggcgcagaagtttattcagtcgaccctgcatctggatatcccggaagattaccaggaacgtctgcaagcggagccatttactgactgcgtgccgatgctgcgtctggagtttaccggtcaatcggtcgatgcctccctgctttctgaaaccgcgcgtcgtttcaacgtcaacaacaacattattagcgcgcagatggattacgccggtggcgttaagttcggcatcatgctgactgaaatgcacggcacacaacaagatacgcaagccgccattgcctggctgcaagaacaccatgtaaaagtagaggtactgggttatgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagttgtatcgtaccatttctgcgattgtgaacattttccgttccatcccgttcattatcttgctggtatggatgattccgtttacccgcgttattgtcggtacatcgattggattgcaggcagcgattgttccgttaaccgttggtgcagcaccgtttattgcccgtatggtcgagaacgctctgctggagatcccaaccgggttaattgaagcttcccgcgcaatgggggccacgccaatgcagatcgtccgtaaagtgctgttaccggaagcgttgccgggtctggtgaatgcggcaactatcaccctgattaccctggttggttattccgcgatgggtggtgcagtcggtgccggtggtttaggtcagattggctatcagtatggctacatcggctacaacgcgacggtgatgaatacggtactggtattgctggtcattctggtttatttaattcagttcgcaggcgaccgcatcgtccgggctgtcactcgcaagtaacgttcaacacaacataaataattgaagaaggaataaggtatggcgttcaaattcaaaacctttgcggcagtgggagccctgattggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacatttgtttatccgattgctggttactccaagaaaatcaaatcactggatgaactgcaagatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaacccaaaaaatctgaaaattgttgaactggaagcaccgcagctaccgcgctctctggacgacgcgcaaatcgctctggcagttatcaataccacctatgccagccagattggcctgactccagcgaaagacggtatctttgtcgaagataaagagtccccgtacgtaaacctgatcgtaacgcgtgaagacaacaaagacgccgaaaacgtgaagaaattcgttcaggcttatcagtctgacgaagtttacgaagcagcaaacaaagtgtttaacggcggcgctgttaaaggctggtaa

SEQ ID NO: 1048 - protein, 557 aa, synthetic construct
MSPTAFPAAETATAPATAVDPGPELDGGDFALPEGGLDDDRRLRALDAVDEYLTRKRKHLVGYQATQDMDGTALDLARFMPHNINNLGDPFQSGGYKPNTKVVERAVLDYYAKLWHAERPHDPADPESYWGYMLSMGSTEGNMYALWNARDYLSGKALIQPPTAPFDAVRYVKADPDRRNPNAHHPVAFYSEDTHYSFAKAVAVLGVETFHAVGLEKYADECPLVDPVTGLRTWPTEVPSRPGPSGLSWDGPGEIDVDALAVLVEFFAAKGHPVFVNLNLGSTFKGAHDDVRAVCERLLPIFERHGLVQREVVYGSCPQTGRPLVDVRRGFWIHVDGALGAGYAPFLRLAAEDPEGYGWTPEAELPEFDFGLRLPTAGHGEVDMVSSIAMSGHKWAGAPWPCGIYMTKVKYQISPPSQPDYIGAPDTTFAGSRNGFSPLILWDHLSRYSYRDQVERIREAQELAAYLERRLTAMERELGVELWPARTPGAVTVRFRKPSAELVAKWSLSSQDVLMVPGDETTRRSYVHVFVMPSVDRAKLDALLAELAEDPVILGAP

SEQ ID NO: 1049 - protein, 557 aa, Streptomyces sp.; identical to SEQ ID NO: 1010

SEQ ID NO: 1050 - protein, 557 aa, synthetic construct
MSPTAFPAAETATAPATAVDPGPELDGGDFALPEGGLDDDRRLRALDAVDEYLTRKRKHLVGYQATQDMQGTALDLARFMPNNINNLGDPFQSGGYKPNTKVVERAVLDYYAKLWHAERPHDPADPESYWGYMLSMGSTEGNMYALWNARDYLSGKALIQPPTAPFDAVRYVKADPDRRNPNAHHPVAFYSEDTHYSFAKAVAVLGVETFHAVGLEKYADECPLVDPVTGLRTWPTEVPSRPGPSGLSWDGPGEIDVDALAVLVEFFAAKGHPVFVNLNLGSTFKGAHDDVRAVCERLLPIFERHGLVQREVVYGSCPQTGRPLVDVRRGFWIHVDGALGAGYAPFLRLAAEDPEGYGWTPEAELPEFDFGLRLPTAGHGEVDMVSSIAMSGHKWAGAPWPCGIYMTKVKYQISPPSQPDYIGAPDTTFAGSRNGFSPLILWDHLSRYSYRDQVERIREAQELAAYLERRLTAMERELGVELWPARTPGALTVRFRKPSPELVAKWSLSSQDVLMVPGDETTRRSYVHVFVMPSVDRAKLDALLAELAEDPVILGAP

SEQ ID NO: 1051 - protein, 557 aa, synthetic construct
MSPTAFPAAETATAPATAVDPGPELDGGDFALPEGGLDDDQRLRALDAVDEYLTRKRKHLVGYQATQDMDGTALDLARFMPNNINNLGDPFQSGGYKPNTKVVERAVLDYYAKLWHAERPHDPADPESYWGYMLSMGSTEGNMYALWNARDYLSGKALIQPPTAPFDAVRYVKADPDRRNPNAHHPVAFYSEDTHYSFAKAVAVLGVETFHAVGLEKYADECPLVDPVTGLRTWPTEVPSRPGPSGLSWDGPGEIDVDALAVLVEFFAAKGHPVFVNLNLGSTFKGAHDDVRAVCERLLPIFERHGLVQREVVYGSCPQTGRPLVDVRRGFWIHVDGALGAGYAPFLRLAAEDPEGYGWTPEAELPEFDFGLRLPTAGHGEVDMVSSIAMSGHKWAGAPWPCGIYMTKVKYQISPPSQPDYIGAPDTTFAGSRNGFSPLILWDHLSRYSYRDQVERIREAQELAAYLERRLTAMERELGVELWPARTPGAVTVRFRKPSAELVAKWSLSSQDVLMVPGDETTRRSYVHVFVMPSVDRAKLDALLAELAEDPVILGAP

SEQ ID NO: 1052 - protein, 557 aa, unidentified
MGFQLLSKHKLSAEDQQKLDRFYRDIQTEAERFLGYPCNELFDYSPLFRFLQYPLNNVGDPYLPSNYHLNTHNFECEVLEIFRTLTEATEGSTWGYVTNGGTEGNHYGLFLARELLPEGLVYYSQDAHYSIDKILRCLNLRSIMIRSHDDGRMDLDDLRETLRIHRDLPPIVCATIGTTMKGAVDDIAGIKKIFKDLAIHRHYIHADAALGGMILPFLDNSPPWNFKAGIDSIAISGHKMVGSPIPCGVVLAKKSNVERIAQSVEYIGTLDTTLSGSRNALTPLFLWYAFHTVGIEGFKRIIPACLKMADYAIAQLNKINRNAWRYPYSNTVVFDRPSPEVTRYWQLACQGNLSHLITMPHVTSTQIDHLVADIIASEPIPPLPTLSVTPACELLTSTPDQDITLIGTANHNLLSEVSTALAAEGLSIENLAAVAVESEDVEVVRLRVNNRERALQILNQNLDIGRCYGQARPFGNEEATQVLSQLEYQSVGEDALLVQLDDCPGSLAELLKDCRNEAVKIRNIRLLWRGHGKGVVAIATTSPDALKTLLKDRILLS

SEQ ID NO: 1053 - protein, 425 aa, Mus musculus
MSTPSEVKKDLLGAAGSLWPSEPITLGPGESAWQLVLKKIQELSDSGHQDPFMVADLDVLVSRHQTFCQALPRVQPFYAVKCNSNPWVLRVLAALGTGFDCASQGELEQVLGLGVAPSRIIFANPCKAVSHIQFAARCGVQLLTFDSEEELIKVAQYHPGARLVLRIQTQDSQSTFPLSTKFGASLEACGHLLQVARELGLAVVGASFHVGSDCHTPQSFRQAIADCHRVFEMGRKAGHDMSLLDLGGGFPGVEGSEAKFEEMARVINAALAQYFPEETGIEVIAEPGRFYAGSVCTAAVNIIAKKSVLEPGGHRKLMYYLNEGHYGSFRLFLRDPVPRIPIVVKEFPSEPPLFPCTLYGPTCDAYDRLFSEEVQLPELDVGDWLIFPDMGAYTSSMSSTFNGFPPATVYCAMSPQLRSLLETVP

SEQ ID NO: 1054 - protein, 425 aa, Mus musculus
MNTPSEVKKDLLGVAEHLRPSEPITLGPGASAWQLVLKKIKELSISGRQDAFMVADLDVLVSRHRTFLQALPRVQPFYAVKCNSNPWVLLVLAALGTGFDCASQGELEQVLGLGVAPSRIIFANPCKAVSHIQFAARCGVQLLTFDNEEELIKLARYHPRARLVLRIQTLDSQSTFPLSTKFGAHLEACGHLLQVARELGLAVVGASFHVGSDCHTPESYRQAIADCHRVFEMGCKAGHHMSLLDLGGGFPGVKGSEAKFEEVARVINTALAQYFPEETGIEVIAEPGRFYAGSVCTAAVNIIAKKSSLDPGGHRKLAYYLNEGHYGVFRLFLRDPVPRIPIVVKEFPSEPPLFPCTLYGPTCDAYDRLFSTEVQLPELDVGDWLIFPDMGAYSSSMSSTFNGFPIATVYDAMSPQLRSLLETVP

SEQ ID NO: 1055 - protein, 413 aa, Entamoeba histolytica
MKQTSLEVKEFALNLISQFEPENQPLGFWIFDTEGVEKAVERWKKNMPTVRPCFAVKCNPEPHLVKLLGELGCGFDCASLNEIKEVLDLGFNPEDITYSQTFKPYNQLIEASHLGINHTIVDSIDEVQKIAKYAPKMGIMIRIMENDTSAGHVFGEKFGLHDDEVEIVLKEIKDKGLNLDGVHFHVGSDSHNSEVFTKALTKARNTVTLAEQFGMKPYLIDIGGGFSQVAPFEEFAATIEKTIKELEFPERTRFIAEPGRYMASNAFHLVSSLHGKRVRIQNGKKQIEYTSGDGLHGSFGCCIWFEKQKSCECITQKVNENTKMYESIIYGPSCNGSDKVATQELPEMEPGKDWLLFPNMGAYTISMATNFNGFEERNHVIYTLPLKSTKIIQIPKSIECNSVPSLNGIPHYA

SEQ ID NO: 1056 - protein, 502 aa, Flavobacterium segetis
MGTINTKIYKYMSIWKTKPLSVLLSEATEDEKGLKRTLSARSLVALGVGAIIGAGLFSLTGIAAADNAGPAVTLSFILASVGCAFAGLCYAEFASMIPVAGSAYTYSYATMGEFVAWIIGWDLVLEYALGAATVAVSWSQYVDKFLQNYGIHIPNSILHGPWDTTPGIINLPSIFIICLLSVLLIRGTKESALINNILVILKVTVVIVFIGLGWGFMNSANHTPFIPVNEGEALLSSGEMSFLNFFSSDYFGHYGWSGILRGAGVVFFAFIGFDAVSTAAQEAKDPQKGMPIGILGSLIICTVLYVLFAFVLTGLENYLNFKGDASPVTTAFAKTGYTFLNSGLTIAIIAGYTSVMLVMLMGQSRVFYSMSVDGLLPKFFSTLHTKNRTPYKTNLLFMVFVSLFAGFVPVSDLGHMVSIGTLFAFCLVCIGVIVMRKTNPDAVRGFRVPFVPVFPIIGVVICLVLMAGLPIESWERLAIWMILGVVIYFFYSKKNSKLNNPE

SEQ ID NO: 1057 - protein, 493 aa, Flavobacterium frigoris
MGTINTKTNKYMSIWKTKPLSVLLNEASEDEKGLKRTLSSRSLVALGVGAIIGAGLFSLTGIAAAEHAGPAVTLSFILAAVGCAFAGLCYAEFASMIPVAGSAYTYSYATMGEFMAWIIGWDLVLEYALGAATVGVSWSRYLLELLNKYGVHLNPKFICSPWETLTLGDGTIIDGGYINLPAILIVSALSLLLIRGTQESASINNILVVLKVIVVIMFIVLGWDYIDPANYSPYIPENTGVKGQFGWSGIAAGAGTVFFAFIGFDAVSTAAQEAKNPQKGMPIGILGSLVICTILYVLFAHVMTGLVPYYKFAGDAKPAATAFAVTGYSFLQTGLIVAILAGYTSVMLVMLMGQSRVFYTMSKDGLLPPLFGQIHSKFRTPYKTNLFFMVFVSLFAGFVPVSDLGHMVSIGTLLAFVLVCIGVLVMRKKMPDAPRSFKTPFVPYVPIAGVLVCTYLMYSLPYESWIRLVLWMAIGVALYFVYGKKHSKLNNPD

SEQ ID NO: 1058 - protein, 343 aa, synthetic construct; identical to SEQ ID NO: 1011

SEQ ID NO: 1059 - protein, 217 aa, synthetic construct; identical to SEQ ID NO: 1012

SEQ ID NO: 1060 - protein, 271 aa, synthetic construct; identical to SEQ ID NO: 1013

SEQ ID NO: 1061 - protein, 222 aa, Bacillus subtilis
MFEKYFPNVDLTELWNATYETLYMTLISLLFAFVIGVILGLLLFLTSKGSLWQNKAVNSVIAAVVNIFRSIPFLILIILLLGFTKFLVGTILGPNAALPALVIGSAPFYARLVEIALREVDKGVIEAAKSMGAKTSTIIFKVLIPESMPALISGITVTAIALIGSTAIAGAIGSGGLGNLAYVEGYQSNNADVTFVATVFILIIVFIIQIIGDLITNIIDKR

SEQ ID NO: 1062 - protein, 343 aa, synthetic construct
MIKLSNITKVFHQGTRTIQALNNVSLHVPAGQIYGVIGASGAGKSTLIRCVNLLERPTEGSVLVDGQELTTLSESELTKARRQIGMIFQHFNLLSSRTVFGNVALPLELDNTPKDEIKRRVTELLSLVGLGDKHDSYPSNLSGGQKQRVAIARALASNPKVLLCDEATSALDPATTRSILELLKDINRRLGLTILLITHEMDVVKRICDCVAVISNGELIEQDTVSEVFSHPKTPLAQKFIQSTLHLDIPEDYQERLQAEPFTDCVPMLRLEFTGQSVDAGLLSETARRFNVNNNIISAQMDYAGGVKFGIMLTEMHGTQQDTQAAIAWLQEHHVKVEVLGYV

SEQ ID NO: 1063 - protein, 343 aa, synthetic construct
MIKLSNITKVFHQGTRTIQALNNVSLHVPAGQIYGVIGASGAGKSTLIRCVNLLERPTEGSVLVDGQELTTLSESELTKARRQIGMIFQHFNLLSSRTVFGNVALPLELDNTPKDEIKRRVTELLSLVGLGDKHDSYPSNLSGGQKQRVAIARALASNPKVLLCDEATSALDPATTRSILELLKDINRRLGLTILLITHEMDVVKRICDCVAVISNGELIEQDTVSEVFSHPKTPLAQKFIQSTLHLDIPEDYQERLQAEPFTDCVPMLRLEFTGQSVDASLLSETARRFNVNNNIISAQMDYAGGVKFGIMLTEMHGTQQDTQAAIAWLQEHHVKVEVLGYV

SEQ ID NO: 1064 - DNA, 9687 bp, Escherichia coli
  source 1..9687: mol_type = genomic DNA
aatcgccggtgtactccgcgtcagaaaggtatacagccacggcagggagatcctgctcttcaagaaaaacagggcgcccgtcaaaccaggtgaccgtgtcggtgatctcggctttcagtttggccagaatggctgcacgaattgcgctgtgtctgttcatcgcttcaggtggatcctcagttggtttttcagggctgcggaaagttctttgggcatatcgctttcaataaggcgctttgaaatagcggtgaaggccacggtgagcggtgtctcaagaggaactttgaccacatcaatcggataacgggcctgacctacgcgccgcatgacctgccagcgcccgttcgcaagctgttggataaaagcgttacgaaaggtatagggcccgattttaaggacgctgcccgctccgtttctggccccttttttacgcgagagcctgacgcgcgccgtgccgagctttatcgcaggaagattaccgcggttgatttttatcgacgcgaccgggcgatcgtgacgggccttgcgcagacgggaacgctggcggaccagacgaaccggaagcccctttttccggttatcatcaactgttgcttctttcgctacagctttgctcccctggcttatcgttcttctggccaccctgttaagtgcttttgcggttgcctcaggaacgattaaccggctgaggctgttcaggttctgaatagccctttccagtcctttcacagacatagcgcctcctcattcgagatggatgcggggttttccgttgaacatgtcatagcgggtaacgatcaggttcttaccgtcgtagtcgacgctgtcgtttcggcgtggctggtaaagctcagagaaaaccaccagcgaagtacctgttcccgacaatggccccatttcctcgagttgctcggcgggaacaacgtcatagctgctgccattgatgatcgctgtctttcccatcttttttatagtggccgcgtccatgcgcgccgccatccggtcaaaggagttaggcattgatcttaacttcaacaacggtggtgtttgcccctgcatcttcccaggcgatgcccgcggcaacggcgtccgtttcttcgatcgtgattttgccgtccttcagatacacctgcgccccggcagtaaccgcatctgcggatacttttggcaggaggaaaacaccctcagtaaaaccgtccccggtatcgccagccgggatatcggtaattgccaccgcgataagttttccaacaacaaccgggtcgccgctgtgaacatcggttgcaccactgtttaccagagggatcgttttcccgtcctgcgcatagttcttagccataacttctccattcagcccctttcgaggctggtttcaggtataaaaaaagcccttacgggcgtctgtttgtcaggactgttttttactgaccagaggatttggtcatgccgcgatagtccagcggcgccacgccagcatcaatacgcactttcgtggcgataccatcagtggtgaagccttcctgctgatcgatgtatggcgtgtcgacgccgttgagataagcgacctcaatggtgtcggtgcccttcgcggcagccagataccaggctttcgcatcagcttcatccagacgtggttcggcaatgacttctgcaaagttctggatagggttaacgatcccggcattgatgtctgcacctttaacactggccgacttgatggtctgatttgccagagtttccagggcgacgggcaccagcatgtaggccggacggatattcagggttcgctccccctccttctgcagacgcatcagcttgcgcgattcgtccaggctggccacagaaattgcacccgagctcaggttcttgtgatcggcatggaacagcgcctttccgtctgagagtttcgggtttttggtcagaatggcgtaaaccagatcgccaatcgttgctttcgccgcgcgccccatcttcatcggtacgtcggtaagctggttcagatcgtcgttgatgatcgcctggcgagttactgagaagatttcaccatacgtggcaagcgcgatggtttcgcctttgtcactggtagtgatgtacttgtactcagccccttcgcgaacctgtcgcagagaagggaacccacccataccgacacgatgcgccgttttgaagtccgacagctggccttttttggtccactgctcgaaggtttcctgcgcctcgtcccagccctgaatcagcgctttgttcgcaacatcaagcagaatgttgccaaagtcagaggtgctgtgggtcagcgccaggccaaccatctgcatcgggttgtagctggccacgccgatacctttttctgtcagggccatacgcgcatactcgcgcagcgtcataccgttataaacgttatcccgctcctgaccttcgaacccggcacgcgccatcagtgcctggcgaataccatccgcgacgaagttaccgttgcccgcatgaatatgcggctgagtggttttattggacggcgtggccgttttaccgagttctgccagcagcaaatctttcgccttatcgacggagcaatcagggtcggccacacactgattctgcagttccatgtgcttattaccgaacatggcaaagagatcgccgatagcgttaacacgggttttctgctcagccaacacctgcgcgcggatcgcattttcatccggtgccgggtctgtttttgcctgcggtgcctgaggctgggtaataaccgggtcacgctgggtagtgttgcgcggcggggtgatcatgttgcgaatgctttttggcattttttcaaattcctcaatacgttttgaatgaatacaggccatagcctgaagggatggtgtcacctggtcggcaaaacccagttcaaggcactcgctgccgttcatccaggtttcgtcctccagcattaccgcaatttcttcggtggattttccggttttctgtgcataagccgggataagaacggattcaaccttgtcgagaagatccgcatagtcgcgcatatcgctcgcgtcaccaccagcaaacccccagggcttatggatcatcatcatcgtgttttcaggcatgatgaccggattgcctaccatcgcaatcaccgaggccatggaggccgccagaccgtcgatatgtacggtaatcgccgcgccgtggtgcttcagcgcgttataaatagcaattccgtcgaagacatcaccaccgggcgagttgatataaaggttgatgtgggtgacgtccccaagtgcccggagatcattgacgaactgtttcgccgttacgccccagtacccgatttcgtcataaataaaaatgtcggcctcactgttattgctggcctgcatgcggaaccacgaattactttttgcgctggctttcggacggtggcgcgcccggttctttggcttcggcactggtgcctcctttatcattggcggggtcggtgtcaaacaccaggccctgttcacggttctcgtcaacctcagctttacggcgtgacttaacatcatccgggttgcgaccgctggcacgtatccagtcggattcagtagcagcaccgccgcggatctgcgttttccaggcattcgcttctttaacgggatcaatccacggcataacgggccccgaataaaccgcgttataaagcgagtccatatcaatgcctctcggcagcttgatttctccggcagcaatagccatcttgagccaggctcggtacatgggccgggtcactgaaccgatgaaccagtcctgaagaatcagatagccgtcggttgactcgacaagctcctgccgctgggcactgtacgttccgttgtagtttctggatgtgctggaaaagctgaggcgactgccggcggacacggcacgcagctgtccgttacgaaaagattcgaggttagggttcgggcgatcggatttaatcatcccgatttcttccccggcctgcagttcgtcatagagcataccgggctgaatcatcagctcgcggtcatcgctgcttgaatcagaatcgaagctctgtccgtcgccttttttgatatacatgccgagtgccgcagcaattctggcagcagtaagctccgagtcctcgtattctttcagcgcgctcagacgcatcagaacaccagacaaaagagacgttccgcgggtctggtgcaggcgtcgggtgaatttgagatgcagcatgttctctgcatctatctctttggtatcaaactgacgcccggatactggcaggcttttatagacctgatattttttcgggcgtccccagttatcgacaaaaacgccctgattgagctgggtggcagcatcgctgttcatcggcacaaagtccggctccagcgcttccagccagaacggcacgccagcaaccggctgaagaccatttccggtaccgcgaaccagctgagcaaatacctcaccgtcccggagccacgttcgcagcatcagccgctccagcattgggcgggtaaactgggttgtgacatctggccttacggaccattcgccccactttcggcggatatcagtggccagctttttagcgatcttcccgttactcagcatcggatgcggttcaactatgatgcccttcgcacccaccaccctttcttccagcttgtcgaaaacgccgatcaccagatcgtggttgttatccagccagcgcgcctgctgcctcagcgaaaccgcccccatctggctgagctgatcggctgaacgattttccttctgggctttgtgggtacgcgtttgctttaccgcctcatacgctttaataactgcgcgggcacgcaggcgtgaggctttccagcctggtgaaaacaggccaatcgcatcatctaaaaaactcatccaaacctcgccagcctgtagccgggtcgcccacggcgtttgttattgagcgttgccagtcgtcgctcccattcctgacggccttttctgatttccgacaggttttcgagcgtcatctgctgcccgttgaaagtgattgatttcccctccagaacagacagctcggctgcagcatagcggtcgatcatgttttgaatatctgctggattcacacccaacctcctgacgaagaccacggattagcctgctcggttacgggcttctcacgttttggttttggtttagatttcggcgcaggcggcggggatggcatttcgccagcttccgtctgcgtgtcctcgatccacgtttcccgccgtgcccactcaggagctgacggccatttgattttttcgtaaccactaaggatggcgagcgcgtcggcataaacgagcaggtcaaatgcttcgtttgcgccccggccgggcttactccatttcccttcattcgagcgttcctcatacgtcagttcgtcatagaaccagctgcccagccaggcggggaaatgcacatagccagggccgggtgaatcacgccacagcgcattattcacccggtctttaagggcatcggtctggagaagataaagaggcacatcaccagtcgcctgtgcgcggcgcgttgatctgcccgtgttgtcgggaaacgttcgctggataagtttgctgcgcctgacgctgtcccctttgaagagatagatacgcttacccagcccctcacggcgacatctgcgccagaacttgtaggcattatccgtcacgccatcttcgccccctgagtccacggccatcgacatcagccgcatgccctttgatgggtcagctgcgagcggccacgttttatcaaagacgtcagtgagtaaaagatcccagtcctccggatagctcgccggatccacctgaatgctttcaccgttgccgtcgcagcgcagcgaatgccggatgttgtaacggtcaactatccagcgctcacccatacttccataacccgtaatctgcacaacaaagcgccggttgcgcccggcctgcacgtccacggtcgcagtgagaaactgcacgccgttcggtaccgaacgttttgggacgtcttcggcacgctgctcgagcaattcacttttacgctgctccatgctggctcgcggcaaatagggcctgccgaaatcggtgttgatcaccgtcttcagggtttcttcgctgcgcgtggattcatattcctgctcggcggtcagaaacttataaataagctgcgcccaggtctggtaagcagctgccggaccttccatccagaaggaggcaatacgggaacgacggccatcaccgctaaccaggcctttcctgtcgatggtttgcccgtcccggagccagacacatttcatgttaagcgcacgcttcatgtccggtgtgatcctgcctttacaggcagggcactgaagaaacgccgcttcgctggcaagcacaggatcgctgctgtcgcggtatccggtcatattgtccatttccggctggaaatattcgccgcaatgcgggcatggccagtaaagacgacggcggtcaccacggttatagagcgataaaattccggtggtcggaggggcttcatggggcgtggagcgccgccattttgtgtctctgatatccctcccgggcgagctctcaaccagcgtcatcccggaggacatgaatgtcgtggttcgtttcgatgccagtgaaaaagcatccccctccccgtcgatatcttccggaaagcggtcataatccgtcagcgccacacttttatagtccgaggacgacatgatattgacggatggccagcccagcttcagatagttaccggcgcggaatgtacggtcgtagacgttgttatcgttacgtcttgggcttagccgggttttaacttcagggctacagcgaaaagtacggtccaggcgttttttggaatgctcgcgcgctttttcctcagatacctgaattacaagcatatctgccggatcgcagacaatgttataaacgatccagccgtcaatcagcccgatggttttacccgttcgcgctgggcccacaaacacaaccgcatcgtattcacgcgatgccagacagttcatcggctcaatcacatagggtgccagatccggatcccacggaactgagtttcccgcccccattggcacgcgcatataagtactgaccgcatcggccaccggcatacgacgcggggctcgtaaaataccggaaacatcgcggcggatgtccctggcggatgcccgctttgccatcagtcctcctcaggctgctcctcctcttttccagcgtcctgcaccttctccgccatctggtcgcgcagatcatcgataacgctttgcacacgaactaccgcagcaggcgttaaagcacagtcgcgctcgagcacatccgggagggtttcaagtaccatgacgacggctttcgccatcaatgagaattctcgcgccacttcatctgcgggtattaactgccccgtatcctgttcgaacttcagcctctcgttctctgctttccagtgggacagcctgtcagaagggggcatatcgtcgatgttggccgaaacggtagggatcatcagttcggtcagaatgtcggtcaccagatagagctttaacttgctattgctgcctggagcaggttcaacatttttcagtctcgcggcaaccgtctgacggtgtacgccggttatccctgccagctggttgatattgagttttaaagtggcaatttcctggtccatgatggtgaacactttttgaacgattcgacatgttgcgaaaatggcctctaattaaatcaaagacctgcgcacatgatgatgatgaccctggatccgaaaaactagccgtttcccgcgagcacgccgccccgtggcagggtccccctccgggagtaccttttgataataattatcaattgcacactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgctggataccaaggctgatgttgtagatattggtcaccggctgaggtgtttcgattgccgctgcgtggatagcaccatttgcgatagcggcgtccttgatgaatgacactccattgcgaataagttcgaaggagacggtgtcacgaatgcgctggtccagctcgtcgattgccttttgtgcagcagaggtatcaatctcaacgccaagcgtcatcgaagcgcaatattgctgctcaccaaaacgcgtattgaccaggtgttcaacggcaaatttctgcccttctgatgtcagaaaggtaaagtgattttctttctggtattcagttgctgtgtgtctggtttcagcaaaaccaagctcgcgcaattcggctgtgccagatttagaaggcagatcaccagacagcaacgcgccacggaaaaacagcgcataaagcacttcattagcagcgccagatagcgtaatgattttgttactcatggaatatttccttttaggcgtgagcctgtcgcacggcaatgccgcccgagaggtaaacgcaacctaacggcatcacccaggctcactactgaaagactctctttgatgtgcgcgtgcgatgcgcgtagaagactgatttatcaacctgtctttatatcaggattcattacctgactatttgtgggtaaagttcgtagtgcgctgatcgtgcaaaatgattttagttgggaacagttcgcaactctgtcccataaaaatcagcatattcccatctatcccatatccagcgcattgaccatcgggatactgaagggagattccatcatctcttagaaagatcaccatctcttttgtttcaatttgcatatagctacctggaggatttatgaatgcaaggattttcatggactattaccatgagattgattttccatctttattcgcgagagcagtggaaagcgatgacgatgtgggtactacattgcgcattcacctactttgtgagcgcatggtcgaagcatggatatgcgcatgctgtgactgccaagatctctttggaagagataaaaacaaacttttaatcgaatgtaatactaaaatatccatggcgggaaacctgggaatccccccggaacttatgaaatcacttaaaaccatcaactcaatgcgtaatgaccttgcacacaatccatcaatacaaagcattgctgattcaaggatccagagcctgaaggatactctgactgaatactttaaacagcatccaacggaacccagcatggaagaatcaaaactgggtatttttaacgccgagaatcaattaaccgaagaagtttccttagatagtgacagttcaaaaaacagacttaagttaatcttgctgttcagcaagttaatgcaggcgttaatgcaattagttgcagctaatcataatgggcgctgggataaccaatttagccaattcgtttaccatgtgaccatgaacgcaacaaagagataaatccaagcccgttttgtacgggctgttgcattatcacaggcactcagtgaatgcctgctgtaatgccgctagtcgtcgagttgcaacacaccgtg

SEQ ID NO: 1065 - DNA, 735 bp, Escherichia coli
  source 1..735: mol_type = genomic DNA
atgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattga

SEQ ID NO: 1066 - DNA, 9621 bp, Escherichia coli
  source 1..9621: mol_type = genomic DNA
atggataatacctctggagattttccatgtaataagatggacacgcgtaagcagttaccgctaacaccaagtcaacaggggtttttattccattccttaaaggataagaaaaggagtaactaccatgagcattttacatgcattttttctcagcatgtagatagcgcccacttcaagtgggcgctggaaacgttatttcgaaagcatgagtgttttcgcactgattataactgggagattgatgagcgcccttgtcaggtggtgaagaccgatgtgttgccggatatatatgtgttagactgtgagcaagaggaaatacgttttctactagctaatgatgacattatcattcctgtcccgcaggatgacggtattgatgctataattcctcaactgctacaggctgatttaaaatacccattttccttgaaaacgatcccagtccgggcctaccttattcagtcaacgaaagaaagtgcttttatactatcataccatcatattgtgatggatggctggagcttatcccttttcattaaacagttgctccaactctatggagcggctgtggtcagtggtgtgagggatgatagcgccattatcccctcatctctgaaaccccttgtagacacactgtcggcccgacgtcacacctttcagcacgactattgggctgcatatcttcgggagggaacaccaacttgtatcgtgccgctgtcacaatatcacacagatactgaagccgagaacaattcttacgttaatcaaacaaatcatgtggagatcaatttgtctccggatgtgtgtcagaaaatacagacgctatgcagcgattatcgtatcacccccgcagtaatcttctatgtggcctggggcatcctgctacaacgttggtgctatgctgacgatgtgttattcggcgcgacaatatcagggcgaaatataccaattgatggtatagaagaaacactagggctatttattaacacgttgccgctgcgtctgcgtgatgatggggcgacactgttgcaacacttacaacgtatgcaccaaacactaatagctcactacagcaatgaacatgatgccttagccagcatacaacggttggtacataaagaaggtcatgctggggatctttttaataccttagtggtgttggagaattatcccgttgatatgacattattgtcatgcgcgtcgcccgtggcaattcgccatctcagtgtacatgaacaaacgcactatccgctgaccttgaccatcactcaacagaagggattccgtttcagtattgcatatgcccttaactacctgaccaacaacatggcgcaagcgttgctgatgcacctgagttatctgcttgaacaactggtggacaatccgcagcgccccattgctgcgttggtaaacttgtccccatgccagcaggcgcaggtacttcaaccctatctggaacgcatggcatgccgggattgggatagtcaatccaacgtcatcgaacaatttcatcaagttgctgcgacttcgccagcacaggtcgcagtggttgatgaattgtgcgcgttgacctattcggagttggcagcacaagctgagcagctcgcggcttatttggtacagcagggtgttatggttggcgatactgtaggcataattagcgaacgccgggtaaacaccgtggttgccatcatcgccatcatgttgatcggggctgcttatgtgcccatcagccccgactacccagtgggtaggatgcaggaaattattgatgacagcggcttggcgctgttgctggtacatggcaaaccgctagatgcattaaacgttgcgcagagtgacctctgtgcatttcccgtcgcgccctcggtggtatttccggttatcacaccagattctcgcgcttatgtgatttattcatcggggtcgacgggtaagccaaaaggtatcgcggtggcgcaccgcggtttattgcgcctgatacaaggcgacagcccgctgaaggtggagagcggtgagacaacgctgctgacctgcccatttgagtttgatgtttcggtgtttgagatgtggtctaccttgctcaaccacggcaaactagtattactcagcaaacaggcattacttgatatcaatcacattcgccgcacgatcgctgatgaacaggtggcgcgcgcctggtttacctcatccttgtttaactcctatgtggcagaaggtgccgatttcttcggtatgttacaacacattacggtgggcggcgaagcagtatctgcgtggcatgtcaacgacgtgatgcaaaaatacccgcatctggtggtgacaaacggttatgggccgacggaaaacactatttttaccaccgcatatcgtttcaacgggttgcaacccgcccgagtcccgataggatacgcggtaccgggcacctcgctctacattaccgatctccatgggcatttgttgcctatcggtgccaccggtgaactagtggcgggtggagtgggggtcgccatcggctatcagaacaacccggcgctaagtgcgacggtttttgtccctgatccttttattcccggcggtatgatgtacaaaactggcgattatgcccggttgctggatgatggctgtgttgactgttttgggcgtaaagacggtcaaattaagatcaatggacaacgaattgaaaccggagagatcgagcagcgcctgctggagtgctccggcattatcgaggcggtagtggttccttaccgcgtacgtgaaacgctgcatattgcggcagtggtctgcgtcaatgatagctatgatgaggtggaagttcgagggcaattggctgacagattgccgccattcgctattccggaatccttggtggtggtgacggagattgcaaaaagccatagtggcaaggccgacttggcgcagttgcggtatctcctgcccgcaactcagtgcaacgccgtgtccaccacgatatcagaggtgcatagtgacatggaacatgcgctgcatgctatctggcaacgcgtacttgatagacaagacattgatagcaatgcctcctttttcgccctcggtggcacctcattggataccatcagggttaaaggggatattaagcggcaacttggcttggagattgatattaccgatctctttaagtacccaacgctcacggcgttagcacattttctcgatactgccgtatcgccggaggatgcaattccaacgcgtgctgttgtctacagcgacatgccggtggcgattgtcggtatggcgggacgtttccccggtgcggcgaatattgcagcgttgtggacgctggttgtaggaggggaatcgggattaacactgttcagtgatgaagagttgcgcgcgcatggtgtgacacctgacacgcttaaacaagcgaattatataaaaaccaaagggattgttgatgatcacgaatggtttgatgcggatttctttggttatactcccaacgaggcggaatgtatggatccgcaaattcgcttattgcatcagtgctgctggcaaactctggaacacgctgggtgcgatcctgccacctttactggtgcgattggcatttatgccggactgctgacatccccccactggcttaatgcggtaatgcaagacactaccgactctaccgccctgtacaaggccagtatcctgaatatccattccgtcacagcattgattgcccatgcgcttaacctcaccggccctgccgtgacgcttgacaccgcctgctccacttcggcagtggctatccaccaggcctgcatcgcactacgtaaccgggattgcgatgcggcattggcgggcggcgtttctatcgagatgcctgcgtaccggggctatgaatatcatgaaggcatgatcaatgcgcgagatggtgtctgtcgtcctttcgatagccaagcctctggcacagtcaccggtgatggcttaggaatgctgctgctaaaacgtctggacgacgcgctggcggatcgggactgcatctatggtgtgatcaaaggttcggcggtcaataatgatggcaataacaaaatcggctataccgcgccgagtgtgatcgggcaatcaacggtaatccgcaccagtttgcgccgtgccggttttgatagcgacagtatcggattggtggaagcgcacggcaccggtacggtgctgggtgatcctattgagttacgtgcgcttaatgaggtgtttggcccaacacctgttccgttttgtgtggtttcagcgctgaaaagtaatattggccacctcaactccgctgcgggagtggcgggcgtcatcaaaacaacgctcgccctgcatcatcaagtgttgccaccaacagcgcactttcgccaactcaatcctgctatagatctatcgcgttctgcattgtacgttaaccagcaagtccaaccgtggccgtcgacgcgtcctcgtcgtgcactggtgagctccttcggcatcggcggcaccaatgccagcatcgcactagaagcacatcaacatgaggacgacccttcagcgacgggggtacgcgacagctatctgttgttgttctccgctaaaacacccgctgcgttagagttgcgcgtggcctccacactggaatatgtcaagcatggagtaggggtgcgcctgccggatgtggcttatacattgcaaactggacgcacagcctttgaccatcggcgggcttatttggtgagtcgtgggtcgaaaatcgatctctcctgtgccacgatattgcaagcggaaatcttcaatggtcagcgcacgacagcggagatctgcttcatgtttcctggtcaaggtagccaatatcacggcatggccagcgcgctctatgctcatcaacccatgtttcgccagcacatggatcgctgctttgctgcattccaacgctattcgacggtcgatctcaaggcgttgttgtttgacgatgaggatacgcgggatattgatcaaacgcagttcacacaaccggcgttgttctgtgtcgaatacagcctagcgcgcaccttgattgatctggggattacgccggacagtatgatcgggcacagtctgggcgagtatgttgcggcctgtattgctggcgtatttactcttgaggatgcgctgcacgtcattgaggcgcgcggacgtttgatgcagtccatgcgtcccggtagcatgatggcggtctaccttagtcgcgaacagttgaccccatggctagctgcagaacggggtattgaactggcagctaataatagcgcgcatttttgcgtagtcgcgggcgagcaggcggccatttcccgtttgagcacacgcttagtcgagggcgggatacagcacaggcgcctgaaaacctctcacgcattccactcggccatgatgacgccgatgctgcacgattttgcacagttgctggggcaaatcccgatgcacgcgccgcacaagcgctttatatctaatgtaagcggtacatggattactgaggagcaagctacctcgccggattactgggtgcagcaagtgcgcaacgcggtgctgtttagcgaaggtgcggcgcaactgttggtacaacccacgctgtttatcgaatgtgggccgggtaatacgctctctacctttattcaaggacataaccaatacagcgatcagccgaccctgttgacgctacgcaaagccaacgcggcgatcgatgatgagcacatgttgcatcgtacgctggcggcgctgtgggtcaggggggagaatattgattggagacgctttaaccagacggcactcggcaagcacattccattgccggattaccccttcgaacagacttattactatcgctatggtgctgcactttccggttatcgccagtatccaaatcctctgcgccgtccgcaagatgagtggctccagcgtgtgctgtggcggatgcacgacacatccttgcgggaggcgttctatgcgccgggcgaattgatcatcattattagtgctgacggcgacaagttacagcagacgctgatgagtagtggtgtcgacagcatcacaatgccgctgccgatatcgtcagaggacgacgtgtgggataatgaccgtattctgacgcatttccacgacatctgcgcacttttagcacacaaaacctaccgacagttacattgcttgtatgctcccggtgcggaggcaggatcatcgttgacacagtcgctctcgggactttatcgtgttgctcgctggtgtatgcacagcaccacgccgctggcgtccttaacagtattgacccatggtgcgtttcgcgtacaggaagaggataatcccgaaccgacgctggcagcgttgtcgggcgcagtaaacgtgtttgcccaagagctgcacccgaccgaagtacgattgatcgatatcgatgcgcagagcagtgatgagaatctgaacttgctaacccagcgtctggccccgaaacaagaaacggtaatggcgctcaggcaggggatgctttacctgcgacgctttatcccgacacggctgttggcgcacttaccccctcaaacagggtgtataccgggcaacgtactgtggattatcggcggtgaaaaggggattggccgcatgatcggcgaagcgcttgctcagcgtgagggagtccgtgtggtactgagcagccgcacaggttatcaccatgaagcggtgcagcaggacgcattagacgttatccactgcgacgtgacgcaggcggaggcggttagagcttgtctggcaactctgctcgaacgctatggacggttggatggcgtgatttttgctgccgacgctaccaccacattgacactgcatcaattgagcgaatctgcgctacgcgacacgctaacggtgaaagaacggggtactgctaatgtgctgcatgcgttagcgcaacggaatctgctggatgagcgtctgctactgctgttctgtaactcgttggctgccgtgaatgcggagattggccagacaggctatgctaccgccagcgcctatttggatgcactggcacagcaactgcgtacccgctacaaggtgaatgcgctcagtatcgggttggatgcattgcgtgagcagggcatgttgttggatgctataaacggcagtgaatacgatgtcttgcgtggactgcgcccattgatgacgggaacgttgctacaggcttataaacaacaaggggctgacaccagctactacgcgcgattatcccccgagtccgattggctgctggatgagcaccggatatccggtatcgccacgctgccgggaaccggctatctggcgctggcgtatgaagctctgcgccattactttgtgcaagaccaaatctgcattgatgaattggtctttttggcaccgttgactgtgatggacaactgcagtgttgacgtttttgttgacatttcacctaacgggcaaggagttagtgtcgaggtgaaatcaatgacggagcgctttagtggcacgttaacaacccatgccagaggcagggcgacgcgtctgatggtagacgataatgttgtgtgcgatctcacggggctgatgcgcgagatgcacactatcactcctccaacaaaggaattatcgagcacgcacttccactatgggccgcgctggcacagcgtacaacaactgtatggcaataccgcccagactcaggttttcgcaacgctggccctgcccaccgttgccgctaatgatacgatcgcactgcaccctgcgctgttggacatcgccagcagtgttgtcgaacaactgcctggttttcatactgattcggtacctttcctttatcaggatttacgcctgtaccgcccgttgccgaacaccttacatgtggcgctgactgtcaatcggcacgatgaggagggtgacagctacgctttcacgctctacgacatggcaggcgagatggttgcccgctgtgcggcaatggtgaagcgcaaggtacagctccacatacaagatgtcgatgacgacacgcgactgcgcgtgcccagtgccgataactaccaactgcggctggccgctgagggggagggggcaggaaagctagcgttgtgccctacgccgcgcttagcgctgggggattcacaagtagagattgaggtactggccaccggactgaactttaaggatgtgctgttcaccacgggattgctccggcagcagccgggtgaggctccgctgcaattgggactggagtgcgccggacgcattactcgcgtgggtaaaaatgtcactgaatttgccccgggagaggatgttatggcggtgctaaacggtggttttgtccagtacgcacgggtagaaagcgattgcgtagtgagaaaaccagcccattgtcgcatcgaacaggcggctgcgctgcctatcgcatacctcaccgcctattacgcactggtggtgcgcgctaatttgcaacccggagaacgagtattgatccacagtgcggcggggggcgttggcttggctgcgctacatattgccaaacgctgcggagcacagattttcgccacagcaggtagcgagcagaaacgcgattacttgctttcgctaggcgtacatgccgtagctgattcacacgacgaacagttcgctgccactctgctgaccgcatcggacggacaggggatggatgtgatccttaactccctcacaggccgtctgcttgacgctagcctcgcgctgctggcaccgctgggccgttttcttgagctgggcagcaaggacattgtggaagacaaagcgctaccgatgcgtttcttcgcccaaggcggcacctttattccgattaattttcacgcggcgcatggtgcgtttagtcgctacctgcaacagattgtcgcttggatagatgataacacgctaccgctccttccatgcaaatccgtaccattgcccgaggttgcacgcgccttcgccaccctgaccacgccgcagcatattggcaaggtggtggtaacgcatcgcactgcggcaggcatggaccggctgaacgccatgatagcagaaaggcgcctcggcggctatgcgctcagcatgagcaatgccgaggtgatgcgccaattgtggccgatactgaacactcgcagtccgtgggcgcaactgctgctctcacctcgggcgatcgatagattagcgcggggcaaccgggtggatcgcggtgtaccgtctgccgctaacgatacgattactcagcagacagtgaaaaagcggccccgtccggaaattggcgtgccttacagccccgcgacacgtgaagtggaacgcgtgctctgccaaatcctggaagagtatctgggtctggatagagtaggcattgacgacaactatgccgaattaggggcaacctcactcgatatggtgcaactgagtgggcaaatggcgcgtcactatccgcaagtgagcgtggtgtcgctgtacaaccacgccaccgtgcgccagctggcgacgttttgccagcccccggagggcgagtcaaatgcgccatcaccacagcctgcagtacagacgaatacgcgcgcgaatcagatagcaaaacgtgctttgcaaattgcgaaaaatactgcgcgcagtcacacgtctttgcattaa

SEQ ID NO: 1067 - DNA, 2601 bp, Escherichia coli
  source 1..2601: mol_type = genomic DNA
atggaatacgcaagcgaaatgaacggcatggaaatcgccattattggtatggcggtccgtttcccgcagtcccggacgttacacgaattctggcataacatcgttcagggtaaagagtgcgtcaccttctttagcgaggaggagttgctggccgaaggcgtcgaacagagtactctggacaacccggcctatgtacgggccaagccctatatcgagggcatatgcgactttgatgctgcatttttcggttacagtcacaaagaggcgcagactctggatccgaaatcccgcgtattacatgaagttgcctaccatgcgctggaagatgcaggctatgcccaacgtaccagcgatctgatcaccggggtattcgtgggggcgtcagaagatgtggattggctacggcgttcgctgtcacagattggcggcgatgcgctgaatcgttttgagtctggcatctatggtcataaggatctgctcgcacatctcattgcctacagtctcaatctcaacggtccggtgtatagtctctacaccagttgttcgacttctctgagtgcaacgcatatcgcttgccgcagcttgttgtttggcgaatgtgatctggcgttggcgggtggaattactatcgatttaccgcagaagtcaggctacttctgtcaacagggcatgatccactccaccgatggccactgccgtccttttgacagtcaggcttctggcaccctgtttggcgatggcgcgggtgtggttgtgcttcggcgtttggaagatgctctggcagcgggcgatcgcatctatgcggtgatccggggtagcgcggtcaacaatgacggtaaacaaaaaatcggttttgtcgcgcctggtcatgagggacagaaggcggtcatttgtgcggcctgtcatctggcagaagtaagcccagaaagcatcggctacgttgaaacccacggtaccggcacccgtattggcgatcctatcgagttcgctgcgttgacggaggcgttcgatacttcacaccgccaatactgtgcactaggggctgtaaaggcgaatattggccacacccacgcggcggcgggcgtggctgggttaatcaagacggcgctggtacttcatcaccggaccattccgccgctcgccaactatcaaatgcctaactcgaagctggatctggcgcattcaccgttttatatcccgatacagccgcaggagtggcccgcatcgcggatgccgccccgcgctggcgtcagttcttttggcattggcggtactaatgtgcatatgattttggaagggctgaatcctgcggtgcgcgatgaccatgaccaagtgcgagcaccggtgtttatccctctctccgcgccgtctttcgagcagttggatgagctgacgcaacagcttaccccgttgctggctacccttgatgcgtcaacgctagcctacacacaacaagtggcgcgccccgtgtttgattgccgccgagtgatacaagtggaaaacgatggtacgcaagcgatgctggcatcgctggataacctaatgcccgacgctccttggggcctacactgtccagatctacgtactacgaacgattgtacttacgcacagtggctggcgcattcggcacattatcaacgcgaagcgactgctctgacggcattactcgacggcatgaatattccacccgcttattgccacgctgaaacgtgggcggcacaagcgaacagcagcctgctaatcagaggctgccagactatcgccgcgctaaaaacctggatgaacctgctaccgacattaacactgctgtcgggtgctggaacaggccttttgcctgccgctgcagccagtggcatgattgcgacgcaagacgtgttgcatttgctgtgggaaatggagcaaaaggcgcttcatctctggcttcctgagcgccatgaacctatccccggctacgtgctggcttggcaaggaaatcccatcaccgatgcacagcgtaacgacagagggttttggagtgaagcgctgttggcagatacccgagaactgggggagggcgttcacagtatcaactgggttaggctgccgccggaaataagagaagacgttgatgtattgcgctacgtggcgcaactgtggtgtgcaggtatcaatgtggattgggccgtgtggtacggcactccgctgccgcaacgcgggagcgcatcagcatatcccttcgcacacaaccactacccattgcctggacgggtaatgggtagtgtggagacccaacccgaagcaggacccgaaacgcaccacccttatcaggcacgtcccgtgttaagcgtacctttcgtagccgcacactcgcggggtatgcagtatatcacaggtctgatggaactgttgctggaaatatcgccggtcggggtggacgacgacttttttgaattaggcgggcattcgctgctggtgacgcaattgacctcccgtttagaacgcgatttcaacgtacatatcgatcttttgaccctgatggaaaaccccaatccgcgcaatatctacgcgcatatcgcggcgcaactggggggcgaagacaacctcgaaatagcctgtcagtaa

SEQ ID NO: 1068 - DNA, 870 bp, Escherichia coli
  source 1..870: mol_type = genomic DNA
atgatgaacgtggcggtaataggtgcaggagtaatgggaactggcgtcgctcataacatggcgcaatacggcatatcaactaacgttgtggatatttctcaatctcagttggataaatgccgacagatgatcgaagcgaacttacgcttatataattttcacccgcagcataaaaaaaagacgcatagcacagcggagataatggaaaatatccgtttcacaactgagttggacgacattgtagaatgtgatttggtgattgagaatattactgaggatatagagaaaaaaaatgcactatacacacggatgaatacgatctgcggcgcgtcgacagtgtttggtgttaatacgtccgccatatctatcactgcgttgtcaaaattaatgcgacatccggagaatgtagtcggcgtccattttatgaacccggtaccgctgatgcacaccgtcgaattaatccgtggtgtacatactgcagagcgtacactgaacatatttcaccacttatttgctcagttgaataaaaccggcatcgtggtcaatgattctccgggctttgtcactaatcgtgccatgatgatttttgttaacgaagccatatttatggtgcaggagcagatcgcccgtgctgaggatatcgatacgttgtttaaaacctgcttcgggcacaagatggggccactacaaactgccgatttgattggcttggatacgattttgcaatcgttgcaggtgctatatgaaagttttaatgatgataagtatcgccccagctttttattaaagaaaatggtcgatgcagggtatttgggcgtgaaatcagggcaggggttttatcgctatcaacagacgtacgccgagcagtga

SEQ ID NO: 1069 - DNA, 249 bp, Escherichia coli
  source 1..249: mol_type = genomic DNA
atgaaaaagcaagatatgaaagccgccattcgggaatttctttcacgctcattacgtgggcatacgttgaacgatgatgacgatattttttctctcgggcttgtccattcgttatttactgtgcaaatcatactgtttatagaaaaaaattttcaggttgagctggaagtgagtgagttgaaaacagaacagattgctaccgtcaataaaatagtggagctcattcagcgacaaacaggcctggagtaa

SEQ ID NO: 1070 - DNA, 1131 bp, Escherichia coli
  source 1..1131: mol_type = genomic DNA
atgtgcactgaaaattatgagctggctcagcaagaggcagtcctgtttgccaaacaacatctcgccctggctgcacagaatattgaacgtcagcagtttattgtgccagacattatttcttgcgtggcgcaggccgggtacttaggtgcgtcaatcccccaaaaatatggcggacgaggttacgattcttatcaactgtgcgctttgcatgaagtgatggctggggttcacggttcgttagaaaatctcataacagtgactggcatggtcagcacgctgctgcaacgcgtgggcagcgcagcgcaaaaagcccattatttgccaaagctggcaacgggggaattgatcggcgcgattgccctcacagagccgaatatcggcagtgacttagtgaacgtggaaacagaattacagcaggatggcgacggttggcggctgaacgggaaaaagaaatggatcacgctagggcagattgctgatttttttattgttttaatccactgtggtaatcaattagcgacagtgcttatcgatcgcaataccgacggctttactatcactccactcaacgacatgttggggttacgcggcaatatgttggcagagctgcattttaatgactgccgcctgaaggaggatgcattgctcggtccgctcacccctggggtaccgctggcggtgaattttgcgctcaatgaagggcggttcaccaccgcttgtggcagcctaggattatgtcgggccgctgtggatgtggcagcgcgctacatccgccagcgtaaacagttcaagcggcggttattttctcatggcattgttcagcacttgtttgccacaatgctgacccaaacgcgcagtgcgcaactaatgtgcttcagcgcggcggaataccgcgaaacgctgcacccggccatgataaaccaaatcctgatggcgaagtatgtcgcttccaaagctgcggtggacgtggccgggaaggcggtgcaactgctcggtgcgaatggttgccatgccgattatgccgtcgaacgttactaccgcgacgccaaaatcatggaaatcatcgaagggacatcgcaaattcatgaaatccaaattgcgatgaattacatgatggggagtgaagcatga

SEQ ID NO: 1071 - DNA, 1269 bp, Escherichia coli
  source 1..1269: mol_type = genomic DNA
atgacgaaggatgtcgcactgatgttccctggctccggttcgcaatatgtaggcatggcacggtggctgtatgagcgttatccgcaggtgcgtactctgtttgatgaagctagccagatcacggaacgggacatggcagcgctgtgcctgtcgggtactttggtacaacttgctgagccgacggcaatggcgctggctatctataccaccagcgtggcccactttgtcgcgtggcagcagtttttggcgcaaaccggtgtccatgtcaacctacgttatatgttgggtcatagtttgggcgaatatgctgcgctgacctgtagtggtgcgctgagcttttcccaagcactggcgctggtggcgatgcgtagccgtttagccagtgagatcgcccgcgaaatggacgcgagtaccactatcatcaagcaagggaatcaggcactggtggcagcggcgtgcgaggtggcggagcgtgaaacccgacagcaggtcggtattgcctgcttcaattcgccgcaacagtttatgttatccggacaaaactcggccattatcgccgccgagcaatacctgctggatcatgatcgccaagtcgaagtagtgccgctgattggtggtgttccttaccacagcccactattgaaaccttgcggacagcaattgcgcaaggcgctggatcgctgcgaatggcgacgtccgtgctgcccagtgatctccaatgtcaacgctcaaccctatcccgatacggtcacgccagtccagtggctagaacaacagctctctcagccagtgcagtggcagcgctctctcacctacctgacaggacacttgtcaccgatcgcgatagagatcggtccgcaaagtgtgctgaaaaacctgctgctggagaatcgctatccagcaccagtttacgcatttgacaatcgtcatgatcgtgcgcaactagcattggtgctgggggataacatggcggtgaagaccgatccggaagccgtgcgccgccaacgcatcacgctgctcactaacgccctgacagccacacgccatcaccgcgccgccgatgtggctgccagtgcgcaattgaaagaattgctcagtcgtttttttgagcgcatccagcagattgagcagcgcggcacatccagcgaagaggacattgcttttttgcatgagcttcttgaacagggatttcaactcaaaggcagtagtcaggcagaaatcgacgcctgccacgcgcggttggcttccgacaacggcggacaggcgtaa

SEQ ID NO: 1072 - DNA, 4797 bp, Escherichia coli
  source 1..4797: mol_type = genomic DNA
atggaacagcaagggattatgagacagttgcctaccgacgaccaaacgatagtcgactatttgtatcgtatcgccggagaatatggggaaaaagccgctgtattgatgggggacgcggcgctgagctatcacgatcttaatgcacgctctaaccaactggcgcattatctgcgtgggctggggatcggcgaggatcgtgtggtagctatccgcctgccgcgcggcatggcaatgctgattgccattttcgctattgtaaaagctggtggtgcctatttaccgctggcgtacaatgcaccgcgcagccgcattgaaaatatactgagcaacagcggcgctgtttgtctgatcggtactgacgatggtgatcgctggccgattcctcgcgtcgaaatcgacagcgcggcggtcagtgccatgccaacgacggatctgcgttaccgaccacatgcgcggcaactggcgtatattatttacacctccggttccaccggtgtccctaaaggggtggcgacggaacatgcagcgctgctaaaccgaattgtctggatgcagaacgcttatcctatcagttctcaggacgtgctgtttcagaagacggtgtacacctttgacgtctctgtctgggagatgttctggtgggcgatgtacggcgcatctgtagtgctgttaccgtccggactggagagcgatccgcgaaccttggctcgactgattcagcgtcaccgcgtgtcggttgtgcactttgtcccttcgatgctgaacctgtttgtcgagtatctggagatgaaacaggatcctcgtttgaccgcctcattgcgattagtgttttccagcggtgagaaactcacggtccacagtgtggctcgcttttatcaatcggtggcgcagggtgatcttataaatctctatggcccgaccgaagcagcaatcgatgtcagtcatcaccgctgcctgcgcgggtacgactacgacgatatccctatcggtcaagcgattgacggttgccgactctatgtgctggatgaccatggtaatccggtagcagacggcgaagagggcgaactgtatctcgctggtatcgggctggcacgtggctatctcaacaacgtggcgttaactgatcgctgttttactatacatccaactttgcgccatttagggaaaccggagcggctgtataaaactggagatctggtgtggcgcgacggggaaagccaacaaattcattacattggccgtaatgattttcagattaaaattagagggttgcgcgttgaattgggagaaatcgaagcccatgcgatgcgtttcccgggggtacagcaggcagtcgtggtggcggatcaggatgatcccgacaatcagttgatttacgcttttgtcgtcagtagcgtgccgctcaatttggcggccttaatggacgcactgtccaaaaacttgcctgcctacatgctgccgaaccgtttgttggcaatgtcagagttaccactctccgacaatggcaagtgctgtcgtaaaacgttgctcgacttggcgcgggcgtactcagccagtcgcgtcgatttacgtgaaactcccgccgtgcgctacctaccgttgtcatcggctcaatcgtcgatgtggtttatgcaacaattggcgccgcatactgcactatacaataaccccaccgccttgctgcttgaaggagaactggatcgcacgcggatggacggcgcgattcgtcaattgatgagtcggcatactctgttacgtgctatggcggaaacccacaatggacaaccagtattggcggtgcctcagtgcgtatcgtcgcaggcgttgctgaccatagtgccactcccctcggtgagtgatgataacgcgctacaggcgatgatcaaccagcgtgcggcacaccccatgcccttaacgtcaggcacaccgctgtgccggtttgaactgttgacgctcgatgacgatcgtagcgtattgttgattcatctgcaccacatcatcagtgatggctggtcgaaaggcgttttgttacgcgaattgcaggccgcatataacggtgaatcgctgacgccagaaccgttgttggagtatgccgactacatggagtaccaagaagagtggcgtcagagtgacgcctatcaggacgcgatgcgttactggcaaaataccctggcggggacgttgccgattcttgatatccccaccgatcaaccgcgtcagaaggtggcacgttaccaaggcgcgtttgttgccttcgcattgtctgccaacacatgcgagcgagtgttggcggcggcgcgtgcgcagcgcgtgtcgttgtataactatctgctgaccgccttcgtcctcctgctgcatcgtaatgcgcgtcaacaggaatacatcgtcggtatgccgattgctgcgcggctgacgaaagaacaggagcatatgatcgcgccgctggtcaacgtactaccgctgcgcttacctcttgacgaagcggcatcgttttccgagttagtacagacgatcagaggtattctgtttgccgctttcaggcaccagcgtctcgaatttaccgacatagtgcgcgcagtgaatgtggatcgcagcgccggacatttcccaatatatcagtgcatgttccaactcgacaatatgcctctggcgagcccgacactaaacggtgtcaacgttacaccattattgttggataccagtgcatctcaggtggatatttcgctaagtatgcagcatatcgatgggcgcatcaccggcactttcgaatacgacgctgggttatacagtgcggatcgtattcagcacctggtggcgcagtggagagaactgctagatgaggccagcagccaacccacgcagttagtccgcgatttgattcgtttcacccctcgagagcacgcttggctggcgcggcacaacgccactgaagttgctctgccgcccgtagataacctgctggcgctagtgttaccgcactgccagcagcgccctacacaggtggctttgcgccacgctgacgacgccatgacctatggcgaattgcaacaggccacgatgcagatgtgtacttggctgcgtgcgcaaggcgtaaaacgcggagaatctgtcgcactgcaactgcctttttgtttcgaattgattatcgcacaattggcgatcctctctttgggggcgagctatgtgccgctggatggtaacgcgccagcggcccgcaatgccctgatcttggcgcaggcaacgccgtgcatgctgctggtggcgcaaccgctggaatcccctcatgggctgacgataccttgggtgctagtgcctgactggcgtagcctgctgacagaaataccgaacctgccagttagtgttgcgccagatgctttagattgcgatgcggtggtgatctttacctccggtactaccgggcagcccaaaggcgtccgactcagccaacgtaatctggtcaacttaaccgcatcctttatatccagctatcaagtgacgcatcaggatgttttgctacccattacctcagtcgcgtctgctagctttgtcggggaggtactgccgctgctcgccgctggcggtaccttagtattggcgcaaaaagcgcagagtttggatagtgatgcgctcattgcgctgctggcatctcagcgggtgactatcctgagcaccaccccatcgctttctgccagcctctctgtgctggctcagtcgatgggatcgctgcgcttgtttctgtgcggtggtgaagcgttagagtatgagcagatagcgccgcttctgccgcacatggcagtggttaacggttatggcctgaccgaaagcggtatctgctcgacctactttcctgtcgcaaagcgtagggagcaagaaacgggagcattgcccattggccgcccgattcagaacacccaagcttatgtggtggatgcatataatcgtttggtaccgccaggtgcctgtggagaactctgtttctctggtctgggcatttcgcccggctaccttgatgcacgacaggatcccgagcgctttgtcgagttgccggaataccctggcgttcgggtgctgaaaactggcgatcgtgctcgttgggcaaccgacgggatgttgttctatctcggcagacaagatcgccaagtgcagatccgtggataccgagttgaactgggcgacatcgaaagcctactgaaacagcatccggatattgctgatgcgtgggtcgatgtgcgacgcaatgcggcggcaacgccgctactagtggccttctattgcagcgtcaacggcgttgcgctggatgctcagcaattgcgcgtatggctcagcctgcggcttccgttgcatatgctgccgttgctctacgtgccgctgagcgccatgccgctaggtgtaaacggcaaaatcgatccccagtgcctgccgctggtcgatctgcggcagttggaagggccgggcgagtatgtccctccggcaaccgaactggaacagcgtctggcggagatctggcagcagttgcttggcctggagcgtgtcggcaccaccacaaatttcttcgatctcggtggacactcacttctgctagtacagatgcagcaatacatcgggcagcagtgcggtcagcacgtggcgttggttgacctgctgcgcttcactaccatcaaacgcttggcggaatttctgctggccccggacgcagcgcaagggaccacaggagatcagacacaactgcgtgcggcgaagcagcgtttggcctttggtcacacgcgctgggcagccacaaccgacagtcatcactga

SEQ ID NO: 1073 - DNA, 3033 bp, Escherichia coli
  source 1..3033: mol_type = genomic DNA
atggcagagaatgattttggtatagctatcattgggatggcggggcgtttccctcaagccgatacggtacaggcgttttgggaaaatcttctggcaagccgagagtgcatttccttttacagcgatgaggaactgctggcgatgggaatcagtcctgaatttgttcagcacccagattacgtcaaggccaaaggggaggtagctgacatcgataaatttgatgctgctttcttcggtatcgcaccccgagaggcggagctgatggatcctcagcaccgtgtcctgttggaaaccgcgtgggcagccttcgaagatgcgggctatgtggctgccgattatccgggggatgtgggtatttttgctggcaaaagcatggattcctatttgatgctgaacttgatgccgcactttaaacgcgttttctcttctggcagtttgcaggcggccattggtaacgacaaagacagtatcactactaccatcgcctatcacctcaacctgcgcggcccggcgatcacggtgcagacttcctcatcgacatccttggtggcggtgtgcgtggcatgccaaagtctgttgacttggcagtgtgacatggcgattgctggcggagtaacgctggggccaccagcaaaaaccggctacttgtcgcaggaaggtgggatcaccgccgccgatggccactgtcgggcgttttccgataacagcagcggatttgtgcccggcaccggcgcggggctggtggtgttaaagcgtgttgatgaagcgctgcgtgatggcgataacatctatgcggtcatcaaaggatttgccgtcaacaacgacggttcggagaagatcagttataccgcgccgagcgtggatgcccaagcgcgcgccattgctcaggcacagcggttggcaggtttgacaccgcaggatatcacttatgtggaagcccacggtaccggtacgcgtttgggcgatccggttgagttttctgcgctaagccaagcctttgcgggcgcgtcacaaaagcagtactgtgcgctggggtcggtaaaaaccaatatcggccatttggatacggcagcgggcgtcgcagggttgataaaaaccgcgctggcagtgcagcagggaataattcccgcaacattgcactttgaacggcccaatgcacagatcgatctgaccaatagtccgttttacatcaacactacctgtcaaccgtggcaaccggaaagcggcatccgtcgcgcgggcgtcacctcgcttggcatgggcggcaccaatgcccatgttgtgctggaacaagcgccagccgttgacttgcaagcgcgagcgcctgtacctgcctacagtattttgccgttttctgccaagaccgacagtgccttatcctcggggctggcgcgttttgccgattttctgcaacatgaatcgctgccggataggcgggatctggcgtggacactctcacagggacgtaaagccttcgcacatcgcgccgcgctcgtaaccagagatctacatgctgccgggacgctgttgcagcaggccgcgacagcgccgtttgctcgtggtgtagcgcagacacagctcgggctggggctgttgttttccgggcagggtagccaatatcagcgcatgggccatcagctctatcaggtttggccggcctacgccgatgccttcgaccgttgcgcgacgttactcgagcgtgaataccaactggatatccgccatgagttgttcagagcagaagtctcgttagcccaaggcgaacgccttgcgcaaacctgcctcacacaaccgctgctgttcagcgtcgagtatgccttagcccaattgtggctcagttggggaatcacgccaacggtaatgattggtcattcgctgggcgaatgggtggcagcgacgttggcaggcgtgttctctttagaggatgcgttgcgcttggtggcgcgtcgagcagagctgatgcatcaggcaccaagcggtgccatgttgatggtggcgctgcccgaagcacagattcgcgcactgattaccgccccgctggcgattgccgctgttaacgcccctgactattcggttatcgccgggcctacgtcggagatcctcgccgtcagccagcgtctgacggagcagaacatcatcaacaaacgattgcatacctctcatgccttccactctagcatgatgcaagatgcggcgcaggcgctacgtcaggcatttgagaatgtacgactgaacccgccgacgctcaccatcatctctaccgtaaccggcgcgcacgtcagtgccgacactctcaccacaccggactactggattgaacagatgctgatgcctgtgcagttctccgctgcactgcaagaggcgcaagctacatttgatgtcgattttctggaaatcgggccaggtgccaccctgacccagctgaccaacggtcatgcattaggtgatcgtctcgcgttcagctcgctgccagcaggtgcccgcagcagcgatgagcacaaacatatcttagataccgtggcagcgctttgggtgcgagggcataacatcgatttgtctgcgtttgcaggtgagcaaccgcgccgtgtctctctaccgacctacgcctttgacaaaatccgttactgggtcgacagtccagaagaacagaggagcgccgtaacgccggtggcggacgcgggaagtgtcatccctagcgaaccgtcggtgcgccgtcagccgcgtccggcgttttcggtgccttacgcggctccagagagcaaaacgcagcgcggcttagtggcgatttgtgaagcattgctgggaattgacggcttgggcattgacgacaacttcttcgaagctggcggacattcattgatgctgggcatgttgctggcgcaggtacaggaacggttcgccgtcactctttcctttttcgacgtgatggaggatgccagcgtgcgcgcgttagcgcagttggtcgagcaggagcagcaggatgatggagggtccgcacttgccgtgctagttaacgacatgattaatgagtga

SEQ ID NO: 1074 - DNA, 6501 bp, Escherichia coli
  source 1..6501: mol_type = genomic DNA
atgacgatacatcatgccgcattggcgcgaatgttaccggcggaaaaaaaggaaaagctattacgacagttggcgcaatcaggcgtgtcgcccagccgcattcccatcatcaaagcggatccggcgcaggcaatcccgctgtcgtttaatcaggagcggttgtggtttttgcaaaaatacgacagtaccgccactaactacaacctttatgtggtgtatcgtttgcatggcgtggtggatatgccgatgctcacggaggcattgcgccatgtgcaagcacgccacgccatcttgcgtacccgtatcatagtacgtaacgatcggccttgccaggtgatagatgatgcgtcttcgttagttctcgatacggtcacgctggcagcgcaggccccaacttcagcgctagatgcggtaattcagcaggtgataaacacccggttcgatctggcacgcggtcctctgtggggggtaactcagattattcaacccgatcaaggttgtcatttggtgttttgcgcgcaccatatcatcatcgacggtatctcgttacggctattgttcgatgagttacagcagcagtatgctcgattgcatgctggtaatgaaacgtcgctgcctccgccgccgctgcaatatgccgattatgccttctggcagcgcgaatggttccaggataccttgctggcgaacgaacttgcctactggcgtgcgcgtttgcaagacgcgcctctgctgtcgacatttccatcgctgcacccgcgcccggcacaaccctctacacatggttcccggttcagcatcaccctggatgaaacattaagcctcgcgttgaaacacgtagcccgcacgcaggaaacaacgccgttcgtgctgatgctgactgccttccaactggtgctgatgcgttacgctcagcaacagcgattggtgataggcatgccagtatcggggcgcattcgcccggaattgcagagtagcattggttattatgccagcactgcggttatctacaccgattttaatggggttgaggtagggcgcgaggcgctccagcgtgtgaaggccagcgtgaaagagacgcaggggcgccagcaactgccgtttgaaaacttggtgaatatgctggatctccctcgcagcctttcccattcaccgttgttccagatcctctatatctaccataaccacgtgaccccacgcgcttttaccttggctggtgcgtattgggagcaggtgacgtatcacaatcagaccgtcaaatacgacatgacagtcgaggtgttccaaaacgacgccacgttcgacgtctcctttgagtacgatttggggttatacgatgctgatgtggtgaagcagattgccgaggcgctgcgtcagcactgcttatcgttgacatcatcactggagaccccgataggggcgatccctctgcatgcaccggagaccgcaacgccgcggcgtgatccgctcaacgccactaacgtcccgtggctcgggccgcaggatgtgctgcgtatcattgaacagcgctgtgtgcagcacccaaagcaactggcaatacagcagcatgacggcacactgacctacgctgagctctgggcgcgtgtgcagttcatcgcgatgcgttttcgagcgcatggcatacaaccgggcgatcgtattggcgtgctgttgccacgtcacagggatgtgattgcaaccatgttggcgacgtggtttgtgggggcgtgctacgtgccgttcgatattcatcagcctgccgcgcgtttgcaacgccttatgcaacgcgcgcgtttggtctgtctggtggtccgtcagccgggagagtggggtgaaattgtacaactgtcgttgccggaattgatgcaggacatgtcgaatgcgatccggtattctacaccttgcgcgctgttgccggatatgcaggcctacctgttgtttacctccggcagtaccggtgaacctaaaggcgtgtgcgttgttcaccgcgggttgctgaacttgttgttggatatgcagcgtacctttgcggttggctcgcaagaccggctgctctcggtgacgacgccaacatttgatatctcattcttggagtatctgttgccgctgatctccggtgcgagtctctatctgacagaggcggaacgcgccgcagacagcttccgtatgattccgctgattgccgactatcgaccaacgctgatgcaggcgacgccctcgttctggcacgggctgttgatggcgggttggcgtggcgacccggaactatgtgtgctggcaggcggtgaagcgctgccaacgaaagtggcggaagaactgttgcgctgttgtggttcattgtggaacctctacggtcccaccgaaacaaccatttggtcgctgaaatcgcagataacccaagcggaaaacatcaccctcggcgctcccattgccaatacccgtatatacattctggacaatgagggccatccagtgccgcaaggcgttgacggcgagctttacatcgccggggatggtgtggcgcaagggtatgatgggcagcctgagttgaacgcacagttcttcttgtcagaaccaggggtccccggtggcaggatgttccgcactggcgatctggtcaggagtgatgcgcaggggcagttgttttttgttgggcgcaaagatagccagatcaaactgcgcggttatcgtattgagttaggcgaaattgaacggacgttggcacggcatccgcatgtggacgccgcggtggtggcctgtattgaaagagcaccgttacacaaagcactggcagcattcatcatcaccagtgagcctccctcgctattcgagcaactgaaaaacgagcttcggcagcaactgccagactatatggtgccaacgctgtggcagagggtggccgacttcccgaacactgacaacggtaaaatcgatcgtaagcggttagcggaaaatttcgttgccgatagctcccttgtgtcgccgcagacgcaagcgctgagcgacacggaacagatgttgctggcgctgtggatgcgctatttgccgataaaaaacgtcgatcctgagtgcgatttctttcgcttggggggacattcgctgttggcggtaacgttggtagcagagatcaaccgcacttttcactgcgccttgaccctgaaggacattttccactactccacactacgggcactgagtgcgcgtattgcacagcaatctatcacggacgctgccgcgtctcaggatgactgggtgatagtgcacgaccctgagcatcgtcatcaaccgttcccattgacggatgttcagcgcgcctactggcttggacgacagacgggtgctacctcgatcgcgacccacatctaccatgaatttgacgtagaacactttaatgttacgcgttttacccatgcggtgaatgcgctgatcgctcgccatgaaatgctacgtgcgcgggtactccccgacggtactcagcagattctggcgcaagtgccggcgtatcagttagagcagcgcgatctgagtgctttgtcccctaacgcacgaaacgatgccttgatggcgatccgcgatcggctgtcgcatcatgtgcatcccgcagatcgttggccgctgtttgatttcagttattcggcttgcacggcgcaacatggccgcttgcatttcagtctcgatctgctgattgccgatgctctgagtatgcgcacgctacagcaggagttgatgatgctgtaccgtgagccccatgtgtcactgccgttgctaccgttctcttttcgtgactacgtgcaggcgctgttggtagagcaggcgagtgaagcctatgcacgcgatcaggcctattggcaacgggcgctgccgcagctgtatggcccaccaacgctgcccgtacagggcgatttggcgcaactgtctgcgatcaggttcgtacgtcgccgtcatcggctgtcagcccacaactggggagtgctgagcgcgctggcccaacgcacacgtatcaccaagacggcattgttgttgacagtctttagccaagtgctggcacgttggagccttagcccgacgtttacgctcaatctgacgttgttcaaccgcccgcagggttaccccaacgcagaggcagttattggtgattttaccgctgtcagcttgctgaatgtttgttacgacagccagcactcttatgcccacaacgctcagcgtattcaggtgcaactgtgggaagatctcgaacatcgtcgtttcagtgggatccgcgccagcgaggcgctgatccatagcggtcgtttccatgcgccgatgccggtggtattcactagtatgttggatatcgacggggagacgactgcgcaagaccctcgggacacaacccgttttactctgtgtccggacgccaatattacccaaacaccgcaggtgtggctcgatcaccaggtgatcgagttggctggggagttgcatttcaactgggacgcggtcgagcaactgtttgataccacgctgctggatcagatgtttggtgcttattgtcatgcgctgcaggcgctggttgccatgccgcaaagttggtggggggtaaatagttctctggcgctgcccaccgttagtgcaccggtcacgcaggctcctgcacctacggccttgttgcaccatggattactgcgtcaggcagcactgacgccacaggaaactgcgctgatcagtcctatccgtgaattgacctatcgccaactgtcgacggcggcggatcatgtggcccgcgccctgttagcgctgggcgtgcagcatggcgaccgcgtggcggtggtgatggaaaaaggctggcagcagattgccgccgtacacggcattttacgactgggtgcggtctatctgccagtggatccggtgctaccgccacagcgtcgccagcttttgctgacggtgggcgaggtgcgggtacaagtaacgcagccgggtctcacgcaattggagccgtcgctgcccgtgctgatcatcgacgacggaatgctggacacgcctgctgcgccgttgcctgaagtggctggggatgtcacggatctggcctatatcattttcacttccggctccaccggtaccccgaaaggagtgatgatcgaccaccgtgcggccatgaacacgctggaagacatcaacgaacgctttggcctcaatgcgcaggatagggtgttcgggctgtcatcattgagctttgacctgtcggtttacgatgcctttgcgccttttatggtgggtgcagcgctggtactgccggaagcaggacgggaaaaagatccgcgtcattggcagacagttatggcacacggtcatgtaagcgtctggaatgcagtgcccgcactgatgcagatgctgtgcgaataccacagcggcgatcggatgagttatccgacgttgcgtctggcactgttgagcggcgactggatcccgctaacgttaccggagcagatgcgcgagcggctcaatgaaacgatggacatcatcagtctgggtggagcgaccgagtgcgccatctggtcggtctactacccgataggtgaggtggaatcgacgtggaccagtattccctacggtcggggcctgcgcaaccagccagtatacgtgctaaatgcgcaactggaggaatgtccggtcggggtggaaggagagatttgcattggcgggatggggctggcacaaggctacctgaacgacgcagagaaaacggcggcgagctttgtctggcgcgaagcgagtggtgagcgaatttaccgcactggggatcgcgggcgctactttgctgacgggcaagtcgcctttttggggcgcaacgatacccaagtgaaggtgaatggttaccgtatcgaactgggggaagtcaaaagccaccttgaacagctcgacagcgtagggagtgccgccgtggtgtgccaccagggacagctgtatgcctttatcactgccgcagaaaacctgcatcctgacgatactgacgccctgttagcgcgtgttcgtgctcagttagccgtgcagttgccgtattacctgctgccccaacatttctttcttctcaaggtgctgccgatgacaggcaacggcaaaattgatcaggcggcaatggttcaagaggttatccaacgtatgtcgcaatctacatcacagaagtcgagggccctcgcgcacgcctcgccctatgaacagcaggtggccgctctctggtgcgaggtactacaacgagaacagatcggactgaatgacaacttttttgaagcggggggcggctcaatccagatcgtgctgttgcatcgccgtattgaggagatttttaaggttacggtacctattgctgagctatttcgcttaaccaccgtgaaaagaattgccggttatttgcaggctatgcaggacaatgcacgggcggtgaatcaaacacagcagcgtgatgcttcccgatcccgcgcccagcaacgacttgtacgtcgtcaccagcgtcagcgttaa

SEQ ID NO: 1075 - DNA, 6465 bp, Escherichia coli
  source 1..6465: mol_type = genomic DNA
atgacttacagtgaaagcgatattgccattgttggcatgaactgccggtaccccggcgtgcacagcgttgcggcattcgaaacggtgcttcgcaccggatgcaacattttggaccccaaagtgaccccgagcaacggccacaatcacattacgctgaacaacgtctatgagcacatggcggagttcgatgccaacttttttggctacagccgtgcagaagcagaaatcatggatccacagcagcgcgtatttctgacctgtgcttgggagatgtttgaacaaagcggttacaacccgaagcagcacgatgcgcgcgtcggcctatatgcgggcgtgagtaccagcttctatttgcttactcacctgatgaacaacccagacaagcttgcacaactcggtggtctccaaatcatggtcggcaatgacaaagatcatttgacgtcccaactggcttaccgcctgaatattaccggcccttgcgtgacggttcaggccagttgtgccacctcgctggtggcagtgcatctggcctgcgaagggctgctgagcggtcagtgcgatatggcgctagcgggtggcgtcacgtttcgcatggaggagcagcgcagctatgaatcgcacggtgatgggctgcaagcagaggatggcctgattcatacttttgatgcgcaggcgagcggtacggtctacagcagtggtttgggcatggtgctgctcaaacgggctacagacgcgcaggtgcaaggggataacatccttgccgtcatcaagggaagcgccatcaacaatgatggcggtgcgcgcagtggttataccgtgccgggtgttgatggtcaggaagctgtgatgattgaggcgcatagcttggcggaggtgacgccgcagcaaattcagtatctagaactgcatggcagcggcacgccgttgggtgacgctatcgaattcgcggccatcaaacgtgtgtttgggacgccagcgccgaatgcgacaccgtggcgtctgggggctgtgaaacctaacgtgggtcacgtagaaatggcatctggcatcaccagtctcataaaaacggtgctgagtcttactaaccgggtgttttaccctacgctgaattttcaacgggctaatccgcaactggggttggaggacagcccgtttgaagtggtgtcccgtttaacgccttggccggagggcaccacgccacgcaccgctggtgtgagtgcgttcggattgggcggcaccaacgcacatctggtggtacaagcgccgttatcgacgccgcaggcgagggcgcagcagatggggccttgtgtcgttgtactctcggcaaaaaaccacaatgccctggaacagatgcaaaacgccctgctggcgaaactggccgcacatccagagatacgcttacaggacgtggcttacaccctgcgtcacgggcgtttctccgccccggtacgtaagtgtgtcatcgcagagaattgcacccagctagcccgacaactccgcgacgcaccgatggtggaggcaaccacgggttgcacgatctactggagattggggcaccgtttcgttgttgcactcgagacgcttagtgactggctggcgtgctctgaggtgttgtcacaggcggtgggacaactgctcgagcattttccgctcgaaccagcctgtctgcaagatctctcccctgcgcaacggacgtttatcagccagtatgcgctgatcgcgttgattgacgaacgggaaacgttgaatgtggtgctatgcggcgacggtgatggtggctatgctgccgccgtactacgaggagactgcacactagagcaagcatggcacaggttgaatgcgggccaaccgtttgatgatgtgccgacgaatcccctcttgcagcccgatgtctgctctttgatgctggacgacgcggcgagtgatgccaatcgcaccgcgctagaggcgttagggcagttatggttggctggcgtcagcctagattggcgctgggtggatgcggcggagcgtatgttgggcagtcaacgcatcgccctgccggggaccgtttttacaccgcagcgctattgggtcgaggccgtacggcctgccacgttctcccacgaatcgtcgaataatctcttatcccgcgcaacaaaatccgatattattgcagtggtgacggagatatgggaacgcacgcttggtgtcagcattgatgatcaccacgccagcttcttcgaactgggtgggcattcgctgttagcgtcaaccattttatatgatattcagcaacgttacggcatcacctgtacgctaagcgcattctttgccgatcccaccatcgagggattaagttgctatctgctcgaacaaggcggcagtgagacagcggtttcagcattgcctgatacggtctttgcccctgaccagcagcacctaccttttccgctgactgacgtgcaacaggcctattgggttgggcggcgaaaatccttgggattgggcaatatttccacccatatctatgtggaatatgaactacaggggctagatgaaacagcgttcaaccgcgcgctcaatgcggtgattgcgcggcacagcatgttgcgtgcaattgtcaacgatgacggtatgcagcagatattgccaaacgtgccggaataccacgttgccttctataccacccagtgcgaggatgcgtttcaacagcgttgccgtgagctgcgtgacaccctctcacatcagatgattgattgtagtcgctggcctctgtttcagatggaggtcgtggttgatccgcagcaaaaagcccggctacatgtatctatcgacctgctgatagccgatgcatggagtctggagttgttcattcgggaactagcttatcactaccgccatccgcaggcggcattgcctacattgacgtacagtttccgcgactatgtgctgaccttaaaatcctacgagaaaacgccgcagtttgaacgggcacgtgactactggcgcgcccgcatcgaaactctgccgccggggccacgattgccgctgcgtaccgaccccacgaagctggaaaaccccacattcgtgcgccgtagctattgtctatctcgtgctatctggcagcgcttgaaaacgcaggcgggtcagatgagtatcacgcctactacactgctgctcacggggtttgctcaggtgttggcgcgctttagttcctcgccgcactttagccttaacctgacgctgtttaatcgtctgccgttgcacgcagatatcaatcacttaattggcgattttactgcactgacgttgctggagattgatatgtcgcagggagaaaccttgcaggcgcgggcaaacgtgatccactctcaactgtggcgcgatttggataaccgcttgtttggcggtatccaggtctctcgactgctggtacaaactcaccgcgatccggcgaaatcggtgattcctatcgtgttcaccagcttgctcaatcaatatgaggcgagctgggaaacggatgatacgctgttcaaccaaccgcaggatgatctgtacagtatttcacagacaccgcaggtgtggctcgatcatcaggtaatggagcgtaacggggaattgcactttaattgggacgtggtggaacaattgtttgaaccggcgctaatggatcagatgttccagtgttattgccaactcctgcacgccctagcccagcgaccacagctctggcatgagactcaagatgtgctggcgctgcccaccgttagtgcaccggtcacgcaggctcctgcacctacggccttgttgcaccatgggttactgcgtcaggcagcactgacgccacaggaaactgcgctgatcagtcctatccgtgaattgacctatcgccaactgtcgacggcggcggatcatgtggcccgcgccctgttagcgctgggcgtgcagcatggcgaccgcgtggcggtggtgatggaaaaaggctggcagcagattgccgccgtacacggcattttacgactgggtgcggtctatctgccagtggatccggtgctaccgccacagcgtcgccagcttttgctgacggtgggcgaggtgcgggtacaagtaacgcagccgggtctcacgcaattggagccgtcgctgcccgtgctgatcatcgacgacggaatgctggacacgcctgctgcgccgttgcctgaagtggctggggatgtcacggatctggcctatatcattttcacttccggctccaccggtaccccgaaaggagtgatgatcgaccaccgtgcggccatgaacacactggaagacatcaacgaacgctttggcctcaatgcgcaggatagggtgttcgggctgtcatcattgagctttgacctgtcggtttacgatgcctttgcgccttttatggtgggtgcagcgctggtactgccggaagcaggacgggaaaaagatccgcgtcattggcagacagttatggcacacggtcatgtaagcgtctggaatgcagtgcccgcactgatgcagatgctgtgcgaataccacagcggcgatcggatgagttatccgacgttgcgtctggcactgttgagcggcgactggatcccgctaacgttaccggagcagatgcgcgagcggctcaatgaaacgatggacatcatcagtctgggtggagcgaccgagtgcgccatctggtcggtctactacccgataggtgaggtggaatcgacgtggaccagtattccctacggtcggggcctgcgcaaccagccagtatacgtgctaaatgcgcaactggaggaatgtccggtcggggtggaaggagagatttgcattggcgggatggggctggcacaaggctacctgaacgacgcagagaaaacggcggcgagctttgtctggcgcgaagcgagtggtgagcgaatttaccgcactggggatcgcgggcgctactttgctgacgggcaagtcgcctttttggggcgcaacgatacccaagtgaaggtgaatggttaccgtatcgaactgggggaaatcgagcgctgcattgcgcgacatcccgatgtggagcagtcagtggtggtggcagtgggtaattctcaacatcgtcggctggtcgcttttgccaaactgcacgatcgccaccaggcgcaggcattgcaagctaaggaagcggaggcggcggcactggcgcagggtattattgtgaatccggcacagcgtctagcgttcaaactcaaggagccacatattcgcgcgctggatggtctgggcattgcactgacggcaccggcggatagcacacgttacatcaaacgccgcagctatcgtcatttcagcgcgcaaaaaaccacgctggcacagttggggcaattgctgtcgggcttggggcagatgcgtctacccggtctaccttttgccaagtatgcctatgcgtccgccggggggctatacccggtgcaaacctacgtgtacctgcatccagacaagatcgaagagggagtatccggtatttactacttcgacccgcgacagagctgtcttatgccggtagcaccagaagtcgagctgaacagtggttttcatgccggacctaatcagtctattgccgatcgggcggcattcacgctgtttatggtggctgatatggcggtgatctcgccattctatgggcaagaggcagcttggcacttctcggtgatggaagcaggtactctctgccatttactggaagaagatgcgccgcgctacggattggggctgtgccaacttgggatggcagacttttccgctgtggcatcgcattttcaattgtcgccacatcatcgctatgtccattgcaccgtggggggcgcgatagggcaagaggcggcaagtgctgcagcattgctgcgcgatttctccacctatgagaaaccgaaggaaaccgctgcgccgctggacatgcagagctacaaagatgccatgctgcgcggcctgcgtcagcaactgcctgactatatggtgccgagtgatctgatgttagcgaccgattttccgttaaccgctaacggcaagctagatcggcaaaaattacagctgcagggcgaacaaattgcccaccagcgtgacggcgtgggtccaatccaggtggacagtgcgttacaacagcggctggtggcgctctggcaggaggtactcggcgtgagccacgtgtcggccgaagacgatttcttctcgctggggggcagttctatagaattggtgcgtattcagcaggcacttgaggcgattatcgggcaggagattcccattgtcgatctgttccgtctgccaaccatcgcggatgtggcgcgttaccttgatgagcaactgcacaatctacccgccgcccacgatatcgtattagcgcaggctgaggtttcgcaggtcagcgctgcgcgcgagaatctggcgttgcggcgtaaacgtgcccaacagggagaaaagggcgatgagtga

SEQ ID NO: 1076 - DNA, 1464 bp, Escherichia coli
  source 1..1464: mol_type = genomic DNA
atgagtgagcagagctatcgtagcgcagggacactgttggcacagttggcgtccggagaaaccacctcagtggcgttggtgaatcactatttttcacgtatggcgcagtttaacaagccgttgaatgcggtggtgcagcagcactatgcgttggcgttggaggccgccgcgcgcgccgatcgtgagcgactggaggggcgcgcaaggggcgtgttgcacggattaccttgtactgttaaggagtcgtttgacgtgcaagggtggttaaccacgtccggtgcccattatctgaaagataaccgggcgacgcaggatgctccttccatcgcacgcttacgcgctgccggggcgatcctgatgggaaaaaccaatgtgccgatgatgactgccgattggcaaacctataacgatctgtatggcaccacccataacctgtgggataggcaacgttcacccggcggatcgtccggaggagcggcggtggcggtggcggcggatttcacccccgtggaatttggcagcgatttgtttggctcactgcgcattcccgcacactacacaggtgtctatgcccatcgttgtagcttggggttgatgtcggtcagaggacatgtgcccggtggtggtccgcaagcgactgatgagccggatctctcgacggcgggccccatggcgcgcagcgcggcggatctgcgtctgatgatgcgcgcattgagcacattctgggtggaaccgccgcgtattcccgattttagccgttatcaggccaaagcaaactaccgcgtgtgcacgtggtttagtgcgccccaccatgagatagatcaacagatcgcacagcgttttcaatcgtttatcgacaagttacgcgcacagcctggcgtggaggttgatgatgctatgcccgccgatatcgatcccgacgcactgtttgatatagcagtcaaactcagtggtcggctggtaagtacggcgctcaatggacgccagcgtcttacagccgggctggctgcgctgggcttcaggcttgtgggcaagctggcggatgtgcctgaggggataacgagctattaccagggcatgctgaaagacagtggcgaacagcggaatacagacaaactccgccacgaatacagccgggtgatagaaacccttttcgcgcgttatgacgtgttgttgacacctgtcagcccggtgctggcgtttgcgcacatgcagcagccggtgcgtaagcgcaagcttatcgtcaatggcgaaccgcaagattacaacgaacatttgttctggaacatgttagcaacggtttttggtttgcctgccaccgtttacccattggcgaaaacgatggatgagcttccgtgcggcatacagatcatttccgggcattttcacgatgatgtgacgattaactttgctgagttctgcgaaagcatcagtggcggattcacggtaccggaagggtactag

SEQ ID NO: 1077 - DNA, 1440 bp, Escherichia coli
  source 1..1440: mol_type = genomic DNA
atgacggtgaataagcttgagccagacaacggcacgccgaacgatgagctgttcacggtggcgggtatgtttgacggctcgctgtataaattactcctgcgcatggcgttgccgatgtttgtcggtatgttaacgcaggtgacctatgccatcgccgatattttttggctgagccacattgatgtcaccaacagcggcatcattgccggtgtggggctggttttccccgttggaatggggttatttgccatcgccaacggcattcaaatcggtatgggatctttgctgtcccgcgccatcggcatgcagcggttggatcgtgcacagcggattttgtcggtcggtatcatcatcgcgctattctttgcgatcgtgatcaccgtacttggctatgtttatgcccagccgctattgcgctcactgggggccacgaagagcatcatcggctacgcaacggagttctattattattcgctgctgaccgtgttcagcatcatgttgattggcgtcatgatggggctgtttcaaggcgcgggtaagatcatggtcatcatgaaggcctcattgttgggggcgttggtaaatattatgctcgatccaatcatgatctttgtattcgattttggtgtgaaaggggtggcattggcgtcgtttctggcgcaactgagtatggtcgcctatttcatctacacattgatgggattacatatcgggttgagtatacgcatcgcgttacgcccattctcctggaaaatctaccgagagtttctctccgtcggcatggcgcagatgctgatgcaactgatcattgcggtcggcatagtgatttataatttttttatcgtcaggctggacgtaaacgcgatggcagcgtttacgctcactggtcgcatcgactatttcatcatcacgccgatgctggctatcgcgaccgcgttgctgactgtggtggggcaaaactggggacacggaaatgtcacccgcacgctgaatgcctattgggccgccgtcgcgctggccttctctattgtgctggtgttagccgttatgcatattgtactggcaccctggatgtatcccctgtttactcgtgttgttgccgtgagtgactatgcggtactgcaaacccgtatcatggcactggcactaccctttgtcgctatcagcctgttagcaagtgaatattatcaggccattggtaagccttggtattcggtgttgttgacgctgatgcgtcacgtgtttatatctgtccctgtcgtatacctgctggcgattgtgttagagatgcgtattaccggcgtctattttggggcgatgagcggcacttttgtggctgcgttgttggcgtggcgtctgctgcgtctttcacctcgtctgctgcgatggaatcaggaggcggtgcgctcccaacacttggatatggaggtggcaccatga

SEQ ID NO: 1078 - DNA, 4368 bp, Escherichia coli
  source 1..4368: mol_type = genomic DNA
atgatgtcgggcaatccgttgtcgtggccacaggaacagtgccacatcattgatcaactgtatccttacagtgccgtaaacatcattggtggtgtggtcaccattgagggaattgtcgatctgccccgactacacgcggccatccagtccgccattcggcaatttgatgcgttgcgtatgtggtttgtgatgggagaggagagtgaagtggtcagccaagtgcagccttatcactggcgcgatatccgccatttgactttttccccagattatgacaaggagaacctacgcccagcggcgatcgaaacatttgttgatgagtggttccggcagccctttaccctgctggcgcacgatttgttcgagtttgtcacctttacgtgcggtgaacaatacagcggttatttatttaaagcccatcacggcattgctgatggttggtcaatggcgttgttgtcaaaccatgtcaagcgagcatacgaacagcaggacgtaccagatgatgcgtcaccagcctatagcgcatttctggcgcagcagcaatcttatcaggcgtcaacgcgctttgccgttgatcgtggctggtggcgtgactatatcgacgaataccgcgactgctttccagacagttcccccatcgttactactgagggcatcagttgttccacttggcttgagccagccatgattaaccgtctctacaggttgtgtaatcgttatggctgtacgcttaatacgctgtttattgcactcttcgctctctatcgcgccagagtatggggggaggaaaaaggcgtaataggcgtgccgttagccaatcggcatactcgcgaggcgcggcgttgctttggcatgtttactaatcaattgccgctggcctatcgactggtgcgcacggaacgattctgtgaacgggtggcctttttccagcgcgaactgaaacgcggttttaaacacagcaaatatcccattaccttattcaaccaggatttggcggaacaggggggagggaagttgcgggcgttcgattattgcgttaattattataactttacctatgaacgccacattgcaggtgccgcgcaacgcgtggagagttactacagcggcgagcagtcctataagctacagattgttttgcaaactgttaataatcataaagaaagccttaggttgagtctggaggcgctgcgtagcgctttcactccgcaccaactgacagcgatgaaaaatgggctgttggatttagtcactgcattggatcggcaacctgacgctcgtctaggcgatctcgaagtctatcctgcgccgcacgtcgcattggcatgtggctcactcaaaccctcatttacttcccgatttgcagcgcaagttgtggagcatggtgatcgcacagcactaattgataatgaacagtctttgacttatcggcagttggatgacgctgttgagcgggttgcgcgttatctgcgccaacaggggatcggacgcggtcaagtggttgggattatcgctgagcatagcgctcaaacggtgatggttatttatggtattttgcgctgcggcgcggcatttctgccgctaaatcccgcgttgccaacgacccgcctttacgccatgtgtcgtaaagcgcaggtagcgcatatcctttacgatccggcgatgcatgaattgacgcaggcgctggcgtttcccgcgtccagcttgctccaggccttagccacttcggcgcttgctagagaaccttggccagctattgaaccgcaggatctggcatatgtgctgtttacctccggatcgaccggcgagcctaagggcgtgcaagtctctcatggcaatctggctaactatctccatttcgccgccgaacgttactttacagcgcaggaccgggctgcgctctactcttcgctgtccttcgatttgaccatcactacgctgttcgcccccttatgtgtgggcgctagcatcagcgtttgtcggcacgccgagagcgaaacattgctgcgcatggccgtggttgatcagcccaacacagtgatcaaactgacgcctgcacacctgcggttgttgtgtgcggctggcataagcagtgagcagattcgtaccttggtggttggcggggaggatttcaagcgggatctggcgcgtaaggcggctgctctctttcctcaggccgtgatctataacgaatatgggccgacagaggccaccgttggttgcatgatctatcgctacaccgggcaagaaacgctgccttcattgcccatcggtatggcgatcgacggttgccaggtagcaatttgctccccttggggctgtccggtaccggaaggcgaaactggtgaattggtgatttatggcgcgtcagtgacgcagggctatatcgatgcgccgcaacaaacggccgccgcctaccttaaagataccaatggggtgatgattggctatcgcagcggtgatattggctacgccatcgctcctaatacgctggtttatcagggacgtaaagacgatcaggtcaaaattaatggctatcgtattgagctgtgcgaaatcgaacaagcgttgttgagcgcacctcaggtagaaagtgcggcggtggcggtgattgatgatgtgcaggggcagcacagcgggttgctagcctgcgtgacaccgtcatctgttgatgtagctaccgtaatgcaacatctgcgtcagcaattacccacctacatgcaacccaagcagtgctgtgctatcgcccaactgccgctatcgcacaatggcaaggtggacgtgcgtcagatggtcgcaacggtccgaaacaccgcgcctgcatcgggtagcgagcggctgggagatgcggcgataaggcattcagttcgggtatgtgtggaaggggcgctggagcaaactgagtttgatgataacgaaaatctttatgtcttggggttggactccatcaagagtatccagattgcagcgcagctcaggcaccacggctggacgatgtcagcggtgcaggtgatggagtgtggaacagtgaacgccatctgtgagtttctcgctagtcatacaacggtgtcacagctcgcgcagtatgctcataacacccgtatcgatctacccgcattgcgttggtttacgcagttggcgttgccagtgcccaatgtttataaccatgtcattgtgctgaaagtgttgccgggctgcccgctcgagcagttacacaacaggctgcatacattaatccagcagcagccagctttgcacagtgcgctggacgctgaggggcggttgctagtatgcgaccctaatgtgtgttatcccaacgaggtgctcacggaatattcgacggcgcagtggacgttggcagaggtgatcgctcagtgcaatagcatgcttgacgtgacaaatggtcgagtctttacggcagctctgttacacgccccgcagcctgcgtctagcacgctggtactctgtgcccatcacttgtgcgtcgatatgcactcttggtatttaatcctcagcacgctggacgccgtcagcactgtcaatggcaccagcaacagcggactgcatcgctggaatgactatctggcgagcaaaacggtagattccgccacacacgaaagctggcgcacagtatgtcaaacgctaccgctgcatttcccacccgtgtcattgccagatgactcattacctcgtacgcgcgcttggcgtgaagactttcgccatccttgcgtcaggaggttgtttgaatccagtggtaataccgcgtacagcgccgaaacctatgtgctaacggcattggcgctggtgctgcgttactacagcgaagagccttggtgtcgcatcgagatggaaggcatgggacgcggctgctggcccgatgagcctgacgttgcggatacggtcgggtggtttactctattttatccttgggcgatccctctgcatggcgatatggcgacactgttatctgcgatcgcttccgatttggcaaaacgcacgcatggtgggggagattacggtctgctgcaaatgcggcatgcaccggaagactctcttgcgcaagggatccgaatgaattacatcggtgtgcaggcgcaaccttcactccgctattttcatattgatcactttaatagtgatatctataccgcgcctgaaaacgcgctcggctgcgtgctggagtttaatattgcgcgctcggcagctgatggtctctccttccactgccgtttcgatcccacgcgcattgcgctgaatgatgtgcaactcctgctggcgcgctacaagaacagcctcactgatctggatgcctggctgtgccaacacagcgccacgctgacgggcgcgccgactttgtggacactataa

SEQ ID NO: 1079 - DNA, 2460 bp, Escherichia coli
  source 1..2460: mol_type = genomic DNA
atggcaaaggatgattttacctgtggctcactggatattgccattattggcatgagcgggcgtttttccggtgcggaatcggtgcccgagtggtgggataagttgctggcgggtgaggagttcactcaaccgacatgcacagaagacgataatgggaacccttggattaggctgcgcaatataatcaccgctccctatgacttcgatgctgcatttttcaatattccgcctggcgaagccctgttgatggatccgcaacaacgaatatttcttgaatgctgctacaacgctcttgagcatgcgggctatattcccacgcaacttaagcgagtcggcgtctatggggcgacctacgccaataactattttatcgatcgcgtctatccatacctgaagatgagcggcgatcaccattatcttcaggcgcagattggtaacgaaaaggattacttgtgtgctcaggttgcctacaagttggggtttaccggacccgctgtgagtgtacagaccgcctgttccagttcgttggtggcagcatatttagcatgcgaggggttattgacttttcaagccgatgttgcgttggcaggtggggtcaccttgggttttttacaggcgcacggctacagtccgcaaggtgataagctggtatcgcaagacggacactgcgccccgtttagcgctgaggcgaccggcacggtatacagcagcggtgcgggagttgtggtgctaaaacgtcttgaggatgcactacgcgaccaagacagagtgtatgcggtgattaaaggaggtgcggtaaataacgacggcgggcgtcggctggggtttgtcgcccccagcgtagaaggacaggtagaagcgatcaatacggcgcttgccgccgcagaagtcgtaccgactgatatcgcactgatcgaaacgcacggtaccggtaccccgttgggtgatgagattgagcttgaagctctgcaccgggttttcgcaccggcatgtgcgccgcacagcattcaattgggtgcggtaaaagccaacttgggtcacctgggagtcgcttccggtatcgtcagcttaatgaaaaccgcactgacgctatatactggtttagttcccccgcagatcaacctagtaaataaacataaaaaactgttgcagccagcctcgccattttatctcagtgatgtagtgacttctgtcccacaaacgaaaaggatccatgccaccgttagttcgtttgggctgggagggaccaatgctcatttggttctgcaaaactggtgcgagacgcctgcgcaagcggtgcaagaaaatgagcgccgcctgtttttcttcagcgccaaaacgccattagcccttagacaacagttggatgctcattatcacgctctggcgacctatgcggaagcagataaagatcgtatcgcctatactctcgcgcaacgccgggcacatttcccgtatcgctgtgcgctggcggcagacagtgtggtagcgttgcgtgcaagtttggcgaaactgcgggatgctgacatgtcttttacccctatcaatatggaaactaccttggtgttcctatatccggaccgggacgataagctggagagtgccctgacgcatttgctggcttgtcaacctaacttgcgccagcggcaccagcgcctgtctcaggatgttgcacaaatctgcgaaccagccgattggacgccagcattgcgtcagtttatccagcaggtgagtcttagtgaatggctgatcgaacaaagtatctcgcctgtgcagcacatcggttacctgacgggggctgcagcagcgcagtatgttgcgcgcatcatctcactggaaaacgctgttcagcaggtgattgtggctgaaacgacaccggagcagacgctggccggcaacagtgaactgagcgaaatattggctaatctggcggtgacggagggaacgctgatgctggaaatcggtcgcgcggggacgttttccatattgtatcaccagcatgcgcagtgggttgggcaaacagtattttcccccatgctgaatacggatacgcctgaggacatcctgccgctgttgggcactctttggcaacgaggagtgactatttgtttaccggaaatgccagccgtgcagactataggcttacccggttattcgtttgatcgcgtccggtatgaaattcagtccagtgatgcgagagagaacgctatgttaccagtgagttatttgtcggtcagcgactttgtggagaaaacctggcgttcattgttatgcatcgatcattatgacgaacacgcggttattttcgaatacggagcgacctcgatgcacgttatctctttcgtcgacagctgtaaccacatttataaaatcggactgactgccgccgatatttatgccagacccgctatccgtgagcacagtgaatttatctccgaatgtgttgatggtattttatga

SEQ ID NO: 1080 - DNA, 1515 bp, Escherichia coli
  source 1..1515: mol_type = genomic DNA
atgacaataatggaacacgttagcattaaaacattatatcatctcctgtgctgtatgctgctctttatttccgctatgtgcgctttggcgcaagaacatgagcctatcggggcgcaagatgagcgcctgtcgacattaattcaccaacggatgcaggaggccaaggtcccagccctttccgtaagtgtgaccattaagggggtacgtcagcgatttgtctacggtgttgccgatgtggctagtcagaaagcgaatactctagacacagtttacgagctgggatcgatgagtaaggcgtttaccggacttgtggtgcaaatactgattcaggaaggcagactccggcaaggggatgatatcattacctatctgccggaaatgcgcttgaattatcagggaaaacctgcttccctgaccgtggctgatttcctttatcatacatcaggattgcctttttcaacactggctcggctggaaaaccctatgcctgggagcgctgtggcacagcaactgcgcaacgagaatctgctgtttgcgccgggtgcgaagtttagctatgcctccgccaattatgatgtgttgggcgcggtgattgaaaatgtgacgggaaaaacctttacagaggtcattgcggaacgactcacgcagccgctgggcatgtcggcgactgtggcagttaagggggatgagattattgtcaacaaggcaagcggctataaactgggattcggcaaacccgttctgtttcatgcgcctctggcccggaaccatgttcctgccgcctatatccatagcactctgcctgatatggaaatatggatagacgcctggttgcacagaaaggctttgccggcaacgctgcgtgaggcgatgagtaacagttggcgtggtaatagtgatgttccgcttgccgcagacaatcgtatcctctatgccagcggttggtttatcgaccagaatcaaggcccttacatcagtcacggtgggcagaatccaaacttttcttcttgcattgcgttgcgaccggatcagcagattggcattgttgcgctggcaaatatgaattcgaatctgatactacagctttgcgcggatatcgataattatctgcgcattggcaaatatgctgacggcgctggtgatgcaattacagccaccgatacccttttcgtctacctcacgttgttgctgtgtttttggggggcggtggttgtagtgcgcggtgctttccgtgtttatcgcgcaacggcgcatggccctggaaaacagcagaggttacgtttacgcgtacgtgactatatcatcgccttggcggttcctgggctcgtggccgccatgctctatgtcgcaccgggtatactatctccaggacttgactggcgttttatcttggtatggggtccatcgagcgtgttggcgataccgttcggaattatcctgttagctttcgttctgacattaaatcatcaaattaaacgaattctattacacaacaaggagtgggacgatgagtaa

SEQ ID NO: 1081 - DNA, 723 bp, Escherichia coli
  source 1..723: mol_type = genomic DNA
atgagtaatatcagtttgtattgtttgccatattcaggtggttctgccgccatgtattataaatggcgtagcgtgctgtcggacaatattactttgcggcctttagaacctgcggggaggggaactagaatacgccagccgctgtgtcttacgatggtggatgccgtcgctgacctttatcaacaatttgtgaaacactacacaggtggagactacgccatttttgggcatagtctcggagggatcatggccttcgaactggtgcattatattctcgatcatggacatgacatgccatgcgcgctgtttttttccggctgtcgcccacccgatcgggcctctcatgaagtaatactgcataccttgcccgatcaggcgtttatggaagagatcgtcaagctgggcggaactccggttgatgtctttcgtaataaagagttaatgacaattttcacccccatcattaaaaacgattatcggctctatgagcagtatgtatttcaggccaaggcgcgcacattaacctgtccgatcgtgctatttcatggcgatgctgacaatctggtaatgcaggatgaattacttgcatgggaaaaattcaccacacgaaagacgcggactattatatttcctgctgccgatcatttttttgtcgataagcattttgaacaggtggtaggttatgtgaaccagacgattgaatcactcgaaatagtagggtag

SEQ ID NO: 1082 - DNA, 213 bp, Escherichia coli
  source 1..213: mol_type = genomic DNA
atggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaa

SEQ ID NO: 1083 - DNA, 513 bp, Escherichia coli
  source 1..513: mol_type = genomic DNA
atggctgttccatcatcaaaagaagagttaattaaagctattaatagtaatttttctttattaaataagaagctagaatctattacgccccaactcgcctttgaacctctattggaagggcacgcgaaggggactacgattagcgtagcgaatctggtttcctatctgattggctggggagagctggtgttacactggcatgaccaagaggcaaaaggaaaaactattatttttcctgaggaaggatttaaatggaatgaattggggcgtttagcacagaaattctaccgtgactatgaggatattacagagtacgaagttttattggcacggttaaaggaaaataagcagcaactcgtggctttgattgaacgattcagtaacgacgagctttacggtaaaccttggtataataaatggacccgaggtcgtatgattcaatttaataccgcctcgccttataaaaatgcttcggggaggttaaataaactgcagaaatgtcttgcagaatag

SEQ ID NO: 1084 - protein, 244 aa, Escherichia coli
MRIDILIGHTSFFHQTSRDNFLHYLNEEEIKRYDQFHFVSDKELYILSRILLKTALKRYQPDVSLQSWQFSTCKYGKPFIVFPQLAKKIFFNLSHTIDTVAVAISSHCELGVDIEQIRDLDNSYLNISQHFFTPQEATNIVSLPRYEGQLLFWKMWTLKEAYIKYRGKGLSLGLDCIEFHLTNKKLTSKYRGSPVYFSQWKICNSFLALASPLITPKITIELFPMQSQLYHHDYQLIHSSNGQN

SEQ ID NO: 1085 - protein, 3206 aa, Escherichia coli
MDNTSGDFPCNKMDTRKQLPLTPSQQGFLFHSLKDKKRSNYHEHFTCIFSQHVDSAHFKWALETLFRKHECFRTDYNWEIDERPCQVVKTDVLPDIYVLDCEQEEIRFLLANDDIIIPVPQDDGIDAIIPQLLQADLKYPFSLKTIPVRAYLIQSTKESAFILSYHHIVMDGWSLSLFIKQLLQLYGAAVVSGVRDDSAIIPSSLKPLVDTLSARRHTFQHDYWAAYLREGTPTCIVPLSQYHTDTEAENNSYVNQTNHVEINLSPDVCQKIQTLCSDYRITPAVIFYVAWGILLQRWCYADDVLFGATISGRNIPIDGIEETLGLFINTLPLRLRDDGATLLQHLQRMHQTLIAHYSNEHDALASIQRLVHKEGHAGDLFNTLVVLENYPVDMTLLSCASPVAIRHLSVHEQTHYPLTLTITQQKGFRFSIAYALNYLTNNMAQALLMHLSYLLEQLVDNPQRPIAALVNLSPCQQAQVLQPYLERMACRDWDSQSNVIEQFHQVAATSPAQVAVVDELCALTYSELAAQAEQLAAYLVQQGVMVGDTVGIISERRVNTVVAIIAIMLIGAAYVPISPDYPVGRMQEIIDDSGLALLLVHGKPLDALNVAQSDLCAFPVAPSVVFPVITPDSRAYVIYSSGSTGKPKGIAVAHRGLLRLIQGDSPLKVESGETTLLTCPFEFDVSVFEMWSTLLNHGKLVLLSKQALLDINHIRRTIADEQVARAWFTSSLFNSYVAEGADFFGMLQHITVGGEAVSAWHVNDVMQKYPHLVVTNGYGPTENTIFTTAYRFNGLQPARVPIGYAVPGTSLYITDLHGHLLPIGATGELVAGGVGVAIGYQNNPALSATVFVPDPFIPGGMMYKTGDYARLLDDGCVDCFGRKDGQIKINGQRIETGEIEQRLLECSGIIEAVVVPYRVRETLHIAAVVCVNDSYDEVEVRGQLADRLPPFAIPESLVVVTEIAKSHSGKADLAQLRYLLPATQCNAVSTTISEVHSDMEHALHAIWQRVLDRQDIDSNASFFALGGTSLDTIRVKGDIKRQLGLEIDITDLFKYPTLTALAHFLDTAVSPEDAIPTRAVVYSDMPVAIVGMAGRFPGAANIAALWTLVVGGESGLTLFSDEELRAHGVTPDTLKQANYIKTKGIVDDHEWFDADFFGYTPNEAECMDPQIRLLHQCCWQTLEHAGCDPATFTGAIGIYAGLLTSPHWLNAVMQDTTDSTALYKASILNIHSVTALIAHALNLTGPAVTLDTACSTSAVAIHQACIALRNRDCDAALAGGVSIEMPAYRGYEYHEGMINARDGVCRPFDSQASGTVTGDGLGMLLLKRLDDALADRDCIYGVIKGSAVNNDGNNKIGYTAPSVIGQSTVIRTSLRRAGFDSDSIGLVEAHGTGTVLGDPIELRALNEVFGPTPVPFCVVSALKSNIGHLNSAAGVAGVIKTTLALHHQVLPPTAHFRQLNPAIDLSRSALYVNQQVQPWPSTRPRRALVSSFGIGGTNASIALEAHQHEDDPSATGVRDSYLLLFSAKTPAALELRVASTLEYVKHGVGVRLPDVAYTLQTGRTAFDHRRAYLVSRGSKIDLSCATILQAEIFNGQRTTAEICFMFPGQGSQYHGMASALYAHQPMFRQHMDRCFAAFQRYSTVDLKALLFDDEDTRDIDQTQFTQPALFCVEYSLARTLIDLGITPDSMIGHSLGEYVAACIAGVFTLEDALHVIEARGRLMQSMRPGSMMAVYLSREQLTPWLAAERGIELAANNSAHFCVVAGEQAAISRLSTRLVEGGIQHRRLKTSHAFHSAMMTPMLHDFAQLLGQIPMHAPHKRFISNVSGTWITEEQATSPDYWVQQVRNAVLFSEGAAQLLVQPTLFIECGPGNTLSTFIQGHNQYSDQPTLLTLRKANAAIDDEHMLHRTLAALWVRGENIDWRRFNQTALGKHIPLPDYPFEQTYYYRYGAALSGYRQYPNPLRRPQDEWLQRVLWRMHDTSLREAFYAPGELIIIISADGDKLQQTLMSSGVDSITMPLPISSEDDVWDNDRILTHFHDICALLAHKTYRQLHCLYAPGAEAGSSLTQSLSGLYRVARWCMHSTTPLASLTVLTHGAFRVQEEDNPEPTLAALSGAVNVFAQELHPTEVRLIDIDAQSSDENLNLLTQRLAPKQETVMALRQGMLYLRRFIPTRLLAHLPPQTGCIPGNVLWIIGGEKGIGRMIGEALAQREGVRVVLSSRTGYHHEAVQQDALDVIHCDVTQAEAVRACLATLLERYGRLDGVIFAADATTTLTLHQLSESALRDTLTVKERGTANVLHALAQRNLLDERLLLLFCNSLAAVNAEIGQTGYATASAYLDALAQQLRTRYKVNALSIGLDALREQGMLLDAINGSEYDVLRGLRPLMTGTLLQAYKQQGADTSYYARLSPESDWLLDEHRISGIATLPGTGYLALAYEALRHYFVQDQICIDELVFLAPLTVMDNCSVDVFVDISPNGQGVSVEVKSMTERFSGTLTTHARGRATRLMVDDNVVCDLTGLMREMHTITPPTKELSSTHFHYGPRWHSVQQLYGNTAQTQVFATLALPTVAANDTIALHPALLDIASSVVEQLPGFHTDSVPFLYQDLRLYRPLPNTLHVALTVNRHDEEGDSYAFTLYDMAGEMVARCAAMVKRKVQLHIQDVDDDTRLRVPSADNYQLRLAAEGEGAGKLALCPTPRLALGDSQVEIEVLATGLNFKDVLFTTGLLRQQPGEAPLQLGLECAGRITRVGKNVTEFAPGEDVMAVLNGGFVQYARVESDCVVRKPAHCRIEQAAALPIAYLTAYYALVVRANLQPGERVLIHSAAGGVGLAALHIAKRCGAQIFATAGSEQKRDYLLSLGVHAVADSHDEQFAATLLTASDGQGMDVILNSLTGRLLDASLALLAPLGRFLELGSKDIVEDKALPMRFFAQGGTFIPINFHAAHGAFSRYLQQIVAWIDDNTLPLLPCKSVPLPEVARAFATLTTPQHIGKVVVTHRTAAGMDRLNAMIAERRLGGYALSMSNAEVMRQLWPILNTRSPWAQLLLSPRAIDRLARGNRVDRGVPSAANDTITQQTVKKRPRPEIGVPYSPATREVERVLCQILEEYLGLDRVGIDDNYAELGATSLDMVQLSGQMARHYPQVSVVSLYNHATVRQLATFCQPPEGESNAPSPQPAVQTNTRANQIAKRALQIAKNTARSHTSLH

SEQ ID NO: 1086 - protein, 866 aa, Escherichia coli
MEYASEMNGMEIAIIGMAVRFPQSRTLHEFWHNIVQGKECVTFFSEEELLAEGVEQSTLDNPAYVRAKPYIEGICDFDAAFFGYSHKEAQTLDPKSRVLHEVAYHALEDAGYAQRTSDLITGVFVGASEDVDWLRRSLSQIGGDALNRFESGIYGHKDLLAHLIAYSLNLNGPVYSLYTSCSTSLSATHIACRSLLFGECDLALAGGITIDLPQKSGYFCQQGMIHSTDGHCRPFDSQASGTLFGDGAGVVVLRRLEDALAAGDRIYAVIRGSAVNNDGKQKIGFVAPGHEGQKAVICAACHLAEVSPESIGYVETHGTGTRIGDPIEFAALTEAFDTSHRQYCALGAVKANIGHTHAAAGVAGLIKTALVLHHRTIPPLANYQMPNSKLDLAHSPFYIPIQPQEWPASRMPPRAGVSSFGIGGTNVHMILEGLNPAVRDDHDQVRAPVFIPLSAPSFEQLDELTQQLTPLLATLDASTLAYTQQVARPVFDCRRVIQVENDGTQAMLASLDNLMPDAPWGLHCPDLRTTNDCTYAQWLAHSAHYQREATALTALLDGMNIPPAYCHAETWAAQANSSLLIRGCQTIAALKTWMNLLPTLTLLSGAGTGLLPAAAASGMIATQDVLHLLWEMEQKALHLWLPERHEPIPGYVLAWQGNPITDAQRNDRGFWSEALLADTRELGEGVHSINWVRLPPEIREDVDVLRYVAQLWCAGINVDWAVWYGTPLPQRGSASAYPFAHNHYPLPGRVMGSVETQPEAGPETHHPYQARPVLSVPFVAAHSRGMQYITGLMELLLEISPVGVDDDFFELGGHSLLVTQLTSRLERDFNVHIDLLTLMENPNPRNIYAHIAAQLGGEDNLEIACQ

SEQ ID NO: 1087 - protein, 289 aa, Escherichia coli
MMNVAVIGAGVMGTGVAHNMAQYGISTNVVDISQSQLDKCRQMIEANLRLYNFHPQHKKKTHSTAEIMENIRFTTELDDIVECDLVIENITEDIEKKNALYTRMNTICGASTVFGVNTSAISITALSKLMRHPENVVGVHFMNPVPLMHTVELIRGVHTAERTLNIFHHLFAQLNKTGIVVNDSPGFVTNRAMMIFVNEAIFMVQEQIARAEDIDTLFKTCFGHKMGPLQTADLIGLDTILQSLQVLYESFNDDKYRPSFLLKKMVDAGYLGVKSGQGFYRYQQTYAEQ

SEQ ID NO: 1088 - protein, 82 aa, Escherichia coli
MKKQDMKAAIREFLSRSLRGHTLNDDDDIFSLGLVHSLFTVQIILFIEKNFQVELEVSELKTEQIATVNKIVELIQRQTGLE

SEQ ID NO: 1089 - protein, 376 aa, Escherichia coli
MCTENYELAQQEAVLFAKQHLALAAQNIERQQFIVPDIISCVAQAGYLGASIPQKYGGRGYDSYQLCALHEVMAGVHGSLENLITVTGMVSTLLQRVGSAAQKAHYLPKLATGELIGAIALTEPNIGSDLVNVETELQQDGDGWRLNGKKKWITLGQIADFFIVLIHCGNQLATVLIDRNTDGFTITPLNDMLGLRGNMLAELHFNDCRLKEDALLGPLTPGVPLAVNFALNEGRFTTACGSLGLCRAAVDVAARYIRQRKQFKRRLFSHGIVQHLFATMLTQTRSAQLMCFSAAEYRETLHPAMINQILMAKYVASKAAVDVAGKAVQLLGANGCHADYAVERYYRDAKIMEIIEGTSQIHEIQIAMNYMMGSEA

SEQ ID NO: 1090 - protein, 422 aa, Escherichia coli
MTKDVALMFPGSGSQYVGMARWLYERYPQVRTLFDEASQITERDMAALCLSGTLVQLAEPTAMALAIYTTSVAHFVAWQQFLAQTGVHVNLRYMLGHSLGEYAALTCSGALSFSQALALVAMRSRLASEIAREMDASTTIIKQGNQALVAAACEVAERETRQQVGIACFNSPQQFMLSGQNSAIIAAEQYLLDHDRQVEVVPLIGGVPYHSPLLKPCGQQLRKALDRCEWRRPCCPVISNVNAQPYPDTVTPVQWLEQQLSQPVQWQRSLTYLTGHLSPIAIEIGPQSVLKNLLLENRYPAPVYAFDNRHDRAQLALVLGDNMAVKTDPEAVRRQRITLLTNALTATRHHRAADVAASAQLKELLSRFFERIQQIEQRGTSSEEDIAFLHELLEQGFQLKGSSQAEIDACHARLASDNGGQA

SEQ ID NO: 1091 - protein, 2310 aa, Escherichia coli
MEQQGIMRQLPTDDQTIVDYLYRIAGEYGEKAAVLMGDAALSYHDLNARSNQLAHYLRGLGIGEDRVVAIRLPRGMAMLIAIFAIVKAGGAYLPLAYNAPRSRIENILSNSGAVCLIGTDDGDRWPIPRVEIDSAAVSAMPTTDLRYRPHARQLAYIIYTSGSTGVPKGVATEHAALLNRIVWMQNAYPISSQDVLFQKTVYTFDVSVWEMFWWAMYGASVVLLPSGLESDPRTLARLIQRHRVSVVHFVPSMLNLFVEYLEMKQDPRLTASLRLVFSSGEKLTVHSVARFYQSVAQGDLINLYGPTEAAIDVSHHRCLRGYDYDDIPIGQAIDGCRLYVLDDHGNPVADGEEGELYLAGIGLARGYLNNVALTDRCFTIHPTLRHLGKPERLYKTGDLVWRDGESQQIHYIGRNDFQIKIRGLRVELGEIEAHAMRFPGVQQAVVVADQDDPDNQLIYAFVVSSVPLNLAALMDALSKNLPAYMLPNRLLAMSELPLSDNGKCCRKTLLDLARAYSASRVDLRETPAVRYLPLSSAQSSMWFMQQLAPHTALYNNPTALLLEGELDRTRMDGAIRQLMSRHTLLRAMAETHNGQPVLAVPQCVSSQALLTIVPLPSVSDDNALQAMINQRAAHPMPLTSGTPLCRFELLTLDDDRSVLLIHLHHIISDGWSKGVLLRELQAAYNGESLTPEPLLEYADYMEYQEEWRQSDAYQDAMRYWQNTLAGTLPILDIPTDQPRQKVARYQGAFVAFALSANTCERVLAAARAQRVSLYNYLLTAFVLLLHRNARQQEYIVGMPIAARLTKEQEHMIAPLVNVLPLRLPLDEAASFSELVQTIRGILFAAFRHQRLEFTDIVRAVNVDRSAGTPCALLPDMQAYLLFTSGSTGEPKGVCVVHRGLLNLLLDMQRTFAVGSQDRLLSVTTPTFDISFLEYLLPLISGASLYLTEAERAADSFRMIPLIADYRPTLMQATPSFWHGLLMAGWRGDPELCVLAGGEALPTKVAEELLRCCGSLWNLYGPTETTIWSLKSQITQAENITLGAPIANTRIYILDNEGHPVPQGVDGELYIAGDGVAQGYDGQPELNAQFFLSEPGVPGGRMFRTGDLVRSDAQGQLFFVGRKDSQIKLRGYRIELGEIERTLARHPHVDAAVVACIERAPLHKALAAFIITSEPPSLFEQLKNELRQQLPDYMVPTLWQRVADFPNTDNGKIDRKRLAENFVADSSLVSPQTQALSDTEQMLLALWMRYLPIKNVDPECDFFRLGGHSLLAVTLVAEINRTFHCALTLKDIFHYSTLRALSARIAQQSITDAAASQDDWVIVHDPEHRHQPFPLTDVQRAYWLGRQTGATSIATHIYHEFDVEHFNVTRFTHAVNALIARHEMLRARVLPDGTQQILAQVPAYQLEQRDLSALSPNARNDALMAIRDRLSHHVHPADRWPLFDFSYSACTAQHGRLHFSLDLLIADALSMRTLQQELMMLYREPHVSLPLLPFSFRDYVQALLVEQASEAYARDQAYWQRALPQLYGPPTLPVQGDLAQLSAIRFVRRRHRLSAHNWGVLSALAQRTRITKTALLLTVFHFPIYQCMFQLDNMPLASPTLNGVNVTPLLLDTSASQVDISLSMQHIDGRITGTFEYDAGLYSADRIQHLVAQWRELLDEASSQPTQLVRDLIRFTPREHAWLARHNATEVALPPVDNLLALVLPHCQQRPTQVALRHADDAMTYGELQQATMQMCTWLRAQGVKRGESVALQLPFCFELIIAQLAILSLGASYVPLDGNAPAARNALILAQATPCMLLVAQPLESPHGLTIPWVLVPDWRSLLTEIPNLPVSVAPDALDCDAVVIFTSGTTGQPKGVRLSQRNLVNLTASFISSYQVTHQDVLLPITSVASASFVGEVLPLLAAGGTLVLAQKAQSLDSDALIALLASQRVTILSTTPSLSASLSVLAQSMGSLRLFLCGGEALEYEQIAPLLPHMAVVNGYGLTESGICSTYFPVAKRREQETGALPIGRPIQNTQAYVVDAYNRLVPPGACGELCFSGLGISPGYLDARQDPERFVELPEYPGVRVLKTGDRARWATDGMLFYLGRQDRQVQIRGYRVELGDIESLLKQHPDIADAWVDVRRNAAATPLLVAFYCSVNGVALDAQQLRVWLSLRLPLHMLPLLYVPLSAMPLGVNGKIDPQCLPLVDLRQLEGPGEYVPPATELEQRLAEIWQQLLGLERVGTTTNFFDLGGHSLLLVQMQQYIGQQCGQHVALVDLLRFTTIKRLAEFLLAPDAAQGTTGDQTQLRAAKQRLAFGHTRWAATTDSHH

SEQ ID NO: 1092 - protein, 1010 aa, Escherichia coli
MAENDFGIAIIGMAGRFPQADTVQAFWENLLASRECISFYSDEELLAMGISPEFVQHPDYVKAKGEVADIDKFDAAFFGIAPREAELMDPQHRVLLETAWAAFEDAGYVAADYPGDVGIFAGKSMDSYLMLNLMPHFKRVFSSGSLQAAIGNDKDSITTTIAYHLNLRGPAITVQTSSSTSLVAVCVACQSLLTWQCDMAIAGGVTLGPPAKTGYLSQEGGITAADGHCRAFSDNSSGFVPGTGAGLVVLKRVDEALRDGDNIYAVIKGFAVNNDGSEKISYTAPSVDAQARAIAQAQRLAGLTPQDITYVEAHGTGTRLGDPVEFSALSQAFAGASQKQYCALGSVKTNIGHLDTAAGVAGLIKTALAVQQGIIPATLHFERPNAQIDLTNSPFYINTTCQPWQPESGIRRAGVTSLGMGGTNAHVVLEQAPAVDLQARAPVPAYSILPFSAKTDSALSSGLARFADFLQHESLPDRRDLAWTLSQGRKAFAHRAALVTRDLHAAGTLLQQAATAPFARGVAQTQLGLGLLFSGQGSQYQRMGHQLYQVWPAYADAFDRCATLLEREYQLDIRHELFRAEVSLAQGERLAQTCLTQPLLFSVEYALAQLWLSWGITPTVMIGHSLGEWVAATLAGVFSLEDALRLVARRAELMHQAPSGAMLMVALPEAQIRALITAPLAIAAVNAPDYSVIAGPTSEILAVSQRLTEQNIINKRLHTSHAFHSSMMQDAAQALRQAFENVRLNPPTLTIISTVTGAHVSADTLTTPDYWIEQMLMPVQFSAALQEAQATFDVDFLEIGPGATLTQLTNGHALGDRLAFSSLPAGARSSDEHKHILDTVAALWVRGHNIDLSAFAGEQPRRVSLPTYAFDKIRYWVDSPEEQRSAVTPVADAGSVIPSEPSVRRQPRPAFSVPYAAPESKTQRGLVAICEALLGIDGLGIDDNFFEAGGHSLMLGMLLAQVQERFAVTLSFFDVMEDASVRALAQLVEQEQQDDGGSALAVLVNDMINE

SEQ ID NO: 1093 - protein, 1454 aa, Escherichia coli
MTIHHAALARMLPAEKKEKLLRQLAQSGVSPSRIPIIKADPAQAIPLSFNQERLWFLQKYDSTATNYNLYVVYRLHGVVDMPMLTEALRHVQARHAILRTRIIVRNDRPCQVIDDASSLVLDTVTLAAQAPTSALDAVIQQVINTRFDLARGPLWGVTQIIQPDQGCHLVFCAHHIIIDGISLRLLFDELQQQYARLHAGNETSLPPPPLQYADYAFWQREWFQDTLLANELAYWRARLQDAPLLSTFPSLHPRPAQPSTHGSRFSITLDETLSLALKHVARTQETTPFVLMLTAFQLVLMRYAQQQRLVIGMPVSGRIRPELQSSIGYYASTAVIYTDFNGVEVGREALQRVKASVKETQGRQQLPFENLVNMLDLPRSLSHSPLFQILYIYHNHVTPRAFTLAGAYWEQVTYHNQTVKYDMTVEVFQNDATFDVSFEYDLGLYDADVVKQIAEALRQHCLSLTSSLETPIGAIPLHAPETATPRRDPLNATNVPWLGPQDVLRIIEQRCVQHPKQLAIQQHDGTLTYAELWARVQFIAMRFRAHGIQPGDRIGVLLPRHRDVIATMLATWFVGACYVPFDIHQPAARLQRLMQRARLVCLVVRQPGEWGEIVQLSLPELMQDMSNAIRYSSQVLARWSLSPTFTLNLTLFNRPQGYPNAEAVIGDFTAVSLLNVCYDSQHSYAHNAQRIQVQLWEDLEHRRFSGIRASEALIHSGRFHAPMPVVFTSMLDIDGETTAQDPRDTTRFTLCPDANITQTPQVWLDHQVIELAGELHFNWDAVEQLFDTTLLDQMFGAYCHALQALVAMPQSWWGVNSSLALPTVSAPVTQAPAPTALLHHGLLRQAALTPQETALISPIRELTYRQLSTAADHVARALLALGVQHGDRVAVVMEKGWQQIAAVHGILRLGAVYLPVDPVLPPQRRQLLLTVGEVRVQVTQPGLTQLEPSLPVLIIDDGMLDTPAAPLPEVAGDVTDLAYIIFTSGSTGTPKGVMIDHRAAMNTLEDINERFGLNAQDRVFGLSSLSFDLSVYDAFAPFMVGAALVLPEAGREKDPRHWQTVMAHGHVSVWNAVPALMQMLCEYHSGDRMSYPTLRLALLSGDWIPLTLPEQMRERLNETMDIISLGGATECAIWSVYYPIGEVESTWTSIPYGRGLRNQPVYVLNAQLEECPVGVEGEICIGGMGLAQGYLNDAEKTAASFVWREASGERIYRTGDRGRYFADGQVAFLGRNDTQVKVNGYRIELGEVKSHLEQLDSVGSAAVVCHQGQLYAFITAAENLHPDDTDALLARVRAQLAVQLPYYLLPQHFFLLKVLPMTGNGKIDQAAMVQEVIQRMSQSTSQKSRALAHASPYEQQVAALWCEVLQREQIGLNDNFFEAGGGSIQIVLLHRRIEEIFKVTVPIAELFRLTTVKRIAGYLQAMQDNARAVNQTQQRDASRSRAQQRLVRRHQRQR

SEQ ID NO: 1094 - protein, 2154 aa, Escherichia coli
MTYSESDIAIVGMNCRYPGVHSVAAFETVLRTGCNILDPKVTPSNGHNHITLNNVYEHMAEFDANFFGYSRAEAEIMDPQQRVFLTCAWEMFEQSGYNPKQHDARVGLYAGVSTSFYLLTHLMNNPDKLAQLGGLQIMVGNDKDHLTSQLAYRLNITGPCVTVQASCATSLVAVHLACEGLLSGQCDMALAGGVTFRMEEQRSYESHGDGLQAEDGLIHTFDAQASGTVYSSGLGMVLLKRATDAQVQGDNILAVIKGSAINNDGGARSGYTVPGVDGQEAVMIEAHSLAEVTPQQIQYLELHGSGTPLGDAIEFAAIKRVFGTPAPNATPWRLGAVKPNVGHVEMASGITSLIKTVLSLTNRVFYPTLNFQRANPQLGLEDSPFEVVSRLTPWPEGTTPRTAGVSAFGLGGTNAHLVVQAPLSTPQARAQQMGPCVVVLSAKNHNALEQMQNALLAKLAAHPEIRLQDVAYTLRHGRFSAPVRKCVIAENCTQLARQLRDAPMVEATTGCTIYWRLGHRFVVALETLSDWLACSEVLSQAVGQLLEHFPLEPACLQDLSPAQRTFISQYALIALIDERETLNVVLCGDGDGGYAAAVLRGDCTLEQAWHRLNAGQPFDDVPTNPLLQPDVCSLMLDDAASDANRTALEALGQLWLAGVSLDWRWVDAAERMLGSQRIALPGTVFTPQRYWVEAVRPATFSHESSNNLLSRATKSDIIAVVTEIWERTLGVSIDDHHASFFELGGHSLLASTILYDIQQRYGITCTLSAFFADPTIEGLSCYLLEQGGSETAVSALPDTVFAPDQQHLPFPLTDVQQAYWVGRRKSLGLGNISTHIYVEYELQGLDETAFNRALNAVIARHSMLRAIVNDDGMQQILPNVPEYHVAFYTTQCEDAFQQRCRELRDTLSHQMIDCSRWPLFQMEVVVDPQQKARLHVSIDLLIADAWSLELFIRELAYHYRHPQAALPTLTYSFRDYVLTLKSYEKTPQFERARDYWRARIETLPPGPRLPLRTDPTKLENPTFVRRSYCLSRAIWQRLKTQAGQMSITPTTLLLTGFAQVLARFSSSPHFSLNLTLFNRLPLHADINHLIGDFTALTLLEIDMSQGETLQARANVIHSQLWRDLDNRLFGGIQVSRLLVQTHRDPAKSVIPIVFTSLLNQYEASWETDDTLFNQPQDDLYSISQTPQVWLDHQVMERNGELHFNWDVVEQLFEPALMDQMFQCYCQLLHALAQRPQLWHETQDVLALPTVSAPVTQAPAPTALLHHGLLRQAALTPQETALISPIRELTYRQLSTAADHVARALLALGVQHGDRVAVVMEKGWQQIAAVHGILRLGAVYLPVDPVLPPQRRQLLLTVGEVRVQVTQPGLTQLEPSLPVLIIDDGMLDTPAAPLPEVAGDVTDLAYIIFTSGSTGTPKGVMIDHRAAMNTLEDINERFGLNAQDRVFGLSSLSFDLSVYDAFAPFMVGAALVLPEAGREKDPRHWQTVMAHGHVSVWNAVPALMQMLCEYHSGDRMSYPTLRLALLSGDWIPLTLPEQMRERLNETMDIISLGGATECAIWSVYYPIGEVESTWTSIPYGRGLRNQPVYVLNAQLEECPVGVEGEICIGGMGLAQGYLNDAEKTAASFVWREASGERIYRTGDRGRYFADGQVAFLGRNDTQVKVNGYRIELGEIERCIARHPDVEQSVVVAVGNSQHRRLVAFAKLHDRHQAQALQAKEAEAAALAQGIIVNPAQRLAFKLKEPHIRALDGLGIALTAPADSTRYIKRRSYRHFSAQKTTLAQLGQLLSGLGQMRLPGLPFAKYAYASAGGLYPVQTYVYLHPDKIEEGVSGIYYFDPRQSCLMPVAPEVELNSGFHAGPNQSIADRAAFTLFMVADMAVISPFYGQEAAWHFSVMEAGTLCHLLEEDAPRYGLGLCQLGMADFSAVASHFQLSPHHRYVHCTVGGAIGQEAASAAALLRDFSTYEKPKETAAPLDMQSYKDAMLRGLRQQLPDYMVPSDLMLATDFPLTANGKLDRQKLQLQGEQIAHQRDGVGPIQVDSALQQRLVALWQEVLGVSHVSAEDDFFSLGGSSIELVRIQQALEAIIGQEIPIVDLFRLPTIADVARYLDEQLHNLPAAHDIVLAQAEVSQVSAARENLALRRKRAQQGEKGDE

SEQ ID NO: 1095 - protein, 487 aa, Escherichia coli
MSEQSYRSAGTLLAQLASGETTSVALVNHYFSRMAQFNKPLNAVVQQHYALALEAAARADRERLEGRARGVLHGLPCTVKESFDVQGWLTTSGAHYLKDNRATQDAPSIARLRAAGAILMGKTNVPMMTADWQTYNDLYGTTHNLWDRQRSPGGSSGGAAVAVAADFTPVEFGSDLFGSLRIPAHYTGVYAHRCSLGLMSVRGHVPGGGPQATDEPDLSTAGPMARSAADLRLMMRALSTFWVEPPRIPDFSRYQAKANYRVCTWFSAPHHEIDQQIAQRFQSFIDKLRAQPGVEVDDAMPADIDPDALFDIAVKLSGRLVSTALNGRQRLTAGLAALGFRLVGKLADVPEGITSYYQGMLKDSGEQRNTDKLRHEYSRVIETLFARYDVLLTPVSPVLAFAHMQQPVRKRKLIVNGEPQDYNEHLFWNMLATVFGLPATVYPLAKTMDELPCGIQIISGHFHDDVTINFAEFCESISGGFTVPEGY

SEQ ID NO: 1096 - protein, 479 aa, Escherichia coli
MTVNKLEPDNGTPNDELFTVAGMFDGSLYKLLLRMALPMFVGMLTQVTYAIADIFWLSHIDVTNSGIIAGVGLVFPVGMGLFAIANGIQIGMGSLLSRAIGMQRLDRAQRILSVGIIIALFFAIVITVLGYVYAQPLLRSLGATKSIIGYATEFYYYSLLTVFSIMLIGVMMGLFQGAGKIMVIMKASLLGALVNIMLDPIMIFVFDFGVKGVALASFLAQLSMVAYFIYTLMGLHIGLSIRIALRPFSWKIYREFLSVGMAQMLMQLIIAVGIVIYNFFIVRLDVNAMAAFTLTGRIDYFIITPMLAIATALLTVVGQNWGHGNVTRTLNAYWAAVALAFSIVLVLAVMHIVLAPWMYPLFTRVVAVSDYAVLQTRIMALALPFVAISLLASEYYQAIGKPWYSVLLTLMRHVFISVPVVYLLAIVLEMRITGVYFGAMSGTFVAALLAWRLLRLSPRLLRWNQEAVRSQHLDMEVAP

SEQ ID NO: 1097 - protein, 1455 aa, Escherichia coli
MMSGNPLSWPQEQCHIIDQLYPYSAVNIIGGVVTIEGIVDLPRLHAAIQSAIRQFDALRMWFVMGEESEVVSQVQPYHWRDIRHLTFSPDYDKENLRPAAIETFVDEWFRQPFTLLAHDLFEFVTFTCGEQYSGYLFKAHHGIADGWSMALLSNHVKRAYEQQDVPDDASPAYSAFLAQQQSYQASTRFAVDRGWWRDYIDEYRDCFPDSSPIVTTEGISCSTWLEPAMINRLYRLCNRYGCTLNTLFIALFALYRARVWGEEKGVIGVPLANRHTREARRCFGMFTNQLPLAYRLVRTERFCERVAFFQRELKRGFKHSKYPITLFNQDLAEQGGGKLRAFDYCVNYYNFTYERHIAGAAQRVESYYSGEQSYKLQIVLQTVNNHKESLRLSLEALRSAFTPHQLTAMKNGLLDLVTALDRQPDARLGDLEVYPAPHVALACGSLKPSFTSRFAAQVVEHGDRTALIDNEQSLTYRQLDDAVERVARYLRQQGIGRGQVVGIIAEHSAQTVMVIYGILRCGAAFLPLNPALPTTRLYAMCRKAQVAHILYDPAMHELTQALAFPASSLLQALATSALAREPWPAIEPQDLAYVLFTSGSTGEPKGVQVSHGNLANYLHFAAERYFTAQDRAALYSSLSFDLTITTLFAPLCVGASISVCRHAESETLLRMAVVDQPNTVIKLTPAHLRLLCAAGISSEQIRTLVVGGEDFKRDLARKAAALFPQAVIYNEYGPTEATVGCMIYRYTGQETLPSLPIGMAIDGCQVAICSPWGCPVPEGETGELVIYGASVTQGYIDAPQQTAAAYLKDTNGVMIGYRSGDIGYAIAPNTLVYQGRKDDQVKINGYRIELCEIEQALLSAPQVESAAVAVIDDVQGQHSGLLACVTPSSVDVATVMQHLRQQLPTYMQPKQCCAIAQLPLSHNGKVDVRQMVATVRNTAPASGSERLGDAAIRHSVRVCVEGALEQTEFDDNENLYVLGLDSIKSIQIAAQLRHHGWTMSAVQVMECGTVNAICEFLASHTTVSQLAQYAHNTRIDLPALRWFTQLALPVPNVYNHVIVLKVLPGCPLEQLHNRLHTLIQQQPALHSALDAEGRLLVCDPNVCYPNEVLTEYSTAQWTLAEVIAQCNSMLDVTNGRVFTAALLHAPQPASSTLVLCAHHLCVDMHSWYLILSTLDAVSTVNGTSNSGLHRWNDYLASKTVDSATHESWRTVCQTLPLHFPPVSLPDDSLPRTRAWREDFRHPCVRRLFESSGNTAYSAETYVLTALALVLRYYSEEPWCRIEMEGMGRGCWPDEPDVADTVGWFTLFYPWAIPLHGDMATLLSAIASDLAKRTHGGGDYGLLQMRHAPEDSLAQGIRMNYIGVQAQPSLRYFHIDHFNSDIYTAPENALGCVLEFNIARSAADGLSFHCRFDPTRIALNDVQLLLARYKNSLTDLDAWLCQHSATLTGAPTLWTL

SEQ ID NO: 1098 - protein, 819 aa, Escherichia coli
MAKDDFTCGSLDIAIIGMSGRFSGAESVPEWWDKLLAGEEFTQPTCTEDDNGNPWIRLRNIITAPYDFDAAFFNIPPGEALLMDPQQRIFLECCYNALEHAGYIPTQLKRVGVYGATYANNYFIDRVYPYLKMSGDHHYLQAQIGNEKDYLCAQVAYKLGFTGPAVSVQTACSSSLVAAYLACEGLLTFQADVALAGGVTLGFLQAHGYSPQGDKLVSQDGHCAPFSAEATGTVYSSGAGVVVLKRLEDALRDQDRVYAVIKGGAVNNDGGRRLGFVAPSVEGQVEAINTALAAAEVVPTDIALIETHGTGTPLGDEIELEALHRVFAPACAPHSIQLGAVKANLGHLGVASGIVSLMKTALTLYTGLVPPQINLVNKHKKLLQPASPFYLSDVVTSVPQTKRIHATVSSFGLGGTNAHLVLQNWCETPAQAVQENERRLFFFSAKTPLALRQQLDAHYHALATYAEADKDRIAYTLAQRRAHFPYRCALAADSVVALRASLAKLRDADMSFTPINMETTLVFLYPDRDDKLESALTHLLACQPNLRQRHQRLSQDVAQICEPADWTPALRQFIQQVSLSEWLIEQSISPVQHIGYLTGAAAAQYVARIISLENAVQQVIVAETTPEQTLAGNSELSEILANLAVTEGTLMLEIGRAGTFSILYHQHAQWVGQTVFSPMLNTDTPEDILPLLGTLWQRGVTICLPEMPAVQTIGLPGYSFDRVRYEIQSSDARENAMLPVSYLSVSDFVEKTWRSLLCIDHYDEHAVIFEYGATSMHVISFVDSCNHIYKIGLTAADIYARPAIREHSEFISECVDGIL

SEQ ID NO: 1099 - protein, 504 aa, Escherichia coli
MTIMEHVSIKTLYHLLCCMLLFISAMCALAQEHEPIGAQDERLSTLIHQRMQEAKVPALSVSVTIKGVRQRFVYGVADVASQKANTLDTVYELGSMSKAFTGLVVQILIQEGRLRQGDDIITYLPEMRLNYQGKPASLTVADFLYHTSGLPFSTLARLENPMPGSAVAQQLRNENLLFAPGAKFSYASANYDVLGAVIENVTGKTFTEVIAERLTQPLGMSATVAVKGDEIIVNKASGYKLGFGKPVLFHAPLARNHVPAAYIHSTLPDMEIWIDAWLHRKALPATLREAMSNSWRGNSDVPLAADNRILYASGWFIDQNQGPYISHGGQNPNFSSCIALRPDQQIGIVALANMNSNLILQLCADIDNYLRIGKYADGAGDAITATDTLFVYLTLLLCFWGAVVVVRGAFRVYRATAHGPGKQQRLRLRVRDYIIALAVPGLVAAMLYVAPGILSPGLDWRFILVWGPSSVLAIPFGIILLAFVLTLNHQIKRILLHNKEWDDE

SEQ ID NO: 1100 - protein, 240 aa, Escherichia coli
MSNISLYCLPYSGGSAAMYYKWRSVLSDNITLRPLEPAGRGTRIRQPLCLTMVDAVADLYQQFVKHYTGGDYAIFGHSLGGIMAFELVHYILDHGHDMPCALFFSGCRPPDRASHEVILHTLPDQAFMEEIVKLGGTPVDVFRNKELMTIFTPIIKNDYRLYEQYVFQAKARTLTCPIVLFHGDADNLVMQDELLAWEKFTTRKTRTIIFPAADHFFVDKHFEQVVGYVNQTIESLEIVG

SEQ ID NO: 1101 - protein, 70 aa, Escherichia coli
MDKFKEKNPLSLRERQVLRMLAQGDEYSQISHNLNISINTVKFHVKNIKHKIQARNTNHAIHIANRNEII

SEQ ID NO: 1102 - protein, 170 aa, Escherichia coli
MAVPSSKEELIKAINSNFSLLNKKLESITPQLAFEPLLEGHAKGTTISVANLVSYLIGWGELVLHWHDQEAKGKTIIFPEEGFKWNELGRLAQKFYRDYEDITEYEVLLARLKENKQQLVALIERFSNDELYGKPWYNKWTRGRMIQFNTASPYKNASGRLNKLQKCLAE

SEQ ID NO: 1103-1104 - skipped (2 entries)

SEQ ID NO: 1105 - DNA, 1083 bp, synthetic construct
  source 1..1083: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcat

SEQ ID NO: 1106 - protein, 360 aa, synthetic construct
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 1107 - DNA, 20 bp, synthetic construct
  source 1..20: mol_type = other DNA
aattgtgagcgctcacaatt

SEQ ID NO: 1108 - DNA, 60 bp, synthetic construct
  source 1..60: mol_type = other DNA
ttgacaattaatcatcggctcgtataatgtgtggaattgtgagcgctcacaattagctgt

SEQ ID NO: 1109 - DNA, 72 bp, synthetic construct
  source 1..72: mol_type = other DNA
gaccagaggtaaggaggtaacaaccatgcgagtgttgaagaaacatcttaatcatgctgcggagggtttcta

SEQ ID NO: 1110 - skipped

SEQ ID NO: 1111 - DNA, 17 bp, synthetic construct
  source 1..17: mol_type = other DNA
taacaccgtgcgtgttg

SEQ ID NO: 1112 - DNA, 17 bp, synthetic construct
  source 1..17: mol_type = other DNA
tacctctggcggtgata

SEQ ID NO: 1113 - DNA, 48 bp, synthetic construct
  source 1..48: mol_type = other DNA
ccgggcgctatcatgccataccgcgaaaggttttgcgccattcgatgg

SEQ ID NO: 1114 - DNA, 1157 bp, synthetic construct
  source 1..1157: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatgg

SEQ ID NO: 1115 - DNA, 29 bp, synthetic construct
  source 1..29: mol_type = other DNA
ttgacaattaatcatcggctcgtataatg

SEQ ID NO: 1116 - DNA, 60 bp, synthetic construct
  source 1..60: mol_type = other DNA
cgcgccgcttcgtcaggccacatagctttcttgttctgatcggaacgatcgttggctgtg

SEQ ID NO: 1117 - DNA, 26 bp, synthetic construct
  source 1..26: mol_type = other DNA
attcaccaccctgaattgactctctt

SEQ ID NO: 1118 - DNA, 1641 bp, synthetic construct
  source 1..1641: mol_type = other DNA
ttgacaattaatcatcggctcgtataatgtgtggaattgtgagcgctcacaattagctgtgaccagaggtaaggaggtaacaaccatgcgagtgttgaagaaacatcttaatcatgctgcggagggtttctaatggggaccattaacacgaagatctataaatacatgagcatctggaaaacaaaacctctgtccgtgctcttgtctgaagcaactgaggatgaaaaaggcctgaagcgcactctgtcggcccgttcacttgttgcgctgggtgtcggtgctattatcggcgctggtttattctctctgaccggcatagctgcggcagacaatgctggaccggcagtaaccctgagctttatcctggcctccgttggttgcgcgttcgctggcctgtgttacgcagaatttgcttctatgattccagttgcgggtagcgcctacacttatagttatgctaccatgggcgagttcgtggcgtggatcatcggttgggatctggtactcgaatacgcattgggcgcagctactgttgccgttagctggtcccagtacgtggacaaattcttgcaaaactacggcatccatattccgaactctatcctccacgggccgtgggataccacccccggtattatcaatttaccgtcgatatttatcatctgcctgctgagcgtgctgctgattcgtggtactaaagaatctgctctgatcaacaacattctggtaatcctgaaagtcacggttgtcatcgtgttcattggcctgggctgggggttcatgaactccgcaaaccacacgccctttatcccggttaacgaaggtgaggctctactgtcttctggtgaaatgagtttcctcaactttttcagcagtgactactttggacactacggatggtccggtattcttcgcggcgctggtgtagtattcttcgcatttatcggcttcgacgcggtgagcactgcggcacaggaggccaaggatccgcagaaaggcatgccaatcggtattctgggctcactgatcatttgcaccgttctgtacgtgcttttcgctttcgttctgaccggtctggaaaactatctaaacttcaaaggtgacgcttctcctgtcaccactgcatttgccaaaacaggctatactttcctgaatagcggtctgacgatcgctatcatagcgggctacacatccgttatgctggtaatgttgatgggtcagtcccgtgtcttttatagtatgtctgtggatggcctgcttccgaagtttttctcgaccctgcataccaaaaacaggactccgtacaaaactaatttgctgttcatggttttcgtaagcctgttcgctggctttgttccggtcagcgacctgggccatatggtatccatcggtaccctcttcgctttctgcctggtgtgtatcggcgttatcgttatgcgaaaaaccaacccagacgccgttcgcggttttcgtgttccttttgtaccggttttcccgattatcggtgtagttatttgtctggttctaatggcgggcctgccgattgaatcttgggaacgtctggcgatctggatgattctgggtgtcgtgatctacttcttctactctaaaaagaactctaaactgaataaccccgaataa

SEQ ID NO: 1119 - DNA, 1766 bp, synthetic construct
  source 1..1766: mol_type = other DNA
ttgacaattaatcatcggctcgtataatgtgtggaattgtgagcgctcacaattaagtgaattgccaataacaattactaaggaggttttttatgtccccgacggcgtttccagcggccgaaacagctactgcccctgcaactgccgtcgatcctgggccagaactggacggcggagatttcgcccttccagagggcgggctggatgacgatcgtcgcttacgtgcattggacgcagttgacgagtatttgacccgcaagcgcaagcatttggttgggtaccaagctacccaggatatggacggaacggccttggatttagcccgtttcatgccccacaacatcaacaacctgggagatcctttccagtcgggtgggtataaaccaaatacgaaagtcgttgagcgtgccgtactggactactatgcaaaattgtggcacgcagaacgtccacacgacccagctgacccagaaagctactggggttacatgttatcgatgggctcaactgagggcaacatgtacgccctgtggaatgcacgtgactacctgtcgggtaaggctttgattcagcctcccacggcaccatttgacgctgttcgctacgtgaaggctgaccccgatcgccgcaatcctaacgcacaccacccagtcgcattctactcggaggatacccactattcttttgctaaagccgttgcggtgctgggtgtcgaaactttccacgctgtgggtctggagaaatacgctgacgagtgccccttggtggatccagtaaccggccttcgtacctggccgaccgaagttccatcgcgcccggggccgtcgggtttaagctgggacggccctggtgagattgatgttgatgcgcttgcagtactggtcgagttcttcgcagcgaagggtcaccccgtcttcgtcaaccttaacttggggtctacatttaaaggagcacatgatgacgtacgtgcggtatgtgaacgcttattaccaatcttcgagcgccatggcttagtacaacgtgaagttgtatatgggagctgtccccaaaccggccgccctttagtggatgtacgtcgcggattttggatccacgtagatggggcacttggggcggggtatgccccttttctgcgtcttgccgccgaagacccggaaggttatggttggacccctgaggcagaattacctgagttcgacttcggcttacgtttgccgacggcggggcatggagaagttgatatggttagcagcatcgccatgagtggacataagtgggcaggcgcgccgtggccatgcggcatctatatgacgaaagtgaaatatcagattagtccaccgtcacagcccgattatattggtgctcctgacacaacatttgccggttcccgtaacggcttttcgccgttaattttgtgggatcatttatcgcgctactcgtaccgcgaccaggtagagcgcatccgcgaagcacaggagcttgcagcatatttggaacgccgccttaccgctatggagcgcgagctgggagtggaactttggccagcccgcacaccgggtgctgtaaccgtacgttttcgcaaaccctctgctgagctggttgcgaagtggtccttgtcgtcgcaggatgttttaatggtgccgggtgatgaaactacgcgtcgtagttacgttcatgtgttcgtgatgccttctgttgatcgtgcaaagttagatgcgttgctggcagaattggccgaagatcccgtcatcttgggtgcgccttaa

SEQ ID NO: 1120 - DNA, 328 bp, synthetic construct
  source 1..328: mol_type = other DNA
aatgtgaatggcacgattatgcgggatacttacaccaccgacggaatatgaaaatcaatattatcgacggctcagaagtgtctagattatccgtggcgatctgacatgggaattagccatggtccatatgaatatcctccttagttcctattccgaagttcctattctctagaaagtataggaacttcgaagcagctccagcctacacaatcgctcaagacgtgtaattccggcgtttcgacattaatcctggcgatcgtctttatgatcaaggcggtcgcggtcatcatcctttcgctggtactcaccatcaaaagtattaccgcca

SEQ ID NO: 1121 - protein, 418 aa, Escherichia coli
MSGLKQELGLAQGIGLLSTSLLGTGVFAVPALAALVAGNNSLWAWPVLIILVFPIAIVFAILGRHYPSAGGVAHFVGMAFGSRLERVTGWLFLSVIPVGLPAALQIAAGFGQAMFGWHSGQLLLAELGTLALVWYIGTRGASSSANLQTVIAGLIVALIVAIWWAGDIKPANIPFPAPGNIELTGLFAALSVMFWCFVGLEAFAHLASEFKNPERDFPRALMIGLLLAGLVYWGCTVVVLHFDAYGEQMAAAASLPKIVVQLFGVGALWIACVIGYLACFASLNIYIQSFARLVWSQAQHNPDHYLARLSSRHIPNNALNAVLGCCVVSTLVIHALEINLDALIIYANGIFIMIYLLCMLAGCKLLQGRYRLLAVVGGLLCVLLLAMVGWKSLYALIMLAGLWLFLPKRKTPENGITT

SEQ ID NO: 1122 - DNA, 32 bp, synthetic construct
  source 1..32: mol_type = other DNA
attgccaataacaattactaaggaggtttttt

(19) United States
(12) Patent Application Publication
(10) Pub. No.: US 2024/0102024 A1
(43) Pub. Date: Mar. 28, 2024

(54) RECOMBINANT BACTERIA ENGINEERED TO TREAT DISEASES ASSOCIATED WITH METHIONINE METABOLISM AND METHODS OF USE THEREOF

(71) Applicants: Synlogic Operating Company, Inc., Cambridge, MA (US); Ginkgo Bioworks, Inc., Boston, MA (US)

(72) Inventors: Dylan Alexander Carlin, Jamaica Plain, MA (US); Vincent M. Isabella, Medford, MA (US); Jonathan McMurry, Boston, MA (US); Theodore Carlton Moore, III, Cambridge, MA (US); Mylene Perreault, Lexington, MA (US); Nathan Schmidt, Milton, MA (US); Mark Simon, Gainesville, FL (US)

(21) Appl. No.: 18/524,651

(22) Filed: Nov. 30, 2023

Related U.S. Application Data
(63) Continuation of application No. 17/819,086, filed on Aug. 11, 2022, now Pat. No. 11,859,189.
(60) Provisional application No. 63/231,773, filed on Aug. 11, 2021.
(60) Provisional application No. 63/281,178, filed on Nov. 19, 2021.
(60) Provisional application No. 63/282,319, filed on Nov. 23, 2021.
(60) Provisional application No. 63/326,323, filed on Apr. 1, 2022.
(60) Provisional application No. 63/355,819, filed on Jun. 27, 2022.

(51) Int. Cl. C12N 15/52; A61K 35/741; C07K 14/245; C12N 9/88; C12N 15/70

(52) U.S. Cl. CPC C12N 15/52; A61K 35/741; C07K 14/245; C12N 9/88; C12N 15/70; C12Y 401/01057; C12Y 404/01011

(57) ABSTRACT

The present disclosure provides host cells, e.g., bacterial cells, that comprise a methionine decarboxylase enzyme for the treatment of diseases and disorders associated with methionine metabolism in a subject. The disclosure further provides pharmaceutical compositions and methods of treating disorders associated with methionine metabolism, such as homocystinuria.

### RELATED APPLICATIONS

The instant application is a continuation application based on U.S. patent application Ser. No. 17/819,086, filed Aug. 11, 2022, which claims priority to U.S. Provisional Application No. 63/231,773, filed on Aug. 11, 2021; U.S. Provisional Application No. 63/281,178 filed Nov. 19, 2021; U.S. Provisional Application No. 63/282,319 filed Nov. 23, 2021; U.S. Provisional Application No. 63/326,323, filed on Apr. 1, 2022; and U.S. Provisional Application No. 63/355,819, filed on Jun. 27, 2022. The entire contents of each of the foregoing applications are expressly incorporated by reference herein.

### SEQUENCE LISTING

The instant application contains a Sequence Listing which has been submitted electronically in XML format and is hereby incorporated by reference in its entirety. Said XML copy, created on Nov. 28, 2023, is named “126046-07007_SL.xml” and is 750,107 bytes in size.

### BACKGROUND

In healthy individuals, acquired dietary methionine is catabolized via the trans-sulfuration pathway, where mammalian cells catabolize methionine into homocysteine via S-Adenosyl-methionine and S-Adenosyl-homocysteine. The cystathionine β-synthase (CBS) enzyme then catalyzes the conversion of homocysteine to cystathionine using vitamin B₆ (pyridoxal 5′-phosphate, PLP) as a co-enzyme. Another PLP-dependent enzyme, cystathionine γ-lyase, converts cystathionine into cysteine. Genetic mutations in one or more of these genes can cause metabolic perturbation in the trans-sulfuration pathway that leads to homocystinuria, also known as cystathionine beta synthase deficiency (“CBS deficiency”) (Garland et al., J. Ped. Child Health, 4(8):557-562, 1999). In homocystinuria patients, CBS enzyme deficiency causes elevated levels of homocysteine and low levels of cystathionine in the serum, which leads to excretion of homocysteine into the urine. Inherited homocystinuria, a serious life-threatening disease, results in high levels of homocysteine in plasma, tissues and urine. Some of the characteristics of the most common form of homocystinuria are myopia (nearsightedness), lens dislocation, higher risk of thromboembolism, and skeletal abnormalities. Homocystinuria may also cause developmental delay/intellectual disability (Mudd et al., Am. J. Hum. Genet., 37:1-31, 1985).

A subpopulation of patients with homocystinuria can be treated with vitamin B6 to increase the residual activity of the CBS enzyme. The B₆ non-responsive patients have to drastically limit the intake of dietary methionine to lower the levels of serum homocysteine. Compliance with a life-long low protein diet combined with methionine-free formula, is often poor, especially among the adults (Gupta et al., J. Inherit Metab. Dis., 2016, 39(1):3946; Mudd et al., Am. J. Hum. Genet., 37:1-31, 1985; Orgeron et al., Prog. Mol. Biol. Transl. Sci., 2014, 121:351-376). Hence, other options for treating homocystinuria are needed.

### SUMMARY

The present disclosure provides novel optimized enzymes and host cells, e.g., recombinant microorganisms, e.g., bacteria, that have been engineered with optimized genetic circuitry, which allows the recombinant microorganism to have improved methionine consumption and/or 3-MTP production. In particular, the host cells, e.g., recombinant microorganisms, disclosed herein have been engineered to comprise gene sequences encoding one or more optimized methionine catabolism enzymes, e.g., methionine decarboxylase (MetDC) enzymes, and/or one or more methionine importer(s), e.g., MetP or MetNIQ. These optimized host cells, e.g., recombinant microorganisms, demonstrate surprising therapeutic efficacy for modulating and treating diseases associated with methionine metabolism, such as homocystinuria, cystinuria, primary and secondary hypermethioninemia.

In one aspect, disclosed herein is a host cell comprising a heterologous methionine catabolism enzyme gene operably linked to a promoter, wherein the heterologous gene encodes a polypeptide that has at least 90%, 91%, 92%, 93%, 94%, 95%, 96%, 97%, 98%, or 99% identity to, comprises, or consists of any one of SEQ ID NOs: 1053, 1048, 1049, 1050, 1051, 1052, 1053, 1054, 1055, 1124, 1126, or 1128. In one embodiment, the heterologous methionine catabolism enzyme gene is a methionine decarboxylase (metDC) gene that encodes a methionine decarboxylase (MetDC) enzyme. In another embodiment, the heterologous methionine catabolism enzyme gene is a leucine decarboxylase (leuDC) gene that encodes a leucine decarboxylase (LeuDC) enzyme, e.g., that has been modified versus a wild-type LeuDC enzyme to have methionine decarboxylase activity.

In another aspect, the disclosure provides a recombinant bacterial cell comprising a heterologous methionine decarboxylase (metDC) gene operably linked to a promoter, wherein the heterologous metDC gene encodes a polypeptide that has at least 90%, 91%, 92%, 93%, 94%, 95%, 96%, 97%, 98%, or 99% identity to, comprises, or consists of any one of SEQ ID NOs: 1053, 1048, 1049, 1050, 1051, 1052, 1053, 1054, 1055, 1124, 1126, or 1128.

In one aspect, disclosed herein is a host cell comprising a heterologous methionine catabolism enzyme gene operably linked to a promoter, wherein the heterologous metDC gene comprises a sequence that has at least 90%, 91%, 92%, 93%, 94%, 95%, 96%, 97%, 98%, or 99% identity to, comprises, or consists of any one of SEQ ID NOs: 1003, 1018, 1034, 1035, 1036, 1037, 1038, 1039, 1040, 1123, 1125, or 1127. In one embodiment, the heterologous methionine catabolism enzyme gene is a methionine decarboxylase (metDC) gene that encodes a methionine decarboxylase (MetDC) enzyme. In another embodiment, the heterologous methionine catabolism enzyme gene is a leucine decarboxylase (leuDC) gene that encodes a leucine decarboxylase (LeuDC) enzyme, e.g., that has been modified versus a wild-type LeuDC enzyme to have methionine decarboxylase activity.

In another aspect, the disclosure provides a recombinant bacterial cell comprising a heterologous methionine decarboxylase (metDC) gene operably linked to a promoter, wherein the heterologous metDC gene comprises a sequence that has at least 90%, 91%, 92%, 93%, 94%, 95%, 96%, 97%, 98%, or 99% identity to, comprises, or consists of any one of SEQ ID NOs: 1003, 1018, 1034, 1035, 1036, 1037, 1038, 1039, 1040, 1123, 1125, or 1127.

In one embodiment, the recombinant bacterial cell further comprising a heterologous gene encoding a methionine importer. In one embodiment, the heterologous gene encoding the methionine importer is a metP gene. In one embodiment, the heterologous metP gene encodes a polypeptide comprising a sequence having at least 90%, 91%, 92%, 93%, 94%, 95%, 96%, 97%, 98%, or 99% identity to, comprises, or consists of any one of SEQ ID NOs: 1056, 1057, 1061, 1130 or 1132. In one embodiment, the heterologous metP gene comprises a sequence having at least 90%, 91%, 92%, 93%, 94%, 95%, 96%, 97%, 98%, or 99% identity to, comprises, or consists of any one of SEQ ID NO: 1041, 1042, 1044, 1129 or 1131.

In one embodiment, the heterologous gene encoding the methionine importer is an metNIQ gene, wherein the metNIQ gene encodes a MetN protein, a MetI protein, and a MetQ protein. In one embodiment, the heterologous metNIQ gene comprises a sequence having at least 90%, 91%, 92%, 93%, 94%, 95%, 96%, 97%, 98%, or 99% identity to, comprises, or consists of any one of SEQ ID NOs: 1043, 1045, 1046, or 1047. In one embodiment, the MetN protein comprises a sequence having at least 90%, 91%, 92%, 93%, 94%, 95%, 96%, 97%, 98%, or 99% identity to, comprises, or consists of any one of SEQ ID NOs: 1062, 1063, or 1058; wherein the MetI protein comprises a sequence having at least 90%, 91%, 92%, 93%, 94%, 95%, 96%, 97%, 98%, or 99% identity to, comprises, or consists of SEQ ID NO: 1059; and wherein the MetQ protein comprises a sequence having at least 90%, 91%, 92%, 93%, 94%, 95%, 96%, 97%, 98%, or 99% identity to, comprises, or consists of SEQ ID NO: 1060.

In another aspect, the disclosure provides a recombinant bacterial cell comprising a heterologous metNIQ gene operably linked to a promoter, wherein the metNIQ gene encodes a MetN protein, a MetI protein, and a MetQ protein, wherein the MetN protein has a sequence that has at least 90%, 91%, 92%, 93%, 94%, 95%, 96%, 97%, 98%, 99% identity to, comprises, or consists of SEQ ID NO: 1062 or 1063; wherein the MetI protein has a sequence that has at least 90%, 91%, 92%, 93%, 94%, 95%, 96%, 97%, 98%, 99% identity to, comprises, or consists of SEQ ID NO: 1059, and wherein the MetQ protein has a sequence that has at least 90%, 91%, 92%, 93%, 94%, 95%, 96%, 97%, 98%, 99% identity to, comprises, or consists of SEQ ID NO: 1060.

In one embodiment, the heterologous metNIQ gene comprises a sequence having at least 90%, 91%, 92%, 93%, 94%, 95%, 96%, 97%, 98%, or 99% identity to, comprises, or consists of any one of SEQ ID NOs: 1043, 1045, 1046, or 1047.

In one embodiment, the recombinant bacterial cell further comprising a heterologous methionine decarboxylase (metDC) gene that encodes a MetDC protein. In one embodiment, the MetDC protein has a sequence that has at least 90%, 91%, 92%, 93%, 94%, 95%, 96%, 97%, 98%, or 99% identity to, comprises, or consists of any one of SEQ ID NOs: 1048, 1049, 1050, 1051, 1052, 1053, 1054, 1055, 1124, 1126, or 1128. In one embodiment, the heterologous metDC gene comprises a sequence having at least 90%, 91%, 92%, 93%, 94%, 95%, 96%, 97%, 98%, or 99% identity to, comprises, or consists of any one of SEQ ID NOs: 1003, 1018, 1034, 1035, 1036, 1037, 1038, 1039, 1040, 1123, 1125, or 1127.

In one embodiment, the recombinant bacterial cell further comprising genetic modification that reduces export of methionine from the bacterial cell. In one embodiment, the genetic modification is a knock-out of an endogenous methionine efflux pump. In one embodiment, the endogenous methionine efflux pump is encoded by a yjeH gene. In one embodiment, the yjeH gene comprises a sequence having at least 90%, 91%, 92%, 93%, 94%, 95%, 96%, 97%, 98%, or 99% identity to, comprises, or consists of SEQ ID NO: 1014.

In one embodiment, the recombinant bacterial cell further comprising an insertion, deletion or mutation of an endogenous phage gene. In one embodiment, the insertion, deletion or mutation is a deletion of the endogenous phage gene comprising a sequence of SEQ ID NO: 1064.

In one embodiment, the recombinant bacterial cell further comprising a modified endogenous colibactin island. In one embodiment, the modified endogenous colibactin island comprises one or more modified clb sequences selected from the group consisting of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), clbR (SEQ ID NO: 1082), and clbS (SEQ ID NO: 1803). In one embodiment, the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and clbR (SEQ ID NO: 1082).

In one embodiment, the recombinant bacterial cell further comprising an auxotrophic modification. In one embodiment, the auxotrophy is a modification or deletion in the dapA gene.

In one embodiment, the recombinant bacterial cell comprises two or three copies of the heterologous metDC gene.

In one embodiment, the promoter is an inducible promoter. In one embodiment, the inducible promoter is directly or indirectly induced by environmental conditions specific to the gut of a human. In one embodiment, the inducible promoter is an IPTG-inducible promoter. In one embodiment, the heterologous gene is located on a plasmid or a chromosome in the bacterial cell.

In another aspect, the disclosure provides a recombinant bacterial cell comprising: a heterologous methionine catabolism enzyme gene, e.g., a methionine decarboxylase (metDC) gene, operably linked to a promoter, wherein the heterologous metDC gene encodes a polypeptide that has at least 90%, 91%, 92%, 93%, 94%, 95%, 96%, 97%, 98%, or 99% identity to, comprises, or consists of any one of SEQ ID NOs: 1048, 1049, 1050, 1051, 1052, 1053, 1054, 1055, 1124, 1126, or 1128, a heterologous gene encoding a methionine importer, wherein the heterologous gene encoding the methionine importer is a metP gene, and wherein the metP gene encodes a polypeptide having at least 90%, 91%, 92%, 93%, 94%, 95%, 96%, 97%, 98%, or 99% identity to, comprises, or consists of any one of a polypeptide encoded by SEQ ID NOs: 1056, 1057, 1061, 1130 or 1132, a knock-out of an endogenous methionine efflux pump, wherein the endogenous methionine efflux pump is encoded by a yjeH gene, a phage deletion, and a ΔdapA auxotrophy.

In another aspect, the disclosure provides a recombinant bacterial cell comprising: a heterologous methionine catabolism enzyme gene, e.g., a methionine decarboxylase (metDC) gene, operably linked to a promoter, wherein the heterologous metDC gene comprises a sequence that has at least 90%, 91%, 92%, 93%, 94%, 95%, 96%, 97%, 98%, or 99% identity to, comprises, or consists of any one of SEQ ID NOs: 1003, 1018, 1034, 1035, 1036, 1037, 1038, 1039, 1040, 1123, 1125, or 1127, a heterologous gene encoding a methionine importer, wherein the heterologous gene encoding the methionine importer is a metP gene, and wherein the metP gene comprises a sequence having at least 90%, 91%, 92%, 93%, 94%, 95%, 96%, 97%, 98%, or 99% identity to, comprises, or consists of any one of SEQ ID NOs: 1041, 1042, 1044, 1129 or 1131, a knock-out of an endogenous methionine efflux pump, wherein the endogenous methionine efflux pump is encoded by a yjeH gene, a phage deletion, and a ΔdapA auxotrophy.

In one embodiment, the recombinant bacterial cell comprises two or three copies of the metDC gene.

In one embodiment, the recombinant bacterial cell further comprising a deletion of an endogenous colibactin island.

In another aspect, the disclosure provides a recombinant bacterial cell comprising: a heterologous methionine catabolism enzyme gene, e.g., a methionine decarboxylase (metDC) gene, operably linked to a promoter, wherein the recombinant bacterial cell comprises two or three copies of the metDC gene, wherein the heterologous metDC gene encodes a polypeptide sequence that has at least 90%, 91%, 92%, 93%, 94%, 95%, 96%, 97%, 98%, or 99% identity to, comprises, or consists of SEQ ID NO: 1048, a heterologous gene encoding a methionine importer, wherein the heterologous gene encoding the methionine importer is a metP gene, wherein the metP gene encodes a polypeptide sequence having at least 90%, 91%, 92%, 93%, 94%, 95%, 96%, 97%, 98%, or 99% identity to, comprises, or consists of SEQ ID NO: 1056, a knock-out of an endogenous methionine efflux pump, wherein the endogenous methionine efflux pump is encoded by a yjeH gene, a phage deletion, a ΔdapA auxotrophy, and a deletion of an endogenous colibactin island.

In one embodiment, the recombinant bacterial cell is SYNB1353.

In one embodiment, the recombinant bacterial cell is a recombinant probiotic bacterial cell. In one embodiment, the recombinant bacterial cell is of the species Escherichia coli strain Nissle.

In another aspect, the disclosure provides a pharmaceutical composition comprising the recombinant bacterial cell disclosed herein and a pharmaceutically acceptable carrier.

In another aspect, the disclosure provides a method for treating a disease associated with methionine metabolism in a subject, the method comprising administering the pharmaceutical composition disclosed herein to the subject.

In another aspect, the disclosure provides a method for reducing the levels of methionine, cysteine and/or homocysteine in a subject, the method comprising administering to the subject the pharmaceutical composition disclosed herein, thereby reducing the levels of methionine, cysteine, and/or homocysteine in the subject.

In another aspect, the disclosure provides a method for reducing the levels of methionine, homocysteine, cysteine and/or cystine in a subject, the method comprising administering to the subject the pharmaceutical composition disclosed herein, thereby reducing the levels of methionine, homocysteine, cysteine, and/or cystine in the subject.

In one embodiment, the subject has homocystinuria, cystinuria, or a metabolic disease. In one embodiment, the subject has cystinuria, and wherein the method reduces the presence, occurrence, or formation of cystine stones in the subject as compared to the presence, occurrence or formation of cystine stones in the subject before administration of the pharmaceutical composition. In one embodiment, the subject has cystinuria, and wherein the method reduces or reduces an increase in cystine stone number, stone volume, stone area or stone weight as compared to before the administration of the pharmaceutical composition.

In one embodiment, the pharmaceutical composition comprises about 1×10¹¹ to about 6×10¹¹ live recombinant bacterial cells/mL.

In one embodiment, about 0.1 g to about 1.5 g of methionine are degraded per day. In one embodiment, methionine is metabolized at a rate of about 1.5 μmol/hr/1e9 cells. In one embodiment, methionine is metabolized at a rate of about 1.3 μmol/hr/1e9 cells. In one embodiment, 3-MTP is produced at a rate of about 1.3 μmol/hr/1e9 cells.

In one embodiment, about 0.1 g to about 1.5 g of methionine are degraded when administered to the subject three times per day. In one embodiment, the subject is fed a meal within one hour of administering the pharmaceutical composition. In one embodiment, the subject is fed a meal concurrently with administering the pharmaceutical composition. In one embodiment, the pharmaceutical composition is administered orally.

In one embodiment, the subject is a human subject.

In one embodiment, consumption of methionine is increased in the subject.

In another aspect, the disclosure provides a method for monitoring the effectiveness of a treatment of a subject, the method comprising administering to the subject the recombinant bacterial cell disclosed herein or the pharmaceutical composition disclosed herein, and measuring a level of 3-MTP in urine of the subject.

In one embodiment, an increase in the level of 3-MTP in the urine of the subject after administration as compared to a level of 3-MTP in the urine of a control subject is an indication that the treatment is effective.

In one embodiment, the increase is at least 1.2-fold, 1.5-fold, 2-fold, 2.5-fold, 3-fold, 3.5-fold, 4-fold, 4.5-fold or 5-fold.

In one embodiment, levels of methionine, homocysteine, cysteine or cystine are measured in the plasma or urine of the subject.

In another aspect, the disclosure provides a non-naturally occurring methionine catabolism enzyme comprising a sequence that has at least 90%, 91%, 92%, 93%, 94%, 95%, 96%, 97%, 98%, or 99% identity to, comprises, or consists of any one of SEQ ID NOs: 1048, 1050, 1051, 1053, 1054, 1124, 1126, or 1128.

In another aspect, the disclosure provides a non-naturally occurring MetN protein comprising a sequence that has at least 90%, 91%, 92%, 93%, 94%, 95%, 96%, 97%, 98%, 99% identity to, comprises, or consists of any one of SEQ ID NOs: 1062 or 1063.

In another aspect, the disclosure provides a host cell that comprises a heterologous polynucleotide encoding a methionine catabolism enzyme, e.g., a methionine decarboxylase (MetDC) enzyme, wherein the heterologous polynucleotide comprises a sequence that is at least 90%, 91%, 92%, 93%, 94%, 95%, 96%, 97%, 98%, or 99% identical to, comprises, or consists of any one of SEQ ID NOs: 1003, 1018, 1034, 1035, 1036, 1037, 1038, 1039, 1040, 1123, 1125, or 1127.

In another aspect, the disclosure provides a host cell that comprises a heterologous polynucleotide encoding methionine catabolism enzyme, e.g., a methionine decarboxylase (MetDC) enzyme, wherein the enzyme comprises an amino acid sequence that is at least 90%, 91%, 92%, 93%, 94%, 95%, 96%, 97%, 98%, or 99% identical to, comprises, or consists of a sequence selected from SEQ ID NOs: 1048, 1049, 1050, 1051, 1052, 1053, 1054, 1055, 1124, 1126, and 1128.

In one embodiment, the sequence of the MetDC enzyme comprises one or more amino acid substitutions relative to the sequence of SEQ ID NO: 1049. In one embodiment, the one or more amino acid substitutions are at positions corresponding to position 41, 66, 70, 82, 203, 379, 491 and/or 500 in SEQ ID NO: 1049. In one embodiment, the sequence of the MetDC enzyme comprises: a glutamine (Q) at a position corresponding to position 41 in the sequence of SEQ ID NO: 1049; an asparagine (N) at a position corresponding to position 66 in SEQ ID NO: 1049; an aspartic acid (D) at a position corresponding to position 70 in the sequence of SEQ ID NO: 1049; a histidine (H) at a position corresponding to position 82 in SEQ ID NO: 1049; a histidine (H) at a position corresponding to position 203 in SEQ ID NO: 1049; a glycine (G) at a position corresponding to position 379 in SEQ ID NO: 1049; a leucine (L) at a position corresponding to position 491 in the sequence of SEQ ID NO: 1049; and/or a proline (P) at a position corresponding to position 500 in the sequence of SEQ ID NO: 1049. In one embodiment, the sequence of the MetDC enzyme comprises amino acid substitutions at a position corresponding to: position 66; position 203; position 379; positions 70 and 82; positions 491 and 500; or positions 41 and 70 in the sequence of SEQ ID NO: 1049. In one embodiment, the sequence of the MetDC enzyme comprises the following amino acid substitutions relative to the sequence of SEQ ID NO: 1049: T66N; A203H; H379G; Q70D and N82H; V491L and A500P; or R41Q and Q70D. In another aspect, the disclosure provides a host cell that comprises a heterologous polynucleotide encoding a methionine decarboxylase (MetDC) enzyme, wherein the sequence of the MetDC enzyme comprises one or more amino acid substitutions relative to the sequence of SEQ ID NO: 1049 at positions corresponding to position 41, 66, 70, 82, 203, 379, 491 and/or 500 in SEQ ID NO: 1049.

In one embodiment, the sequence of the MetDC enzyme comprises: a glutamine (Q) at a position corresponding to position 41 in the sequence of SEQ ID NO: 1049; an asparagine (N) at a position corresponding to position 66 in SEQ ID NO: 1049; an aspartic acid (D) at a position corresponding to position 70 in the sequence of SEQ ID NO: 1049; a histidine (H) at a position corresponding to position 82 in SEQ ID NO: 1049; a histidine (H) at a position corresponding to position 203 in SEQ ID NO: 1049; a glycine (G) at a position corresponding to position 379 in SEQ ID NO: 1049; a leucine (L) at a position corresponding to position 491 in the sequence of SEQ ID NO: 1049; and/or a proline (P) at a position corresponding to position 500 in the sequence of SEQ ID NO: 1049. In one embodiment, the sequence of the MetDC enzyme comprises amino acid substitutions at a position corresponding to: position 66; position 203; position 379; positions 70 and 82; positions 491 and 500; or positions 41 and 70 in the sequence of SEQ ID NO: 1049. In one embodiment, the sequence of the MetDC enzyme comprises the following amino acid substitutions relative to the sequence of SEQ ID NO: 1049: T66N; A203H; H379G; Q70D and N82H; V491L and A500P; or R41Q and Q70D.

In one embodiment, the host cell further comprising a heterologous polynucleotide encoding a methionine importer. In one embodiment, the methionine importer is a MetP importer.

In one embodiment, the heterologous polynucleotide encoding the MetP importer comprises a sequence that is at least 90%, 91%, 92%, 93%, 94%, 95%, 96%, 97%, 98%, or 99% identical to, comprises, or consists of any one of SEQ ID NOs: 1041, 1042, 1044, 1129, or 1131.

In one embodiment, the MetP importer comprises an amino acid sequence that is at least 90%, 91%, 92%, 93%, 94%, 95%, 96%, 97%, 98%, or 99% identical to, comprises, or consists of any one of SEQ ID NOs: 1056, 1057, 1061, 1130 or 1132.

In another aspect, the disclosure provides a host cell that comprises a heterologous polynucleotide encoding a MetP importer, wherein the MetP importer comprises an amino acid sequence that is at least 90%, 91%, 92%, 93%, 94%, 95%, 96%, 97%, 98%, or 99% identical to, comprises, or consists of any one of SEQ ID NOs: 1056, 1057, 1061, 1130, or 1132.

In one embodiment, the methionine importer is an MetNIQ importer, wherein the MetNIQ importer comprises a MetN protein, a MetI protein, and a MetQ protein.

In one embodiment, a heterologous polynucleotide encoding the MetNIQ importer comprises a sequence that is at least 90%, 91%, 92%, 93%, 94%, 95%, 96%, 97%, 98%, or 99% identical to, comprises, or consists of SEQ ID NO: 1043, or wherein a heterologous polynucleotide encoding the MetN protein comprises a sequence that is at least 90%, 91%, 92%, 93%, 94%, 95%, 96%, 97%, 98%, or 99% identical to, comprises, or consists of any one of SEQ ID NOs: 1004, 1045, 1046, or 1047; wherein the a heterologous polynucleotide encoding the MetI protein comprises a sequence that is at least 90%, 91%, 92%, 93%, 94%, 95%, 96%, 97%, 98%, or 99% identical to, comprises, or consists of SEQ ID NO: 1005; and wherein a heterologous polynucleotide encoding the MetQ protein comprises a sequence that is at least 90%, 91%, 92%, 93%, 94%, 95%, 96%, 97%, 98%, or 99% identical to, comprises, or consists of SEQ ID NO: 1006.

In one embodiment, the MetN protein comprises an amino acid sequence that is at least 90%, 91%, 92%, 93%, 94%, 95%, 96%, 97%, 98%, or 99% identical to, comprises, or consists of any one of SEQ ID NOs: 1058, 1062, and 1063; wherein the MetI protein comprises an amino acid sequence that is at least 90%, 91%, 92%, 93%, 94%, 95%, 96%, 97%, 98%, or 99% identical to, comprises, or consists of SEQ ID NO: 1059; and wherein the MetQ protein comprises an amino acid sequence that is at least 90%, 91%, 92%, 93%, 94%, 95%, 96%, 97%, 98%, or 99% identical to, comprises, or consists of SEQ ID NO: 1060.

In another aspect, the disclosure provides a host cell comprising a heterologous polynucleotide encoding an MetNIQ importer, wherein the MetNIQ importer comprises MetN protein, a MetI protein, and a MetQ protein, wherein a heterologous polynucleotide encoding the MetN protein comprises a sequence that is at least 90%, 91%, 92%, 93%, 94%, 95%, 96%, 97%, 98%, or 99% identical to, comprises, or consists of any one of SEQ ID NOs: 1045, 1046, or 1047; wherein the a heterologous polynucleotide encoding the MetI protein comprises a sequence that is at least 90%, 91%, 92%, 93%, 94%, 95%, 96%, 97%, 98%, or 99% identical to, comprises, or consists of SEQ ID NO: 1005; and wherein a heterologous polynucleotide encoding the MetQ protein comprises a sequence that is at least 90%, 91%, 92%, 93%, 94%, 95%, 96%, 97%, 98%, or 99% identical to, comprises, or consists of SEQ ID NO: 1006.

In another aspect, the disclosure provides a host cell comprising a heterologous polynucleotide encoding a MetNIQ importer, wherein the MetNIQ importer comprises a MetN protein, a MetI protein, and a MetQ protein, wherein the MetN protein comprises an amino acid sequence that is at least 90%, 91%, 92%, 93%, 94%, 95%, 96%, 97%, 98%, or 99% identical to, comprises, or consists of any one of SEQ ID NOs: 1062 or 1063; wherein the MetI protein comprises an amino acid sequence that is at least 90%, 91%, 92%, 93%, 94%, 95%, 96%, 97%, 98%, or 99% identical to, comprises, or consists of SEQ ID NO: 1059; and wherein the MetQ protein comprises an amino acid sequence that is at least 90%, 91%, 92%, 93%, 94%, 95%, 96%, 97%, 98%, or 99% identical to, comprises, or consists of SEQ ID NO: 1060.

In one embodiment, the host cell further comprising a heterologous polynucleotide encoding a methionine catabolism enzyme, e.g., a methionine decarboxylase (MetDC) enzyme. In one embodiment, the heterologous polynucleotide encoding the enzyme comprises a sequence that is at least 90%, 91%, 92%, 93%, 94%, 95%, 96%, 97%, 98%, or 99% identical to, comprises, or consists of a sequence selected from SEQ ID NOs: 1003, 1018, 1034, 1035, 1036, 1037, 1038, 1039, 1040, 1123, 1125, or 1127.

In one embodiment, the enzyme comprises an amino acid sequence that is at least 90%, 91%, 92%, 93%, 94%, 95%, 96%, 97%, 98%, or 99% identical to, comprises, or consists of a sequence selected from SEQ ID NOs: 1048, 1049, 1050, 1051, 1052, 1053, 1054, 1055, 1124, 1126, or 1128.

In one embodiment, the heterologous polynucleotide is operably linked to a promoter. In one embodiment, the promoter is an inducible promoter. In one embodiment, the inducible promoter is an IPTG-inducible promoter or an inducible promoter that is directly or indirectly induced by environmental conditions specific to the gut of a human.

In one embodiment, the host cell is a plant cell, an algal cell, a yeast cell, a bacterial cell, or an animal cell.

In one embodiment, the host cell is a yeast cell. In one embodiment, the yeast cell is an Saccharomyces cell, a Yarrowia cell or a Pichia cell.

In one embodiment, the host cell is a bacterial cell. In one embodiment, the bacterial cell is a recombinant probiotic bacterial cell.

In one embodiment, the bacterial cell is an E. coli cell or a Bacillus cell. In one embodiment, the bacterial cell is of the species Escherichia coli strain Nissle.

In another aspect, the disclosure provides a method comprising culturing the host cell disclosed herein.

In another aspect, the disclosure provides a method of metabolizing methionine comprising culturing the host cell disclosed herein.

In another aspect, the disclosure provides a non-naturally occurring nucleic acid comprising a sequence that is at least 90%, 91%, 92%, 93%, 94%, 95%, 96%, 97%, 98%, or 99% identical to, comprises, or consists of any one of SEQ ID NOs: 1034, 1035, 1036, 1038, 1039, 1123, 1125, or 1127.

In another aspect, the disclosure provides a non-naturally occurring nucleic acid comprising a sequence that is at least 90%, 91%, 92%, 93%, 94%, 95%, 96%, 97%, 98%, or 99% identical to any one of SEQ ID NOs: 1045, 1046, and 1047.

In another aspect, the disclosure provides a non-naturally occurring nucleic acid comprising a sequence that is at least 90%, 91%, 92%, 93%, 94%, 95%, 96%, 97%, 98%, or 99% identical to any one of SEQ ID NOs: 1129 or 1131.

In another aspect, the disclosure provides a vector comprising the non-naturally occurring nucleic acid disclosed herein.

In another aspect, the disclosure provides an expression cassette comprising the non-naturally occurring nucleic acid disclosed herein.

### BRIEF DESCRIPTION OF THE DRAWINGS

FIG. 1 provides an overview for homocystinuria, a disorder of methionine metabolism caused by a defect in cystathionine beta-synthase (CBS), which leads to the accumulation of homocysteine in blood and urine.

FIG. 2 provides an overview of the synthetic biology for methionine consumption in E. coli Nissle (ECN).

FIG. 3 is a graph depicting methionine disappearance from minimal media in E. coli Nissle harboring methionine gamma lyase (MGL) or methionine decarboxylase (MDC) under the control of an anhydrotetracycline (ATC)-inducible promoter. EcN control is wild-type E. coli Nissle with no methionine catabolism enzymes. BA CGL/MGL is MGL from Brevibacterium aurantiacum (DOI 10.1124/jpet.119.256537). CF MGL is MGL from Citrobacter freundii. PG MGL is MGL from Porphyromonas gingivalis. EcN-MetDC is MDC from Streptomyces sp. 590.

FIG. 4 is a graph depicting L-Met consumption by E. coli Nissle, SYN7344 (SpmetDC (SEQ ID NO: 1049), harbouring a medium-copy plasmid encoding an anhydrotetracycline-inducible MetDC compared to the control strain, SYN094.

FIG. 5 is a graph depicting L-Met consumption by E. coli Nissle strains, SYN094 (control), SYN7328 (metNIQ (SEQ ID NOs: 1058, 1059, and 1060)), SYN7344 (SpmetDC (SEQ ID NO: 1049)), SYN7345 (ΔyjeH), SYN7346 (ΔyjeH, SpmetDC (SEQ ID NO: 1049)), SYN7347 (ΔyjeH, metNIQ (SEQ ID NOs: 1058, 1059, and 1060)), SYN7348 (SpmetDC (SEQ ID NO: 1049), metNIQ (SEQ ID NOs: 1058, 1059, and 1060)), and SYN7349 (ΔyjeH, SpmetDC (SEQ ID NO: 1049), metNIQ (SEQ ID NOs: 1058, 1059, and 1060)).

FIG. 6A depicts the plasma exposure of D4-Met (labeled methionine) in mice after receiving an oral dose of 200 mg/kg D4-Met.

FIG. 6B depicts the level of D4-Met and D4-homocysteine (Hey) in urine samples. Bl: baseline; T5: 5 hours after dosing.

FIG. 6C depicts the level of D4-Met in intestinal effluent samples (upper small intestine, middle small intestine, lower small intestine, colon).

FIG. 6D depicts the plasma level of endogenous methionine in mice.

FIG. 6E depicts the level of endogenous methionine and homocysteine in urine samples. Bl: baseline; T5: 5 hours after dosing.

FIG. 6F depicts the level of endogenous methionine in intestinal effluent samples (upper small intestine, middle small intestine, lower small intestine, colon).

FIG. 7A depicts the level of D4-3MTP detected in urine samples collected from mice after receiving a dose of 2.8×10¹⁰ recombinant bacterial strain (SYN094 or SYN7349) or a vehicle solution. A dose of 200 mg/kg D4-Met (labeled methionine), was administered to the mice 10 minutes after the bacterial strains.

FIG. 7B depicts the level of 3MTP in urine samples after receiving a dose of 2.8×10¹⁰ recombinant bacterial strain (SYN094 or SYN7349) or a vehicle solution.

FIG. 7C depicts the level of 3MTP in intestinal effluent samples (upper small intestine, middle small intestine, lower small intestine, colon) after receiving a dose of 2.8×10¹⁰ recombinant bacterial strain (SYN094 or SYN7349) or a vehicle solution.

FIG. 8 depicts increased in vitro methionine consumption by strains SYN7344 (metDC (SEQ ID NO: 1049)), SYN7346 (ΔyjeH, metDC (SEQ ID NO: 1049)), and SYN7349 (ΔyjeH, metDC (SEQ ID NO: 1049), metNIQ (SEQ ID NOs: 1058, 1059, 1060)) compared to SYN094 (control).

FIG. 9A depicts levels of 3-MTP produced by 173 different metDC candidate genes.

FIG. 9B depicts levels of 3-MTP produced by different metDC genes identified in the MetDC screen.

FIG. 9C depicts results from a secondary screen identifying methionine catabolism enzymes (e.g., MetDCs). MetDCs identified in the screen included SpMetDC codon optimized (SEQ ID NO: 1049), SpMetDC H379G (SEQ ID NO: 1128; engineered library), SpMetDC A203H (SEQ ID NO: 1126; engineered library), SpMetDC T66N (SEQ ID NO: 1124; engineered library), SpMetDC Q70D N82H (SEQ ID NO: 1048; engineered library), SpMetDC R41Q Q70D (SEQ ID NO: 1051; engineered library), and SpMetDC V491L A500P (SEQ ID NO: 1050; engineered library).

FIG. 9D depicts 3MTP produced by different strains expressing different MetDC proteins identified in the metagenomic screen.

FIG. 10A depicts minimum inhibitory concentrations of a toxic Met analog (norleucine) for metagenomic (MetP) and protein engineered (MetNIQ) importers. Lower MICs are taken to imply higher importer activity.

FIG. 10B depicts 3-MTP production by E. coli strains with methionine importers, MetP or MetNIQ, added to a MetDC expressing background (SYN7346 (ΔyjeH, metDC (SEQ ID NO: 1049)). Strains with added MetP or MetNIQ improved activity over prototype (SYN7349 (ΔyjeH, metDC (SEQ ID NO: 1049), metNIQ (SEQ ID NOs: 1058, 1059, 1060))). Strains with MetP added are SYN7818 (metP (metagenomics library; F. frigoris; SEQ ID NO: 1057)) and SYN7819 (metP (metagenomics library; F. segetis; SEQ ID NO: 1056)). Strains with MetNIQ added are SYN7816 (metN(P281G)IQ; SEQ ID NOs: 1062, 1059, and 1060), SYN7817 (metN(P281G)IQ; SEQ ID NOs: 1058, 1059, and 1060), and SYN7815 (metN(P281S)IQ; SEQ ID NOs: 1063, 1059, and 1060).

FIG. 10C depicts 3-MTP production comparing SYN7349 (ΔyjeH, metDC (SEQ ID NO: 1049), metNIQ (endogenous)), SYN7818 (ΔyjeH, metDC (SEQ ID NO: 1049), metP (metagenomics library; F. frigoris; SEQ ID NO: 1057)), and SYN7819 (ΔyjeH, metDC (SEQ ID NO: 1049), metP (metagenomics library; F. segetis; SEQ ID NO: 1056)).

FIGS. 11A and 11B depict graphs showing Met-d4 consumption (FIG. 11A) and 3-MTP-d4 production (FIG. 11B) by E. coli Nissle strains: SYN094 (control), SYN7346 (ΔyjeH, metDC SEQ ID NO: 1049), SYN7640 (metDC (SEQ ID NO: 1049), ΔyjeH), SYN7641 (metDC (V491L A500P; SEQ ID NO: 1050; engineered library), ΔyjeH), SYN7642 (metDC (Q70D N82H; SEQ ID NO: 1048; engineered library), ΔyjeH), SYN7643 (metDC (R41Q Q70D; SEQ ID NO: 1051; engineered library), ΔyjeH), SYN7644 (metDC (Stanieria sp. NIES-3757; metagenomic library), ΔyjeH), SYN7689 (engineered metDC (Mus musculus; SEQ ID NO: 1054; metagenomic library), ΔyjeH), SYN7690 (engineered leuDC (Mus musculus; SEQ ID NO: 1053; metagenomic library), ΔyjeH), and SYN7691 (metDC (Entamoeba histolytica; SEQ ID NO: 1055; metagenomics library), ΔyjeH).

FIG. 11C depicts 3-MTP production of top performing library MetDCs compared to wildtype EcN, and the prototype; the final MetDC selected to move forward (Streptomyces sp. 590; Q70D, N82H), a member of the protein engineered library, is in pink. IVS=in vitro gastric simulation.

FIG. 12 depicts 3-MTP production by E. coli Nissle strains SYN094 (control), SYN7642 (metDC (Q70D N82H; SEQ ID NO: 1048; engineered library)), SYN7970 (2 copies metDC (Q70D N82H; SEQ ID NO: 1048; engineered library), metP (metagenomics library; F. segetis; SEQ ID NO: 1056), ΔyjeH, Δdap, ΔΦ), SYN8002 (3 copies metDC (Q70D N82H; SEQ ID NO: 1048; engineered library), metP (metagenomics library; F. segetis; SEQ ID NO: 1056), ΔyjeH, Δdap, ΔΦ), and SYN8003 (3 copies metDC (Q70D N82H; SEQ ID NO: 1048; engineered library), metP (metagenomics library; F. segetis; SEQ ID NO: 1056), ΔyjeH, Δdap, ΔΦ). Lyo: lyophilized strains. SYN8003 has stronger RBS sites than SYN8002.

FIG. 13 depicts Met consumption by E. coli strains SYN094 (control), SYN7349 (ΔyjeH, metDC (SEQ ID NO: 1049), metNIQ (endogenous)), SYN8003 (3 copies metDC (Q70D N82H; SEQ ID NO: 1048; engineered library), metP (metagenomics library; F. segetis; SEQ ID NO: 1056), ΔyjeH, Δdap, ΔΦ), and SYN8070 (3 copies metDC (Q70D N82H; SEQ ID NO: 1048; engineered library), metP (metagenomics library; F. segetis; SEQ ID NO: 1056), ΔyjeH, Δdap, ΔΦ, Δpks).

FIG. 14A is a schematic of an exemplary engineered E. coli Nissle capable of consuming methionine. Optimal metP and metDC were identified from metagenomic, codebase and protein engineering libraries.

FIG. 14B is a graph showing in vitro methionine consumption (solid line) and 3-MTP production (dotted line) by EcN (unengineered bacteria) or SYNB1353 ((lacI-Ptac, IPTG) 3× metDC (Q70D N82H; SEQ ID NO: 1048; engineered library); 1× metP (metagenomics library; F. segetis; SEQ ID NO: 1056); ΔΦ; Δdap; ΔyjeH; Δpks). Cells were incubated for the indicated time in M9 medium with 0.5% glucose and 10 mM methionine at 37° C., supernatant was collected for methionine (HPLC) and 3-MTP (LC-MS/MS) measurements. *p<0.05 versus EcN. Met: methionine, metP: methionine importer, metDC: methionine decarboxylase, YjeH: methionine exporter.

FIG. 15A depicts a schematic of the study design in mice, including administration of EcN or SYNB1353 ((lacI-Ptac, IPTG) 3× metDC (Q70D N82H; SEQ ID NO: 1048; engineered library); 1× metP (metagenomics library; F. segetis; SEQ ID NO: 1056); ΔΦ; Δdap; ΔyjeH; Δpks) and time points of feces collection.

FIG. 15B depicts kinetics of fecal excretion in healthy male mice. Antibiotic resistant EcN or SYNB1353 ((lacI-Ptac, IPTG) 3× metDC (Q70D N82H; SEQ ID NO: 1048; engineered library); 1× metP (metagenomics library; F. segetis; SEQ ID NO: 1056); ΔΦ; Δdap; ΔyjeH; Δpks) were orally administered at 1e10 CFU and fecal pellets collected at the indicated timepoints for CFU enumeration.

FIG. 15C depicts a schematic of the study design in mice, including administration of EcN or SYNB1353 ((lacI-Ptac, IPTG) 3× metDC (Q70D N82H; SEQ ID NO: 1048; engineered library); 1× metP (metagenomics library; F. segetis; SEQ ID NO: 1056); ΔΦ; Δdap; ΔyjeH; Δpks) and time points for urine collection.

FIG. 15D is a graph showing the effects of SYNB1353 ((lacI-Ptac, IPTG) 3× metDC (Q70D N82H; SEQ ID NO: 1048; engineered library); 1× metP (metagenomics library; F. segetis; SEQ ID NO: 1056); ΔΦ; Δdap; ΔyjeH; Δpks) on urinary recovery of 3-MTP in healthy mice. Mice received a single oral dose of bacteria followed by 200 mg/kg D4-methionine 30 minutes later. Mice were immediately placed in metabolic cages (n=3/cage) and urine collected 5 hours later. *p<0.05 versus EcN. 3-MTP: 3-methylthiopropylamine. Data presented as mean urinary 3-MTP recovery±standard error of the mean (n=18 mice/strain with 3 mice/cage). Statistical analysis was performed using one-way ANOVA followed by Dunnett's multiple comparison test. *p<0.05.

FIG. 15E depicts methionine load challenge at 200 mg/kg. D4-methionine was given at 200 mg/kg to mice via oral gavage at TO. SYNB1353 or SYN094 was administered at TO and T1. Urine was collected at 2 hours and urinary D4-3MTP was measured.

FIGS. 16A and 16B are graphs showing the effects of methionine load (administration of 100 mg/kg or 300 mg/kg methionine (Met)) on plasma Methionine (FIG. 16A) and plasma total homocysteine (FIG. 16B) in healthy nonhuman primates. Data presented as mean±standard error of the mean (n=6/group). Statistical analysis was performed using two-way repeated ANOVA with Sidak's multiple comparison test. *p<0.05 versus 100 mg/kg methionine.

FIG. 17A depicts nonhuman primate study design. Male cynomolgus monkeys (2-5 years old) were fasted overnight and received an oral methionine load (100 or 300 mg/kg) and vehicle or bacteria (1e12 live cells). Plasma was collected throughout, and urine was recovered 6 hours post dosing.

FIGS. 17B-17D are graphs showing SYNB1353 ((lacI-Ptac, IPTG) 3× metDC (Q70D N82H; SEQ ID NO: 1048; engineered library); 1× metP (metagenomics library; F. segetis; SEQ ID NO: 1056); ΔΦ; Δdap; ΔyjeH; Δpks) is active in a nonhuman primate model of acute homocystinuria as evidenced by (1) urinary 3-MTP recovery at 6 hours post-dosing with vehicle, or SYNB1353 at 1e12 live cells administered concomitantly with 100 mg/kg or 300 mg/kg methionine (FIG. 17B); (2) plasma methionine (FIG. 17C) and plasma homocysteine (FIG. 17D) at 0.5, 1.0, 2.0, 4.0, and 6.0 hours post-dosing with vehicle, or SYNB1353 at 1e12 live cells administered concomitantly with 100 mg/kg or 300 mg/kg. Data presented as mean±standard error of the mean (n=12/group for 100 mg/kg methionine, n=6/group for 300 mg/kg methionine). Statistical analysis was performed using unpaired t-test with Welch's correction (FIG. 17B) and two-way ANOVA with Sidak's multiple comparison test (FIG. 17C, FIG. 17D). *p<0.05.

FIGS. 18A-18D depict schematics showing the experimental outline (FIG. 18A) and dose-dependent effect of SYNB1353 ((lacI-Ptac, IPTG) 3× metDC (Q70D N82H; SEQ ID NO: 1048; engineered library); 1× metP (metagenomics library; F. segetis; SEQ ID NO: 1056); ΔΦ; Δdap; ΔyjeH; Δpks) as evidenced by (1) increased urinary recovery of 3-MTP (FIG. 18B) and decreased plasma methionine (FIG. 18C) and plasma total homocysteine (FIG. 18D) in a nonhuman primate model of acute homocystinuria. 3-MTP: 3-methylthiopropylamine. Data was normalized to the study-respective vehicle and presented as mean±standard error of the mean (n=12/group). Statistical analysis was performed using paired t-test. *p<0.05.

FIGS. 19A-19D are graphs showing levels of D4-methionine over time in the effluents of the cecum (FIG. 19A), and effluents of the colon (FIG. 19B), in the plasma (FIG. 19C) and small intestine effluents (FIG. 19D) of C57BL/6 mice having received a single IP dose of D4-methionine (100 mg/kg).

FIG. 20A is a graph showing levels of D4-3-MTP in the urine over time in C57BL/6 mice having received a single IP dose of D4-methionine (100 mg/kg) followed by 2 oral doses of SYN094 (wild type E. coli Nissle) or SYNB1353 ((lacI-Ptac, IPTG) 3× metDC (Q70D N82H; SEQ ID NO: 1048; engineered library); 1× metP (metagenomics library; F. segetis; SEQ ID NO: 1056); ΔΦ; Δdap; ΔyjeH; Δpks) at 0.5 and 1.5 hours post IP injection.

FIGS. 20B and 20C are graphs showing levels of plasma D4-methionine (FIG. 20B) and D4-homocysteine (FIG. 20C) over time in C57BL/6 mice having received a single IP dose of D4-methionine (100 mg/kg) followed by 2 oral doses of SYN094 (wild type E. coli Nissle) or SYNB1353 ((lacI-Ptac, IPTG) 3× metDC (Q70D N82H; SEQ ID NO: 1048; engineered library); 1× metP (metagenomics library; F. segetis; SEQ ID NO: 1056); ΔΦ; Δdap; ΔyjeH; Δpks) at 0.5 and 1.5 hours post IP injection.

FIG. 21A depicts a graph modeling predicted methionine consumption by SYNB1353 ((lacI-Ptac, IPTG) 3× metDC (Q70D N82H; SEQ ID NO: 1048; engineered library); 1×metP (metagenomics library; F. segetis; SEQ ID NO: 1056); ΔΦ; Δdap; ΔyjeH; Δpks) as a function of TID dose for an HCU patient consuming 300 mg/day of methionine. Solid line: best-guess SYNB1353 activity; shaded region: uncertainty in SYNB1353 activity.

FIG. 21B depicts a graph showing modeling of simulated protein liberalization by SYNB1353 ((lacI-Ptac, IPTG) 3× metDC (Q70D N82H; SEQ ID NO: 1048; engineered library); 1×metP (metagenomics library; F. segetis; SEQ ID NO: 1056); ΔΦ; Δdap; ΔyjeH; Δpks) dosing at 5e11 or 1e12 live cells TID as a function of current protein intake. Solid lines: best-guess SYNB1353 activity; shaded regions: uncertainty in SYNB1353 activity.

FIG. 21C depicts a graph showing modeling of plasma homocysteine at baseline and after SYNB1353 ((lacI-Ptac, IPTG) 3× metDC (Q70D N82H; SEQ ID NO: 1048; engineered library); 1×metP (metagenomics library; F. segetis; SEQ ID NO: 1056); ΔΦ; Δdap; ΔyjeH; Δpks) dosing at 5e11 or 1e12 live cells TID as a function of disease severity. Error bars: uncertainty in HCU patient physiology; annotations: percent lowering of plasma homocysteine.

FIG. 22A depicts a schematic showing the pathway for cysteine and cystine production in humans via the metabolism of methionine through a trans-sulfuration pathway.

FIG. 22B is a schematic showing the formation of cystine, a cysteine dimer, which builds up in the urine of subjects having cystinuria and forms crystals or stones (urolithiasis).

FIG. 23 depicts a schematic showing the outline of a mouse study in which SLC3A1 KO fed a high- (0.62%) or low-methionine (0.12%) diet for 8 weeks. Time points are indicated at which body weight and food intake are measured, urine is collected, and CT scans are performed.

FIGS. 24A-24D depict graphs showing measurements obtained in the mouse study outlined in FIG. 23. FIG. 24A depicts a graph showing body weight measured. FIG. 24B depicts a graph showing food intake normalized to body weight. FIG. 24C depicts a graph showing stone volume as obtained by CT scan. FIG. 24D depicts a graph showing bladder weight normalized to body weight.

FIG. 25 depicts photographs showing bladder size of SLC3A1 KO mice on low-methionine diet (0.12%) as compared to non-restricted diet (0.62%) at 8 weeks.

FIGS. 26A-26C depict graphs showing stone number (FIG. 26A), average stone area (FIG. 26B) and stone weight (FIG. 26C) in SLC3A1 KO mice on low-methionine diet (0.12%) and non-restricted diet (0.62%) at 8 weeks.

FIGS. 27A-27C depict graphs showing metabolite measurements comparing urinary cystine (FIG. 27A), urinary cysteine (FIG. 27B) and urinary methionine (FIG. 27C) in SLC3A1 KO mice on low-methionine Diet (0.12%), non-restricted diet (0.62%), and wild-type mice non-restricted diet (0.62%).

FIG. 28 depicts a graph comparing plasma cysteine in SLC3A1 KO mice on low-methionine diet (0.12%), non-restricted diet (0.62%) and wild-type mice on non-restricted diet (0.62%).

FIG. 29A depicts Western blot showing CBS protein levels in CBS knockdown mice versus mice treated with vehicle control. FIG. 29B depicts a bar graph showing significant elevations in plasma homocysteine levels in CBS knockdown mice versus mice treated with vehicle control. Mice received 1×10¹² genomic copies of an adeno-associated virus containing a shRNA targeting CBS or vehicle intravenously and blood was collected on days 21 and 28 and liver on day 35. Data presented as mean plasma Hey±SEM (n=9). Statistical analysis was performed using a mixed-effects analysis followed by Sidak's multiple comparison test. *p<0.05, **p<0.01.

FIGS. 30A and 30B show the effect of SYNB1353 ((lacI-Ptac, IPTG) 3× metDC (Q70D N82H; SEQ ID NO: 1048; engineered library); 1× metP (metagenomics library; F. segetis; SEQ ID NO: 1056); ΔΦ; Δdap; ΔyjeH; Δpks) (2 doses for a total of 5.4×10¹⁰ live cells) on plasma labeled methionine (FIG. 30A) and plasma labeled homocysteine (FIG. 30B) after an oral bolus of D4-methionine (50 mg/kg). Mice received 2 doses of SYNB1353 (one hour apart) and blood was collected at 0, 0.5, or 2 hours post methionine (n=8/group). Statistical analysis was performed using two-way ANOVA followed by Sidak's multiple comparison test or unpaired t-test. *p<0.05.

FIGS. 31A and 31B depict whey protein challenge. CBS-knockdown mice were orally dosed with 2750 mg/kg whey protein (˜50 mg/kg Met) and either SYNB1353 or vehicle, followed by a second dose of strain or vehicle 2 hours later. Urine was collected at 4 hours (FIG. 31A) and 28 hours (FIG. 31B) to measure 3MTP.

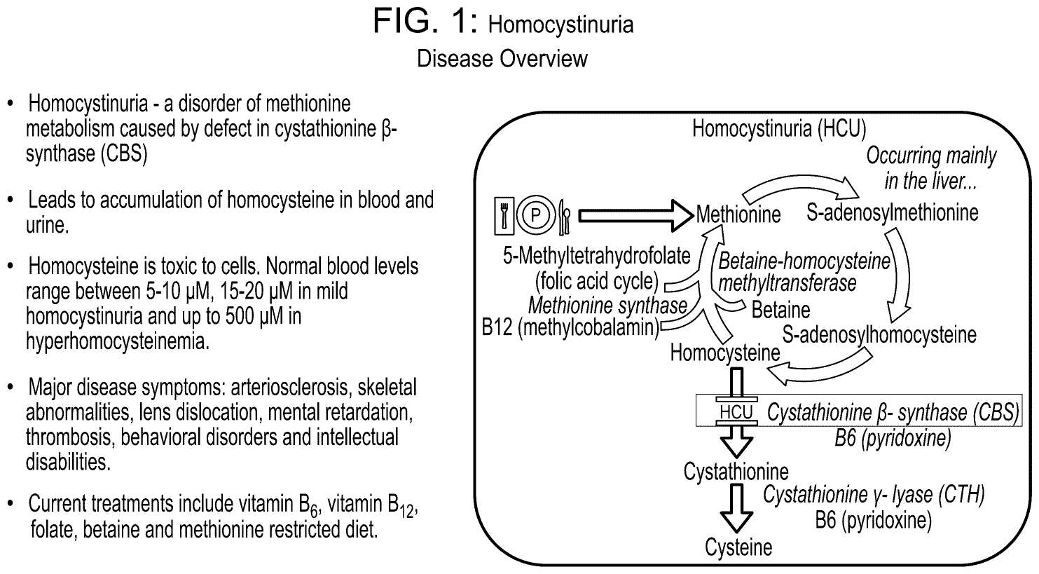

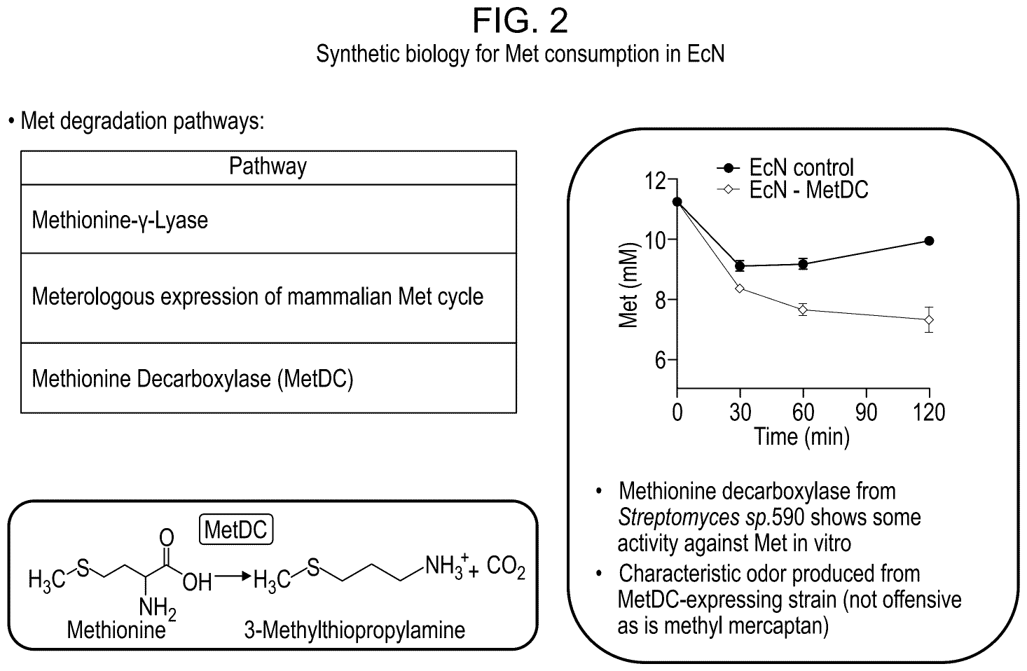

### DETAILED DESCRIPTION

The present disclosure provides recombinant bacterial cells that have been engineered with optimized genetic circuitry which allow the recombinant bacterial cells to turn on and off an engineered metabolic pathway by sensing a patient's internal environment or by chemical induction during, for example, manufacturing. When turned on, the recombinant bacterial cells complete all of the steps in a metabolic pathway to achieve a therapeutic effect in a host subject and are designed to drive therapeutic effects throughout the body of a host from a point of origin of the microbiome.

Specifically, the present disclosure provides recombinant bacterial cells, pharmaceutical compositions thereof, and methods of modulating and treating diseases associated with amino acid metabolism, such as homocystinuria. Specifically, the recombinant bacteria disclosed herein have been constructed to comprise genetic circuits composed of, for example, a methionine decarboxylase to treat disease, as well as other circuitry in order to guarantee the safety and non-colonization of the subject that is administered the recombinant bacteria, such as auxotrophies, etc. These recombinant bacteria are safe and well tolerated and augment the innate activities of the subject's microbiome to achieve a therapeutic effect.

In some embodiments, a bacterial cell disclosed herein has been genetically engineered to comprise a heterologous gene sequence encoding one or more methionine decarboxylases and is capable of processing (e.g., metabolizing) and reducing levels of methionine. In some embodiments, a bacterial cell disclosed herein has been genetically engineered to comprise a heterologous gene sequence encoding one or more methionine decarboxylases and is capable of processing and reducing levels of methionine in low-oxygen environments, e.g., the gut. Thus, the genetically engineered bacterial cells and pharmaceutical compositions comprising the bacterial cells disclosed herein may be used to convert excess methionine into non-toxic molecules in order to treat and/or prevent diseases associated with amino acid metabolism, such as homocystinuria, cystinuria, primary and secondary hypermethioninemia, cancer, and metabolic syndromes/diseases.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “recombinant bacterial cell” or “recombinant bacteria” (also referred to herein as a “genetically engineered bacterial cell”) refers to a bacterial cell or bacteria that have been genetically modified from their native state. Similarly, “recombinant microorganism” (or genetically engineered microorganism), or “recombinant host cell” (or genetically engineered host cell), refers to a microorganism or host cell that has been genetically modified from their native state. For instance, a recombinant bacterial cell, microorganism, or host cell may have nucleotide insertions, nucleotide deletions, nucleotide rearrangements, and nucleotide modifications introduced into their DNA. These genetic modifications may be present in the chromosome of the bacteria, bacterial cell, microorganism, or host cell or on a plasmid in the bacteria, bacterial cell, microorganism, or host cell. Recombinant bacterial cells, microorganisms, or host cells of the disclosure may comprise exogenous or heterologous nucleotide sequences on plasmids. Alternatively, recombinant bacterial cells, microorganisms, or host cells may comprise exogenous or heterologous nucleotide sequences stably incorporated into their chromosome(s).

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature. As used herein the term “gene” is also meant to include a codon-optimized gene sequence, which is modified from a native gene sequence, e.g., to reflect the typical codon usage of the host organism, without altering the polypeptide encoded by a gene or nucleic acid molecule. As used herein, the term “gene” may also refer to a gene sequence which encodes a polypeptide that is not naturally occurring. For example, a gene may encode a polypeptide which is derived from a library of engineered, non-naturally occurring polypeptides. As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

As used herein, a “heterologous gene” or “heterologous sequence” refers to a nucleotide sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. For example, a heterologous gene may include a native coding sequence that is a portion of a chimeric gene that is reintroduced into the host cell. A heterologous gene may also include a native gene, or fragment thereof, introduced into a non-native host cell. Alternatively, a heterologous gene may also include a native gene, or fragment thereof, which has been edited within a host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature.

As used herein, the term “endogenous gene” refers to a native gene in its natural location in the genome of an organism.

As used herein, the term “transgene” refers to a gene that has been introduced into the host organism, e.g., host bacterial cell, genome.

As used herein, the term “bacteriostatic” or “cytostatic” refers to a molecule or protein which is capable of arresting, retarding, or inhibiting the growth, division, multiplication or replication of a recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, and stem-loop structures. In one embodiment, the regulatory sequence comprises a promoter, e.g., an FNR responsive promoter.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene encoding at least one methionine decarboxylase, that is joined to a regulatory sequence in a manner which allows expression of the nucleic acid sequence, e.g., the gene(s) encoding the methionine decarboxylase. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive.

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Examples of inducible promoters include, but are not limited to, an FNR promoter, a P_{araC} promoter, a P_{araBAD} promoter, a propionate promoter, and a P_{TetR} promoter, each of which are described in more detail herein. Examples of other inducible promoters are provided herein below.

As used herein, a “stably maintained” or “stable” host cell, such as a bacterium, is used to refer to a host cell, such as a bacterial host cell, carrying non-native genetic material, e.g., a methionine decarboxylase, that is incorporated into the host genome or propagated on a self-replicating extra-chromosomal plasmid, such that the non-native genetic material is retained, expressed, and propagated. The stable host cell, such as a stable bacterium, is capable of survival and/or growth in vitro, e.g., in medium, and/or in vivo, e.g., in the gut. For example, the stable host cell, such as a stable bacterium, may be a genetically engineered host cell, such as a bacterium, comprising an amino acid catabolism gene, in which the plasmid or chromosome carrying the amino acid catabolism gene is stably maintained in the host cell, such as a bacterium, such that the methionine decarboxylase can be expressed in the host cell, such as a bacterium, and the host cell, such as a bacterium, is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide

As used herein, the term “plasmid” or “vector” refers to an extrachromosomal nucleic acid, e.g., DNA, construct that is not integrated into the genome of a host cell, such as a bacterial host cell. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for host cells, such as bacterial host cells, containing the plasmid and which ensures that the plasmid is retained in the host cell, such as a bacterial host cell. A plasmid disclosed herein may comprise a nucleic acid sequence encoding a heterologous gene, e.g., a gene encoding at least one methionine decarboxylase.

As used herein, the term “transform” or “transformation” refers to the transfer of a nucleic acid fragment into a host cell, such as a host bacterial cell, resulting in genetically-stable inheritance. Host cells, such as host bacterial cells, comprising the transformed nucleic acid fragment are referred to as “recombinant” or “transgenic” or “transformed” cells or organisms. In some instances where one or more nucleic acid fragments are introduced into a host cell, such as on a plasmid or vector, one or more of the nucleic acid fragments may be retained in the cell, such as by integration into the genome of the cell, while the plasmid or vector itself may be removed from the cell. In such instances, the host cell is considered to be transformed with the nucleic acid fragments that were introduced into the cell regardless of whether the plasmid or vector is retained in the cell or not.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising at least one methionine decarboxylase operably linked to a promoter, into a host cell, such as a bacterial host cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of an asparagine. Mutagenesis and directed evolution methods are well known in the art for creating variants. See, e.g., U.S. Pat. Nos. 7,783,428; 6,586,182; 6,117,679; and Ling, et al., 1999, “Approaches to DNA mutagenesis: an overview,” Anal. Biochem., 254(2):157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated” as applied to a gene refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

“Exogenous environmental condition(s)” or “environmental conditions” refer to settings or circumstances under which the promoter described herein is directly or indirectly induced. The phrase is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions refer to the presence of molecules or metabolites that are specific to the mammalian gut in a healthy or disease-state, e.g., propionate. In some embodiments, the exogenous environmental condition is a tissue-specific or disease-specific metabolite or molecule(s). In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprises an oxygen level-dependent promoter. In some aspects, bacteria have evolved transcription factors that are capable of sensing oxygen levels. Different signaling pathways may be triggered by different oxygen levels and occur with different kinetics.

As used herein, “exogenous environmental conditions” or “environmental conditions” also refers to settings or circumstances or environmental conditions external to the engineered microorganism, which relate to in vitro culture conditions of the microorganism. “Exogenous environmental conditions” may also refer to the conditions during growth, production, and manufacture of the organism. Such conditions include aerobic culture conditions, anaerobic culture conditions, low oxygen culture conditions and other conditions under set oxygen concentrations. Such conditions also include the presence of a chemical and/or nutritional inducer, such as tetracycline, arabinose, IPTG, rhamnose, and the like in the culture medium. Such conditions also include the temperatures at which the microorganisms are grown prior to in vivo administration. For example, using certain promoter systems, certain temperatures are permissive to expression of a payload, while other temperatures are non-permissive. Oxygen levels, temperature and media composition influence such exogenous environmental conditions. Such conditions affect proliferation rate, rate of induction of the payload or gene of interest, e.g., amino acid catabolism gene, other regulators (e.g., FNRS24Y), and overall viability and metabolic activity of the strain during strain production.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut).

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR, ANR, and DNR. Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003). Non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic and/or low oxygen conditions by the global transcriptional regulator FNR that is naturally found in Nissle. Under anaerobic and/or low oxygen conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrS, FNR, P-FNRS promoter and other such related designations to indicate the promoter PfnrS.

TABLE 1
Examples of transcription factors and
responsive genes and regulatory regions

| Transcription | Examples of responsive genes, |
|---|---|
| factor | promoters, and/or regulatory regions: |
| FNR | nirB, ydfZ, pdhR, focA, ndH, hlyE, |
| | narK, narX, narG, yfiD, tdcD |
| DNR | norb, norC |

As used herein, a “non-native” nucleic acid sequence refers to a nucleic acid sequence not normally present in a host cell, such as a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. The non-native nucleic acid sequence may be present on a plasmid or chromosome. In addition, multiple copies of any regulatory region, promoter, gene, and/or gene cassette may be present in the host cell, such as a bacterium, wherein one or more copies of the regulatory region, promoter, gene, and/or gene cassette may be mutated or otherwise altered as described herein. In some embodiments, the genetically engineered host cell, such as genetically engineered bacteria, are engineered to comprise multiple copies of the same regulatory region, promoter, gene, and/or gene cassette in order to enhance copy number or to comprise multiple different components of a gene cassette performing multiple different functions. In some embodiments, the genetically engineered host cell, such as genetically engineered bacteria, of the invention comprise a gene encoding a phenylalanine-metabolizing enzyme that is operably linked to a directly or indirectly inducible promoter that is not associated with said gene in nature, e.g., an FNR promoter operably linked to a gene encoding an amino acid metabolism gene.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli σ^{s} promoter (e.g., an osmY promoter (International Genetically Engineered Machine (iGEM) Registry of Standard Biological Parts Name BBa_J45992; BBa_J45993)), a constitutive Escherichia coli σ² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis GB promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), a bacteriophage SP6 promoter (e.g., SP6 promoter (BBa_J64998)), and functional fragments thereof.

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

In some embodiments, the genetically engineered bacteria are active in the gut. In some embodiments, the genetically engineered bacteria are active in the large intestine. In some embodiments, the genetically engineered bacteria are active in the small intestine. In some embodiments, the genetically engineered bacteria are active in the small intestine and in the large intestine. In some embodiments, the genetically engineered bacteria transit through the small intestine. In some embodiments, the genetically engineered bacteria have increased residence time in the small intestine. In some embodiments, the genetically engineered bacteria colonize the small intestine. In some embodiments, the genetically engineered bacteria do not colonize the small intestine. In some embodiments, the genetically engineered bacteria have increased residence time in the gut. In some embodiments, the genetically engineered bacteria colonize the small intestine. In some embodiments, the genetically engineered bacteria do not colonize the gut.

As used herein, the term “low oxygen” is meant to refer to a level, amount, or concentration of oxygen (O₂) that is lower than the level, amount, or concentration of oxygen that is present in the atmosphere (e.g., <21% O₂; <160 torr O₂)). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg O₂, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table 2 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (p mole) (1 μmole O₂=0.022391 mg/L O₂). Fondriest Environmental, Inc., “Dissolved Oxygen”, Fundamentals of Environmental Measurements, 19 Nov. 2013. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE 2

| Compartment | Oxygen Tension |
|---|---|
| stomach | ~60 torr (e.g., 58 +/− 15 torr) |
| duodenum and first | ~30 torr (e.g., 32 +/− 8 torr); |
| part of jejunum | ~20% oxygen in ambient air |
| Ileum (mid-small | ~10 torr; ~6% oxygen in ambient |
| intestine) | air (e.g., 11 +/− 3 torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

“Microorganism” refers to an organism or microbe of microscopic, submicroscopic, or ultramicroscopic size that typically consists of a single cell. Examples of microorganisms include bacteria, yeast, viruses, parasites, fungi, certain algae, and protozoa. In some aspects, the microorganism is engineered (“engineered microorganism”) to produce one or more therapeutic molecules or proteins of interest. In certain aspects, the microorganism is engineered to take up and catabolize certain metabolites or other compounds from its environment, e.g., the gut. In certain aspects, the microorganism is engineered to synthesize certain beneficial metabolites or other compounds (synthetic or naturally occurring) and release them into its environment. In certain embodiments, the engineered microorganism is an engineered bacterium. In certain embodiments, the engineered microorganism is an engineered virus. In certain embodiments, the engineered microorganism is an engineered yeast. When referring to bacteria, engineered bacteria or recombinant bacteria, the embodiments also contemplate other types of microorganisms.

“Host cell” refers to a cell that can be used to express a polynucleotide, such as a polynucleotide that encodes a methionine catabolism enzyme, such as a methionine decarboxylase, and/or a methionine importer.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria are commensal bacteria, which are present in the indigenous microbiota of the gut. Examples of non-pathogenic bacteria include, but are not limited to, Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, the term “auxotroph” or “auxotrophic” refers to an organism that requires a specific factor, e.g., an amino acid, a sugar, or other nutrient, to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Disorders associated with or involved with amino acid metabolism, e.g., homocystinuria or cystinuria, may be caused by inborn genetic mutations for which there are no known cures. Diseases can also be secondary to other conditions, e.g., an intestinal disorder or a bacterial infection. Treating diseases associated with amino acid metabolism may encompass reducing normal levels of one or more amino acids, reducing excess levels of one or more amino acids, or eliminating one or more amino acids, and does not necessarily encompass the elimination of the underlying disease.

As used herein the terms “disease associated with amino acid metabolism” or a “disorder associated with amino acid metabolism” is a disease or disorder involving the abnormal, e.g., increased, levels of one or more amino acids, e.g., methionine, in a subject. In one embodiment, a disease or disorder associated with amino acid metabolism, e.g., methionine metabolism, is homocystinuria. In another embodiment, a disease or disorder associated with amino acid metabolism, e.g., methionine metabolism, is cancer. In another embodiment, a disease or disorder associated with amino acid metabolism, e.g., methionine metabolism, is a metabolic disease or a metabolic syndrome.

As used herein, the term “amino acid” refers to a class of organic compounds that contain at least one amino group and one carboxyl group. Amino acids include leucine, isoleucine, valine, arginine, lysine, asparagine, serine, glycine, glutamine, tryptophan, methionine, threonine, cysteine, tyrosine, phenylalanine, glutamic acid, aspartic acid, alanine, histidine, and proline.

As used herein, the term “amino acid catabolism” or “amino acid metabolism” refers to the processing, breakdown and/or degradation of an amino acid molecule (e.g., methionine, asparagine, lysine or arginine) into other compounds that are not associated with the disease associated with amino acid metabolism, such as homocystinuria, or other compounds which can be utilized by the bacterial cell.

In another embodiment, the term “methionine catabolism” refers to the processing, breakdown, and/or degradation of methionine into 3-methylthiopropylamine. In yet another embodiment, the term “methionine catabolism” refers to the processing, breakdown, and/or degradation of methionine to sulfate. In one embodiment, the term “methionine catabolism” refers to the processing, breakdown, and/or degradation of methionine into methanethiol and 2-aminobut-2-enoate. In another embodiment, the term “methionine catabolism” refers to the processing, breakdown, and/or degradation of methionine into 3-methylthio-2-oxobutyric acid.

As used herein, the term “importer” is meant to refer to a mechanism, e.g., protein, proteins, or protein complex, for importing a molecule, e.g., amino acid, peptide (di-peptide, tri-peptide, polypeptide, etc), toxin, metabolite, substrate, as well as other biomolecules into the microorganism from the extracellular milieu. For example, a methionine importer such as MetP imports methionine into the microorganism.

As used herein, “payload” refers to one or more molecules of interest to be produced by a genetically engineered microorganism, such as bacteria or a virus. In some embodiments, the payload is a therapeutic payload, e.g., an amino acid catabolic enzyme or an amino acid importer polypeptide. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments the payload comprises a repressor element, such as a kill switch. In some embodiments, the payload is encoded by a gene or multiple genes or an operon. In alternate embodiments, the payload is produced by a biosynthetic or biochemical pathway, wherein the biosynthetic or biochemical pathway may optionally be endogenous to the microorganism. In some embodiments, the genetically engineered microorganism comprises two or more payloads.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition. A therapeutically effective amount may, for example, be sufficient to treat, prevent, reduce the severity, delay the onset, and/or reduce the risk of occurrence of one or more symptoms of a disease or condition associated with excess amino acid levels. A therapeutically effective amount, as well as a therapeutically effective frequency of administration, can be determined by methods known in the art and discussed below.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “dipeptide” refers to a peptide of two linked amino acids. The term “tripeptide” refers to a peptide of three linked amino acids. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. A polypeptide may be a naturally occurring polypeptide or alternatively may be a polypeptide not naturally occurring, such as a polypeptide identified from a library of engineered polypeptides. In other embodiments, the polypeptide is produced by the genetically engineered bacteria or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: Ala, Pro, Gly, Gln, Asn, Ser, Thr, Cys, Ser, Tyr, Thr, Val, Ile, Leu, Met, Ala, Phe, Lys, Arg, His, Phe, Tyr, Trp, His, Asp, and Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein, the term “percent identity” refers to a quantitative measurement of the similarity between two sequences (e.g., nucleic acid or amino acid sequence). In some embodiments, the “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al., J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. For example, a first nucleic acid sequence may have at least 70%, at least 75%, at least 80%, at least 85%, at least 90%, at least 91%, at least 92%, at least 93%, at least 94%, at least 95%, at least about 96%, at least 97%, at least 98%, at least 99%, or at least 99.5% identity to the sequence of a second nucleic acid. In another example, a first polypeptide may comprise an amino acid sequence that has at least 70%, at least 75%, at least 80%, at least 85%, at least 90%, at least 91%, at least 92%, at least 93%, at least 94%, at least 95%, at least about 96%, at least 97%, at least 98%, at least 99%, or at least 99.5% identity to the amino acid sequence of a second polypeptide.

As used herein, the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein, the term “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. The term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. In some embodiments, the improvement of transcription and/or translation involves increasing the level of transcription and/or translation. In some embodiments, the improvement of transcription and/or translation involves decreasing the level of transcription and/or translation. In some embodiments, codon optimization is used to fine-tune the levels of expression from a construct of interest. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent, inter alia, on the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

The terms “phage” and “bacteriophage” are used interchangeably herein. Both terms refer to a virus that infects and replicates within a bacterium. As used herein “phage” or bacteriophage” collectively refers to prophage, lysogenic, dormant, temperate, intact, defective, cryptic, and satellite phage, phage tail bacteriocins, tailiocins, and gene transfer agents. As used therein the term “prophage” refers to the genomic material of a bacteriophage, which is integrated into a replicon of the host cell and replicates along with the host. The prophage may be able to produce phages if specifically activated. In some cases, the prophage is not able to produce phages or has never done so (i.e., defective or cryptic prophages). In some cases, prophage also refers to satellite phages. The terms “prophage” and “endogenous phage” are used interchangeably herein. “Endogenous phage” or “endogenous prophage” also refers to a phage that is present in the natural state of a bacterium (and its parental strain). As used herein the term “phage knockout” or “inactivated phage” refers to a phage which has been modified so that it can either no longer produce and/or package phage particles or it produces fewer phage particles than the wild type phage sequence. In some embodiments, the inactivated phage or phage knockout refers to the inactivation of a temperate phage in its lysogenic state, i.e., to a prophage. Such a modification refers to a mutation in the phage; such mutations include insertions, deletions (partial or complete deletion of phage genome), substitutions, inversions, at one or more positions within the phage genome, e.g., within one or more genes within the phage genome. As used herein the adjectives “phage-free”, “phage free” and “phageless” are used interchangeably to characterize a bacterium or strain which contains one or more prophages, one or more of which have been modified. The modification can result in a loss of the ability of the prophage to be induced or release phage particles. Alternatively, the modification can result in less efficient or less frequent induction or less efficient or less frequent phage release as compared to the isogenic strain without the modification. Ability to induce and release phage can be measured using a plaque assay as described herein. As used herein phage induction refers to the part of the life cycle of a lysogenic prophage, in which the lytic phage genes are activated, phage particles are produced and lysis occurs.

As used herein a “pharmaceutical composition” refers to a preparation of bacterial cells disclosed herein with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The articles “a” and “an,” as used herein, should be understood to mean “at least one,” unless clearly indicated to the contrary. For example, as used herein, “a heterologous gene encoding a methionine decarboxylase” should be understood to mean “at least one heterologous gene encoding at least one methionine decarboxylase.” Similarly, as used herein, “a heterologous gene encoding an amino acid importer” should be understood to mean “at least one heterologous gene encoding at least one amino acid importer.”

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Host Cells

Any suitable host cell may be used to express any of the enzymes disclosed herein, such as methionine catabolism enzymes (e.g., methionine decarboxylases) and methionine importers. Suitable host cells include, but are not limited to, bacterial cells (e.g., E. coli cells), fungal cells (e.g., yeast cells), algal cells, plant cells, insect cells, and animal cells, including mammalian cells.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as Chlamydomonas (e.g., C. Reinhardtii) and Phormidium (P. sp. ATCC29409).

In some embodiments, the host cell is an animal cell. In some embodiments, the host cell is a mammalian cell, including, for example, a human cell (e.g., 293, HeLa, W138, PER.C6 or Bowes melanoma cells), a mouse cell (e.g., 3T3, NS0, NS1 or Sp2/0), a hamster cell (e.g., CHO or BHK), or a monkey cell (e.g., COS, FRhL or Vero). In some embodiments, the cell is a hybridoma cell line.

In some embodiments, the host cell is a bacterial cell, e.g., a recombinant bacterial cell. The disclosure provides a bacterial cell that comprises a heterologous gene encoding a methionine catabolism enzyme. In some embodiments, the bacterial cell is a non-pathogenic bacterial cell. In some embodiments, the bacterial cell is a commensal bacterial cell. In some embodiments, the bacterial cell is a probiotic bacterial cell.

In certain embodiments, the bacterial cell is selected from the group consisting of a Bacteroides fragilis, Bacteroides thetaiotaomicron, Bacteroides subtilis, Bifidobacterium animalis, Bifidobacterium bifidum, Bifidobacterium infantis, Bifidobacterium lactis, Clostridium butyricum, Clostridium scindens, Escherichia coli, Lactobacillus acidophilus, Lactobacillus plantarum, Lactobacillus reuteri, Lactococcus lactis, and Oxalobacter formigenes bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium animalis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is a Clostridium scindens bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell. In one embodiment, the bacterial cell is a Oxalobacter formigenes bacterial cell. In another embodiment, the bacterial cell does not include Oxalobacter formigenes.

In one embodiment, the bacterial cell is a Gram positive bacterial cell. In another embodiment, the bacterial cell is a Gram negative bacterial cell.

In some embodiments, the bacterial cell is Escherichia coli strain Nissle 1917 (E. coli Nissle), a Gram-positive bacterium of the Enterobacteriaceae family that “has evolved into one of the best characterized probiotics” (Ukena et al., 2007). The strain is characterized by its “complete harmlessness” (Schultz, 2008), and “has GRAS (generally recognized as safe) status” (Reister et al., 2014, emphasis added). Genomic sequencing confirmed that E. coli Nissle “lacks prominent virulence factors (e.g., E. coli α-hemolysin, P-fimbrial adhesins)” (Schultz, 2008), and E. coli Nissle “does not carry pathogenic adhesion factors and does not produce any enterotoxins or cytotoxins, it is not invasive, not uropathogenic” (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's “therapeutic efficacy and safety have convincingly been proven” (Ukena et al., 2007).

In one embodiment, the recombinant bacterial cell does not colonize the subject.

One of ordinary skill in the art would appreciate that the genetic modifications disclosed herein may be adapted for other species, strains, and subtypes of bacteria. Furthermore, genes from one or more different species can be introduced into one another, e.g., an amino acid catabolism gene from Klebsiella quasipneumoniae can be expressed in Escherichia coli.

In some embodiments, the bacterial cell is a genetically engineered bacterial cell. In another embodiment, the bacterial cell is a recombinant bacterial cell. In some embodiments, the disclosure comprises a colony of bacterial cells.

In another aspect, the disclosure provides a recombinant bacterial culture which comprises bacterial cells disclosed herein. In one aspect, the disclosure provides a recombinant bacterial culture which reduces levels of an amino acid, e.g., methionine, in the media of the culture. In one embodiment, the levels of an amino acid are reduced by about 50%, about 75%, or about 100% in the media of the cell culture. In another embodiment, the levels of an amino acid are reduced by about two-fold, three-fold, four-fold, five-fold, six-fold, seven-fold, eight-fold, nine-fold, or ten-fold in the media of the cell culture. In one embodiment, the levels of an amino acid, e.g., methionine, are reduced below the limit of detection in the media of the cell culture.

In some embodiments of the above described genetically engineered bacteria, the gene encoding a methionine decarboxylase is present on a plasmid in the bacterium and operatively linked on the plasmid to the promoter that is induced under low-oxygen or anaerobic conditions. In other embodiments, the gene encoding a methionine decarboxylase is present in the bacterial chromosome and is operatively linked in the chromosome to the promoter that is induced under low-oxygen or anaerobic conditions.

In some embodiments of the above described genetically engineered bacteria, the gene encoding a methionine decarboxylase is present on a plasmid in the bacterium and operatively linked on the plasmid to the promoter that is chemically induced. In other embodiments, the gene encoding a methionine decarboxylase is present in the bacterial chromosome and is operatively linked in the chromosome to the promoter that is chemically induced.

In some embodiments of the above described genetically engineered bacteria, the gene encoding a methionine importer is present on a plasmid in the bacterium and operatively linked on the plasmid to the promoter that is chemically induced. In other embodiments, the gene encoding a methionine importer is present in the bacterial chromosome and is operatively linked in the chromosome to the promoter that is chemically induced.

In some embodiments of the above described genetically engineered bacteria, the gene encoding a methionine decarboxylase is present on a plasmid in the bacterium and operatively linked on the plasmid to the promoter that is chemically induced and the gene encoding a methionine importer is present on a plasmid in the bacterium and operatively linked on the plasmid to the promoter that is chemically induced. In other embodiments, the gene encoding a methionine decarboxylase is present in the bacterial chromosome and is operatively linked in the chromosome to the promoter that is chemically induced and the gene encoding a methionine importer is present in the bacterial chromosome and is operatively linked in the chromosome to the promoter that is chemically induced.

In some embodiments of the above described genetically engineered bacteria, the gene encoding a methionine decarboxylase is present on a plasmid in the bacterium and operatively linked on the plasmid to the promoter that is chemically induced and the gene encoding a methionine importer is present in the bacterial chromosome and is operatively linked in the chromosome to the promoter that is chemically induced. In other embodiments, the gene encoding a methionine decarboxylase is present in the bacterial chromosome and is operatively linked in the chromosome to the promoter that is chemically induced and the gene encoding a methionine importer is present on a plasmid in the bacterium and operatively linked on the plasmid to the promoter that is chemically induced.

In some embodiments, the genetically engineered bacteria is an auxotroph. In one embodiment, the genetically engineered bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thiI auxotroph. In some embodiments, the engineered bacteria have more than one auxotrophy, for example, they may be a ΔthyA and ΔdapA auxotroph.

In some embodiments of the above described genetically engineered bacteria, the gene encoding a methionine decarboxylase is present on a plasmid in the bacterium and operatively linked on the plasmid to the promoter that is induced under low-oxygen or anaerobic conditions. In other embodiments, the gene encoding a methionine decarboxylase is present in the bacterial chromosome and is operatively linked in the chromosome to the promoter that is induced under low-oxygen or anaerobic conditions.

A. Methionine Catabolism Enzymes

Methionine catabolism enzymes may be expressed or modified in host cells, such as the bacteria disclosed herein, in order to enhance catabolism of methionine. For example, the genetically engineered bacteria comprising at least one heterologous gene encoding a methionine catabolism enzyme can catabolize methionine to treat a disease associated with methionine, including, but not limited to homocystinuria, cystinuria, primary and secondary hypermethioninemia, cystathionine β-synthase (CBS) deficiency, or cancer, e.g., lymphoblastic leukemia.

As used herein, the term “methionine catabolism enzyme” refers to an enzyme involved in the catabolism of methionine. Specifically, when a methionine catabolism enzyme is expressed in a recombinant bacterial cell, the bacterial cell hydrolyzes more methionine into 3-methylthiopropylamine (3-MTP) when the catabolism enzyme is expressed than unmodified bacteria of the same bacterial subtype under the same conditions. In some embodiments, methionine importers may also be expressed or modified in the recombinant bacteria to enhance methionine import into the cell in order to increase the catabolism of methionine by the methionine catabolism enzyme. In other embodiments, methionine exporters may be knocked-out in the recombinant bacteria to decrease export of methionine and/or increase cytoplasmic concentration of methionine.

In one embodiment, the methionine catabolism enzyme is a methionine decarboxylase (MetDC). In another embodiment, the methionine catabolism enzyme is a leucine decarboxylase (LeuDC) which has been modified to further comprise methionine catabolism activity, e.g., methionine decarboxylase activity. For example, SEQ ID NO: 1053 is a leucine decarboxylase which has been modified as compared to a wild-type leucine decarboxylase sequence at positions N2S, V14A, E16G, H17S, R19W, A30E, K41Q, 145D, R48H, A51P, R65Q, L68C, L90R, N147S, L154V, R156Q, R160G, L170Q, H179S, H185S, E218Q, Y220F, C235R, H240D, K254E, V263M, T269A, V304A, S308V, D310E, A318M, V328S, T372E, S394T, I406P, and D411C as compared to a wild type polypeptide. In one embodiment, the leucine decarboxylase gene encodes a polypeptide that has point mutations N2S, V14A, E16G, H17S, R19W, A30E, K41Q, I45D, R48H, A51P, R65Q, L68C, L90R, N147S, L154V, R156Q, R160G, L170Q, H179S, H185S, E218Q, Y220F, C235R, H240D, K254E, V263M, T269A, V304A, S308V, D310E, A318M, V328S, T372E, S394T, 1406P, and D411C and comprises methionine decarboxylase activity. Accordingly, in some embodiments herein, a leucine decarboxylase enzyme that has been modified to have methionine decarboxylase activity may be referred to as a “methionine decarboxylase (MetDC)” which is encoded by a “methionine decarboxylase (metDC) gene.”

In one embodiment, the methionine catabolism enzyme increases the rate of methionine catabolism in the cell. In one embodiment, the methionine catabolism enzyme decreases the level of methionine in the cell. In another embodiment, the methionine catabolism enzyme increases the level of 3-methylthiopropylamine in the cell. In one embodiment, 3-methylthiopropylamine is not toxic to the cell.

Methionine catabolism enzymes are well known to those of skill in the art (see, e.g., Huang et al., Mar. Drugs, 13(8):5492-5507, 2015). For example, the adenosylmethionine synthase pathway has been identified in Anabaena cylindrica. In the adenosylmethionine synthase pathway, methionine is catabolized into S-adenosyl-L-homocysteine by an S-adenosylmethionine synthase enzyme, followed by conversion of the S-adenosyl-L-homocysteine into L-homocysteine by an adenosylhomocysteinase enzyme. As another example, two methionine aminotransferase enzymes (including Aro8 and Aro9), and one decarboxylase gene (Aro10) have been identified in Saccharomyces cerevisiae which catabolize methionine (Yin et al. (2015) FEMS Microbiol. Lett. 362(5) pii: fnu043). Methionine aminotransferase enzymes catabolize methionine and 2-oxo carboxylate into 2-oxo-4-methylthiobutanoate and an L-amino acid.

In some embodiments, a methionine catabolism enzyme is encoded by a gene encoding a methionine catabolism enzyme derived from a bacterial species. In some embodiments, a methionine catabolism enzyme is encoded by a gene encoding a methionine catabolism enzyme derived from a non-bacterial species. In some embodiments, a methionine catabolism enzyme is encoded by a gene derived from a eukaryotic species, e.g., a yeast species or a plant species. In one embodiment, the gene encoding the methionine catabolism enzyme is derived from an organism of the genus or species that includes, but is not limited to, Klebsiella quasipneumoniae, Bacillus subtilis, Caenorhabditis elegans, Entamoeba histolytica, Bacillus halodurans, Methylobacterium aquaticum, Saccharomyces cerevisiae, Escherichia coli, and Anabaena cylindrica.

In one embodiment, the methionine catabolism enzyme is a methionine decarboxylase (MDC). In one embodiment, the methionine decarboxylase gene is a MDC gene from Streptomyces sp. 590. On example of such a MDC gene is described, for example, in Misono et al., Bull. Inst. Chem. Res., Kyoto Univ., 58(3):323-333, 1980. In one embodiment, the methionine decarboxylase gene is a metDC from Stanieria sp. NIES-3757. In one embodiment, the methionine decarboxylase gene is a metDC from Mus musculus. In one embodiment, the methionine decarboxylase gene is a metDC from Entamoeba histolytica.

In one embodiment, the methionine decarboxylase gene encodes a polypeptide with a Q70D mutation referenced by the polypeptide encoded by the gene sequence having the sequence of SEQ ID NO: 1003. In one embodiment, the methionine decarboxylase gene encodes a polypeptide with a N82H mutation referenced by the polypeptide encoded by the gene sequence having the sequence of SEQ ID NO: 1003. In one embodiment, the methionine decarboxylase gene encodes a polypeptide with Q70D N82H mutations referenced by the polypeptide encoded by the gene sequence having the sequence of SEQ ID NO: 1003.

In one embodiment, the methionine decarboxylase gene encodes a polypeptide with a Q70D mutation referenced by the polypeptide encoded by the gene sequence having the sequence of SEQ ID NO: 1018. In one embodiment, the methionine decarboxylase gene encodes a polypeptide with a N82H mutation referenced by the polypeptide encoded by the gene sequence having the sequence of SEQ ID NO: 1018. In one embodiment, the methionine decarboxylase gene encodes a polypeptide with Q70D N82H mutations referenced by the polypeptide encoded by the gene sequence having the sequence of SEQ ID NO: 1018.

In one embodiment, the methionine decarboxylase gene encodes a polypeptide with a V491L mutation referenced by the polypeptide encoded by the gene sequence having the sequence of SEQ ID NO: 1003. In one embodiment, the methionine decarboxylase gene encodes a polypeptide with a A500P mutation referenced by the polypeptide encoded by the gene sequence having the sequence of SEQ ID NO: 1003. In one embodiment, the methionine decarboxylase gene encodes a polypeptide with V491L A500P mutations referenced by the polypeptide encoded by the gene sequence having the sequence of SEQ ID NO: 1003.

In one embodiment, the methionine decarboxylase gene encodes a polypeptide with a V491L mutation referenced by the polypeptide encoded by the gene sequence having the sequence of SEQ ID NO: 1018. In one embodiment, the methionine decarboxylase gene encodes a polypeptide with a A500P mutation referenced by the polypeptide encoded by the gene sequence having the sequence of SEQ ID NO: 1018. In one embodiment, the methionine decarboxylase gene encodes a polypeptide with V491L A500P mutations referenced by the polypeptide encoded by the gene sequence having the sequence of SEQ ID NO: 1018.

In one embodiment, the methionine decarboxylase gene encodes a polypeptide with a R41Q mutation referenced by the polypeptide encoded by the gene sequence having the sequence of SEQ ID NO: 1003. In one embodiment, the methionine decarboxylase gene encodes a polypeptide with a Q70D mutation referenced by the polypeptide encoded by the gene sequence having the sequence of SEQ ID NO: 1003. In one embodiment, the methionine decarboxylase gene encodes a polypeptide with R41Q Q70D mutations referenced by the polypeptide encoded by the gene sequence having the sequence of SEQ ID NO: 1003.

In one embodiment, the methionine decarboxylase gene encodes a polypeptide with a R41Q mutation referenced by the polypeptide encoded by the gene sequence having the sequence of SEQ ID NO: 1018. In one embodiment, the methionine decarboxylase gene encodes a polypeptide with a Q70D mutation referenced by the polypeptide encoded by the gene sequence having the sequence of SEQ ID NO: 1018. In one embodiment, the methionine decarboxylase gene encodes a polypeptide with R41Q Q70D mutations referenced by the polypeptide encoded by the gene sequence having the sequence of SEQ ID NO: 1018.

In one embodiment, the methionine decarboxylase gene encodes a polypeptide with T66N mutation referenced by the polypeptide encoded by the gene sequence having the sequence of SEQ ID NO: 1003. In one embodiment, the methionine decarboxylase gene encodes a polypeptide with A203H mutation referenced by the polypeptide encoded by the gene sequence having the sequence of SEQ ID NO: 1003. In one embodiment, the methionine decarboxylase gene encodes a polypeptide with H379G mutation referenced by the polypeptide encoded by the gene sequence having the sequence of SEQ ID NO: 1003.

In one embodiment, the methionine decarboxylase gene encodes a polypeptide with T66N mutation referenced by the polypeptide encoded by the gene sequence having the sequence of SEQ ID NO: 1018. In one embodiment, the methionine decarboxylase gene encodes a polypeptide with A203H mutation referenced by the polypeptide encoded by the gene sequence having the sequence of SEQ ID NO: 1018. In one embodiment, the methionine decarboxylase gene encodes a polypeptide with H379G mutation referenced by the polypeptide encoded by the gene sequence having the sequence of SEQ ID NO: 1018.

In one embodiment, the methionine decarboxylase gene has at least about 80% identity with the sequence of SEQ ID NO: 1003. Accordingly, in one embodiment, the methionine decarboxylase gene has at least about 90% identity with the sequence of SEQ ID NO: 1003. Accordingly, in one embodiment, the methionine decarboxylase gene has at least about 95% identity with the sequence of SEQ ID NO: 1003. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1003. In another embodiment, the methionine decarboxylase gene comprises the sequence of SEQ ID NO: 1003. In yet another embodiment the methionine decarboxylase gene consists of the sequence of SEQ ID NO: 1003.

In one embodiment, the methionine decarboxylase gene has at least about 80% identity with the sequence of SEQ ID NO: 1018. Accordingly, in one embodiment, the methionine decarboxylase gene has at least about 90% identity with the sequence of SEQ ID NO: 1018. Accordingly, in one embodiment, the methionine decarboxylase gene has at least about 95% identity with the sequence of SEQ ID NO: 1018. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1018. In another embodiment, the methionine decarboxylase gene comprises the sequence of SEQ ID NO:1018. In yet another embodiment the methionine decarboxylase gene consists of the sequence of SEQ ID NO: 1018.

In one embodiment, the methionine decarboxylase gene has at least about 80% identity with the sequence of SEQ ID NO: 1034. Accordingly, in one embodiment, the methionine decarboxylase gene has at least about 90% identity with the sequence of SEQ ID NO: 1034. Accordingly, in one embodiment, the methionine decarboxylase gene has at least about 95% identity with the sequence of SEQ ID NO: 1034. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1034. In another embodiment, the methionine decarboxylase gene comprises the sequence of SEQ ID NO: 1034. In yet another embodiment the methionine decarboxylase gene consists of the sequence of SEQ ID NO: 1034.

In one embodiment, the methionine decarboxylase gene has at least about 80% identity with the sequence of SEQ ID NO: 1035. Accordingly, in one embodiment, the methionine decarboxylase gene has at least about 90% identity with the sequence of SEQ ID NO: 1035. Accordingly, in one embodiment, the methionine decarboxylase gene has at least about 95% identity with the sequence of SEQ ID NO: 1035. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1035. In another embodiment, the methionine decarboxylase gene comprises the sequence of SEQ ID NO: 1035. In yet another embodiment the methionine decarboxylase gene consists of the sequence of SEQ ID NO: 1035.

In one embodiment, the methionine decarboxylase gene has at least about 80% identity with the sequence of SEQ ID NO: 1036. Accordingly, in one embodiment, the methionine decarboxylase gene has at least about 90% identity with the sequence of SEQ ID NO: 1036. Accordingly, in one embodiment, the methionine decarboxylase gene has at least about 95% identity with the sequence of SEQ ID NO: 1036. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1036. In another embodiment, the methionine decarboxylase gene comprises the sequence of SEQ ID NO: 1036. In yet another embodiment the methionine decarboxylase gene consists of the sequence of SEQ ID NO: 1036.

In one embodiment, the methionine decarboxylase gene has at least about 80% identity with the sequence of SEQ ID NO: 1037. Accordingly, in one embodiment, the methionine decarboxylase gene has at least about 90% identity with the sequence of SEQ ID NO: 1037. Accordingly, in one embodiment, the methionine decarboxylase gene has at least about 95% identity with the sequence of SEQ ID NO: 1037. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1037. In another embodiment, the methionine decarboxylase gene comprises the sequence of SEQ ID NO: 1037. In yet another embodiment the methionine decarboxylase gene consists of the sequence of SEQ ID NO: 1037.

In one embodiment, the methionine decarboxylase gene has at least about 80% identity with the sequence of SEQ ID NO: 1039. Accordingly, in one embodiment, the methionine decarboxylase gene has at least about 90% identity with the sequence of SEQ ID NO: 1039. Accordingly, in one embodiment, the methionine decarboxylase gene has at least about 95% identity with the sequence of SEQ ID NO: 1039. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1039. In another embodiment, the methionine decarboxylase gene comprises the sequence of SEQ ID NO: 1039. In yet another embodiment the methionine decarboxylase gene consists of the sequence of SEQ ID NO: 1039.

In one embodiment, the methionine decarboxylase gene has at least about 80% identity with the sequence of SEQ ID NO: 1040. Accordingly, in one embodiment, the methionine decarboxylase gene has at least about 90% identity with the sequence of SEQ ID NO: 1040. Accordingly, in one embodiment, the methionine decarboxylase gene has at least about 95% identity with the sequence of SEQ ID NO: 1040. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1040. In another embodiment, the methionine decarboxylase gene comprises the sequence of SEQ ID NO: 1040. In yet another embodiment the methionine decarboxylase gene consists of the sequence of SEQ ID NO: 1040.

In one embodiment, the methionine decarboxylase gene has at least about 80% identity with the sequence of SEQ ID NO: 1123. Accordingly, in one embodiment, the methionine decarboxylase gene has at least about 90% identity with the sequence of SEQ ID NO: 1123. Accordingly, in one embodiment, the methionine decarboxylase gene has at least about 95% identity with the sequence of SEQ ID NO: 1123. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1123. In another embodiment, the methionine decarboxylase gene comprises the sequence of SEQ ID NO: 1123. In yet another embodiment the methionine decarboxylase gene consists of the sequence of SEQ ID NO: 1123.

In one embodiment, the methionine decarboxylase gene has at least about 80% identity with the sequence of SEQ ID NO: 1125. Accordingly, in one embodiment, the methionine decarboxylase gene has at least about 90% identity with the sequence of SEQ ID NO: 1125. Accordingly, in one embodiment, the methionine decarboxylase gene has at least about 95% identity with the sequence of SEQ ID NO: 1125. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1125. In another embodiment, the methionine decarboxylase gene comprises the sequence of SEQ ID NO: 1125. In yet another embodiment the methionine decarboxylase gene consists of the sequence of SEQ ID NO: 1125.

In one embodiment, the methionine decarboxylase gene has at least about 80% identity with the sequence of SEQ ID NO: 1127. Accordingly, in one embodiment, the methionine decarboxylase gene has at least about 90% identity with the sequence of SEQ ID NO: 1127. Accordingly, in one embodiment, the methionine decarboxylase gene has at least about 95% identity with the sequence of SEQ ID NO: 1127. Accordingly, in one embodiment, the methionine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1127. In another embodiment, the methionine decarboxylase gene comprises the sequence of SEQ ID NO: 1127. In yet another embodiment the methionine decarboxylase gene consists of the sequence of SEQ ID NO: 1127.

In one embodiment, the recombinant bacteria comprise a gene sequence encoding a methionine catabolism enzyme, wherein the methionine catabolism enzyme is a leucine decarboxylase. In some cases, the leucine decarboxylase may have been modified from a wild-type leucine catabolism enzyme to also catabolize methionine, as described herein. Indeed, in some embodiments disclosed herein, a leucine catabolism enzyme that can metabolize methionine is referred to as a “methionine catabolism enzyme” or a “methionine decarboxylase.”

In some embodiment, the leucine decarboxylase gene has at least about 80% with the sequence of SEQ ID NO: 1038. Accordingly, in one embodiment, the leucine decarboxylase gene has at least about 90% identity with the sequence of SEQ ID NO: 1038. Accordingly, in one embodiment, the leucine decarboxylase gene has at least about 95% identity with the sequence of SEQ ID NO: 1038. Accordingly, in one embodiment, the leucine decarboxylase gene has at least about 85%, 86%, 87%, 88%, 89%, 90%, 91%, 92%, 93%, 94%, 95%, 96%, 97%, 98%, or 99% identity with the sequence of SEQ ID NO: 1038. In another embodiment, the leucine decarboxylase gene comprises the sequence of SEQ ID NO: 1038. In yet another embodiment the leucine decarboxylase gene consists of the sequence of SEQ ID NO: 1038.

In one embodiment, the leucine decarboxylase gene encodes a polypeptide has at least about 80% identity with SEQ ID NO: 1053. Accordingly, in one embodiment, the leucine decarboxylase gene encodes a polypeptide has at least about 90% identity with SEQ ID NO: 1053. Accordingly, in one embodiment, the leucine decarboxylase gene encodes a polypeptide has at least about 95% identity with SEQ ID NO: 1053. Accordingly, in one embodiment, the leucine decarboxylase gene encodes a polypeptide has at least about 85%, 86%, 87%, 88%, 89%, 90%, 91%, 92%, 93%, 94%, 95%, 96%, 97%, 98%, or 99% identity with SEQ ID NO: 1053. In another embodiment, the leucine decarboxylase gene encodes a polypeptide comprises SEQ ID NO: 1053. In yet another embodiment the leucine decarboxylase gene encodes a polypeptide consists of SEQ ID NO: 1053.

In one embodiment, the leucine decarboxylase gene encodes a polypeptide that has 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 point mutations as compared to a wild type leucine decarboxylase polypeptide. In one embodiment, the leucine decarboxylase gene encodes a polypeptide that has one or more point mutations selected from N2S, V14A, E16G, H17S, R19W, A30E, K41Q, 145D, R48H, A51P, R65Q, L68C, L90R, N147S, L154V, R156Q, R160G, L170Q, H179S, H185S, E218Q, Y220F, C235R, H240D, K254E, V263M, T269A, V304A, S308V, D310E, A318M, V328S, T372E, S394T, 1406P, and D411C as compared to a wild type polypeptide. In one embodiment, the leucine decarboxylase gene encodes a polypeptide that has point mutations N2S, V14A, E16G, H17S, R19W, A30E, K41Q, 145D, R48H, A51P, R65Q, L68C, L90R, N147S, L154V, R156Q, R160G, L170Q, H179S, H185S, E218Q, Y220F, C235R, H240D, K254E, V263M, T269A, V304A, S308V, D310E, A318M, V328S, T372E, S394T, 1406P, and D411C as compared to a wild type polypeptide.

In some embodiments, the sequence of a methionine decarboxylase associated with the disclosure comprises one or more amino acid substitutions relative to SEQ ID NO: 1049. In some embodiments, the one or more amino acid substitutions are at a position corresponding to position 41, 66, 70, 82, 203, 379, 491 and/or 500 in SEQ ID NO: 1049.

In some embodiments, a methionine decarboxylase comprises: a glutamine (Q) at a position corresponding to position 41 in the sequence of SEQ ID NO: 1049; an asparagine (N) at a position corresponding to position 66 in SEQ ID NO: 1049; an aspartic acid (D) at a position corresponding to position 70 in the sequence of SEQ ID NO: 1049; a histidine (H) at a position corresponding to position 82 in SEQ ID NO: 1049; a histidine (H) at a position corresponding to position 203 in SEQ ID NO: 1049; a glycine (G) at a position corresponding to position 379 in SEQ ID NO: 1049; a leucine (L) at a position corresponding to position 491 in the sequence of SEQ ID NO: 1049; and/or a proline (P) at a position corresponding to position 500 in the sequence of SEQ ID NO: 1049.

In some embodiments, the sequence of a methionine decarboxylase associated with the disclosure comprises substitutions at a position corresponding to: position 66; position 203; position 379; positions 70 and 82; positions 491 and 500; or positions 41 and 70 in the sequence of SEQ ID NO: 1049. In some embodiments, the sequence of a methionine decarboxylase comprises the following amino acid substitutions relative to the sequence of SEQ ID NO: 1049: T66N; A203H; H379G; Q70D and N82H; V491L and A500P; or R41Q and Q70D.

In one embodiment, the methionine decarboxylase polypeptide has at least about 80% identity with the sequence of any one of SEQ ID NOs: 1048, 1049, 1050, 1051, 1052, 1054, 1055, 1124, 1126, or 1128. Accordingly, in one embodiment, the methionine decarboxylase polypeptide has at least about 90% identity with the sequence of any one of SEQ ID NOs: 1048, 1049, 1050, 1051, 1052, 1054, 1055, 1124, 1126, or 1128. Accordingly, in one embodiment, the methionine decarboxylase polypeptide has at least about 95% identity with the sequence of any one of SEQ ID NOs: 1048, 1049, 1050, 1051, 1052, 1054, 1055, 1124, 1126, or 1128. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with the sequence of any one of SEQ ID NOs: 1048, 1049, 1050, 1051, 1052, 1054, 1055, 1124, 1126, or 1128. In another embodiment, the methionine decarboxylase polypeptide comprises the sequence of any one of SEQ ID NOs: 1048, 1049, 1050, 1051, 1052, 1054, 1055, 1124, 1126, or 1128. In yet another embodiment the methionine decarboxylase polypeptide consists of the sequence of any one of SEQ ID NOs: 1048, 1049, 1050, 1051, 1052, 1054, 1055, 1124, 1126, or 1128.

In one embodiment, the methionine decarboxylase polypeptide has at least about 80% identity with SEQ ID NO: 1048. Accordingly, in one embodiment, the methionine decarboxylase polypeptide has at least about 90% identity with SEQ ID NO: 1048. Accordingly, in one embodiment, the methionine decarboxylase polypeptide has at least about 95% identity with SEQ ID NO: 1048. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with SEQ ID NO: 1048. In another embodiment, the methionine decarboxylase polypeptide comprises SEQ ID NO: 1048. In yet another embodiment the methionine decarboxylase polypeptide consists of SEQ ID NO: 1048.

In one embodiment, the methionine decarboxylase polypeptide has at least about 80% identity with SEQ ID NO: 1049. Accordingly, in one embodiment, the methionine decarboxylase polypeptide has at least about 90% identity with SEQ ID NO: 1049. Accordingly, in one embodiment, the methionine decarboxylase polypeptide has at least about 95% identity with SEQ ID NO: 1049. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with SEQ ID NO: 1049. In another embodiment, the methionine decarboxylase polypeptide comprises SEQ ID NO: 1049. In yet another embodiment the methionine decarboxylase polypeptide consists of SEQ ID NO: 1049.

In one embodiment, the methionine decarboxylase polypeptide has at least about 80% identity with SEQ ID NO: 1050. Accordingly, in one embodiment, the methionine decarboxylase polypeptide has at least about 90% identity with SEQ ID NO: 1050. Accordingly, in one embodiment, the methionine decarboxylase polypeptide has at least about 95% identity with SEQ ID NO: 1050. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with SEQ ID NO: 1050. In another embodiment, the methionine decarboxylase polypeptide comprises SEQ ID NO: 1050. In yet another embodiment the methionine decarboxylase polypeptide consists of SEQ ID NO: 1050.

In one embodiment, the methionine decarboxylase polypeptide has at least about 80% identity with SEQ ID NO: 1051. Accordingly, in one embodiment, the methionine decarboxylase polypeptide has at least about 90% identity with SEQ ID NO: 1051. Accordingly, in one embodiment, the methionine decarboxylase polypeptide has at least about 95% identity with SEQ ID NO: 1051. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with SEQ ID NO: 1051. In another embodiment, the methionine decarboxylase polypeptide comprises SEQ ID NO: 1051. In yet another embodiment the methionine decarboxylase polypeptide consists of SEQ ID NO: 1051.

In one embodiment, the methionine decarboxylase polypeptide has at least about 80% identity with SEQ ID NO: 1052. Accordingly, in one embodiment, the methionine decarboxylase polypeptide has at least about 90% identity with SEQ ID NO: 1052. Accordingly, in one embodiment, the methionine decarboxylase polypeptide has at least about 95% identity SEQ ID NO: 1052. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with SEQ ID NO: 1052. In another embodiment, the methionine decarboxylase polypeptide comprises SEQ ID NO: 1052. In yet another embodiment the methionine decarboxylase polypeptide consists of SEQ ID NO: 1052.

In one embodiment, the methionine decarboxylase polypeptide has at least about 80% identity with SEQ ID NO: 1054. Accordingly, in one embodiment, the methionine decarboxylase polypeptide has at least about 90% identity with SEQ ID NO: 1054. Accordingly, in one embodiment, the methionine decarboxylase polypeptide has at least about 95% identity with SEQ ID NO: 1054. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with SEQ ID NO: 1054. In another embodiment, the methionine decarboxylase polypeptide comprises SEQ ID NO: 1054. In yet another embodiment the methionine decarboxylase polypeptide consists of SEQ ID NO: 1054.

In one embodiment, the methionine decarboxylase polypeptide has at least 1 or 2 point mutations as compared to a wild type polypeptide. In one embodiment, the methionine decarboxylase polypeptide has point mutations H179S and/or V304A as compared to a wild type polypeptide. For example, SEQ ID NO: 1054 (and SEQ ID NO: 1039 encoding SEQ ID NO: 1054) is a methionine decarboxylase with two amino acid substitutions relative to a wild-type sequence.

In one embodiment, the methionine decarboxylase polypeptide has at least about 80% identity with SEQ ID NO: 1055. Accordingly, in one embodiment, the methionine decarboxylase polypeptide has at least about 90% identity with SEQ ID NO: 1055. Accordingly, in one embodiment, the methionine decarboxylase polypeptide has at least about 95% identity with SEQ ID NO: 1055. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with SEQ ID NO: 1055. In another embodiment, the methionine decarboxylase polypeptide comprises SEQ ID NO: 1055. In yet another embodiment the methionine decarboxylase polypeptide consists of SEQ ID NO: 1055.

In one embodiment, the methionine decarboxylase polypeptide has at least about 80% identity with SEQ ID NO: 1124. Accordingly, in one embodiment, the methionine decarboxylase polypeptide has at least about 90% identity with SEQ ID NO: 1124. Accordingly, in one embodiment, the methionine decarboxylase polypeptide has at least about 95% identity with SEQ ID NO: 1124. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with SEQ ID NO: 1124. In another embodiment, the methionine decarboxylase polypeptide comprises SEQ ID NO: 1124. In yet another embodiment the methionine decarboxylase polypeptide consists of SEQ ID NO: 1124.

In one embodiment, the methionine decarboxylase polypeptide has at least about 80% identity with SEQ ID NO: 1126. Accordingly, in one embodiment, the methionine decarboxylase polypeptide has at least about 90% identity with SEQ ID NO: 1126. Accordingly, in one embodiment, the methionine decarboxylase polypeptide has at least about 95% identity with SEQ ID NO: 1126. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with SEQ ID NO: 1126. In another embodiment, the methionine decarboxylase polypeptide comprises SEQ ID NO: 1126. In yet another embodiment the methionine decarboxylase polypeptide consists of SEQ ID NO: 1126.

In one embodiment, the methionine decarboxylase polypeptide has at least about 80% identity with SEQ ID NO: 1128. Accordingly, in one embodiment, the methionine decarboxylase polypeptide has at least about 90% identity with SEQ ID NO: 1128. Accordingly, in one embodiment, the methionine decarboxylase polypeptide has at least about 95% identity with SEQ ID NO: 1128. Accordingly, in one embodiment, the methionine decarboxylase polypeptide has at least about 85%, 86%, 87%, 88%, 89%, 90%, 91%, 92%, 93%, 94%, 95%, 96%, 97%, 98%, or 99% identity with SEQ ID NO: 1128. In another embodiment, the methionine decarboxylase polypeptide comprises SEQ ID NO: 1128. In yet another embodiment the methionine decarboxylase polypeptide consists of SEQ ID NO: 1128.

The present disclosure further comprises genes encoding functional fragments of a methionine decarboxylase enzyme.

Assays for testing the activity of a methionine catabolism enzyme, a methionine catabolism enzyme functional variant, or a methionine catabolism enzyme functional fragment are well known to one of ordinary skill in the art. For example, methionine catabolism can be assessed by expressing the protein, functional variant, or fragment thereof, in a recombinant bacterial cell that lacks endogenous methionine catabolism enzyme activity. Other methods are also well known to one of ordinary skill in the art (see, e.g., Dolzan et al., FEBS Letters, 574:141-146, 2004, the entire contents of which are incorporated by reference). Additional methods, i.e., for measuring methionine decarboxylase activity in vitro or in vivo are described in the examples herein.

In some embodiments, the genetically engineered host cell, such as genetically engineered bacteria, comprise a stably maintained plasmid or chromosome carrying a gene for producing a methionine decarboxylase, such that the methionine decarboxylase can be expressed in the host cell, and the host cell is capable of survival and/or growth in vitro, e.g., in medium, and/or in vivo. In some embodiments, a host cell, such as a bacterium, may comprise multiple copies of the gene encoding the methionine decarboxylase. In some embodiments, the gene encoding the methionine decarboxylase is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the methionine decarboxylase is expressed on a high-copy plasmid. In some embodiments, the high-copy plasmid may be useful for increasing expression of the methionine decarboxylase. In some embodiments, the gene encoding the methionine decarboxylase is expressed on a chromosome.

In some embodiments, the host cells, such as bacteria host cells are genetically engineered to include multiple mechanisms of action (MOAs), e.g., circuits producing multiple copies of the same product (e.g., to enhance copy number) or circuits performing multiple different functions. For example, the genetically engineered host cells, such as genetically engineered bacteria, may include four copies of the gene encoding a particular methionine decarboxylase inserted at four different insertion sites. Alternatively, the genetically engineered host cells, such as genetically engineered bacteria, may include three copies of the gene encoding a particular methionine decarboxylase inserted at three different insertion sites and three copies of the gene encoding a different methionine decarboxylase inserted at three different insertion sites.

In some embodiments, under conditions where the methionine decarboxylase is expressed, the genetically engineered host cells, such as genetically engineered bacteria, of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the methionine decarboxylase, and/or transcript of the gene(s) in the operon as compared to unmodified host cells, such as unmodified bacteria, of the same subtype under the same conditions.

In some embodiments, quantitative PCR (qPCR) is used to amplify, detect, and/or quantify mRNA expression levels of the methionine decarboxylase gene(s). Primers specific for methionine decarboxylase the gene(s) may be designed and used to detect mRNA in a sample according to methods known in the art. In some embodiments, a fluorophore is added to a sample reaction mixture that may contain methionine decarboxylase mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the methionine decarboxylase gene(s).

In one embodiment, the host cell, such as a bacterial host cell, comprises a heterologous gene encoding a methionine catabolism enzyme. In one embodiment, the host cell, such as a bacterial host cell, comprises a heterologous gene encoding an importer of methionine. In one embodiment, the host cell, such as a bacterial host cell, comprises a heterologous gene encoding an importer of methionine and a heterologous gene encoding a methionine catabolism enzyme. In one embodiment, the host cell, such as a bacterial host cell, comprises a heterologous gene encoding a methionine catabolism enzyme and a genetic modification that reduces export of methionine. In one embodiment, the host cell, such as a bacterial host cell, comprises a heterologous gene encoding an importer of methionine, a heterologous gene encoding a methionine catabolism enzyme, and a genetic modification that reduces export of methionine. Importers and exporters are described in more detail in the subsections, below.

B. Importers of Methionine

Methionine importers may be expressed or modified in the recombinant host cells, such as recombinant bacteria, described herein in order to enhance methionine import into the cell. Specifically, when the importer of methionine is expressed in the recombinant host cells, such as recombinant bacterial cells, described herein, the bacterial cells import more methionine into the cell when the importer is expressed than unmodified bacteria of the same bacterial subtype under the same conditions. Thus, the genetically engineered bacteria comprising a heterologous gene encoding importer of methionine which may be used to import methionine into the bacteria so that any gene encoding a methionine catabolism enzyme expressed in the organism can catabolize the methionine to treat a disease associated with methionine, such as homocystinuria.

The uptake of methionine into bacterial cells is mediated by proteins well known to those of skill in the art. For example, a methionine importer operon has been identified in Corynebacterium glutamicum (Trotschel et al., J. Bacteriology, 187(11):3786-3794, 2005). In addition, the high affinity MetD ABC importer system has been characterized in Escherichia coli (Kadaba et al. (2008) Science 5886: 250-253; Kadner and Watson (1974) J. Bacteriol. 119: 401-9). The MetD importer system is capable of mediating the translocation of several substrates across the bacterial membrane, including methionine. The MetD system of Escherichia coli consists of MetN (encoded by metN), which comprises the ATPase domain, MetI (encoded by metI), which comprises the transmembrane domain, and MetQ (encoded by metQ), the cognate binding protein which is located in the periplasm. Orthologues of the genes encoding the E. coli MetD importer system have been identified in multiple organisms including, e.g., Yersinia pestis, Vibrio cholerae, Pasteurella multocida, Haemophilus influenza, Agrobacterium tumefaciens, Sinorhizobium meliloti, Brucella meliloti, and Mesorhizobium loti (Merlin et al. (2002) J. Bacteriol. 184: 5513-7).

In one embodiment, the at least one gene encoding an importer of methionine is a metN gene, a metI gene, and/or a metQ gene from Corynebacterium glutamicum, Escherichia coli, and Bacillus subtilis (Trotschel et al., J. Bacteriology, 187(11):3786-3794, 2005).

In one embodiment, the metN gene has at least about 80% identity with the sequence of SEQ ID NO: 1004. Accordingly, in one embodiment, the metN gene has at least about 90% identity with the sequence of SEQ ID NO: 1004. Accordingly, in one embodiment, the metN gene has at least about 95% identity with the sequence of SEQ ID NO: 1004. Accordingly, in one embodiment, the metN gene has at least about 85%, 86%, 87%, 88%, 89%, 90%, 91%, 92%, 93%, 94%, 95%, 96%, 97%, 98%, or 99% identity with the sequence of SEQ ID NO: 1004. In another embodiment, the metN gene comprises the sequence of SEQ ID NO: 1004. In yet another embodiment the metN gene consists of the sequence of SEQ ID NO: 1004.

In one embodiment, the metI gene has at least about 80% identity with the sequence of SEQ ID NO: 1005. Accordingly, in one embodiment, the metI gene has at least about 90% identity with the sequence of SEQ ID NO: 1005. Accordingly, in one embodiment, the metI gene has at least about 95% identity with the sequence of SEQ ID NO: 1005. Accordingly, in one embodiment, the metI gene has at least about 85%, 86%, 87%, 88%, 89%, 90%, 91%, 92%, 93%, 94%, 95%, 96%, 97%, 98%, or 99% identity with the sequence of SEQ ID NO: 1005. In another embodiment, the metI gene comprises the sequence of SEQ ID NO: 1005. In yet another embodiment the metI gene consists of the sequence of SEQ ID NO: 1005.

In one embodiment, the metQ gene has at least about 80% identity with the sequence of SEQ ID NO: 1006. Accordingly, in one embodiment, the metQ gene has at least about 90% identity with the sequence of SEQ ID NO: 1006. Accordingly, in one embodiment, the metQ gene has at least about 95% identity with the sequence of SEQ ID NO: 1006. Accordingly, in one embodiment, the metQ gene has at least about 85%, 86%, 87%, 88%, 89%, 90%, 91%, 92%, 93%, 94%, 95%, 96%, 97%, 98%, or 99% identity with the sequence of SEQ ID NO: 1006. In another embodiment, the metQ gene comprises the sequence of SEQ ID NO: 1006. In yet another embodiment the metQ gene consists of the sequence of SEQ ID NO: 1006.

In one embodiment, the metNIQ gene has at least about 80% identity with the sequence of SEQ ID NO: 1043, 1045, 1046, or 1047. Accordingly, in one embodiment, the metNIQ gene has at least about 90% identity with the sequence of SEQ ID NO: 1043, 1045, 1046, or 1047. Accordingly, in one embodiment, the metNIQ gene has at least about 95% identity with the sequence of SEQ ID NO: 1043, 1045, 1046, or 1047. Accordingly, in one embodiment, the metNIQ gene has at least about 85%, 86%, 87%, 88%, 89%, 90%, 91%, 92%, 93%, 94%, 95%, 96%, 97%, 98%, or 99% identity with the sequence of SEQ ID NO: 1043, 1045, 1046, or 1047. In another embodiment, the metNIQ gene comprises the sequence of SEQ ID NO: 1043, 1045, 1046, or 1047. In yet another embodiment the metNIQ gene consists of the sequence of SEQ ID NO: 1043, 1045, 1046, or 1047.

In one embodiment, the metNIQ gene encodes a polypeptide with a P281G mutation in the MetN polypeptide referenced by the MetN polypeptide encoded the gene sequence having the sequence of SEQ ID NO: 1004. In one embodiment, the metNIQ gene encodes a polypeptide with a P281S mutation in the MetN polypeptide referenced by the MetN polypeptide encoded the gene sequence having the sequence of SEQ ID NO: 1004.

In one embodiment, at least one gene encoding an importer of methionine is a metP gene. In one embodiment, the metP gene is from Flavobacterium segetis. In one embodiment, the metP gene is from Flavobacterium frigoris. In one embodiment, the metP gene is from Bacillus subtilis. In one embodiment, the metP gene is from Sporomusa termitida. In one embodiment, the metP gene is from Bacteroidetes bacterium 43-16.

In one embodiment, the metP gene has at least about 80% identity with the sequence of SEQ ID NO: 1041. Accordingly, in one embodiment, the metP gene has at least about 90% identity with the sequence of SEQ ID NO: 1041. Accordingly, in one embodiment, the metP gene has at least about 95% identity with the sequence of SEQ ID NO: 1041. Accordingly, in one embodiment, the metP gene has at least about 85%, 86%, 87%, 88%, 89%, 90%, 91%, 92%, 93%, 94%, 95%, 96%, 97%, 98%, or 99% identity with the sequence of SEQ ID NO: 1041. In another embodiment, the metP gene comprises the sequence of SEQ ID NO: 1041. In yet another embodiment the metP gene consists of the sequence of SEQ ID NO: 1041.

In one embodiment, the metP gene has at least about 80% identity with the sequence of SEQ ID NO: 1042. Accordingly, in one embodiment, the metP gene has at least about 90% identity with the sequence of SEQ ID NO: 1042. Accordingly, in one embodiment, the metP gene has at least about 95% identity with the sequence of SEQ ID NO: 1042. Accordingly, in one embodiment, the metP gene has at least about 85%, 86%, 87%, 88%, 89%, 90%, 91%, 92%, 93%, 94%, 95%, 96%, 97%, 98%, or 99% identity with the sequence of SEQ ID NO: 1042. In another embodiment, the metP gene comprises the sequence of SEQ ID NO: 1042. In yet another embodiment the metP gene consists of the sequence of SEQ ID NO: 1042.

In one embodiment, the metP gene has at least about 80% identity with the sequence of SEQ ID NO: 1044. Accordingly, in one embodiment, the metP gene has at least about 90% identity with the sequence of SEQ ID NO: 1044. Accordingly, in one embodiment, the metP gene has at least about 95% identity with the sequence of SEQ ID NO: 1044. Accordingly, in one embodiment, the metP gene has at least about 85%, 86%, 87%, 88%, 89%, 90%, 91%, 92%, 93%, 94%, 95%, 96%, 97%, 98%, or 99% identity with the sequence of SEQ ID NO: 1044. In another embodiment, the metP gene comprises the sequence of SEQ ID NO: 1044. In yet another embodiment the metP gene consists of the sequence of SEQ ID NO: 1044.

In one embodiment, the metP gene has at least about 80% identity with the sequence of SEQ ID NO: 1129. Accordingly, in one embodiment, the metP gene has at least about 90% identity with the sequence of SEQ ID NO: 1129. Accordingly, in one embodiment, the metP gene has at least about 95% identity with the sequence of SEQ ID NO: 1129. Accordingly, in one embodiment, the metP gene has at least about 85%, 86%, 87%, 88%, 89%, 90%, 91%, 92%, 93%, 94%, 95%, 96%, 97%, 98%, or 99% identity with the sequence of SEQ ID NO: 1129. In another embodiment, the metP gene comprises the sequence of SEQ ID NO: 1129. In yet another embodiment the metP gene consists of the sequence of SEQ ID NO: 1129.

In one embodiment, the metP gene has at least about 80% identity with the sequence of SEQ ID NO: 1131. Accordingly, in one embodiment, the metP gene has at least about 90% identity with the sequence of SEQ ID NO: 1131. Accordingly, in one embodiment, the metP gene has at least about 95% identity with the sequence of SEQ ID NO: 1131. Accordingly, in one embodiment, the metP gene has at least about 85%, 86%, 87%, 88%, 89%, 90%, 91%, 92%, 93%, 94%, 95%, 96%, 97%, 98%, or 99% identity with the sequence of SEQ ID NO: 1131. In another embodiment, the metP gene comprises the sequence of SEQ ID NO: 1131. In yet another embodiment the metP gene consists of the sequence of SEQ ID NO: 1131.

In one embodiment, the MetP is from Flavobacterium segetis. In one embodiment, the MetP is from Flavobacterium frigoris. In one embodiment, MetP gene is from Bacillus subtilis. In one embodiment, MetP gene is from Sporomusa termitida. In one embodiment, MetP gene is from Bacteroidetes bacterium 43-16.

In one embodiment, the MetP polypeptide has at least about 80% identity with the sequence of any one of SEQ ID NOs: 1056, 1057, 1061, 1130, or 1132. Accordingly, in one embodiment, the MetP polypeptide has at least about 90% identity with the sequence of any one of SEQ ID NOs: 1056, 1057, 1061, 1130, or 1132. Accordingly, in one embodiment, the MetP polypeptide has at least about 95% identity with the sequence of any one of SEQ ID NOs: 1056, 1057, 1061, 1130, or 1132. Accordingly, in one embodiment, the MetP polypeptide has at least about 85%, 86%, 87%, 88%, 89%, 90%, 91%, 92%, 93%, 94%, 95%, 96%, 97%, 98%, or 99% identity with the sequence of any one of SEQ ID NOs: 1056, 1057, 1061, 1130, or 1132. In another embodiment, the MetP polypeptide comprises the sequence of any one of SEQ ID NOs: 1056, 1057, 1061, 1130, or 1132. In yet another embodiment the MetP polypeptide consists of the sequence of any one of SEQ ID NOs: 1056, 1057, 1061, 1130, or 1132.

In one embodiment, the MetP polypeptide has at least about 80% identity with the sequence of SEQ ID NO: 1056. Accordingly, in one embodiment, the MetP polypeptide has at least about 90% identity with the sequence of SEQ ID NO: 1056. Accordingly, in one embodiment, the MetP polypeptide has at least about 95% identity with the sequence of SEQ ID NO: 1056. Accordingly, in one embodiment, the MetP polypeptide has at least about 85%, 86%, 87%, 88%, 89%, 90%, 91%, 92%, 93%, 94%, 95%, 96%, 97%, 98%, or 99% identity with the sequence of SEQ ID NO: 1056. In another embodiment, the MetP polypeptide comprises the sequence of SEQ ID NO: 1056. In yet another embodiment the MetP polypeptide consists of the sequence of SEQ ID NO: 1056.

In one embodiment, the MetP polypeptide has at least about 80% identity with the sequence of SEQ ID NO: 1057. Accordingly, in one embodiment, the MetP polypeptide has at least about 90% identity with the sequence of SEQ ID NO: 1057. Accordingly, in one embodiment, the MetP polypeptide has at least about 95% identity with the sequence of SEQ ID NO: 1057. Accordingly, in one embodiment, the MetP polypeptide has at least about 85%, 86%, 87%, 88%, 89%, 90%, 91%, 92%, 93%, 94%, 95%, 96%, 97%, 98%, or 99% identity with the sequence of SEQ ID NO: 1057. In another embodiment, the MetP polypeptide comprises the sequence of SEQ ID NO: 1057. In yet another embodiment the MetP polypeptide consists of the sequence of SEQ ID NO: 1057.

In one embodiment, the MetP polypeptide has at least about 80% identity with the sequence of SEQ ID NO: 1061. Accordingly, in one embodiment, the MetP polypeptide has at least about 90% identity with the sequence of SEQ ID NO: 1061. Accordingly, in one embodiment, the MetP polypeptide has at least about 95% identity with the sequence of SEQ ID NO: 1061. Accordingly, in one embodiment, the MetP polypeptide has at least about 85%, 86%, 87%, 88%, 89%, 90%, 91%, 92%, 93%, 94%, 95%, 96%, 97%, 98%, or 99% identity with the sequence of SEQ ID NO: 1061. In another embodiment, the MetP polypeptide comprises the sequence of SEQ ID NO: 1061. In yet another embodiment the MetP polypeptide consists of the sequence of SEQ ID NO: 1061.

In one embodiment, the MetP polypeptide has at least about 80% identity with the sequence of SEQ ID NO: 1130. Accordingly, in one embodiment, the MetP polypeptide has at least about 90% identity with the sequence of SEQ ID NO: 1130. Accordingly, in one embodiment, the MetP polypeptide has at least about 95% identity with the sequence of SEQ ID NO: 1130. Accordingly, in one embodiment, the MetP polypeptide has at least about 85%, 86%, 87%, 88%, 89%, 90%, 91%, 92%, 93%, 94%, 95%, 96%, 97%, 98%, or 99% identity with the sequence of SEQ ID NO: 1130. In another embodiment, the MetP polypeptide comprises the sequence of SEQ ID NO: 1130. In yet another embodiment the MetP polypeptide consists of the sequence of SEQ ID NO: 1130.

In one embodiment, the MetP polypeptide has at least about 80% identity with the sequence of SEQ ID NO: 1132. Accordingly, in one embodiment, the MetP polypeptide has at least about 90% identity with the sequence of SEQ ID NO: 1132. Accordingly, in one embodiment, the MetP polypeptide has at least about 95% identity with the sequence of SEQ ID NO: 1132. Accordingly, in one embodiment, the MetP polypeptide has at least about 85%, 86%, 87%, 88%, 89%, 90%, 91%, 92%, 93%, 94%, 95%, 96%, 97%, 98%, or 99% identity with the sequence of SEQ ID NO: 1132. In another embodiment, the MetP polypeptide comprises the sequence of SEQ ID NO: 1132. In yet another embodiment the MetP polypeptide consists of the sequence of SEQ ID NO: 1132.

In one embodiment, the MetN polypeptide has at least about 80% identity with the sequence of SEQ ID NO: 1058. Accordingly, in one embodiment, the MetN polypeptide has at least about 90% identity with the sequence of SEQ ID NO: 1058. Accordingly, in one embodiment, the MetN polypeptide has at least about 95% identity with the sequence of SEQ ID NO: 1058. Accordingly, in one embodiment, the MetN polypeptide has at least about 85%, 86%, 87%, 88%, 89%, 90%, 91%, 92%, 93%, 94%, 95%, 96%, 97%, 98%, or 99% identity with the sequence of SEQ ID NO: 1058. In another embodiment, the MetN polypeptide comprises the sequence of SEQ ID NO: 1058. In yet another embodiment the MetN polypeptide consists of the sequence of SEQ ID NO: 1058.

In one embodiment, the MetN polypeptide has at least about 80% identity with the sequence of SEQ ID NO: 1062. Accordingly, in one embodiment, the MetN polypeptide has at least about 90% identity with the sequence of SEQ ID NO: 1062. Accordingly, in one embodiment, the MetN polypeptide has at least about 95% identity with the sequence of SEQ ID NO: 1062. Accordingly, in one embodiment, the MetN polypeptide has at least about 85%, 86%, 87%, 88%, 89%, 90%, 91%, 92%, 93%, 94%, 95%, 96%, 97%, 98%, or 99% identity with the sequence of SEQ ID NO: 1062. In another embodiment, the MetN polypeptide comprises the sequence of SEQ ID NO: 1062. In yet another embodiment the MetN polypeptide consists of the sequence of SEQ ID NO: 1062.

In one embodiment, the MetN polypeptide has at least about 80% identity with the sequence of SEQ ID NO: 1063. Accordingly, in one embodiment, the MetN polypeptide has at least about 90% identity with the sequence of SEQ ID NO: 1063. Accordingly, in one embodiment, the MetN polypeptide has at least about 95% identity with the sequence of SEQ ID NO: 1063. Accordingly, in one embodiment, the MetN polypeptide has at least about 85%, 86%, 87%, 88%, 89%, 90%, 91%, 92%, 93%, 94%, 95%, 96%, 97%, 98%, or 99% identity with the sequence of SEQ ID NO: 1063. In another embodiment, the MetN polypeptide comprises the sequence of SEQ ID NO: 1063. In yet another embodiment the MetN polypeptide consists of the sequence of SEQ ID NO: 1063.

In one embodiment, the MetI polypeptide has at least about 80% identity with the sequence of SEQ ID NO: 1059. Accordingly, in one embodiment, the MetI polypeptide has at least about 90% identity with the sequence of SEQ ID NO: 1059. Accordingly, in one embodiment, the MetI polypeptide has at least about 95% identity with the sequence of SEQ ID NO: 1059. Accordingly, in one embodiment, the MetI polypeptide has at least about 85%, 86%, 87%, 88%, 89%, 90%, 91%, 92%, 93%, 94%, 95%, 96%, 97%, 98%, or 99% identity with the sequence of SEQ ID NO: 1059. In another embodiment, the MetI polypeptide comprises the sequence of SEQ ID NO: 1059. In yet another embodiment the MetI polypeptide consists of the sequence of SEQ ID NO: 1059.

In one embodiment, the MetQ polypeptide has at least about 80% identity with the sequence of SEQ ID NO: 1060. Accordingly, in one embodiment, the MetQ polypeptide has at least about 90% identity with the sequence of SEQ ID NO: 1060. Accordingly, in one embodiment, the MetQ polypeptide has at least about 95% identity with the sequence of SEQ ID NO: 1060. Accordingly, in one embodiment, the MetQ polypeptide has at least about 85%, 86%, 87%, 88%, 89%, 90%, 91%, 92%, 93%, 94%, 95%, 96%, 97%, 98%, or 99% identity with the sequence of SEQ ID NO: 1060. In another embodiment, the MetQ polypeptide comprises the sequence of SEQ ID NO: 1060. In yet another embodiment the MetQ polypeptide consists of the sequence of SEQ ID NO: 1060.

In some embodiments, the importer of methionine is encoded by an importer of methionine gene derived from a bacterial genus or species, including but not limited to, Corynebacterium glutamicum, Escherichia coli, and Bacillus subtilis. In some embodiments, the bacterial species is Escherichia coli strain Nissle.

Assays for testing the activity of an importer of methionine, a functional variant of an importer of methionine, or a functional fragment of importer of methionine are well known to one of ordinary skill in the art. For example, import of methionine may be determined using the methods as described in Trotschel et al., J. Bacteriology, 187(11):3786-3794, 2005, the entire contents of which are expressly incorporated by reference herein.

In one embodiment, when the importer of a methionine is expressed in the recombinant bacterial cells described herein, the bacterial cells import 10% more methionine into the bacterial cell when the importer is expressed than unmodified bacteria of the same bacterial subtype under the same conditions. In another embodiment, when the importer of methionine is expressed in the recombinant bacterial cells described herein, the bacterial cells import 20%, 30%, 40%, 50%, 60%, 70%, 80%, 90% or 100% more methionine into the bacterial cell when the importer is expressed than unmodified bacteria of the same bacterial subtype under the same conditions. In yet another embodiment, when the importer of methionine is expressed in the recombinant bacterial cells described herein, the bacterial cells import two-fold more methionine into the cell when the importer is expressed than unmodified bacteria of the same bacterial subtype under the same conditions. In yet another embodiment, when the importer of methionine is expressed in the recombinant bacterial cells described herein, the bacterial cells import three-fold, four-fold, five-fold, six-fold, seven-fold, eight-fold, nine-fold, or ten-fold more methionine into the cell when the importer is expressed than unmodified bacteria of the same bacterial subtype under the same conditions.

C. Exporters of Methionine

Methionine exporters may be modified in the recombinant bacteria described herein in order to reduce methionine export from the cell. Specifically, when the recombinant bacterial cells described herein comprise a genetic modification that reduces export of methionine, the bacterial cells retain more methionine in the bacterial cell than unmodified bacteria of the same bacterial subtype under the same conditions. Thus, the recombinant bacteria comprising a genetic modification that reduces export of methionine may be used to retain more methionine in the bacterial cell so that any methionine catabolism enzyme expressed in the organism, e.g., co-expressed methionine catabolism enzyme, can catabolize the methionine.

Exporters of methionine are well known to one of ordinary skill in the art. For example, the MetE methionine exporter from Bacillus atrophaeus, and the BrnFE methionine exporter from Corynebacterium glutamicum have been described (Trotschel et al., J. Bacteriology, 187(11):3786-3794, 2005). The YjeH methionine exporter from E. coli has also been described (Liu et al., 2015: Applied and Environmental Microbiology, 81(22):7753-7766).

In one embodiment, the methionine exporter is yjeH. In one embodiment, the yjeH gene has at least about 80% identity with the sequence of SEQ ID NO: 1014. Accordingly, in one embodiment, the yjeH gene has at least about 90% identity with the sequence of SEQ ID NO:1014. Accordingly, in one embodiment, the yjeH gene has at least about 95% identity with the sequence of SEQ ID NO:1014. Accordingly, in one embodiment, the yjeH gene has at least about 85%, 86%, 87%, 88%, 89%, 90%, 91%, 92%, 93%, 94%, 95%, 96%, 97%, 98%, or 99% identity with the sequence of SEQ ID NO:1014. In another embodiment, the yjeH gene comprises the sequence of SEQ ID NO:1014. In yet another embodiment the yjeH gene consists of the sequence of SEQ ID NO:1014. In one embodiment, the yjeH gene is deleted. In another embodiment, a point mutation in the yjeH gene prevents export of methionine from the cell.

In one embodiment, the genetic modification is a mutation in an endogenous gene encoding an exporter of methionine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export methionine from the bacterial cell.

In another embodiment, the genetic modification is a mutation in a promoter of an endogenous gene encoding an exporter of methionine.

In yet another embodiment, the genetic modification is an overexpression of a repressor of an exporter of methionine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

D. Inducible Promoters

In some embodiments, the host cell, such as a bacterial host cell, comprises a stably maintained plasmid or chromosome carrying the gene(s) encoding the methionine decarboxylase(s) and/or the methionine importers, such that the methionine decarboxylase(s) and/or the methionine importers can be expressed in the host cell, and the host cell is capable of survival and/or growth in vitro, e.g., in medium, and/or in vivo, e.g., in the gut. In some embodiments, the host cell, such as a bacterial host cell, comprises two or more distinct methionine decarboxylase and/or the methionine importer genes or operons. In some embodiments, the host cell, such as a bacterial host cell, comprises three or more distinct methionine decarboxylase and/or methionine importer genes or operons. In some embodiments, the host cell, such as a bacterial host cell, comprises 4, 5, 6, 7, 8, 9, 10, or more distinct methionine decarboxylase and/or methionine importer genes or operons.

In some embodiments, the genetically engineered host cell, such as a genetically engineered bacteria, comprise multiple copies of the same methionine decarboxylase gene(s) or methionine importer genes. In some embodiments, the gene encoding the methionine decarboxylase or methionine importer genes is present on a plasmid and operably linked to a directly or indirectly inducible promoter. In some embodiments, the gene encoding the methionine decarboxylase or methionine importer genes is present on a plasmid and operably linked to a promoter that is induced under low-oxygen or anaerobic conditions. In some embodiments, the gene encoding the methionine decarboxylase or methionine importer genes is present on a chromosome and operably linked to a directly or indirectly inducible promoter. In some embodiments, the gene encoding the methionine decarboxylase or methionine importer genes is present in the chromosome and operably linked to a promoter that is induced under low-oxygen or anaerobic conditions. In some embodiments, the gene encoding the methionine decarboxylase or methionine importer genes is present on a plasmid and operably linked to a promoter that is induced by exposure to tetracycline, arabinose or Isopropyl β-D-1-thiogalactopyranoside (IPTG).

In some embodiments, the inducible promoter is a IPTG inducible promoter, e.g., Ptac. In one embodiment, the IPTG inducible promoter comprises a sequence having at least 90%, 91%, 92%, 93%, 94%, 95%, 96%, 97%, 98%, or 99% identity to, comprises, or consists of SEQ ID NO: 1108. In some embodiments, the recombinant bacterium further comprises a gene sequence encoding a gene sequence encoding a transcriptional regulator, e.g., a repressor IPTG inducible promoter. In some embodiments, the gene sequence encoding a repressor comprises a sequence having at least 90%, 91%, 92%, 93%, 94%, 95%, 96%, 97%, 98%, or 99% identity to, comprises, or consists of SEQ ID NO: 1105. In some embodiments, the repressor comprises a sequence having at least 90%, 91%, 92%, 93%, 94%, 95%, 96%, 97%, 98%, or 99% identity to, comprises, or consists of SEQ ID NO: 1106.

TABLE 3
IPTG inducible promoter and LacI sequences

| Description | |
|---|---|
| SEQ ID NO | Sequences |
| LacI in reverse | TCACTGCCCGCTTTCCAGTCGGGAAACCTGTCGTGCCAGCTGCATTA |
| orientation | ATGAATCGGCCAACGCGCGGGGAGAGGCGGTTTGCGTATTGGGCGC |
| SEQ ID NO: | CAGGGTGGTTTTTCTTTTCACCAGTGAGACTGGCAACAGCTGATTGC |
| 1105 | CCTTCACCGCCTGGCCCTGAGAGAGTTGCAGCAAGCGGTCCACGCTG |
| | GTTTGCCCCAGCAGGCGAAAATCCTGTTTGATGGTGGTTAACGGCGG |
| | GATATAACATGAGCTATCTTCGGTATCGTCGTATCCCACTACCGAGA |
| | TATCCGCACCAACGCGCAGCCCGGACTCGGTAATGGCGCGCATTGC |
| | GCCCAGCGCCATCTGATCGTTGGCAACCAGCATCGCAGTGGGAACG |
| | ATGCCCTCATTCAGCATTTGCATGGTTTGTTGAAAACCGGACATGGC |
| | ACTCCAGTCGCCTTCCCGTTCCGCTATCGGCTGAATTTGATTGCGAG |
| | TGAGATATTTATGCCAGCCAGCCAGACGCAGACGCGCCGAGACAGA |
| | ACTTAATGGGCCCGCTAACAGCGCGATTTGCTGGTGACCCAATGCGA |
| | CCAGATGCTCCACGCCCAGTCGCGTACCGTCCTCATGGGAGAAAAT |
| | AATACTGTTGATGGGTGTCTGGTCAGAGACATCAAGAAATAACGCC |
| | GGAACATTAGTGCAGGCAGCTTCCACAGCAATGGCATCCTGGTCATC |
| | CAGCGGATAGTTAATGATCAGCCCACTGACGCGTTGCGCGAGAAGA |
| | TTGTGCACCGCCGCTTTACAGGCTTCGACGCCGCTTCGTTCTACCATC |
| | GACACCACCACGCTGGCACCCAGTTGATCGGCGCGAGATTTAATCG |
| | CCGCGACAATTTGCGACGGCGCGTGCAGGGCCAGACTGGAGGTGGC |
| | AACGCCAATCAGCAACGACTGTTTGCCCGCCAGTTGTTGTGCCACGC |
| | GGTTGGGAATGTAATTCAGCTCCGCCATCGCCGCTTCCACTTTTTCCC |
| | GCGTTTTCGCAGAAACGTGGCTGGCCTGGTTCACCACGCGGGAAAC |
| | GGTCTGATAAGAGACACCGGCATACTCTGCGACATCGTATAACGTTA |
| | CTGGTTTCAT |
| LacI | MKPVTLYDVAEYAGVSYQTVSRVVNQASHVSAKTREKVEAAMAELN |
| SEQ ID NO: | YIPNRVAQQLAGKQSLLIGVATSSLALHAPSQIVAAIKSRADQLGASVV |
| 1106 | VSMVERSGVEACKAAVHNLLAQRVSGLIINYPLDDQDAIAVEAACTNV |
| | PALFLDVSDQTPINSIIFSHEDGTRLGVEHLVALGHQQIALLAGPLSSVSA |
| | RLRLAGWHKYLTRNQIQPIAEREGDWSAMSGFQQTMQMLNEGIVPTA |
| | MLVANDQMALGAMRAITESGLRVGADISVVGYDDTEDSSCYIPPLTTIK |
| | QDFRLLGQTSVDRLLQLSQGQAVKGNQLLPVSLVKRKTTLAPNTQTAS |
| | PRALADSLMQLARQVSRLESGQ |
| PlacI (promoter | CCGGGCGCTATCATGCCATACCGCGAAAGGTTTTGCGCCATTCGATG |
| for lacI in | G |
| reverse | |
| orientation) | |
| SEQ ID NO: | |
| 1113 | |
| PlacI-RBS - lacI | TCACTGCCCGCTTTCCAGTCGGGAAACCTGTCGTGCCAGCTGCATTA |
| (reverse | ATGAATCGGCCAACGCGCGGGGAGAGGCGGTTTGCGTATTGGGCGC |
| orientation) | CAGGGTGGTTTTTCTTTTCACCAGTGAGACTGGCAACAGCTGATTGC |
| SEQ ID NO: | CCTTCACCGCCTGGCCCTGAGAGAGTTGCAGCAAGCGGTCCACGCTG |
| 1114 | GTTTGCCCCAGCAGGCGAAAATCCTGTTTGATGGTGGTTAACGGCGG |
| | GATATAACATGAGCTATCTTCGGTATCGTCGTATCCCACTACCGAGA |
| | TATCCGCACCAACGCGCAGCCCGGACTCGGTAATGGCGCGCATTGC |
| | GCCCAGCGCCATCTGATCGTTGGCAACCAGCATCGCAGTGGGAACG |
| | ATGCCCTCATTCAGCATTTGCATGGTTTGTTGAAAACCGGACATGGC |
| | ACTCCAGTCGCCTTCCCGTTCCGCTATCGGCTGAATTTGATTGCGAG |
| | TGAGATATTTATGCCAGCCAGCCAGACGCAGACGCGCCGAGACAGA |
| | ACTTAATGGGCCCGCTAACAGCGCGATTTGCTGGTGACCCAATGCGA |
| | CCAGATGCTCCACGCCCAGTCGCGTACCGTCCTCATGGGAGAAAAT |
| | AATACTGTTGATGGGTGTCTGGTCAGAGACATCAAGAAATAACGCC |
| | GGAACATTAGTGCAGGCAGCTTCCACAGCAATGGCATCCTGGTCATC |
| | CAGCGGATAGTTAATGATCAGCCCACTGACGCGTTGCGCGAGAAGA |
| | TTGTGCACCGCCGCTTTACAGGCTTCGACGCCGCTTCGTTCTACCATC |
| | GACACCACCACGCTGGCACCCAGTTGATCGGCGCGAGATTTAATCG |
| | CCGCGACAATTTGCGACGGCGCGTGCAGGGCCAGACTGGAGGTGGC |
| | AACGCCAATCAGCAACGACTGTTTGCCCGCCAGTTGTTGTGCCACGC |
| | GGTTGGGAATGTAATTCAGCTCCGCCATCGCCGCTTCCACTTTTTCCC |
| | GCGTTTTCGCAGAAACGTGGCTGGCCTGGTTCACCACGCGGGAAAC |
| | GGTCTGATAAGAGACACCGGCATACTCTGCGACATCGTATAACGTTA |
| | CTGGTTTCATATTCACCACCCTGAATTGACTCTCTTCCGGGCGCTAT |
| | CATGCCATACCGCGAAAGGTTTTGCGCCATTCGATGG |
| Ptac (minimal | ttgacaattaatcatcggctcgtataatg |
| promoter for | |
| gene expression - | |
| includes −10 | |
| and −35 region) | |
| SEQ ID NO: | |
| 1115 | |
| Exemplary | CGCGCCGCTTCGTCAGGCCACATAGCTTTCTTGTTCTGATCGGAACG |
| spacer region | ATCGTTGGCTGtg |
| SEQ ID NO: | |
| 1116 | |
| Exemplary Lac | aattgtgagcgctcacaatt |
| operator | |
| SEQ ID NO: | |
| 1107 | |
| Exemplary pTac | ttgacaattaatcatcggctcgtataatgtgtggaattgtgagcgctcacaattagctgt |
| promoter | |
| comprising −10 | |
| and −35 regions | |
| and Lac operon | |
| SEQ ID NO: | |
| 1108 | |
| Exemplary | taacaccgtgcgtgttg |
| Operator 1 | |
| SEQ ID NO: | |
| 1111 | |
| Exemplary | Tacctctggcggtgata |
| Operator 2 | |
| SEQ ID NO: | |
| 1112 | |
| Exemplary LacI | ATTCACCACCCTGAATTGACTCTCTT |
| RBS (reverse | |
| orientation) | |
| SEQ ID NO: | |
| 1117 | |

In some embodiments, the bacterial cells comprise endogenous gene(s) encoding the IPTG sensing transcriptional regulator, LacI. In some embodiments, the lacI gene is heterologous. In some embodiments, the gene encoding the IPTG level-sensing transcriptional regulator, e.g., LacI, is present on a plasmid. In some embodiments, the gene encoding the IPTG level-sensing transcriptional regulator, e.g., LacI, and the gene encoding the methionine decarboxylase/methionine importer are present on different plasmids. In some embodiments, the gene encoding the IPTG level-sensing transcriptional regulator, e.g., LacI, and the gene encoding the methionine decarboxylase or methionine importer are present on the same plasmid. In some embodiments, the gene encoding the IPTG level-sensing transcriptional regulator, e.g., LacI, is present on a chromosome. In some embodiments, the gene encoding the IPTG level-sensing transcriptional regulator, e.g., LacI, and the gene encoding the methionine decarboxylase or methionine importer are present on different chromosomes. In some embodiments, the gene encoding the IPTG level-sensing transcriptional regulator, e.g., LacI, and the gene encoding the methionine decarboxylase or methionine importer are present on the same chromosome, either at the same or a different insertion site. In some embodiments, expression of the transcriptional regulator is controlled by a different promoter than the promoter that controls expression of the gene encoding the methionine decarboxylase or methionine importer, e.g., a constitutive promoter. In some embodiments, expression of the transcriptional regulator is controlled by the same promoter that controls expression of the methionine decarboxylase or methionine importer. In some embodiments, the transcriptional regulator and the methionine decarboxylase or methionine importer are divergently transcribed from a promoter region.

In some embodiments, the promoter that is operably linked to the gene encoding the methionine decarboxylase or methionine importer is directly induced by exogenous environmental conditions. In some embodiments, the promoter that is operably linked to the gene encoding the methionine decarboxylase or methionine importer is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal. In some embodiments, the promoter is directly or indirectly induced by a molecule that is co-administered with the bacterial cell. In one embodiment, the inducible promoter is an anhydrotetracycline (ATC)-inducible promoter. In one embodiment, the inducible promoter is an IPTG promoter. In one embodiment, the IPTG promoter is Ptac.

As used herein the term “pTac” or “pTac promoter” includes the minimal promoter having −35 and −10 regions and at least the lac operator region. As used herein in certain instances, the term “pTac” or “pTac promoter” may also include an RBS in addition the minimal promoter and the Lac operator region. Non-limiting examples of suitable RBSs are listed herein. In a non-limiting example, pTac promoter sequence comprises SEQ ID NO: 1108. In some instances an RBS may be included at the 3′ end of SEQ ID NO: 1108. In a non-limiting example, the RBS comprises SEQ ID NO: 1107.

In certain embodiments, the bacterial cell comprises a gene encoding a methionine decarboxylase and/or a methionine importer expressed under the control of a fumarate and nitrate reductase regulator (FNR) responsive promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. FNR responsive promoters include, but are not limited to, the FNR responsive promoters listed in the chart, below. Underlined sequences are predicted ribosome binding sites, and bolded sequences are restriction sites used for cloning.

TABLE 4

| FNR Re- | |
|---|---|
| sponsive | |
| Promoter | Sequence |
| SEQ ID | GTCAGCATAACACCCTGACCTCTCATTAATTGTTCATGCCG |
| NO: 1 | GGCGGCACTATCGTCGTCCGGCCTTTTCCTCTCTTACTCTG |
| | CTACGTACATCTATTTCTATAAATCCGTTCAATTTGTCTGT |
| | TTTTTGCACAAACATGAAATATCAGACAATTCCGTGACTTA |
| | AGAAAATTTATACAAATCAGCAATATACCCCTTAAGGAGTA |
| | TATAAAGGTGAATTTGATTTACATCAATAAGCGGGGTTGCT |
| | GAATCGTTAAGGTAGGCGGTAATAGAAAAGAAATCGAGGCA |
| | AAA |
| SEQ ID | ATTTCCTCTCATCCCATCCGGGGTGAGAGTCTTTTCCCCCG |
| NO: 2 | ACTTATGGCTCATGCATGCATCAAAAAAGATGTGAGCTTGA |
| | TCAAAAACAAAAAATATTTCACTCGACAGGAGTATTTATAT |
| | TGCGCCCGTTACGTGGGCTTCGACTGTAAATCAGAAAGGAG |
| | AAAACACCT |
| SEQ ID | GTCAGCATAACACCCTGACCTCTCATTAATTGTTCATGCCG |
| NO: 3 | GGCGGCACTATCGTCGTCCGGCCTTTTCCTCTCTTACTCTG |
| | CTACGTACATCTATTTCTATAAATCCGTTCAATTTGTCTGT |
| | TTTTTGCACAAACATGAAATATCAGACAATTCCGTGACTTA |
| | AGAAAATTTATACAAATCAGCAATATACCCCTTAAGGAGTA |
| | TATAAAGGTGAATTTGATTTACATCAATAAGCGGGGTTGCT |
| | GAATCGTTAAGGATCCCTCTAGAAATAATTTTGTTTAACTT |
| | TAAGAAGGAGATATACAT |
| SEQ ID | CATTTCCTCTCATCCCATCCGGGGTGAGAGTCTTTTCCCCC |
| NO: 4 | GACTTATGGCTCATGCATGCATCAAAAAAGATGTGAGCTTG |
| | ATCAAAAACAAAAAATATTTCACTCGACAGGAGTATTTATA |
| | TTGCGCCCGGATCCCTCTAGAAATAATTTTGTTTAACTTTA |
| | AGAAGGAGATATACAT |
| SEQ ID | AGTTGTTCTTATTGGTGGTGTTGCTTTATGGTTGCATCGTA |
| NO: 5 | GTAAATGGTTGTAACAAAAGCAATTTTTCCGGCTGTCTGTA |
| | TACAAAAACGCCGTAAAGTTTGAGCGAAGTCAATAAACTCT |
| | CTACCCATTCAGGGCAATATCTCTCTTGGATCCCTCTAGAA |
| | ATAATTTTGTTTAACTTTAAGAAGGAGATATACAT |

In one embodiment, the FNR responsive promoter comprises SEQ ID NO: 1. In another embodiment, the FNR responsive promoter comprises SEQ ID NO: 2. In another embodiment, the FNR responsive promoter comprises SEQ ID NO: 3. In another embodiment, the FNR responsive promoter comprises SEQ ID NO: 4. In yet another embodiment, the FNR responsive promoter comprises SEQ ID NO: 5.

In some embodiments, multiple distinct FNR nucleic acid sequences are inserted in the genetically engineered bacteria. In alternate embodiments, the genetically engineered bacteria comprise a gene encoding a methionine decarboxylase and/or a gene encoding a methionine importer expressed under the control of an alternate oxygen level-dependent promoter, e.g., DNR (Trunk et al., 2010) or ANR (Ray et al., 1997). In these embodiments, expression of the methionine decarboxylase gene or methionine importer gene is particularly activated in a low-oxygen or anaerobic environment, such as in the gut. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene encoding the methionine decarboxylase or the gene encoding a methionine importer, in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the methionine decarboxylase or the methionine importer, in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the methionine decarboxylase or the gene encoding the methionine importer, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., (2006).

In some embodiments, the bacterial cells comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the methionine decarboxylase or methionine importer are present on different plasmids. In some embodiments, the gene encoding the oxygen level-sensing transcriptional regulator and the gene encoding the methionine decarboxylase or methionine importer are present on the same plasmid. In some embodiments, the gene encoding the oxygen level-sensing transcriptional regulator is present on a chromosome. In some embodiments, the gene encoding the oxygen level-sensing transcriptional regulator and the gene encoding the methionine decarboxylase or methionine importer are present on different chromosomes. In some embodiments, the gene encoding the oxygen level-sensing transcriptional regulator and the gene encoding the methionine decarboxylase or methionine importer are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the methionine decarboxylase or methionine importer. In some embodiments, expression of the transcriptional regulator is controlled by the same promoter that controls expression of the methionine decarboxylase or methionine importer. In some embodiments, the transcriptional regulator and the methionine decarboxylase or methionine importer are divergently transcribed from a promoter region.

In some embodiments, any of the gene(s) of the present disclosure may be integrated into the chromosome of a host cell, such as a bacterial chromosome, at one or more integration sites. For example, one or more copies of one or more gene(s) encoding a methionine decarboxylase or methionine importer may be integrated into the chromosome of a host cell, such as a bacterial chromosome. Having multiple copies of the gene or gene(s) integrated into the chromosome allows for greater production of the methionine decarboxylase(s) and also permits fine-tuning of the level of expression. Alternatively, different circuits described herein, such as any of the secretion or exporter circuits, in addition to the therapeutic gene(s) or gene cassette(s) could be integrated into the chromosome of a host cell, such as a bacterial chromosome, at one or more different integration sites to perform multiple different functions.

E. Temperature Dependent Regulation

In some instances, thermoregulators may be advantageous because of strong transcriptional control without the use of external chemicals or specialized media. Thermoregulated protein expression using the mutant cI857 repressor and the pL and/or pR phage λ promoters have been used to engineer recombinant bacterial strains. For example, a gene of interest cloned downstream of the λ promoters can be efficiently regulated by the mutant thermolabile cI857 repressor of bacteriophage λ. At temperatures below 37° C., cI857 binds to the oL or oR regions of the pR promoter and inhibits transcription by RNA polymerase. At higher temperatures, the functional cI857 dimer is destabilized, binding to the oL or oR DNA sequences is abrogated, and mRNA transcription is initiated. In certain instances, it may be advantageous to reduce, diminish, or shut off production of one or more protein(s) of interest. This can be done in a thermoregulated system by growing a bacterial strain at temperatures at which the temperature regulated system is not optimally active. Temperature regulated expression can then be induced as desired by changing the temperature to a temperature where the system is more active or optimally active.

For example, a thermoregulated promoter may be induced in culture, e.g., grown in a flask, fermenter or other appropriate culture vessel, e.g., used during cell growth, cell expansion, fermentation, recovery, purification, formulation, and/or manufacture. Bacteria comprising gene sequences or gene cassettes either indirectly or directly operably linked to a temperature sensitive system or promoter may, for example, could be induced by temperatures between 37° C. and 42° C. In some instances, the cultures may be grown aerobically. Alternatively, the cultures are grown anaerobically.

In some embodiments, the host cell, such as a bacterial host cell, described herein comprise one or more gene sequence(s) or gene cassette(s) which are directly or indirectly operably linked to a temperature regulated promoter. In some embodiments, the gene sequence(s) or gene cassette(s) are induced in vitro during growth, preparation, or manufacturing of the strain prior to in vivo administration. In some embodiments, the gene sequence(s) are induced upon or during in vivo administration. In some embodiments, the gene sequence(s) are induced during in vitro growth, preparation, or manufacturing of the strain prior to in vivo administration and upon or during in vivo administration. In some embodiments, the genetically engineered host cell, such as genetically engineered bacteria, further comprise gene sequence (s) encoding a transcription factor which is capable of binding to the temperature sensitive promoter. In some embodiments, the transcription factor is a repressor of transcription.

In one embodiment, the thermoregulated promoter is operably linked to a construct having gene sequence(s) or gene cassette(s) encoding one or more protein(s) of interest jointly with a second promoter, e.g., a second constitutive or inducible promoter. In some embodiments, two promoters are positioned proximally to the construct and drive its expression, wherein the thermoregulated promoter is induced under a first set of exogenous conditions, and the second promoter is induced under a second set of exogenous conditions. In a non-limiting example, the first and second conditions may be two sequential culture conditions (i.e., during preparation of the culture in a flask, fermenter or other appropriate culture vessel, e.g., thermoregulation and arabinose or IPTG). In another non-limiting example, the first inducing conditions may be culture conditions, e.g., permissive temperature, and the second inducing conditions may be in vivo conditions. Such in vivo conditions include low-oxygen, microaerobic, or anaerobic conditions, presence of gut metabolites, and/or metabolites administered in combination with the bacterial strain. In some embodiments, one or more thermoregulated promoters drive expression of one or more protein(s) of interest in combination with an oxygen regulated promoter, e.g., FNR, driving the expression of the same gene sequence(s).

In some embodiments, the thermoregulated promoter drives the expression of one or more protein(s) of interest from a low-copy plasmid or a high copy plasmid or a biosafety system plasmid described herein. In some embodiments, the thermoregulated promoter drives the expression of one or more protein(s) of interest from a construct which is integrated into the host cell chromosome, such as a bacterial chromosome. Exemplary insertion sites are described herein.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 309. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 313. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 316. In some embodiments, the thermoregulated construct further comprises a gene encoding mutant cI857 repressor, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 310. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 312. In some embodiments, the thermoregulated construct further comprises a gene encoding mutant cI38 repressor, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 314. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 315.

SEQ ID NOs: 309, 310, and 312-316 are shown in Table 5.

TABLE 5
Inducible promoter construct sequences and related elements

| Description | SEQ ID NO |
|---|---|
| Region comprising Temperature | SEQ ID NO: 309 |
| sensitive promoter | |
| mutant cI857 repressor | SEQ ID NO: 310 |
| nucleotide sequence | |
| mutant cI857 repressor polypeptide | SEQ ID NO: 312 |
| sequence | |
| Pr/Pl promoter | SEQ ID NO: 313 |
| mutant cI38 repressor nucleotide | SEQ ID NO: 314 |
| sequence | |
| mutant cI38 repressor polypeptide | SEQ ID NO: 315 |
| sequence | |
| Temperature sensitive promoter | SEQ ID NO: 316 |

In some embodiments, the bacterial cells comprise gene(s) encoding a temperature sensing transcriptional regulator/repressor described herein, e.g., cI857 or a mutant thereof. In some embodiments, the gene encoding the temperature sensing transcriptional regulator, is present on a plasmid. In some embodiments, the gene encoding the temperature sensing transcriptional regulator, and the gene encoding the methionine decarboxylase/methionine importer are present on different plasmids. In some embodiments, the gene encoding the temperature sensing transcriptional regulator, and the gene encoding the methionine decarboxylase or methionine importer are present on the same plasmid. In some embodiments, the gene encoding the temperature sensing transcriptional regulator is present on a chromosome. In some embodiments, the gene encoding the temperature sensing transcriptional regulator, and the gene encoding the methionine decarboxylase or methionine importer are present on different chromosomes. In some embodiments, the gene encoding the temperature sensing transcriptional regulator, and the gene encoding the methionine decarboxylase or methionine importer are present on the same chromosome, either at the same or at different insertion sites. In some embodiments, expression of temperature sensing transcriptional regulator is controlled by a different promoter than the promoter that controls expression of the gene encoding the methionine decarboxylase or methionine importer, e.g., a constitutive promoter. In some embodiments, expression of the temperature sensing transcriptional regulator is controlled by the same promoter that controls expression of the methionine decarboxylase or methionine importer. In some embodiments, the temperature sensing transcriptional regulator and the methionine decarboxylase or methionine importer are divergently transcribed from a promoter region.

In any of these embodiments, gene expression may be further optimized by methods known in the art, e.g., by optimizing ribosomal binding sites and/or increasing mRNA stability.

F. Phage Deletion

In some embodiments, the genetically engineered bacteria comprise one or more E. coli Nissle bacteriophage, e.g., Phage 1, Phage 2, and Phage 3. In some embodiments, the genetically engineered bacteria comprise one or mutations in Phage 3. Such mutations include deletions, insertions, substitutions and inversions and are located in or encompass one or more Phage 3 genes. In some embodiments, the one or more insertions comprise an antibiotic cassette. In some embodiments, the mutation is a deletion. In some embodiments, the genetically engineered bacteria comprise one or more deletions, which are located in or comprise one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 1064 is deleted from the Phage 3 genome. In one embodiment, a sequence comprising SEQ ID NO: 1064 is deleted from the Phage 3 genome.

G. Colibactin Island (Also Known as pks Island)

In some embodiments, the engineered bacterium further comprises a modified pks island (colibactin island). Non-limiting examples are described in PCT/US2021/061579, the contents of which are herein incorporated by reference in their entirety. Colibactin is a cyclomodulin that is synthetized by enzymes encoded by the pks genomic island. See Fais 2018. The pks genomic island is “highly conserved” in Enterobacteriaceae. Id. In Escherichia coli, a 54-kilobase pks genomic island contains 19 genes, clbA to clbS, and encodes various enzymes that have been described as an “assembly line responsible for colibactin synthesis.” Id. The pks genomic island assembly line for colibactin synthesis includes three polyketide synthases (ClbC, ClbI, ClbO), three non-ribosomal peptide synthases (ClbH, ClbJ, ClbN), two hybrid non-ribosomal peptide/polyketide synthases (ClbB, ClbK), and nine accessory, tailoring, and editing proteins. The polyketide synthases, non-ribosomal peptide synthases, and hybrid enzymes “are usually organized in mega-complexes as an assembly line, in which the synthesized compound is transferred from one enzymatic module to the following one.” Id. Colibactin undergoes a prodrug activation mechanism that incorporates an N-terminal structural motif, which is removed during the final stage of biosynthesis. In some embodiments, the engineered microorganism, e.g., engineered bacterium, comprises a modified pks island (colibactin island). In some embodiments, the engineered microorganism, e.g., engineered bacterium, comprises a modified clb sequence selected from one or more of the clbA, clbB, clbC, clbD, clbE, clbF, clbG, clbH, clbI, clbJ, clbK, clbL, clbM, clbN, clbO, clbP, clbQ, clbR, and clbS gene sequences, as compared to a suitable control, e.g., the native pks island in an unmodified bacterium of the same strain and/or subtype. In some embodiments, the modified clb sequence is an insertion, a substitution, and/or a deletion as compared to the control. In some embodiments, the modified clb sequence is a deletion of the clb island, e.g., clbA, clbB, clbC, clbD, clbE, clbF, clbG, clbH, clbI, clbJ, clbK, clbL, clbM, clbN, clbO, clbP, clbQ, clbR, and clbS. In one embodiment, the colibactin deletion is the whole island except for the clbS gene, e.g., a deletion of clbA, clbB, clbC, clbD, clbE, clbF, clbG, clbH, clbI, clbJ, clbK, clbL, clbM, clbN, clbO, clbP, clbQ, and clbR.

In some embodiments, the modified endogenous colibactin island comprises one or more modified clb sequences selected from clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), clbR (SEQ ID NO: 1082), or clbS (SEQ ID NO: 1803) gene. In some embodiments, the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and clbR (SEQ ID NO: 1082).

Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37: D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in a gene required for cell survival and/or growth. In one embodiment, the essential gene is an oligonucleotide synthesis gene, for example, thyA. In another embodiment, the essential gene is a cell wall synthesis gene, for example, dapA. In yet another embodiment, the essential gene is an amino acid gene, for example, serA or metA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria. For example, thymine is a nucleic acid that is required for bacterial cell growth; in its absence, bacteria undergo cell death. The thyA gene encodes thimidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound importer that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, lpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, mc, ftsB, eno, pyrG, chpR, lgt, fbaA, pgk, yqgD, metK, yqgF, plsC, ygiT, pare, ribB, cca, ygjD, tdcF, yraL, yihA, ftsN, murI, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, ispA, ispH, dapB, folA, imp, yabQ, ftsL, ftsI, murE, murF, mraY, murD, ftsW, murG, murC, ftsQ, ftsA, ftsZ, lpxC, secM, secA, can, folK, hemL, yadR, dapD, map, rpsB, infB, nusA, ftsH, obgE, rpmA, rplU, ispB, murA, yrbB, yrbK, yhbN, rpsl, rplM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, lpxA, lpxB, dnaE, accA, tilS, proS, yafF, tsf, pyrH, olA, rlpB, leuS, lnt, glnS, fidA, cydA, infA, cydC, ftsK, lolA, serS, rpsA, msbA, lpxK, kdsB, mukF, mukE, mukB, asnS, fabA, mviN, rne, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabl, racR, dicA, ydjB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain,” ACS Synthetic Biology (2015) DOI: 10.1021/acssynbio.5b00085, the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and 1188L. In some embodiments, the essential gene is pheS comprising the mutations F125G, P183T, P184A, R186A, and 1188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, 149G, and A51C. In some embodiments, the essential gene is metG comprising the mutations E45Q, N47R, 149G, and A51C. In some embodiments, the essential gene is adk comprising one or more of the following mutations: I4L, L5I and L6G. In some embodiments, the essential gene is adk comprising the mutations 14L, L5I and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, 149G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, 1317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (14L, L5I and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, 149G, and A51C). In other embodiments, the bacterial cell comprises mutations in three essential genes. For example, in some embodiments, the bacterial cell comprises mutations in tyrS (L36V, C38A, and F40G), metG (E45Q, N47R, 149G, and A51C), and pheS (F125G, P183T, P184A, R186A, and I188L).

In some embodiments, the genetically engineered bacterium is a conditional auxotroph whose essential gene(s) is replaced using the arabinose system described herein.

In some embodiments, the genetically engineered bacterium of the disclosure is an auxotroph and also comprises kill-switch circuitry, such as any of the kill-switch components and systems described herein. For example, the recombinant bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., “GeneGuard: A Modular Plasmid System Designed for Biosafety,” ACS Synthetic Biology (2015) 4: 307-16, the entire contents of which are expressly incorporated herein by reference). In some embodiments, the genetically engineered bacterium of the disclosure is an auxotroph and also comprises kill-switch circuitry, such as any of the kill-switch components and systems described herein, as well as another biosecurity system, such a conditional origin of replication (see Wright et al., supra).

Isolated Plasmids

In other embodiments, the disclosure provides an isolated plasmid comprising a first nucleic acid encoding a methionine decarboxylase or methionine importer operably linked to a first inducible promoter. In another embodiment, the disclosure provides an isolated plasmid comprising a second nucleic acid encoding at least one additional methionine decarboxylase or methionine importer. In one embodiment, the first nucleic acid and the second nucleic acid are operably linked to the first promoter. In another embodiment, the second nucleic acid is operably linked to a second inducible promoter. In one embodiment, the first inducible promoter and the second inducible promoter are separate copies of the same inducible promoter. In another embodiment, the first inducible promoter and the second inducible promoter are different inducible promoters. In one embodiment, the first promoter, the second promoter, or the first promoter and the second promoter, are each directly or indirectly induced by low-oxygen or anaerobic conditions. In another embodiment, the first promoter, the second promoter, or the first promoter and the second promoter, are each IPTG inducible. In another embodiment, the first promoter, the second promoter, or the first promoter and second promoter are each regulated by changes in temperature.

In any of the above-described embodiments, the plasmid is a high-copy plasmid. In another embodiment, the plasmid is a low-copy plasmid.

In another aspect, the disclosure provides a recombinant host cell, such as a recombinant bacterial cell, comprising an isolated plasmid described herein. In another embodiment, the disclosure provides a pharmaceutical composition comprising the recombinant bacterial cell.

Integration

In some embodiments, any of the gene(s) or gene cassette(s) of the present disclosure may be integrated into the host cell chromosome, such as a bacterial chromosome, at one or more integration sites. One or more copies of the gene (for example, an amino acid catabolism gene) or gene cassette (for example, a gene cassette comprising an amino acid catabolism gene and an amino acid importer gene) may be integrated into the host cell chromosome, such as a bacterial chromosome. Having multiple copies of the gene or gene cassette integrated into the chromosome allows for greater production of the methionine decarboxylase, and other enzymes of the gene cassette, and also permits fine-tuning of the level of expression. Alternatively, different circuits described herein, such as any of the kill-switch circuits, in addition to the therapeutic gene(s) or gene cassette(s) could be integrated into the host cell chromosome, such as a bacterial chromosome, at one or more different integration sites to perform multiple different functions.

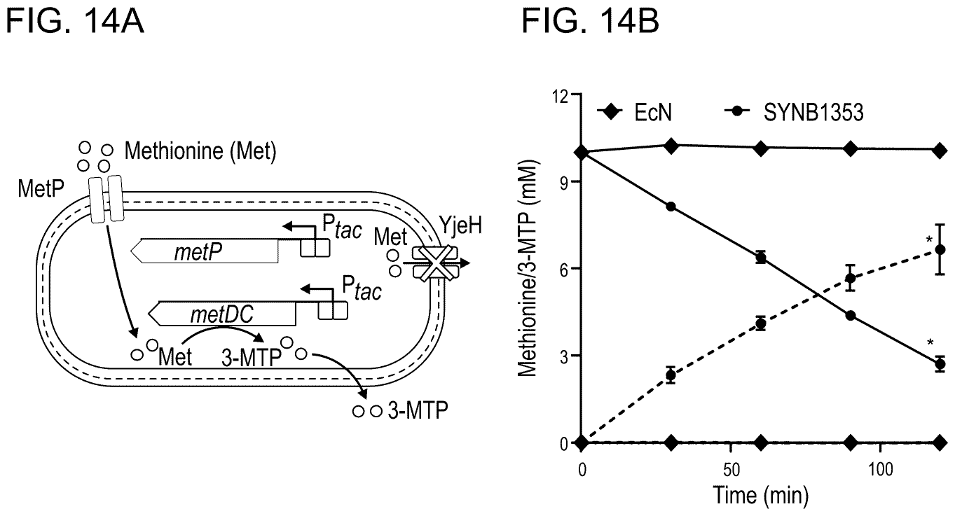

In one non-limiting example, gene sequences encoding MetP and MetDC are integrated to facilitate Met import and metabolism. In one embodiment, metP is derived from Flavobacterium segetis and facilitates the uptake of Met into the cell. In one embodiment, MetDC is derived from Streptomyces sp. 590 and includes two modifications (Q70D and N82H). In one embodiment, MetP is derived from Flavobacterium segetis. In one embodiment, both genes or gene sequences are operably linked to a chemically inducible promoter. In some embodiments, the promoter is induced by the compound Isopropyl β-D-1-thiogalactopyranoside (IPTG) e.g., PTac promoter (see e.g., FIG. 14A).

In some embodiments, the genetically engineered host cell, such as a genetically engineered bacterium, comprises a single integrated copy of metDC. In some embodiments, the genetically engineered host cell, such as a genetically engineered bacterium, comprises two or more integrated copies of metDC. In some embodiments, two or more copies of the metDC gene are present at the same integration site, arranged in a cassette, and operably linked to the same promoter. In some embodiments, two or more copies of the metDC gene are present at the same integration site, arranged in a cassette, and one or more copies of the metDC gene are operably linked to different copies of same promoter or different promoters.

Alternatively, in some embodiments, a genetically engineered host cell, such as a genetically engineered bacterium, may comprise two or more copies of the metDC gene and each copy of the metDC gene may be integrated at distinct sites. In some embodiments, each copy of the metDC gene is linked to a separate promoter at each integration site. In some embodiments, each copy of the metDC gene is operably linked to a different copy of the same promoter. In some embodiments, the promoters are different between two or more copies of the metDC gene. In some embodiments, the promoter is an inducible promoter, e.g., an IPTG inducible promoter. In some embodiments, the genetically engineered host cell, such as a genetically engineered bacterium, comprises a gene cassette comprising a metDC gene operatively linked to an IPTG inducible promoter, e.g., a pTac promoter. In some embodiments, the genetically engineered host cell, such as a genetically engineered bacterium, comprise a Ptac-metDC cassette.

In some embodiments, the genetically engineered host cell, such as a genetically engineered bacterium, comprises a single integrated copy of metP. In some embodiments, the genetically engineered host cell, such as a genetically engineered bacterium, comprises two or more integrated copies of metP. In some embodiments, two or more copies of the metP gene are present at the same integration site, arranged in a cassette, and operably linked to the same promoter. In some embodiments, multiple copies of the metP gene are present at the same integration site, arranged in a cassette, and one or more copies of the metP gene are operably linked to different copies of same promoter or different promoters.

Alternatively, in some embodiments, a genetically engineered host cell, such as a genetically engineered bacterium, may comprise two or more copies of the metP gene and each copy of the metP gene may be integrated at distinct sites. In some embodiments, each copy of the metP gene is linked to a separate promoter at each integration site. In some embodiments, each copy of the metP gene is operably linked to a different copy of the same promoter. In some embodiments, the promoters are different between two or more copies of the metP gene. In some embodiments, the promoter is an inducible promoter, e.g., an IPTG inducible promoter. In some embodiments, the genetically engineered bacterium comprises a gene cassette comprising a metP gene operatively linked to an IPTG inducible promoter, e.g., Ptac. In some embodiments, the genetically engineered bacteria comprise a Ptac-metP cassette.

In one embodiment, both metDC and metP genes are arranged in a cassette.

In some embodiments, the genetically engineered host cell, such as a genetically engineered bacterium, comprises a single integrated copy of metDC and/or metP. In some embodiments, the genetically engineered host cell, such as a genetically engineered bacterium, comprises one or more integrated copies of metDC and one or more integrated copies of metP. In some embodiments, the metDC gene and the metP gene are present at the same integration site, arranged in a cassette, and operably linked to the same promoter. In some embodiments, the metDC gene and the metP gene are present at the same integration site, arranged in a cassette, and the metDC gene and the metP gene are operably linked to different copies of same promoter or different promoters.

In some embodiments, the genetically engineered host cell, such as a genetically engineered bacterium or microorganism, comprises a single integrated copy of a gene cassette comprising metDC and metP. In some embodiments, the genetically engineered host cell, such as a genetically engineered bacterium, comprises two or more integrated copies a gene cassette comprising metDC and metP. In some embodiments, multiple copies of a gene cassette comprising metDC and metP are present at the same integration site, and operably linked to the same promoter. In some embodiments, multiple copies of the gene cassette comprising metDC and metP are present at the same integration site, and one or more copies of the cassette comprising metDC and metP are operably linked to different copies of same promoter or different promoters. In any of these embodiments, the metDC gene and the metP within the gene cassette may be operably linked to the same promoter. Alternatively, in any of these embodiments, the metDC gene and the metP may be each operably linked to a different copy of the same promoter. In another alternative, in any of these embodiments, the metDC gene and the metP may each be operably linked to a different promoter.

Alternatively, in some embodiments, a genetically engineered host cell, such as a genetically engineered bacterium or microorganism, may comprise two or more copies of the gene cassette comprising metDC and metP and each copy of the gene cassette comprising metDC and metP may be integrated at distinct sites. In some embodiments, each copy of the gene cassette comprising metDC and metP is linked to one or more separate promoters at each integration site. In some embodiments, each copy of the gene cassette comprising metDC and metP is operably linked to one or more different copies of the same promoter. In some embodiments, the promoters are different between two or more copies of gene cassette comprising metDC and metP gene. In any of these embodiments, the metDC gene and the metP within the gene cassette may be operably linked to the same promoter. Alternatively, in any of these embodiments, the metDC gene and the metP may be each operably linked to a different copy of the same promoter. In another alternative, in any of these embodiments, the metDC gene and the metP may each be operably linked to a different promoter.

In some embodiments, the promoter is an inducible promoter, e.g., an IPTG inducible promoter, e.g., a Ptac promoter. In some embodiments, the genetically engineered host cell, such as a genetically engineered bacterium or microorganism, comprises gene cassette comprising metDC and metP, wherein metDC and metP are operably linked to the same IPTG inducible promoter. In some embodiments, the genetically engineered host cell, such as a genetically engineered bacterium, comprises gene cassette comprising metDC and metP, wherein metDC and metP are each operably linked to a different copy of an IPTG inducible promoter. In some embodiments metDC and metP are each operably linked to a different copy of a Ptac promoter. In some embodiments, the genetically engineered host cell, such as a genetically engineered bacterium, comprise a Ptac-metP-metDC cassette or a Ptac-metDC-metP-cassette. In some embodiments, the genetically engineered host cell, such as a genetically engineered bacterium or microorganism, comprise a Ptac-metP-Ptac-metDC cassette or a Ptac-metDC-Ptac-metP-cassette.

Alternatively, in some embodiments, a genetically engineered host cell, such as a genetically engineered bacterium, may comprise one or more copies of the metDC gene and one or more copies of the metP gene and the metDC gene and the metP gene may be integrated at distinct sites. In some embodiments, the metP gene and the metDC gene are linked to a separate promoter at each integration site. In some embodiments, the metP gene and the metDC gene are operably linked to a different copy of the same promoter. In some embodiments, the promoters are different between the metP gene and the metDC gene. In some embodiments, the promoter is an inducible promoter, e.g., an IPTG inducible promoter. In some embodiments, the genetically engineered host cell, such as a genetically engineered bacterium, comprises a metDC gene operatively linked to an IPTG inducible promoter, e.g., a pTac promoter and a metP gene operatively linked to a different IPTG inducible promoter, e.g., a pTac promoter. In some embodiments, the genetically engineered host cell, such as a genetically engineered bacterium, comprise a cassette comprising Ptac-metDC cassette and a cassette comprising Ptac-metP.

In some embodiments, the genetically engineered host cell, such as a genetically engineered bacterium or microorganism, further comprise a LacI gene. In some embodiments, the lacI gene is non-native or heterologous. In some embodiments, the lacI gene is native.

In some embodiments, the genetically engineered host cell, such as a genetically engineered bacterium or microorganism, comprises a single integrated copy of lacI. In some embodiments, the genetically engineered bacterium comprises two or more integrated copies of lacI. In some embodiments, two or more copies of the lacI gene are present at the same integration site, arranged in a cassette, and operably linked to the same promoter. In some embodiments, two or more copies of the lacI gene are present at the same integration site, arranged in a cassette, and one or more copies of the lacI gene are operably linked to different copies of same promoter or different promoters.

Alternatively, in some embodiments, a genetically engineered host cell, such as a genetically engineered bacterium or microorganism, may comprise two or more copies of the lacI gene and each copy of the lacI gene may be integrated at distinct sites. In some embodiments, each copy of the lacI gene is linked to a separate promoter at each integration site. In some embodiments, each copy of the lacI gene is operably linked to a different copy of the same promoter. In some embodiments, the promoters are different between two or more copies of the lacI gene. In some embodiments, the promoter is a native lacI promoter. In some embodiments, the promoter is a non-native promoter. In some embodiments, the promoter is a constitutive promoter. In some embodiments, the genetically engineered bacterium comprises a gene cassette comprising a lacI gene operatively linked to a constitutive promoter, e.g., a Plac promoter. In some embodiments, the genetically engineered bacteria or microorganism comprise a Plac-lacI cassette.

In some embodiments, the genetically engineered host cell, such as a genetically engineered bacterium or microorganism, comprise a gene cassette having a lacI gene and a metDC and/or metP gene. In some embodiments, the PlacI promoter and the lacI gene sequences are located upstream of a metDC gene, a metP gene or a gene cassette comprising metDC and metP. In some embodiments, the lacI gene is in reverse orientation relative to the metDC gene, metP gene or gene cassette comprising metDC and metP, i.e., Lac is divergently transcribed from a promoter region relative to the metDC gene, metP gene or gene cassette comprising metDC and met. Accordingly, in some embodiments, the genetically engineered host cell, such as a genetically engineered bacterium or microorganism, comprise a lacI-PlacI cassette.

In some embodiments, the genetically engineered host cell, such as a genetically engineered bacterium or microorganism, comprise a lacI-PlacI-Ptac-metDC cassette. In some embodiments, the genetically engineered host cell, such as a genetically engineered bacterium or microorganism, comprise a lacI-PlacI-Ptac-metP cassette. In some embodiments, the genetically engineered host cell, such as a genetically engineered bacterium or microorganism, comprise a lacI-PlacI-Ptac-metP-metDC cassette. In some embodiments, the genetically engineered host cell, such as a genetically engineered bacterium or microorganism, comprise a lacI-PlacI-Ptac-metDC-metP cassette. In some embodiments, the genetically engineered host cell, such as a genetically engineered bacterium or microorganism, comprise a lacI-PlacI-Ptac-metP-Ptac-metDC cassette. In some embodiments, the genetically engineered host cell, such as a genetically engineered bacterium or microorganism, comprise a lacI-PlacI-Ptac-metDC-Ptac-metP-cassette.

In some embodiments, the genetically engineered host cell, such as a genetically engineered bacterium or microorganism, comprise a PlacI-lacI-Ptac-metDC cassette. In some embodiments, the genetically engineered host cell, such as a genetically engineered bacterium or microorganism, comprise a PlacI-lacI-Ptac-metP cassette. In some embodiments, the genetically engineered bacteria or microorganism comprise a PlacI-lacI-Ptac-metP-metDC cassette. In some embodiments, the genetically engineered host cell, such as a genetically engineered bacterium or microorganism, comprise a PlacI-lacI-Ptac-metDC-metP cassette. In some embodiments, the genetically engineered host cell, such as a genetically engineered bacterium or microorganism, comprise a PlacI-lacI-Ptac-metP-Ptac-metDC cassette In some embodiments, the genetically engineered host cell, such as a genetically engineered bacterium or microorganism, comprise a PlacI-lacI-Ptac-metDC-Ptac-metP-cassette.

In more specific embodiments, the genetically engineered host cell, such as a genetically engineered bacterium or microorganism, comprises three integrated copies of a metDC gene. In some embodiments, each of the three copies are integrated at separate integration sites. In some embodiments, the genetically engineered host cell, such as a genetically engineered bacterium, comprises a single integrated copy of metP. In some embodiments, the genetically engineered host cell, such as a genetically engineered bacterium or microorganism, comprises three integrated copies of metDC and one integrated copy of metP. In some embodiments, the genetically engineered host cell, such as a genetically engineered bacterium or microorganism, comprises a single integrated copy of a gene cassette comprising metDC and metP and further comprises a two integrated copies of a metDC gene. In some embodiments, each copy of metDC and metP are operably linked to different copies of the same promoter. In some embodiments, the promoter is an IPTG inducible promoter, e.g., Ptac. In some embodiments, the metDC gene encodes metDC from Streptomyces sp. 590, having Q70D and N82H mutations. In some embodiments, the metP gene encodes MetP from Flavobacterium segetis. In some embodiments, the gene cassette comprising metDC and metP further comprises lacI. In some embodiments, one copy of a metDC gene is present in a gene cassette which further comprises lacI. In some embodiments, a second copy of a metDC gene is not in a gene cassette further comprising lacI. In some embodiments, the lacI promoter is a constitutive promoter, e.g., Plac.

In one embodiment, the genetically engineered host cell, such as a genetically engineered bacterium or microorganism, comprises three integrated copies of a metDC gene, integrated at three separate integration sites, wherein one of the three metDC gene copies is present in a cassette further comprising a metP gene. In some embodiments, each of the three copies of the metDC genes and the metP genes, are linked to different copies of the same promoter. In some embodiments, the promoter is an inducible promoter, such as an IPTG inducible promoter, e.g. pTac. In some embodiments, the metDC gene encodes MetDC from Streptomyces sp. 590, having Q70D and N82H mutations and the metP gene encodes MetP from Flavobacterium segetis.

In any of these embodiments, the genetically engineered host cell, such as a genetically engineered bacterium or microorganism, may further comprise one or more of (1) a deletion in yjeH gene that encodes a Met/branched chain amino acid exporter (2) a deletion of the dapA gene that encodes for dihydrodipicolinate synthase (3) a deletion in the pks island which encodes colibactin and (4) an endogenous Nissle prophage gene deletion.

In one specific embodiment, the host cell, such as a genetically engineered bacterium or microorganism, comprises three copies of a metDC gene derived from Streptomyces sp. 590 and comprising two modifications (Q70D and N82H), each integrated at separate integration sites, wherein one of the three metDC gene copies is present in a cassette further comprising metP derived from Flavobacterium segetis, wherein the three copies of the metDC gene and the metP gene are each operably linked to separate copies of the same IPTG inducible promoter, wherein the host cell, such as a genetically engineered bacterium or microorganism, further comprises two non-native copies of the lacI gene each operably linked to separate copies of the same constitutive Plac promoter, wherein a first copy is of the lacI gene is present in reverse orientation upstream of the metP-metDC gene cassette and the second copy is present in reverse orientation upstream of a-metDC gene, and wherein the host cell, such as a genetically engineered bacterium or microorganism, further comprises a deletion in yjeH gene, a deletion of the dapA gene, a deletion in the pks island, and an endogenous Nissle prophage gene deletion.

In one embodiment, the genetically engineered host cell, such as a genetically engineered bacterium or microorganism, comprises lacI-PlacI-Ptac-metDC, Ptac-metDC, and lacI-PlacI-Ptac-metP-Ptac-metDC, wherein the metDC genes encode MetDC from Streptomyces sp. 590, having Q70D and N82H mutations and metP gene encoding MetP from Flavobacterium segetis, and wherein the bacterium further comprises a deletion in yjeH gene, a deletion of the dapA gene, a deletion in the pks island, and an endogenous Nissle prophage gene deletion.

In Vivo Models

The recombinant host cells, such as recombinant bacteria, may be evaluated in vivo, e.g., in an animal model. Any suitable animal model of a disease or condition associated with amino acid metabolism, such as homocystinuria, may be used.

Pharmaceutical Compositions and Formulations

Pharmaceutical compositions comprising the genetically engineered bacteria described herein may be used to treat, manage, ameliorate, and/or prevent a disorder associated with amino acid catabolism, e.g., homocystinuria. Pharmaceutical compositions comprising one or more genetically engineered bacteria, alone or in combination with prophylactic agents, therapeutic agents, and/or pharmaceutically acceptable carriers are provided.

Pharmaceutical compositions comprising the genetically engineered microorganisms of the invention may be used to treat, manage, ameliorate, and/or prevent a disorder associated with amino acid catabolism or symptom(s) associated with diseases or disorders associated with amino acid catabolism. Pharmaceutical compositions of the invention comprising one or more genetically engineered bacteria, and/or one or more genetically engineered virus, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise the genetic modifications described herein, e.g., to express a methionine decarboxylase. In alternate embodiments, the pharmaceutical composition comprises two or more species, strains, and/or subtypes of bacteria that are each engineered to comprise the genetic modifications described herein, e.g., to express a methionine decarboxylase.

The pharmaceutical compositions of the invention described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 104 to 1012 bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal

The genetically engineered bacteria may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection.

The genetically engineered microorganisms of the disclosure may be administered intrathecally. In some embodiments, the genetically engineered microorganisms of the invention may be administered orally. The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, PA. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the host cell may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The host cells disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, tale, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to adult subjects or pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered viruses are prepared for delivery, taking into consideration the need for efficient delivery and for overcoming the host antiviral immune response. Approaches to evade antiviral response include the administration of different viral serotypes as part of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Methods of Treatment

Further disclosed herein are methods of treating diseases associated with methionine metabolism. In some embodiments, disclosed herein are methods for reducing, ameliorating, or eliminating one or more symptom(s) associated with these diseases or disorders.

As used herein the terms “disease associated with amino acid metabolism” or a “disorder associated with amino acid metabolism” is a disease or disorder involving the abnormal, e.g., increased, levels of one or more amino acids in a subject. In one embodiment, a disease or disorder associated with amino acid metabolism is homocystinuria, cancer, or a metabolic syndrome/disease. For example, for metabolic indications, a methionine-restricted diet has been shown to increase lifespan, reduce adiposity, decrease systemic inflammation, and improve insulin sensitivity in rodent and some large animal models (see, for example, Dong et al., EClinicalMedicine, 2019). For indications in immune-oncology and cancer, there is preclinical data supporting a link between tumoral methionine restriction and antitumor activity (see, for example, Gay et al., Cancer Medicine, 2017, 6(6):1437-1452).

In some embodiments, a disease or disorder associated with amino acid metabolism is cystinuria. Cystinuria is a condition in which stones made from cysteine dimers (known as cystine) form in the kidney, ureter, and bladder. The condition is inherited in an autosomal recessive manner. Normally, most cystine dissolves and returns to the bloodstream after entering the kidneys. Subjects with cystinuria have a genetic defect in SLC3A1 or SLC7A9. As a result, cystine builds up in the urine and forms crystals or stones (˜10× increase vs healthy subjects). The goal of current treatments is to relieve symptoms and prevent more stones from forming. Treatment involves drinking plenty of water, use drugs that make urine more alkaline, dietary salt/animal protein restriction, kidney/bladder surgery. A large proportion of patients fail to achieve therapeutic success even with adherence to current SOC. Cystinuria is a lifelong condition. Stones often return.

In some embodiments, the disclosure provides methods for reducing, ameliorating, or eliminating one or more symptom(s) associated with these diseases.

The method may comprise preparing a pharmaceutical composition with at least one genetically engineered species, strain, or subtype of bacteria described herein, and administering the pharmaceutical composition to a subject in a therapeutically effective amount. In some embodiments, the genetically engineered bacteria disclosed herein are administered orally, e.g., in a liquid suspension. In some embodiments, the genetically engineered bacteria are lyophilized in a gel cap and administered orally. In some embodiments, the genetically engineered bacteria are administered via a feeding tube or gastric shunt. In some embodiments, the genetically engineered bacteria are administered rectally, e.g., by enema. In some embodiments, the genetically engineered bacteria are administered topically, intraintestinally, intrajejunally, intraduodenally, intraileally, and/or intracolically. In one embodiment, the genetically engineered bacteria are injected directly into a tumor.

In certain embodiments, administering the pharmaceutical composition to the subject reduces the level of an amino acid, e.g., methionine, homocysteine, cysteine or cystine in a subject. In some embodiments, the methods of the present disclosure may reduce the level of an amino acid, e.g., methionine, homocysteine, cysteine or cystine in a subject by at least about 10%, 20%, 25%, 30%, 40%, 50%, 60%, 70%, 75%, 80%, 85%, 90%, 95%, or more as compared to levels in an untreated or control subject. In some embodiments, reduction is measured by comparing the amino acid concentration in a subject before and after administration of the pharmaceutical composition. In some embodiments, the method of treating or ameliorating a disease or disorder allows one or more symptoms of the condition or disorder to improve by at least about 10%, 20%, 30%, 40%, 50%, 60%, 70%, 80%, 90%, 95%, or more as compared to levels in an untreated or control subject, or as compared to levels in the subject prior to administration. Amino acid levels may be measured by methods known in the art (see methionine decarboxylase section, supra).

Before, during, and after the administration of the pharmaceutical composition, methionine concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to reduce amino acid, e.g., methionine concentrations in a subject to undetectable levels, or to less than about 1%, 2%, 5%, 10%, 20%, 25%, 30%, 40%, 50%, 60%, 70%, 75%, or 80% of the subject's amino acid concentration(s) prior to treatment.

Before, during, and after the administration of the pharmaceutical composition, homocysteine concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to reduce amino acid, e.g., homocysteine concentrations in a subject to undetectable levels, or to less than about 1%, 2%, 5%, 10%, 20%, 25%, 30%, 40%, 50%, 60%, 70%, 75%, or 80% of the subject's amino acid concentration(s) prior to treatment.

Before, during, and after the administration of the pharmaceutical composition, cysteine concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to reduce amino acid, e.g., cysteine concentrations in a subject to undetectable levels, or to less than about 1%, 2%, 5%, 10%, 20%, 25%, 30%, 40%, 50%, 60%, 70%, 75%, or 80% of the subject's amino acid concentration(s) prior to treatment.

Before, during, and after the administration of the pharmaceutical composition, cystine concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to reduce amino acid, e.g., cystine concentrations in a subject to undetectable levels, or to less than about 1%, 2%, 5%, 10%, 20%, 25%, 30%, 40%, 50%, 60%, 70%, 75%, or 80% of the subject's amino acid concentration(s) prior to treatment.

The methods disclosed herein may further comprise isolating a sample from the subject prior to administration of a composition and determining the level of the amino acid(s) in the sample. In some embodiments, the methods may further comprise isolating a sample from the subject after to administration of a composition and determining the level of amino acid(s) in the sample.

In certain embodiments, administering the pharmaceutical composition to the subject prevents or reduces formation, occurrence, or presence of stones in a subject. In some embodiments, the stones are present in the kidney, bladder or urether. In some embodiments, the stones are cystine stones. In some embodiments, the methods of the present disclosure may reduce or reduce/prevent an increase in the formation, occurrence, or presence of a stone, e.g., a cystine stone, in a subject by at least about 10%, 20%, 25%, 30%, 40%, 50%, 60%, 70%, 75%, 80%, 85%, 90%, 95%, or more as compared to levels in an untreated or control subject. In some embodiments, levels of formation, occurrence, or presence of a stone, e.g., a cystine stone, is measured by comparing stone formation, occurrence or presence, respectively, in a subject before and after administration of the pharmaceutical composition, e.g., within a certain time span. In some embodiments, the formation, occurrence, or presence of a stone, e.g., a cystine stone, in a subject may be prevented completely, or completely within a certain time span. In some embodiments, the methods of the present disclosure may reduce or reduce or prevent an increase in cystine stone number, stone volume, stone area or stone weight, e.g., over time, in a subject by at least about 10%, 20%, 25%, 30%, 40%, 50%, 60%, 70%, 75%, 80%, 85%, 90%, 95%, or more as compared to levels in an untreated or control subject. In some embodiments, levels of cystine stone number, volume, area or weight over time are measured by comparing cystine stone number, stone volume, stone area or stone weight, respectively, in a subject before and after administration of the pharmaceutical composition, e.g., within a certain time span. In some embodiments, the methods of the present disclosure may prevent a change, e.g., an increase, in number of cystine stones, stone volume, stone area or stone weight, e.g., over time, in a subject completely, or completely within a certain time span.

In some embodiments, the method of treating or ameliorating a disease or disorder allows the symptom of stone formation, e.g., cystine stone formation, e.g., in a subject having cystinuria, to improve by at least about 10%, 20%, 30%, 40%, 50%, 60%, 70%, 80%, 90%, 95%, or more as compared to levels in an untreated or control subject, or as compared to levels in the subject prior to administration. Presence of stones and stone attributes, e.g. cystine stone number, volume, area and weight may be measured by methods known in the art, e.g., CT scan ultrasound or MRI.

In certain embodiments, the genetically engineered bacteria comprising a methionine decarboxylase is E. coli Nissle. The genetically engineered bacteria may be destroyed, e.g., by defense factors in the gut or blood serum (Sonnenborn et al., 2009), or by activation of a kill switch, several hours or days after administration. Thus, the pharmaceutical composition comprising the methionine decarboxylase may be re-administered at a therapeutically effective dose and frequency. In alternate embodiments, the genetically engineered bacteria are not destroyed within hours or days after administration and may propagate and colonize the gut.

The methods disclosed herein may comprise administration of a composition alone or in combination with one or more additional therapies. The pharmaceutical composition may be administered alone or in combination with one or more additional therapeutic agents, including but not limited to, sodium phenylbutyrate, sodium benzoate, and glycerol phenylbutyrate. The methods may also comprise following an amino acid, e.g., methionine, restricted diet, and/or administration of betaine, pyridoxine, and/or other enzyme replacement-based therapies such as OT-58 or AGLE-177. OT-58 represents a therapeutic approach incorporating the use of a modified version of the native human CBS enzyme. The goal of this treatment is to introduce the CBS enzyme into circulation, resulting in reduced Hey levels, increased crystalthionine levels, and normalized cysteine levels. AGLE-177 is an engineered human enzyme designed to degrade both homocysteine and homocysteine (two homocysteine molecules bound together) to lower abnormally high levels of homocysteine in the blood.

Methionine abundance in natural sources of protein ranges from 1-2% (or 1-2 g/100 g protein intake). Assuming the average human subject needs to degrade about 1.0 g methionine per day with meals, and assuming the recombinant bacteria provides 3 hours of activity per dose, that leaves 3× doses per day at 5×10¹¹ dose and 1.0 g methionine per day (0.33 g/dose). 0.33 g methionine/dose=2230 μmol methionine. 2230 μmol/3 hours/5×10¹¹ cells leads to 1.49 μmol/hr/1×10⁹ cells. The target dose is 5×10¹¹ live recombinant bacterial cells/mL.

For human subjects on a low protein diet eating 10 g protein/day, the subject needs to degrade about 0.1-1 g, e.g. 0.1 g, 0.2 g, 0.3 g, 0.4 g, 0.5 g, 0.6 g, 0.7 g, 0.8 g, 0.9 g or 1 g, methionine per day with meals. Assuming the recombinant bacteria provides 3 hours of activity per dose, that leaves 3× doses per day at 5×10¹¹ dose and 0.1 g per day (0.033 g/dose). 0.033 g methionine/dose=223 μmol methionine. 223 μmol/3 hours/5×10¹¹ cells leads to 0.15 μmol/hr/1×10⁹ cells. The target dose is 5×10¹¹ live recombinant bacterial cells/mL.

Accordingly, in one embodiment, the recombinant bacteria disclosed herein has a methionine degradation activity of about 0.1 μmol/hr/1×10⁹ cells. Accordingly, in one embodiment, the recombinant bacteria disclosed herein has a methionine degradation activity of about 0.15 μmol/hr/1×10⁹ cells. Accordingly, in one embodiment, the recombinant bacteria disclosed herein has a methionine degradation activity of about 0.2 μmol/hr/1×10⁹ cells. Accordingly, in one embodiment, the recombinant bacteria disclosed herein has a methionine degradation activity of about 0.25 μmol/hr/1×10⁹ cells. Accordingly, in one embodiment, the recombinant bacteria disclosed herein has a methionine degradation activity of about 0.3 μmol/hr/1×10⁹ cells. Accordingly, in one embodiment, the recombinant bacteria disclosed herein has a methionine degradation activity of about 0.4 μmol/hr/1×10⁹ cells. Accordingly, in one embodiment, the recombinant bacteria disclosed herein has a methionine degradation activity of about 0.5 μmol/hr/1×10⁹ cells. Accordingly, in one embodiment, the recombinant bacteria disclosed herein has a methionine degradation activity of about 0.6 μmol/hr/1×10⁹ cells. Accordingly, in one embodiment, the recombinant bacteria disclosed herein has a methionine degradation activity of about 0.7 μmol/hr/1×10⁹ cells. Accordingly, in one embodiment, the recombinant bacteria disclosed herein has a methionine degradation activity of about 0.8 μmol/hr/1×10⁹ cells. Accordingly, in one embodiment, the recombinant bacteria disclosed herein has a methionine degradation activity of about 0.9 μmol/hr/1×10⁹ cells. Accordingly, in one embodiment, the recombinant bacteria disclosed herein has a methionine degradation activity of about 1.0 μmol/hr/1×10⁹ cells. Accordingly, in one embodiment, the recombinant bacteria disclosed herein has a methionine degradation activity of about 1.10 μmol/hr/1×10⁹ cells. Accordingly, in one embodiment, the recombinant bacteria disclosed herein has a methionine degradation activity of about 1.30 μmol/hr/1×10⁹ cells. Accordingly, in one embodiment, the recombinant bacteria disclosed herein has a methionine degradation activity of about 1.30 μmol/hr/1×10⁹ cells. Accordingly, in one embodiment, the recombinant bacteria disclosed herein has a methionine degradation activity of about 1.40 μmol/hr/1×10⁹ cells. Accordingly, in one embodiment, the recombinant bacteria disclosed herein has a methionine degradation activity of about 1.45 μmol/hr/1×10⁹ cells. Accordingly, in one embodiment, the recombinant bacteria disclosed herein has a methionine degradation activity of about 1.50 μmol/hr/1×10⁹ cells.

Accordingly, in one embodiment, the recombinant bacteria disclosed herein has a methionine degradation activity of about 0.1 μmol/hr/1×10⁹ cells to about 1.5 μmol/hr/1×10⁹ cells. Accordingly, in one embodiment, the recombinant bacteria disclosed herein has a methionine degradation activity of about 0.2 μmol/hr/1×10⁹ cells to about 1.5 μmol/hr/1×10⁹ cells. Accordingly, in one embodiment, the recombinant bacteria disclosed herein has a methionine degradation activity of about 0.1 μmol/hr/1×10⁹ cells to about 1.4 μmol/hr/1×10⁹ cells. Accordingly, in one embodiment, the recombinant bacteria disclosed herein has a methionine degradation activity of about 0.4 μmol/hr/1×10⁹ cells to about 1.1 μmol/hr/1×10⁹ cells. Accordingly, in one embodiment, the recombinant bacteria disclosed herein has a methionine degradation activity of about 0.1 μmol/hr/1×10⁹ cells to about 1.0 μmol/hr/1×10⁹ cells. Accordingly, in one embodiment, the recombinant bacteria disclosed herein has a methionine degradation activity of about 0.5 μmol/hr/1×10⁹ cells to about 1.5 μmol/hr/1×10⁹ cells. Accordingly, in one embodiment, the recombinant bacteria disclosed herein has a methionine degradation activity of about 0.75 μmol/hr/1×10⁹ cells to about 1.25 μmol/hr/1×10⁹ cells.

In one embodiment, about 0.1 g to about 1.0 g of methionine are degraded per day. In one embodiment, about 0.01 to about 1.5 g of methionine are degraded per day. In one embodiment, about 0.1 g of methionine are degraded per day. In one embodiment, about 0.2 g of methionine are degraded per day. In one embodiment, about 0.3 g of methionine are degraded per day. In one embodiment, about 0.4 g of methionine are degraded per day. In one embodiment, about 0.5 g of methionine are degraded per day. In one embodiment, about 0.6 g of methionine are degraded per day. In one embodiment, about 0.7 g of methionine are degraded per day. In one embodiment, about 0.8 g of methionine are degraded per day. In one embodiment, about 0.9 g of methionine are degraded per day. In one embodiment, about 1.0 g of methionine are degraded per day. In one embodiment, about 1.1 g of methionine are degraded per day. In one embodiment, about 1.2 g of methionine are degraded per day. In one embodiment, about 1.3 g of methionine are degraded per day. In one embodiment, about 1.4 g of methionine are degraded per day. In one embodiment, about 1.5 g of methionine are degraded per day.

An important consideration in the selection of the one or more additional therapeutic agents is that the agent(s) should be compatible with the genetically engineered bacteria disclosed herein, e.g., the agent(s) must not kill the bacteria. In some embodiments, the pharmaceutical composition is administered with food. In alternate embodiments, the pharmaceutical composition is administered before or after eating food. The pharmaceutical composition may be administered in combination with one or more dietary modifications, e.g., low-protein diet or amino acid supplementation. The dosage of the pharmaceutical composition and the frequency of administration may be selected based on the severity of the symptoms and the progression of the disorder. The appropriate therapeutically effective dose and/or frequency of administration can be selected by a treating clinician.

### EXAMPLES

The present disclosure is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Example 1: Strain Development and Testing

All strains in this example utilize medium copy plasmids. These plasmids contain either Methionine gamma lyase (MGL) or Methionine decarboxylase (MDC) under the control of an anhydrotetracycline (ATC)-inducible promoter. Plasmids were constructed through TypeIIS cloning of synthesized gBlock fragments (IDT, Coralville, IA) containing these genes, followed by Sanger sequencing for sequence verification. Plasmids were used to transform E. coli Nissle (EcN). EcN strains harboring either MGL or MDC plasmids were grown to early log phase and induced for expression with 200 ng/mL ATC. Induction was allowed to proceed for 4 h, at which time cells were harvested by centrifugation and biomass stored in PBS containing 15% glycerol at −80° C. For testing of Methionine degradation activity, frozen biomass was thawed on ice and brought to an OD600=1 in M9 minimal media containing 0.5% glucose and 10 mM methionine and incubated at 37° C. statically. Supernatant samples were removed at 0, 30, 60, and 120 mins to determine the concentration of Methionine remaining over time.

For activated biomass, 2 mL cultures were grown overnight in LB media. Overnight cultures were back-diluted 1:100 in 10-20 mL fresh LB media in 50 mL baffled flasks and grown for 2 hours at 37° C. with shaking at 250 rpm. After 2 hours of growth, induction with 2× anhydrotetracycline (ATC) occurred, and cells were grown an additional four hours at 37° C. with shaking at 250 rpm. After 6 hours of total growth, bacterial cells were pelleted by centrifugation at 8000 rpm for five minutes. The supernatant was removed, cells were placed on ice, and cells were resuspended in PBS buffer. The cells were either frozen at −80° C. or the consumption assay was run.

For the methionine consumption assay, cells were thawed on ice and OD₆₀₀ was measured. The volume of cells equivalent to an OD of 1 were added to 1 mL of M9 minimal media containing 5% glucose in an 1.7 mL tube. The tube was vortexed briefly to evenly distribute the cells, and the tubes were placed at 37° C. with no shaking. 150 μL of cell/media suspension was removed at 0.5, 1.0, 1.5, 2.0 and 4.0 hour time points, spun at high speed for about 1 minute to pellet cells, and 100 μL was added to the well of a 96-well plate (avoiding pellet). The amount of L-methionine was measured using HPLC.

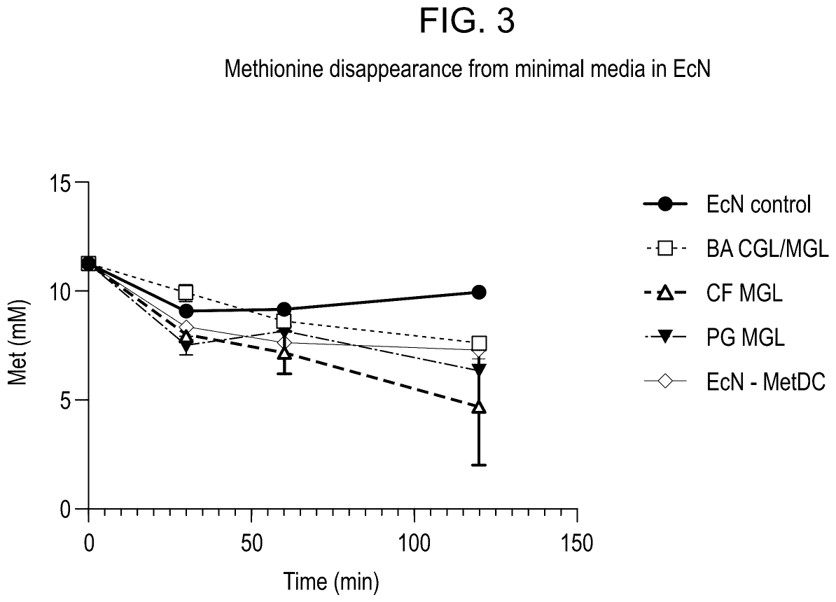

FIG. 3 is a graph depicting methionine disappearance from minimal media in E. coli Nissle harboring methionine gamma lyase (MGL) or methionine decarboxylase (MDC also referred to herein as MetDC) under the control of an anhydrotetracycline (ATC)-inducible promoter. EcN control is wild-type E. coli Nissle with no methionine catabolism enzymes. BA CGL/MGL is MGL from Brevibacterium aurantiacum (DOI 10.1124/jpet.119.256537). CF MGL is MGL from Citrobacter freundii. PG MGL is MGL from Poprhyromonas gingivalis. EcN-MetDC is MDC from Streptomyces sp. 590. These data demonstrate increased disappearance of methionine in the strains comprising a methionine catabolism enzyme as compared to EcN control.

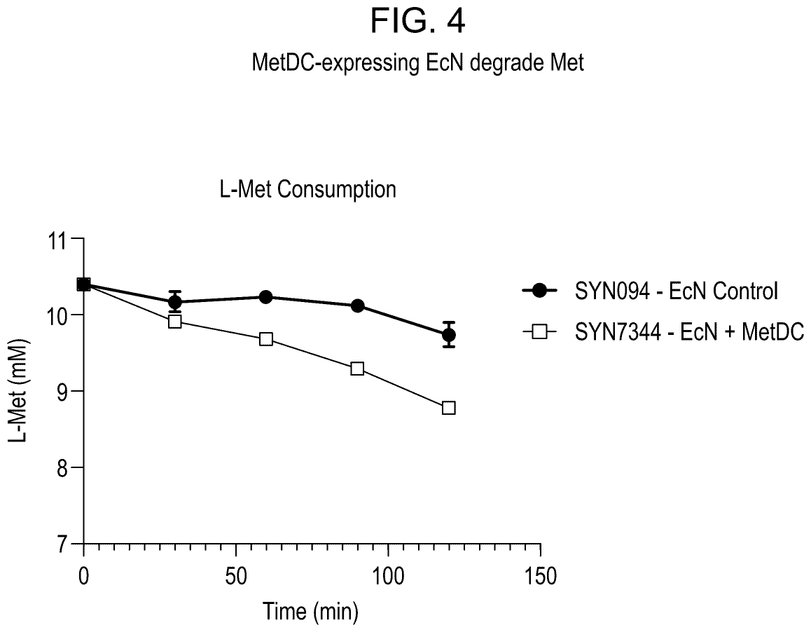

FIG. 4 is a graph depicting L-Met consumption over time. E. coli Nissle, SYN7344, containing a medium copy plasmid (p15A ori) encoding an anhydrotetracycline-inducible MetDC were grown in LB to early log phase followed by induction of MetDC expression for 4 hours. Activated cells were harvested and frozen in PBS buffer containing 15% glycerol at −80° C. On the day of testing, activated biomass was thawed and resuspended to an OD₆₀₀=1 in M9 minimal media containing 0.5% glucose and 10 mM Met. Supernatant samples were removed over 2 hours to quantify Met disappearance. These data demonstrate increased consumption of methionine in the E. coli Nissle strains comprising MetDC as compared to E. coli Nissle control strains.

The recombinant bacteria may be further modified by knocking out methionine exporters, such as yjeH, an efflux pump known to import methionine out of the cell. Such a knockout will increase the cytoplasmic concentration of methionine to assist in driving methionine degradation reactions. In addition, E. coli contains an ABC importer, encoded in the metNIQ operon, known to import methionine into the cell. This importer may also be expressed or over expressed to increase availability of methionine to the recombinant bacteria.

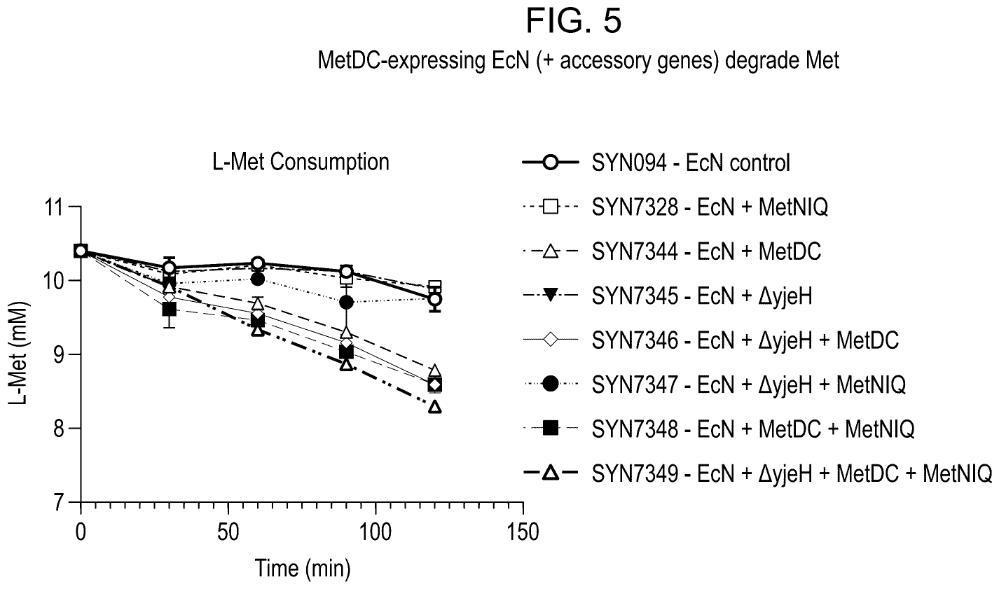

FIG. 5 is a graph depicting L-Met consumption over time. Strains SYN094 (control), SYN7328 (metNIQ (SEQ ID NOs: 1058, 1059, and 1060)), SYN7344 (SpmetDC (SEQ ID NO: 1049)), SYN7345 (ΔyjeH), SYN7346 (ΔyjeH, SpmetDC (SEQ ID NO: 1049), SYN7347 (ΔyjeH, metNIQ (SEQ ID NOs: 1058, 1059, and 1060)), SYN7348 (SpmetDC (SEQ ID NO: 1049), metNIQ (SEQ ID NOs: 1058, 1059, and 1060)), and SYN7349 (ΔyjeH, SpmetDC (SEQ ID NO: 1049), metNIQ (SEQ ID NOs: 1058, 1059, and 1060)).

The strains used are shown in Table 6 and results in Table 7. EcN containing a medium copy plasmid (p15A ori) encoding an anhydrotetracycline-inducible MetDC and/or a low copy plasmid (pSC101 ori) encoding an anhydrotetracycline-inducible MetNIQ were grown in LB to early log phase followed by induction of MetDC and/or MetNIQ expression for 4 hours. Activated cells were harvested and frozen in formulation buffer containing 15% glycerol at −80° C. On the day of testing, activated biomass was resuspended to an OD₆₀₀=1 in M9 minimal media containing 0.5% glucose and 10 mM Met. Supernatant samples were removed over 2 hours to quantify Met disappearance. Deletion of yjeH and/or addition of metNIQ show an additive effect when tested in combination with metDC.

Expression of the Met importer, metNIQ, increased Met consumption. Similarly, deletion of the Met exporter, yjeH, increased Met consumption. Combining expression of metNIQ and deletion of yjeH with the expression of MetDC lead to an additive effect and greater Met consumption. Increasing internal Met concentration by increasing uptake and decreasing release of Met surprisingly increases whole cell activity.

TABLE 6
E. coli Strains

| Strain | | Antibiotic |
|---|---|---|
| No. | Background/genotype | resistance |
| SYN7328 | SYN001 (WT EcN); Logic2375(pSC101; | carbeni- |
| | Ptet: metNIQ (SEQ ID NOs: 1058, | cillin |
| | 1059, and 1060) | (carb) |
| SYN7344 | SYN001; Logic2279(p15a; Ptet: metDC | kanamycin |
| | (SEQ ID NO: 1049)) | (kan) |
| SYN7345 | SYN001; ΔyjeH | chloram- |
| | | phenicol |
| | | (cam) |
| SYN7346 | SYN001; ΔyjeH; Logic2279(p15a; | cam, kan |
| | Ptet: metDC (SEQ ID NO: 1049)) | |
| SYN7347 | SYN001; ΔyjeH; Logic2375(pSC101; | cam, carb |
| | Ptet: metNIQ (SEQ ID NOs: 1058, | |
| | 1059, and 1060)) | |
| SYN7348 | SYN001; Logic2279(p15a; Ptet: metDC | kan, carb |
| | (SEQ ID NO: 1049)); Logic2375(pSC101; | |
| | Ptet: metNIQ (SEQ ID NOs: 1058, | |
| | 1059, and 1060)) | |
| SYN7349 | SYN001; ΔyjeH; Logic2279(p15a; Ptet: | cam, carb, |
| | metDC (SEQ ID NO: 1049)); | kan |
| | Logic2375(pSC101; Ptet: metNIQ (SEQ | |
| | ID NOs: 1058, 1059, and 1060)) | |

TABLE 7
Met Consumption

| Time (min) | SYN094 - EcN Control | | | SYN7346 - EcN + ΔyjeH + MetDC | | |
|---|---|---|---|---|---|---|
| 0 | 10.4384571 | 10.38598365 | 10.4006508 | 10.4384571 | 10.38598365 | 10.4006508 |
| 30 | 10.3239792 | 10.15822805 | 10.04925695 | 9.83632735 | 9.79290835 | 9.71764875 |
| 60 | 10.3105299 | 10.24432475 | 10.17411305 | 9.5354478 | 9.5360832 | 9.59974675 |
| 90 | 10.20976605 | 10.07170775 | 10.104272 | 9.27272755 | 9.1298508 | 9.08238995 |
| 120 | 9.66106285 | 9.9430569 | 9.6527497 | 8.5659333 | 8.59143755 | 8.62101895 |
| | SYN7328 - EcN + MetNIQ | | | SYN7347 - EcN + ΔyjeH + MetNIQ | | |
| 0 | 10.4384571 | 10.38598365 | 10.4006508 | 10.4384571 | 10.38598365 | 10.4006508 |
| 30 | 10.07675565 | 10.1295468 | 10.0716901 | 9.95274675 | 9.9415743 | 9.99951925 |
| 60 | 10.24053 | 10.15007375 | 10.2517907 | 10.0627592 | 9.9935712 | 10.0276357 |
| 90 | 10.1566219 | 10.0433089 | 9.916476 | 9.2044044 | 9.9672374 | 9.96630195 |
| 120 | 9.92157685 | 9.9778627 | 9.8607373 | 9.7831832 | 9.7370814 | 9.76205615 |
| | SYN7344 - EcN + MetDC | | | SYN7348 - EcN + MetDC + MetNIQ | | |
| 0 | 10.4384571 | 10.38598365 | 10.4006508 | 10.4384571 | 10.38598365 | 10.4006508 |
| 30 | 9.94551025 | 9.9600009 | 9.85775445 | 9.3271425 | 9.8031983 | 9.72354385 |
| 60 | 9.59423995 | 9.75471375 | 9.73166285 | 9.5032895 | 9.4215347 | 9.482286 |
| 90 | 9.33654995 | 9.2780402 | 9.3016559 | 9.0206679 | 9.0718176 | 9.02739255 |
| 120 | 8.725101 | 8.8207993 | 8.822176 | 8.4898971 | 8.58314205 | 8.7146875 |
| | SYN7345 - EcN + ΔyjeH | | | SYN7349 - EcN + ΔyjeH + MetDC + MetNIQ | | |
| 0 | 10.4384571 | 10.38598365 | 10.4006508 | 10.4384571 | 10.38598365 | 10.4006508 |
| 30 | 10.1282407 | 10.1477616 | 10.1049427 | 9.9559767 | 9.89644325 | 9.92164745 |
| 60 | 10.1581398 | 10.1230163 | 10.238765 | 9.3027855 | 9.3405565 | 9.37746265 |
| 90 | 10.1263698 | 10.15964005 | 10.1112967 | 8.77976305 | 8.8848688 | 8.9463261 |
| 120 | 9.856113 | 9.90039685 | 9.91575235 | 8.31878035 | 8.3108202 | 8.2659539 |

### Example 2: Strain Activity Calculation

Methionine abundance in natural sources of protein ranges from 1-2% (or 1-2 g/100 g protein intake). Assuming the average human subject needs to degrade about 1.0 g methionine per day with meals, and assuming the recombinant bacteria provides 3 hours of activity per dose, that leaves 3× doses per day at 5×10¹¹ dose and 1.0 g per day (0.33 g/dose). 0.33 g methionine/dose=2230 μmol methionine. 2230 μmol/3 hours/5×10¹¹ cells leads to 1.49 μmol/hr/1×10⁹ cells. The target dose is 5×10¹¹ live recombinant bacterial cells/mL.

For human subjects on a low protein diet eating 10 g protein/day, the subject needs to degrade about 0.1 g methionine per day with meals. Assuming the recombinant bacteria provides 3 hours of activity per dose, that leaves 3× doses per day at 5×10¹¹ dose and 0.1 g per day (0.033 g/dose). 0.033 g methionine/dose=223 μmol methionine. 223 μmol/3 hours/5×10¹¹ cells leads to 0.15 μmol/hr/1×10⁹ cells. The target dose is 5×10¹¹ live recombinant bacterial cells/mL.

The target performance is about 4.0 μmol Met degraded per hour per 1×10⁹ live cells. In one embodiment, the target performance is about 3.5 μmol Met degraded per hour per 1×10⁹ live cells. In one embodiment, the target performance is about 4.5 μmol Met degraded per hour per 1×10⁹ live cells. In one embodiment, the target performance is about 3.5 to about 4.5 μmol Met degraded per hour per 1×10⁹ live cells. In one embodiment, the target performance is about 3.75 to about 4.25 μmol Met degraded per hour per 1×10⁹ live cells. This is approximately five-fold what previous bacteria were able to degrade.

The Met degradation rate for the SYN7344 strain is 0.81 μmol/hr/1×10⁹ cells. The Met degradation rate for the SYN7346 strain is 0.91 μmol/hr/1×10⁹ cells. The Met degradation rate for the SYN7348 strain is 0.91 μmol/hr/1×10⁹ cells. The Met degradation rate for the SYN7349 strain is 1.05 μmol/hr/1×10⁹ cells.

### Example 3: Production and Formulation

Recombinant bacteria are cultured in LB media. 2 mL cultures were grown shaking overnight in 14 mL culture tubes. On the day of biomass preparation, 10 mL of fresh LB in a 50 mL baffled flask was inoculated with overnight culture at a 1:100 back-dilution. Cells were grown for 2 h at 37° C. in a shaking incubator (250 rpm). At 2 h, 200 ng/mL ATC was added for induction of recombinant genes. The induction phase was allowed to continue for 4 h. After induction, cells were spun down in a centrifuge at 5000×g for 10 min, and resuspended in PBS containing 15% glycerol and stored at −80° C. until the day of testing. The current formulation comprises biomass stored in PBS comprising 15% glycerol.

### Example 4: Design of In Vivo Study with Acute Mouse Model of Homocystinuria

An in vivo study was designed to evaluate the activity of the recombinant bacterial strains in a mouse model of acute hypermethionemia. Briefly, mice were fasted overnight and orally gavaged with a dose of 200 mg/kg of D4-Met (labeled methionine) the following day. Blood samples were taken at 20 minutes, 1, 2 and 5 hours and urine samples at 5 hours post administration. Baseline samples were collected prior to the administration of D4-Met. Mice were kept fasting throughout the study. Intestinal effluent samples were collected at the end of the study after euthanization.

Samples were analyzed using LC-MS/MS for primarily labelled and unlabeled Met and Homocysteine.

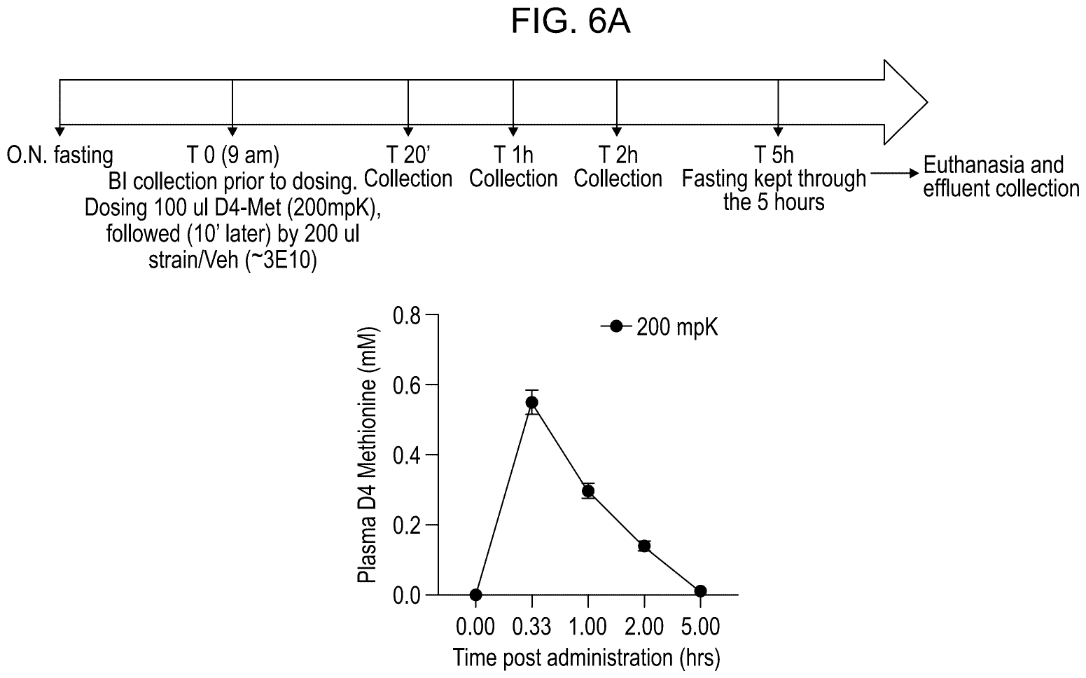

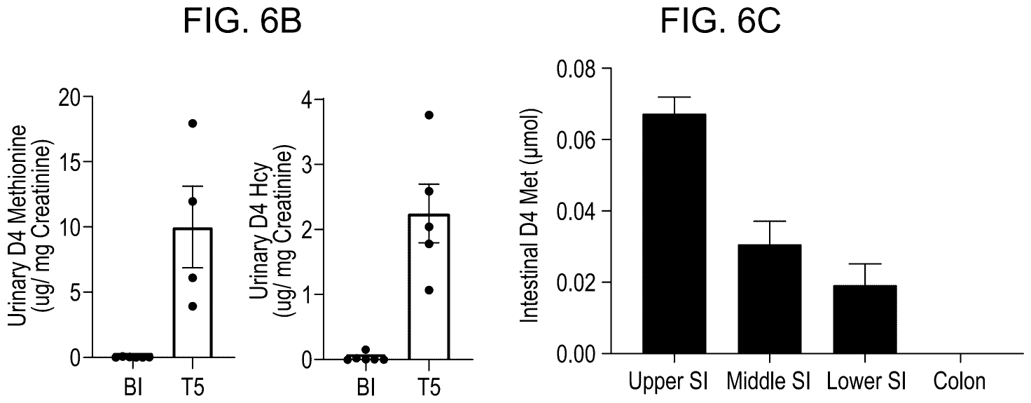

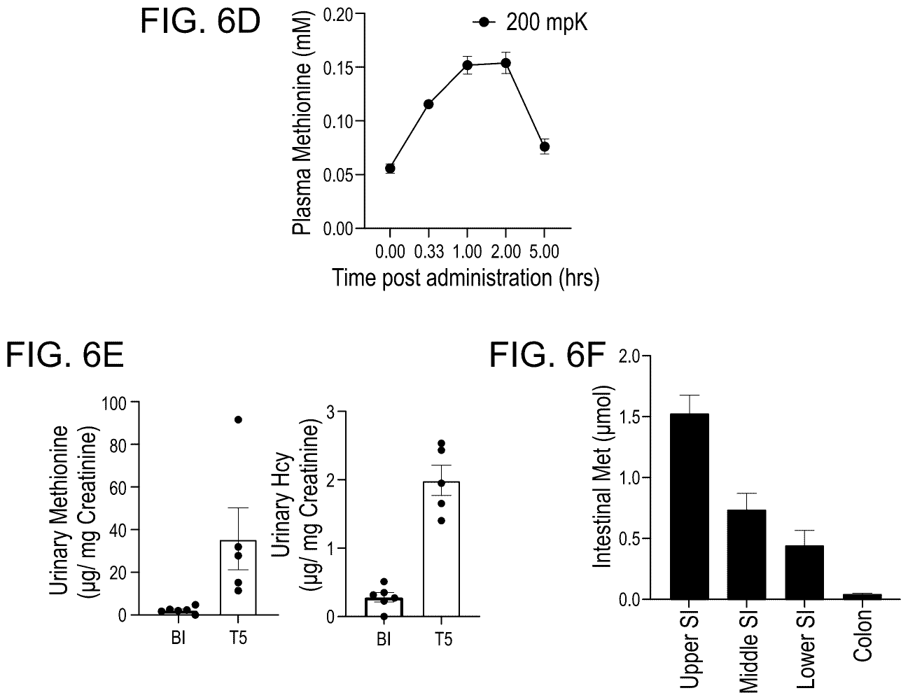

As shown in FIG. 6A, the plasma level of D4-Met reached a peak at about 20 minutes post administration, and the level dropped back to the baseline within 5 hours. A significant increase in D4-Met and D4-Hey urinary excretion was observed in mice at 5 hours post administration (FIG. 6B). The level of D4-Met in different gastrointestinal segments were also measured. As shown in FIG. 6C, the level of D4-Met in the gastrointestinal segments correlated with the expected absorption gradient of methionine, where the upper small intestine had the highest level of D4-Met, followed by the middle small intestine, the lower small intestine and the colon. Similar patterns were observed for the level of endogenous methionine in plasma, urine and intestinal effluent samples (FIGS. 6D-6F). However, the level of endogenous methionine in plasma samples reached a peak at about 2 hours post administration.

### Example 5. Evaluation of the Activity of Strain SYN7349 in HCU Mouse Model

The activity of the recombinant bacterial strain, SYN7349 (ΔyjeH; metDC (SEQ ID NO: 1049)); metNIQ (SEQ ID NOs: 1058, 1059, and 1060))), was evaluated in the previously described acute mouse model of HCU based on the oral administration of labeled methionine (D4-Met). Briefly, mice were fasted overnight prior to dose and administered orally using a flexible feeding tube attached to a sterile single use syringe with 100 pL of D4-Met (200 mg/kg) and 200 μl of the recombinant bacterial strains, SYN7349 and/or SYN094 (about 2.8×10¹⁰ live cells), or the glycerol/PBS vehicle. D4-Met was dosed 10 minutes after administration of the bacterial strains.

Urine samples were collected before dose and 5 hours post dose using free catch method. Gastrointestinal samples were collected at the end of the study after euthanization. Samples were flushed with PBS and effluents were collected from small intestines and colon were collected. The small intestine was divided into three equal sections and effluents from the sections were collected into separate tubes. All samples were kept on ice and stored on 96-well plates at −80° C. for quantification.

Samples were analyzed using LC-MS/MS for primarily labelled and unlabeled Met, Homocysteine and methylthiopropinoic acid (3MTP).

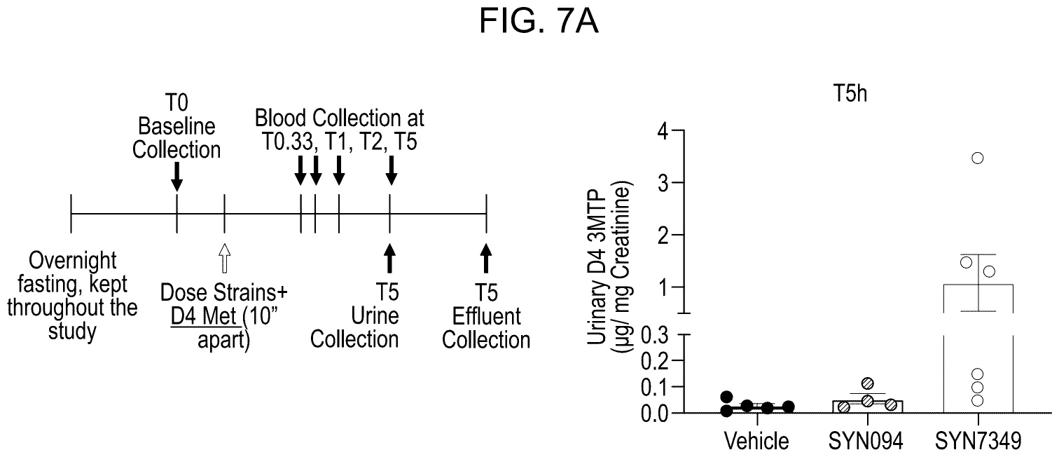

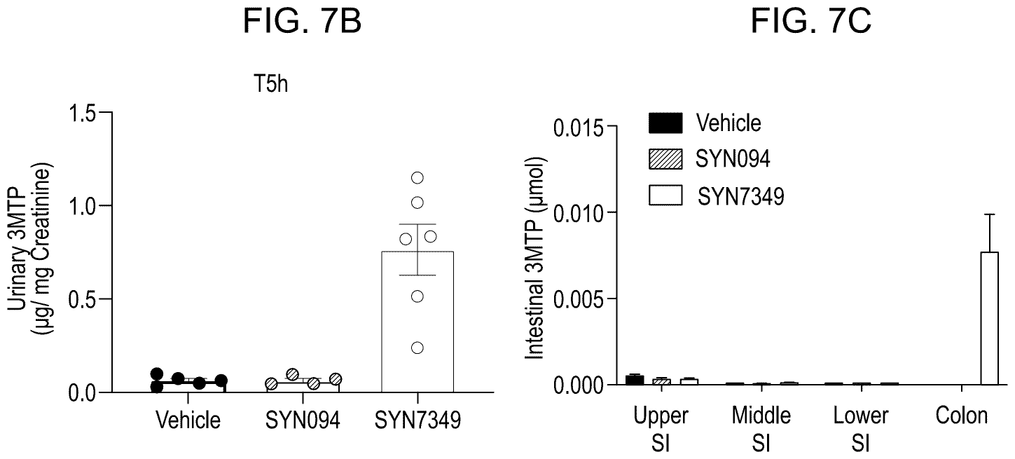

Mice receiving the SYN7349 strain excreted a significant higher level of D4-3-methylthiopropinoic acid (3MTP) in urine samples than mice receiving the SYN094 strain or the vehicle control (FIG. 7A). A similar pattern was shown for the elevated endogenous methionine, which is converted into 3MTP (FIG. 7B). In addition, a significant increase in the level of 3MTP in the colon sample of mice receiving the SYN7349 strain was also observed at 5 hours (FIG. 7C), suggesting that mice receiving the SYN7349 strain had a better capacity to consume methionine and excrete the decarboxylated product.

These data demonstrate that the SYN7349 strains are capable of consuming methionine in vivo and are promising therapeutic treatment for metabolic diseases involving dysregulation of methionine metabolism, such as homocystinuria.

### Example 6. Evaluation of Methionine Consumption and 3-MTP Production Activities of Engineered E. coli Strains

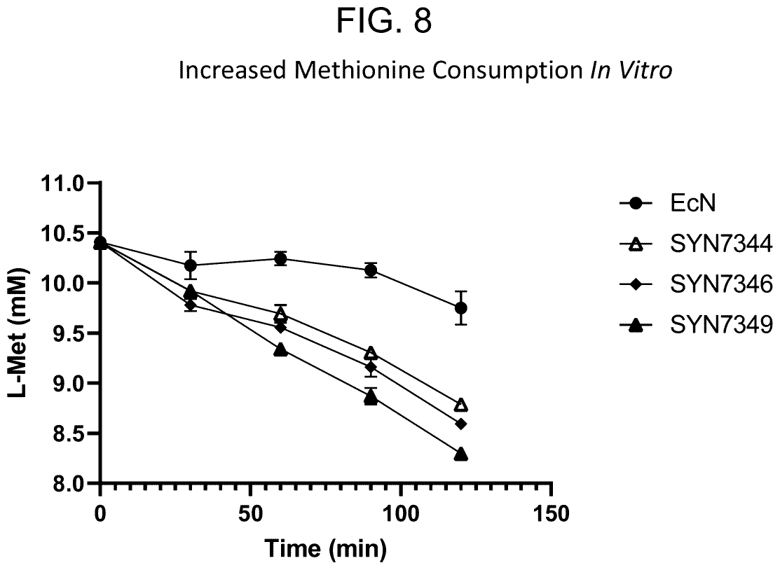

FIG. 8 is a graph depicting L-Met consumption over time. E. coli Nissle strains, SYN7349 (containing a medium copy plasmid (p15A ori) encoding an MetDC (Streptomyces sp. 590), a low copy plasmid (pSC101) encoding an endogenous methionine importer (metNIQ), and a knockout of yjeH) and SYN7346 (containing a medium copy plasmid (p15A ori) encoding an MetDC (Streptomyces sp. 590), and a knockout of yjeH), were grown in LB to early log phase followed by induction of MetDC expression for 4 hours. Activated cells were harvested and frozen in formulation buffer containing 15% glycerol at −80° C. On the day of testing, activated biomass was resuspended to an OD₆₀₀=1 in M9 minimal media containing 0.5% glucose and 10 mM Met. Supernatant samples were removed over 2 hours to quantify Met disappearance. These data demonstrate increased consumption of methionine in the E. coli Nissle strains comprising MetDC or MetDC/MetNIQ as compared to E. coli Nissle control strains (SYN094).

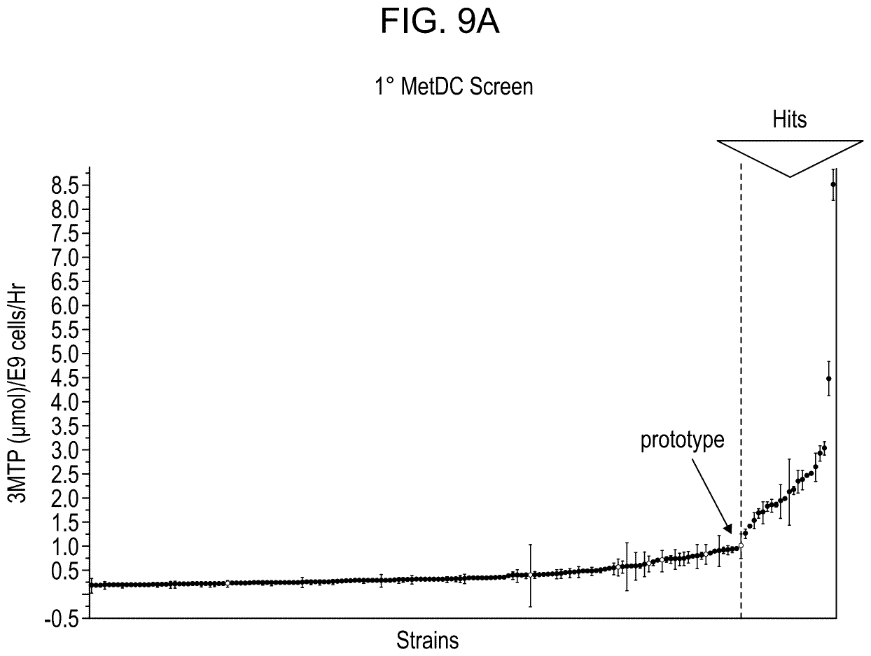

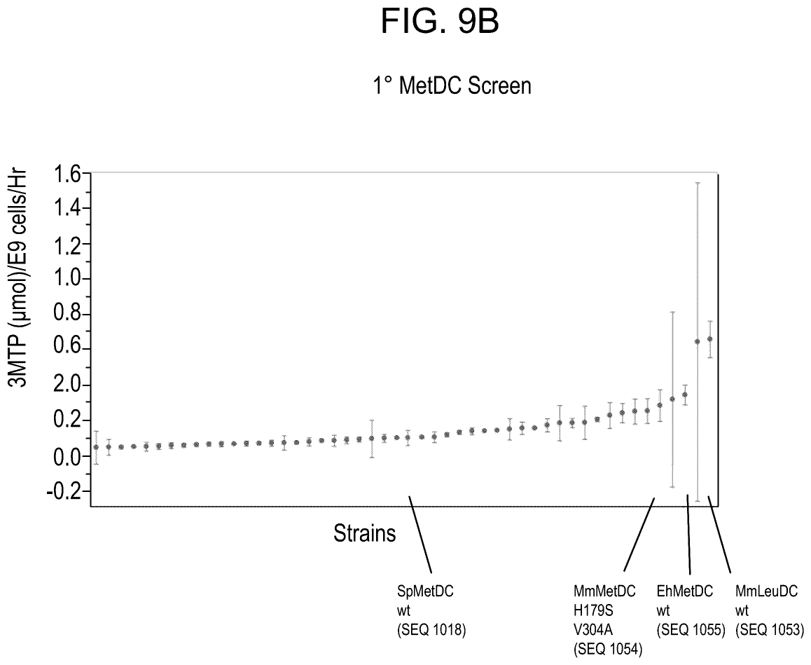

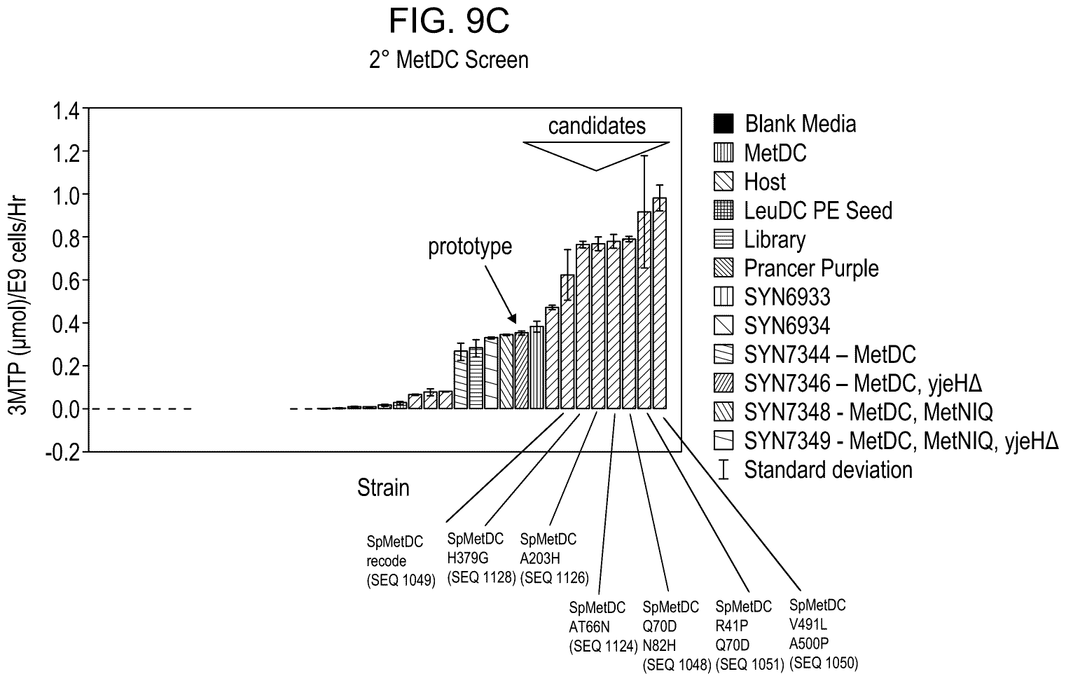

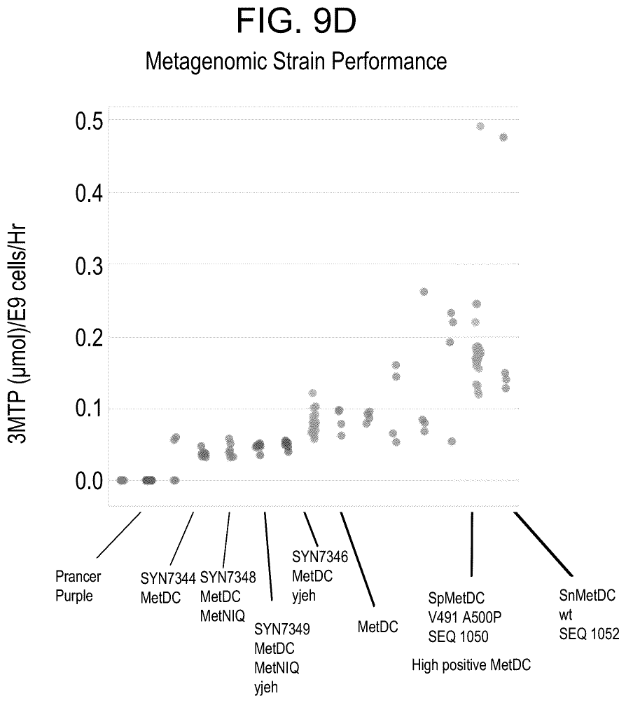

A metagenomic and a protein engineered library were screened for MetDC candidate enzymes having improved activity and for proteins that facilitate import of methionine. FIGS. 9A and 9C depict a primary screen (FIG. 9A) and a secondary screen (FIG. 9C) to identify optimal MetDC candidates. MetDC amino acid decarboxylases were expressed from plasmids in E. coli Nissle. These strains were incubated with L-methionine for a set period of time. Samples were taken at the endpoint and analyzed using LCMS for the presence of the decarboxylation product of methionine, 3-methylthiopropylamine (3-MTP), which was used as a measure of activity. Surprisingly, multiple MetDC enzymes identified in the screen showed a multiple fold increase in activity relative to baseline.

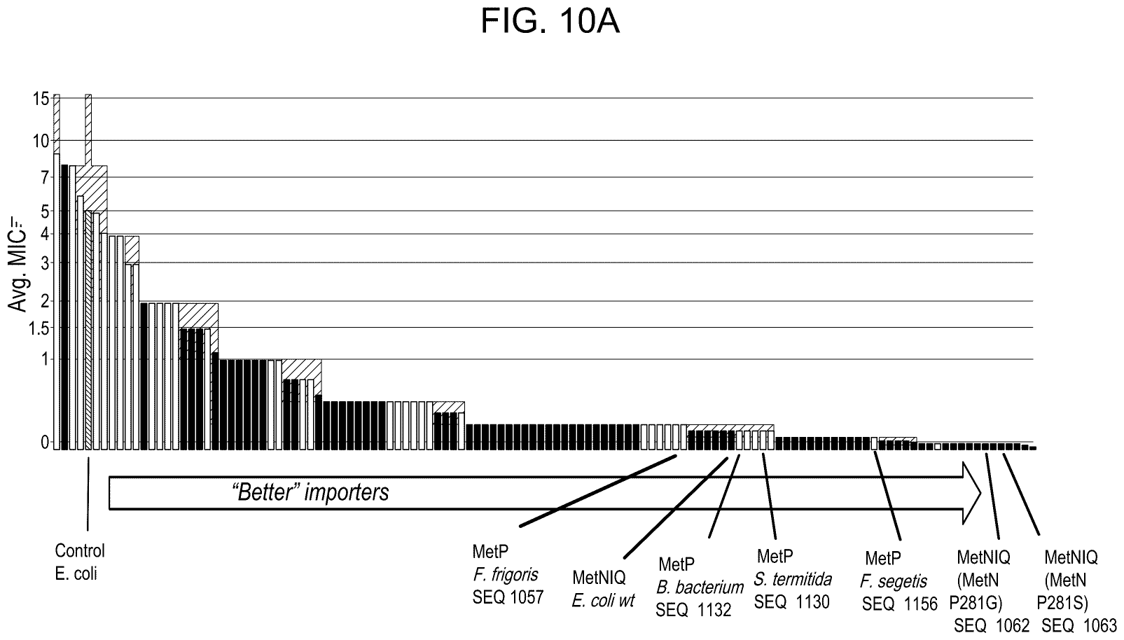

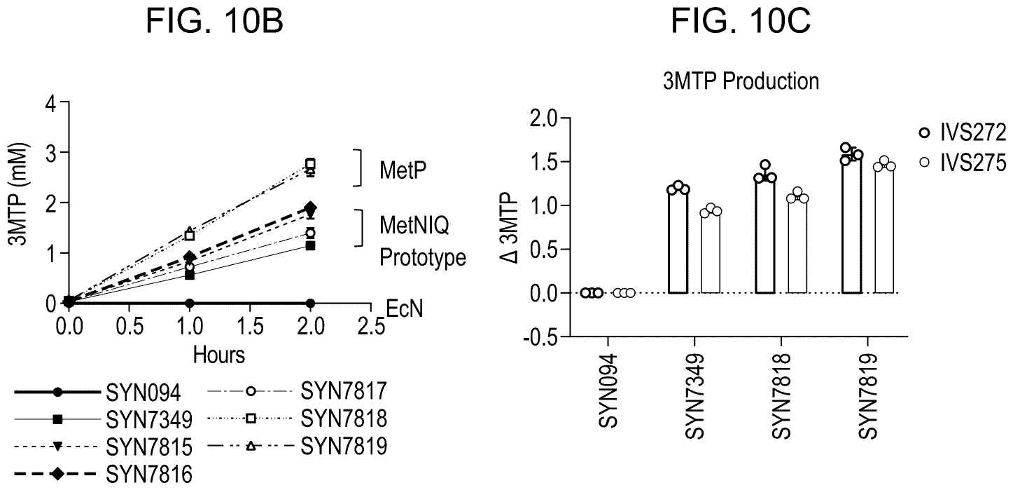

Screens were also conducted to identify optimal Met importer (MetP or MetNIQ) candidates are shown in FIG. 10A. MetP and MetNIQ amino acid importers were expressed from plasmids in WT E. coli Nissle. These strains were subsequently incubated with serial dilutions of norleucine, a toxic methionine analog, in liquid medium. These plates were used to calculate minimum inhibitory concentration of norleucine, under the hypothesis that lower MICs (higher sensitivity to toxin) would correspond to more active methionine importers.

FIG. 10B is a graph depicting 3-MTP production over time when methionine importers were added to MetDC expressing strains. Genes encoding MetP (SYN7818 or SYN7819) or MetNIQ (SYN7815, SYN7816, or SYN7817) were added to strain expressing MetDC (SYN7346). Both MetP and MetNIQ increased 3-MTP production in comparison to strain containing only MetDC. Assays were performed as described herein.

In vitro Simulation (IVS) assays were performed with SYN094 (control), SYN7349 (ΔyjeH, metDC, metNIQ), SYN7818 (ΔyjeH, metDC, metP((F. frigoris)), and SYN7819 (ΔyjeH, metDC, metP((F. segetis)) (FIG. 10C). 3-MTP production was increased in SYN7349, SYN7818, and SYN7819 compared to the control strain. Strains with added MetP genes (SYN7818 and SYN7819) showed an increase of 3-MTP production in comparison to SYN7349 with only MetDC. SYN7819 showed approximately a 2-fold increase of 3-MTP production when compared to SYN7349.

Briefly, frozen aliquots were thawed at room temperature, mixed, and placed on ice. Each strain was prepared in 1 mL aliquot at 5×10⁹ live cells/mL in 0.077 M sodium bicarbonate buffer, pH 7, and 400 uL were aliquoted into 3 wells in a 96-well plate. Samples were incubated in an Anoxic chamber set at 4% O₂. 400 uL of Simulated Intestinal Fluid (SIF), with 10 mM Methionine, was added to each well. The plate was incubated for 2 hours at 37° C. with shaking with a breathable plate seal. 100 uL samples were collected at time points 0, 30, 60, 120, and 180 minutes. Each sample was centrifuged at 400 rpm for 5 mins and 90 uL supernatant was collected. The supernatants were stored at −80° C. until LC-MS/MS analysis.

TABLE 8
SIF Experimental Mixture

| | | Volume for |
|---|---|---|
| | | One Sample |
| Component | Concentration | [uL] |
| Simulated Gastric Chyme | 1× | 500 |
| Simulated Intestinal Fluid | 1.25× | 275 |
| Pancreatin Solution | 800 Trypsin U/ | 125 |
| | mL in SIF | |
| Bile Salts | 160 mM in SIF | 62.5 |
| CaCl2 Stock Solution | 0.3M | 1 |
| HCl Stock Solution | 1M | 7.5 |
| Water | | 29 |
| | Total Volume | 1000 |

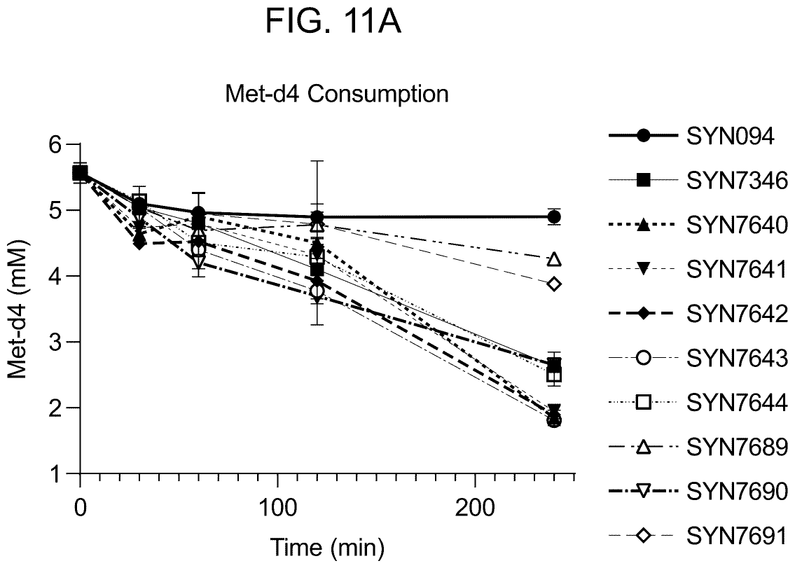

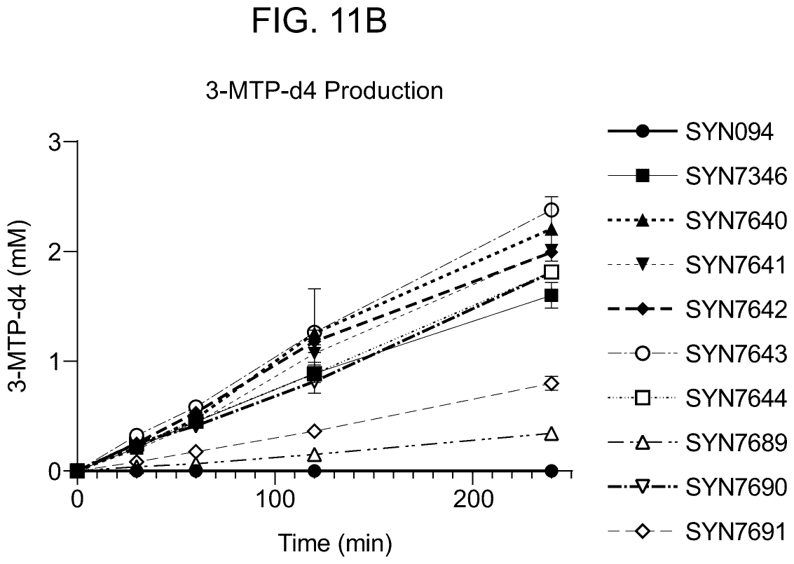

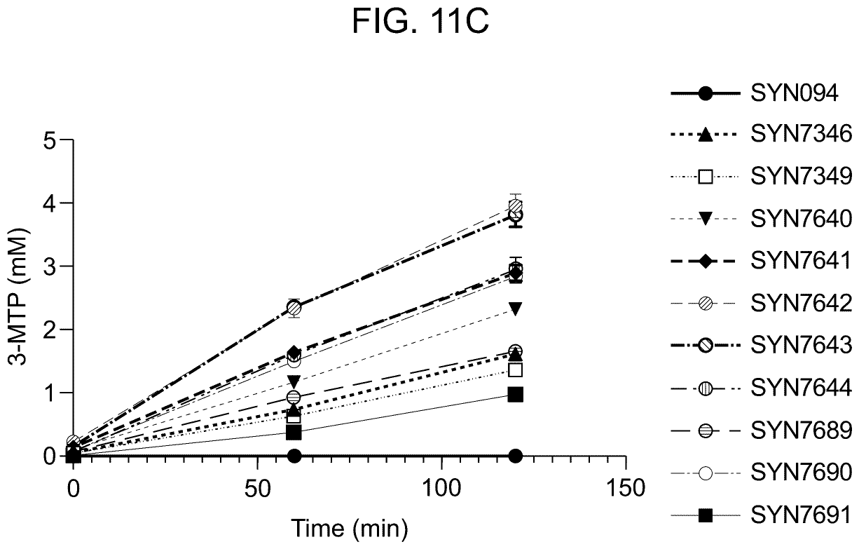

FIG. 11A is a graph depicting Met-d4 consumption and FIG. 11B is a graph depicting 3-MTP-d4 production by strains containing the identified MetDC proteins from the MetDC screen. SYN094 (control), SYN7346 (ΔyjeH, metDC SEQ ID NO: 1049), SYN7640 (metDC (SEQ ID NO: 1049), ΔyjeH), SYN7641 (metDC (V491L A500P; SEQ ID NO: 1050; engineered library), ΔyjeH), SYN7642 (metDC (Q70D N82H; SEQ ID NO: 1048; engineered library), ΔyjeH), SYN7643 (metDC (R41Q Q70D; SEQ ID NO: 1051; engineered library), ΔyjeH), SYN7644 (metDC (Stanieria sp. NIES-3757; metagenomic library), ΔyjeH), SYN7689 (engineered metDC (Mus musculus; SEQ ID NO: 1054; metagenomic library), ΔyjeH), SYN7690 (engineered leuDC (Mus musculus; SEQ ID NO: 1053; metagenomic library), ΔyjeH), and SYN7691 (metDC (Entamoeba histolytica; SEQ ID NO: 1055; metagenomics library), ΔyjeH). MetDC (Q70D N82H) from Streptomyces sp. 590 (SYN7642) showed both increased Met-d4 consumption and 3-MTP-d4 production when compared to the control strain and/or prototype strain.

### Example 7. Gene Integration into E. coli Nissle Genome

Genes encoding MetP and MetDC were integrated to facilitate methionine import and metabolism. The importer metP is derived from Flavobacterium segetis and facilitates the uptake of methionine into the cell. MetDC is derived from Streptomyces sp. 590 and includes two modifications (Q70D and N82H) to improve its activity at converting methionine to 3-MTP and CO₂. Both genes are under the regulatory control of a chemically inducible promoter (Ptac), which is induced by IPTG. To prevent release of methionine from the bacteria in the GI tract once it enters the cell, the yjeH gene that encodes a methionine/branched chain amino acid exporter was deleted. To control growth in vivo and in the environment, strains were engineered to be an auxotrophic strain through deletion of the dapA gene that encodes for dihydrodipicolinate synthase, which is essential for the cell wall. This deletion renders SYNB1353 unable to synthesize DAP, thereby preventing the proper formation of bacterial cell wall unless the strain is supplemented with DAP exogenously.

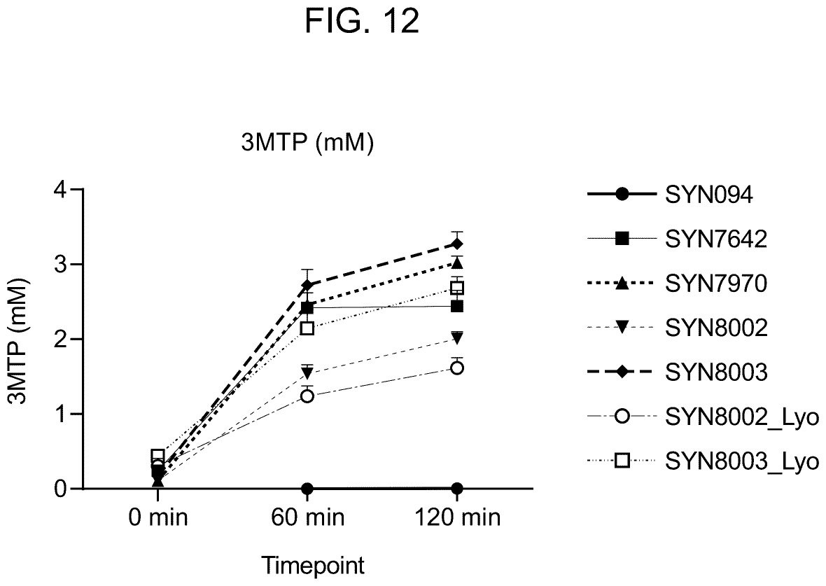

IVS assays for 3-MTP production by SYN094 (control), SYN7642 (metDC (Q70D N82H; SEQ ID NO: 1048; engineered library), SYN7970 (2 copies metDC (Q70D N82H; SEQ ID NO: 1048; engineered library), metP (metagenomics library; F. segetis; SEQ ID NO: 1056), ΔyjeH, Δdap, ΔΦ), SYN8002 (3 copies metDC (Q70D N82H; SEQ ID NO: 1048; engineered library), metP (metagenomics library; F. segetis; SEQ ID NO: 1056), ΔyjeH, Δdap, ΔΦ), and SYN8003 (3 copies metDC (Q70D N82H; SEQ ID NO: 1048; engineered library), metP (metagenomics library; F. segetis; SEQ ID NO: 1056), ΔyjeH, Δdap, ΔΦ) showed 3-MTP production (FIG. 12). SYN8003 has three copies of MetDC and one copy of MetP and was shown produce approximately 3-fold more 3-MTP than the control strain. Lyophilization of SYN8003 decreased 3-MTP production by approximately by 1.2-fold. (Note: SYN8002 differs from SYN8003 by the RBS for MetDC. SYN8003 has a stronger RBS).

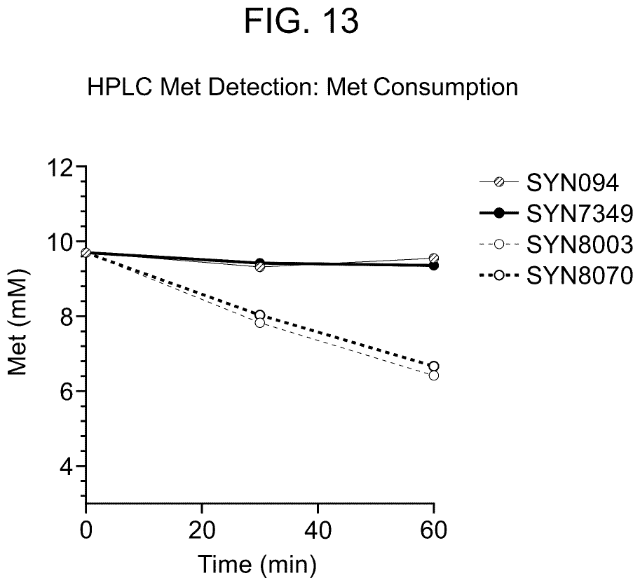

The pks island (aka colibactin island or clb island), and an endogenous Nissle prophage gene, have also been deleted from the genome. FIG. 13 depicts Met consumption by E. coli strains: SYN094 (control), SYN7349 (ΔyjeH, metDC (SEQ ID NO: 1049), metNIQ (endogenous)), SYN8003 (3 copies metDC (Q70D N82H; SEQ ID NO: 1048; engineered library), metP (metagenomics library; F. segetis; SEQ ID NO: 1056), ΔyjeH, Δdap, ΔΦ), and SYN8070 (3 copies metDC (Q70D N82H; SEQ ID NO: 1048; engineered library), metP (metagenomics library; F. segetis; SEQ ID NO: 1056), ΔyjeH, Δdap, ΔΦ, Δpks). Both SYN8003 and SYN8070 (aka SYNB1353) showed approximately 1.4 fold to about 1.5-fold decrease of methionine compared to the control strain (SYN094). Deletion of the endogenous clb (colibactin) island (also referred to as pks island) in SYN8070 did not impact methionine consumption in comparison to SYN8003 with an intact clb gene. Strain names SYN8070 and SYNB1353 are used interchangeably herein. Additionally, SYNB1353 was designed as a DAP auxotroph strain by deleting the dapA gene that encodes 4-hydroxy-tetrahydropicolinate synthase, which is essential for bacterial growth. This deletion renders SYNB1353 unable to synthesize DAP, thereby preventing the proper formation of bacterial cell wall unless the strain is supplemented with DAP exogenously.

There are no antibiotic resistance genes in SYNB1353, and whole genome sequencing (PacBio) confirmed that all insertions and deletions in SYNB1353 were created in the proper chromosomal locations and contained the expected sequence identities. Plating on selective media confirmed that SYNB1353 did not grow in the presence of any of the antibiotics used during strain construction and did not grow without exogenously supplied diaminopimelic acid.

### Example 8. Analysis of Methionine Degradation and 3-MTP Production with SYNB1353 In Vitro

SYNB1353 comprises a metP gene, metDC gene, and deletion of the yjeH gene, as shown in FIG. 14A. The ability of SYNB1353 to degrade methionine to 3-MTP and CO₂ by its engineered pathway was measured.

SYNB1353 and SYN094 were grown and activated in a bioreactor following optimized processes intended to be used for the scale-up of drug product. Activated cell batches were resuspended to the specified live cell count in assay media, and cells were statically incubated at 37° C. Supernatants were collected at defined timepoints, and the quantity of each analyte (methionine and 3-MTP) in each sample was determined by liquid chromatography mass spectrometry (LC-MS/MS). As observed in FIG. 14B, SYNB1353 degraded methionine and produced 3-MTP de novo, as designed. The control strain, SYN094, consumed methionine at a low rate and did not produce any 3-MTP.

In vitro Met consumption assays, as described above, show consumption of methionine and production of 3-MTP by SYNB1353 and not the EcN control (FIG. 14B). In vitro, SYNB1353 consumed methionine at a rate of 1.3±0.13 μmol/h/1×10⁹ live cells and concomitantly produced 3-MTP at a rate of 1.3±0.087 μmol/h/1×10⁹ live cells.

### Example 9. Dose-Response of SYNB1353 in Healthy Mice Receiving a Bolus of D4-Methionine

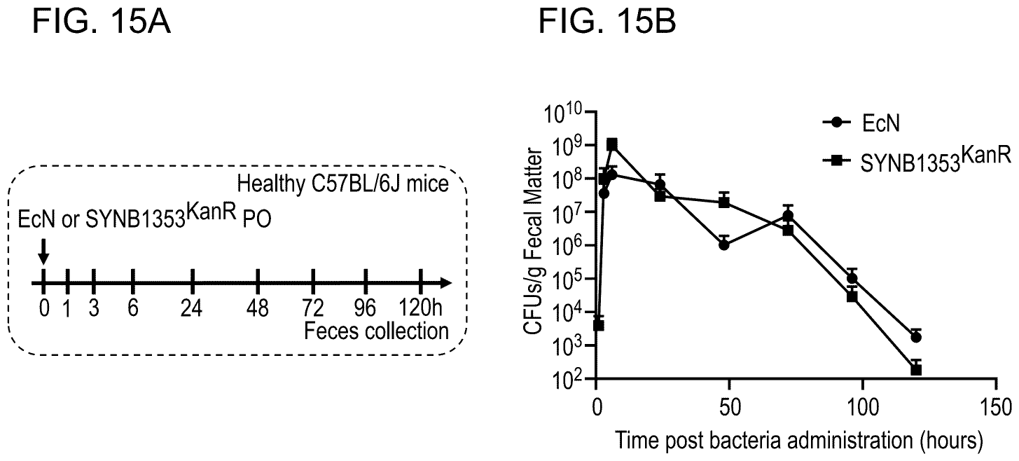

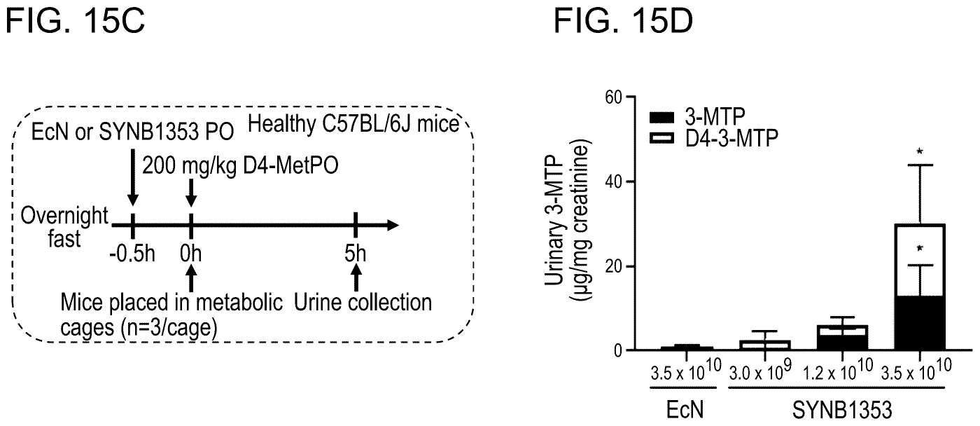

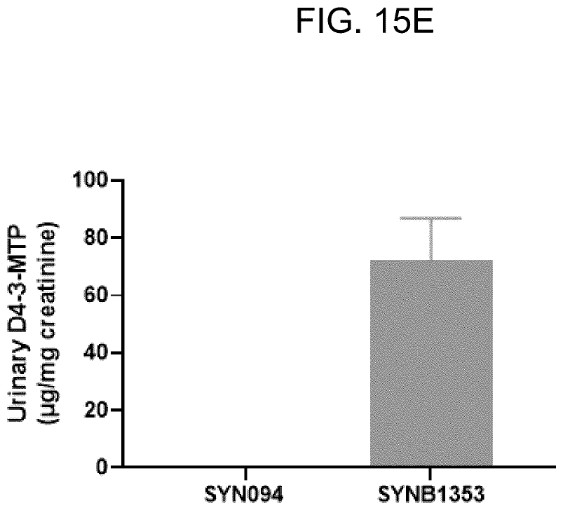

The ability of SYNB1353 to metabolize dietary and gastrointestinal methionine to produce 3-MTP in healthy mice was assessed. Two identical studies were performed, and the data from both studies were combined and shown in FIG. 15D. For each study, male C57BL/6J mice of approximately 8 weeks of age were acclimated for at least 4 days before being placed on study. Mice were fasted overnight and orally administered a single dose of SYN094 (3.5×10\hat{ }10 live cells, n=18/group) or SYNB1353 (3.0×10\hat{ }9, 1.0×10\hat{ }10, 3.5×10\hat{ }10 live cells, n=18/group). Thirty (30) minutes later, mice received a bolus of 200 mg of D4-methionine (PO) and were immediately placed in metabolic cages (3 per cage) without access to food for a total of 5 hours. Cumulative urine was collected for 3-MTP and creatinine measurements.

The effects of SYNB1353 on urinary recovery of 3-MTP and D4-3-MTP are shown in FIG. 15D. Urinary 3-MTP, D4-3-MTP, and creatinine were quantified by LC-MS/MS, and the ratio of metabolites to creatinine determined. Overall, the urinary recovery of 3-MTP in healthy mice was low but detectable, and only mice receiving SYNB1353 at 3.5×10\hat{ }10 live cells registered 3-MTP concentrations above the lower limit of quantitation (LLOQ) of the assay (0.16 μg/mL). SYNB1353 dose-dependently increased the recovery of both 3-MTP and D4-3-MTP, indicating conversion of both endogenous and orally administered methionine by SYNB1353 in the gut. At the highest dose tested, SYNB1353 resulted in a statistically significant 25- and 61-fold increase in urinary recovery of 3-MTP and D4-3-MTP, respectively, as compared to SYN094.

In conclusion, this study indicates that SYNB1353 can dose-dependently convert both endogenous and dietary methionine into 3-MTP.

### Example 10. Development of an Acute Model of Homocystinuria in Healthy Nonhuman Primates

The objective of this study was to develop an acute model of homocystinuria in nonhuman primates. Male cynomolgus monkeys of approximately 2-5 years of age (average weight of 3.4 kg) were fasted overnight and orally administered a methionine load at 100 or 300 mg/kg, and plasma was collected at 0-, 0.5-, 1-, 2-, 4-, 6-, and 24-hours post-dose for methionine and total homocysteine measurements by LC-MS/MS.

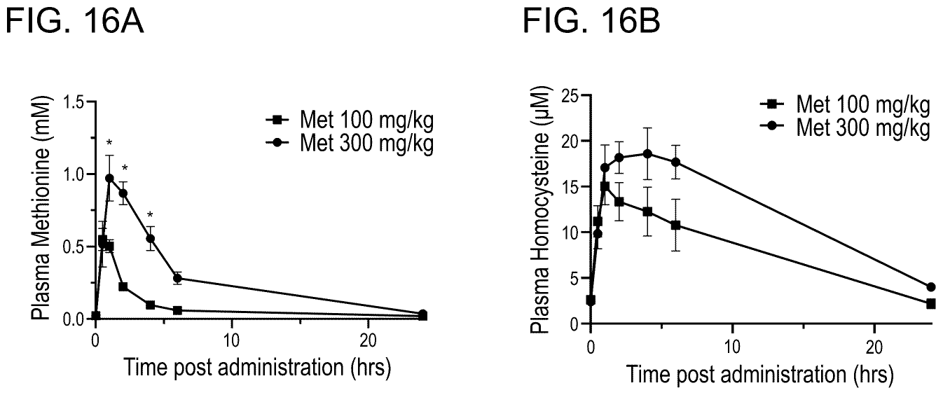

Oral administration of methionine (100 or 300 mg/kg) resulted in a dose-dependent increase in plasma methionine levels, with peak concentration recorded at 30 minutes and 1 hour post dose for 100 mg/kg and 300 mg/kg, respectively (FIG. 16A). Plasma methionine concentrations gradually decreased over time and reached pre-dose levels by 24 hours. The oral methionine load also resulted in a significant elevation in total plasma homocysteine by 30 minutes post dose, but no statistically significant difference between groups was noted (FIG. 16B). By 24 hours, total homocysteine levels had returned to baseline values for both groups. In conclusion, this study indicates that oral administration of a methionine load to nonhuman primates leads to acute homocystinuria.

### Example 11. Activity of SYNB1353 on Plasma Methionine and Plasma Homocysteine Levels in Nonhuman Primates Receiving Different Methionine Loads

The objective of this study was to assess the ability of SYNB1353 to metabolize methionine in an acute model of homocystinuria in nonhuman primates. SYNB1353 activity was assessed following a single dose of 1×10\hat{ }12 live cells compared to vehicle in order to determine the impact of the combined EcN chassis and methionine engineering in this model.

Male cynomolgus monkeys were fasted overnight and orally administered a methionine load (100 or 300 mg/kg) followed by sodium bicarbonate (1.8 mmol), and formulation buffer (vehicle), or SYNB1353 at 1×10\hat{ }12 live cells. Plasma was collected at 0, 0.5, 1, 2, 4, and 6-hours post-dose for methionine and total homocysteine measurements and cumulative urine was collected at 6 hours for 3-MTP recovery (and normalized to creatinine levels to account for differences in urinary volumes). Metabolites were measured using LC-MS/MS.

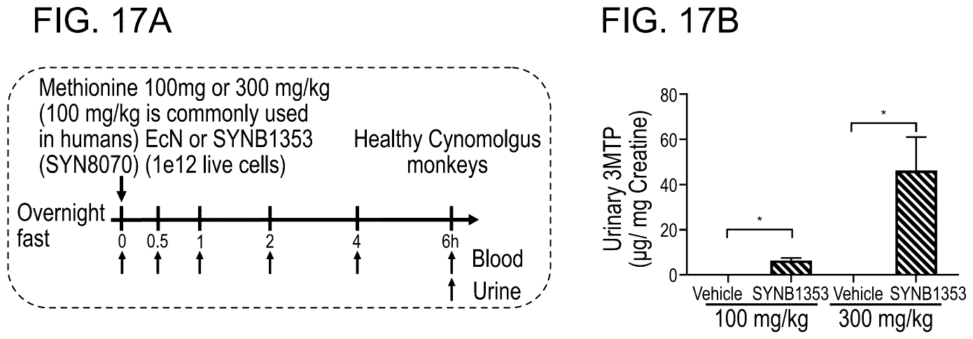

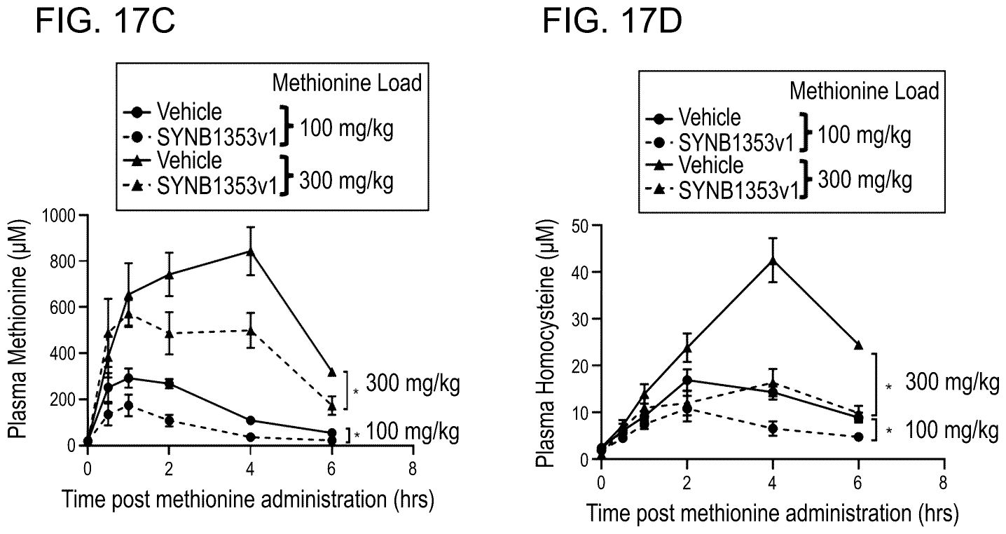

Administration of SYNB1353 resulted in a significant and treatment-related elevation in urinary 3-MTP levels as compared to vehicle, suggesting conversion of methionine by the strain in the gut (FIG. 17B). As shown in FIG. 17C and FIG. 17D, SYNB1353 significantly blunted the plasma appearance of methionine and total homocysteine at both methionine loads, with a 42% and 55% reduction in plasma total homocysteine area under the curve (AUC) compared to vehicle. In conclusion, these data indicate that SYNB1353 is capable of consuming methionine in the gut of nonhuman primates with acute homocystinuria.

### Example 12. Dose-Response of SYNB1353 in Nonhuman Primates Receiving a Methionine Load

The objective of this study was to evaluate the dose-response of SYNB1353 and assess the specific effect of methionine engineering against a control strain (SYN094) in an acute model of homocystinuria in nonhuman primates.

Male cynomolgus monkeys were fasted overnight and orally administered a methionine load (100 mg/kg) followed by sodium bicarbonate (1.8 mmol), and formulation buffer (vehicle), SYN094, or SYNB1353 at 5×10\hat{ }11 or 1×10\hat{ }12 live cells. Two sets of 3 studies were conducted in a cross-over manner so that all 12 colony animals received each treatment. Plasma was collected at 0-, 0.5-, 1-, 2-, 4-, and 6-hours post-dose for methionine and total homocysteine measurements and cumulative urine was collected at 6 hours for 3-MTP recovery (and normalized to creatinine levels to account for differences in urinary volumes). Each set of 3 studies was normalized to its respective vehicle for data representation.

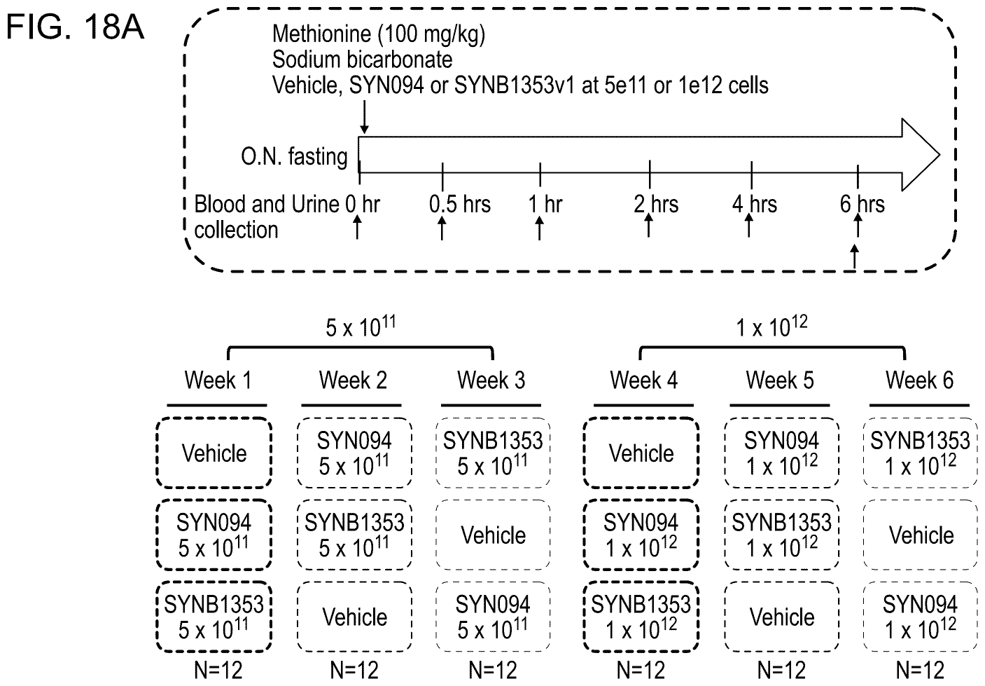

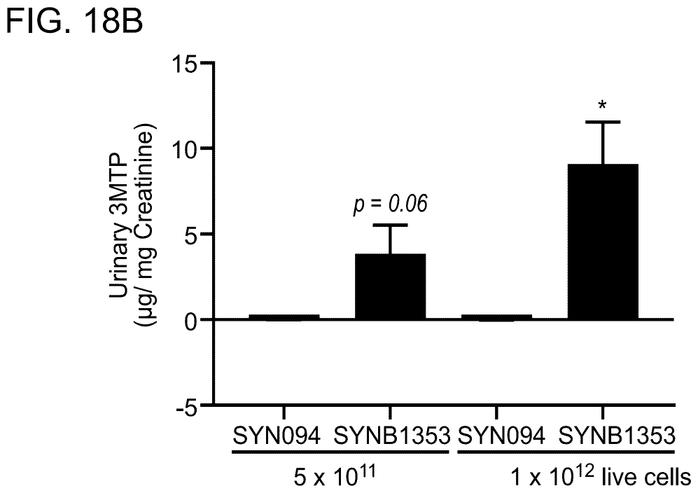

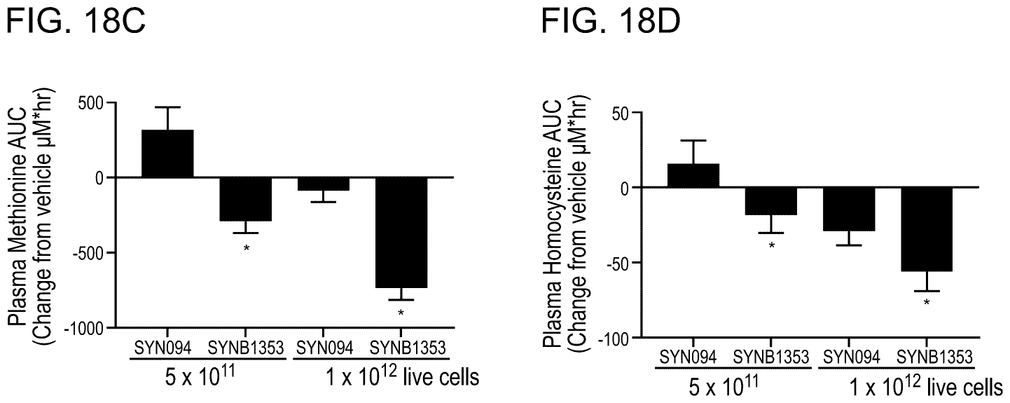

Administration of SYNB1353 resulted in a dose-dependent elevation in urinary 3-MTP levels as compared to SYN094, suggesting that the methionine-metabolizing pathway engineered in SYNB1353, and not the bacterial chassis itself, was responsible for methionine metabolism in the gut (FIG. 18B). As shown in FIG. 18C and FIG. 18D, SYNB1353 dose-dependently blunted the plasma appearance of methionine and total homocysteine in this model as compared to SYN094. In conclusion, these data indicate that the engineering of SYNB1353 is responsible for methionine consumption in the gut of nonhuman primates with acute homocystinuria and results in a dose-dependent blunting of plasma total homocysteine appearance in the blood.

### Example 13. In Vivo Evaluation of Methionine Consumption of Engineered E. coli Strains after Systemic Administration of Methionine

The objectives of this study were to determine (1) whether enterorecirculation of methionine occurs, and (2) whether orally-administered SYNB1353 can consume peripherally administered (IP) labeled methionine in mice.

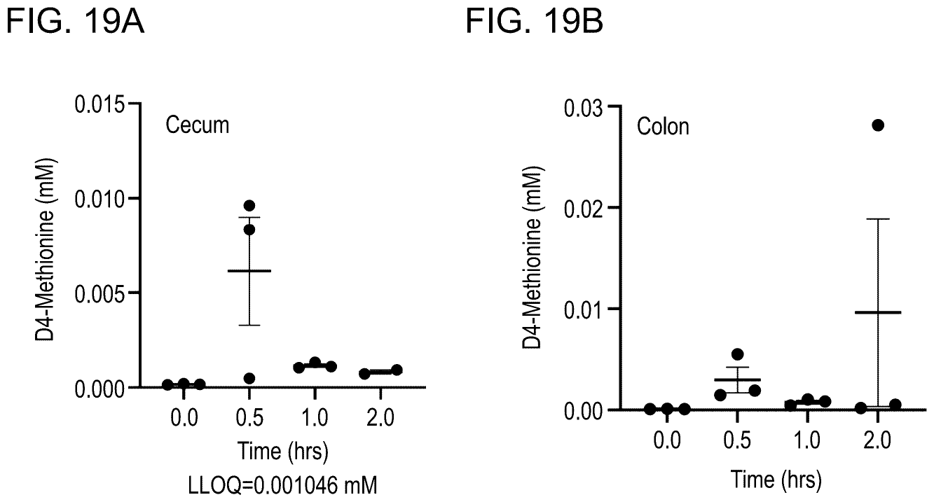

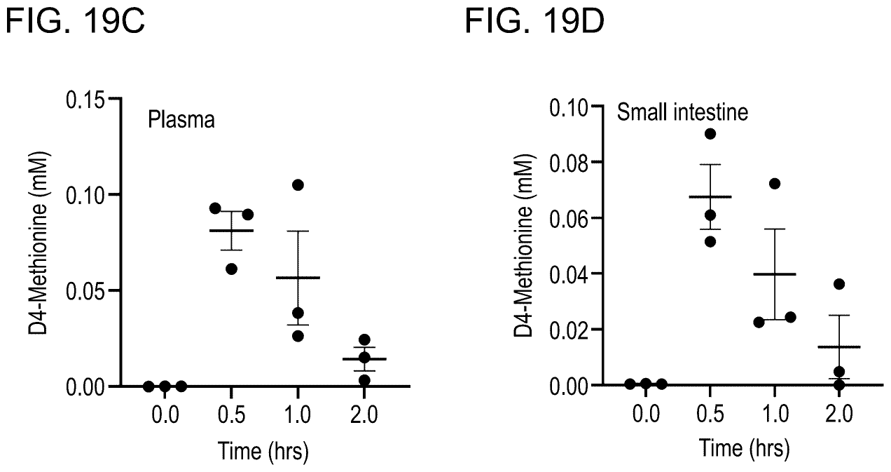

In a first study, healthy male C57BL/6 mice (n=3/group) were fasted overnight and received a single IP dose of D4-methionine (100 mg/kg). Blood and gut effluents (SI, cecum or colon) were collected at 0, 0.5, 1, or 2 hours post dosing for D4-methionine measurements. Results shown in FIGS. 19A-19D indicate that there is enterorecirculation of methionine from the plasma into the gut.

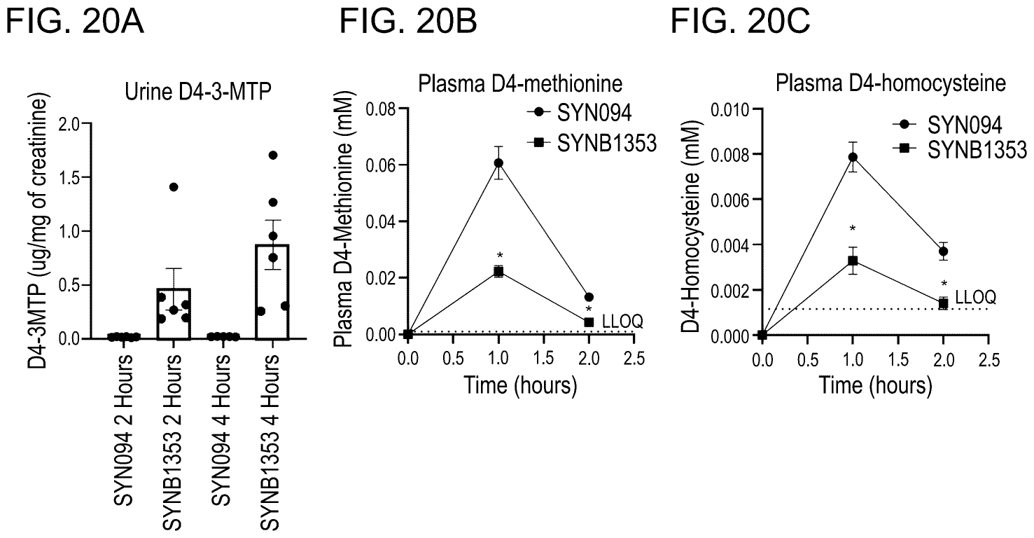

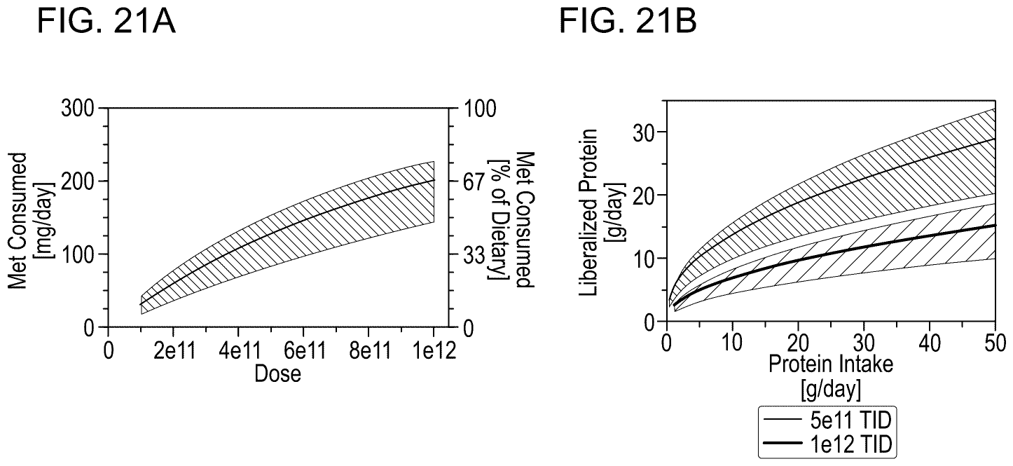

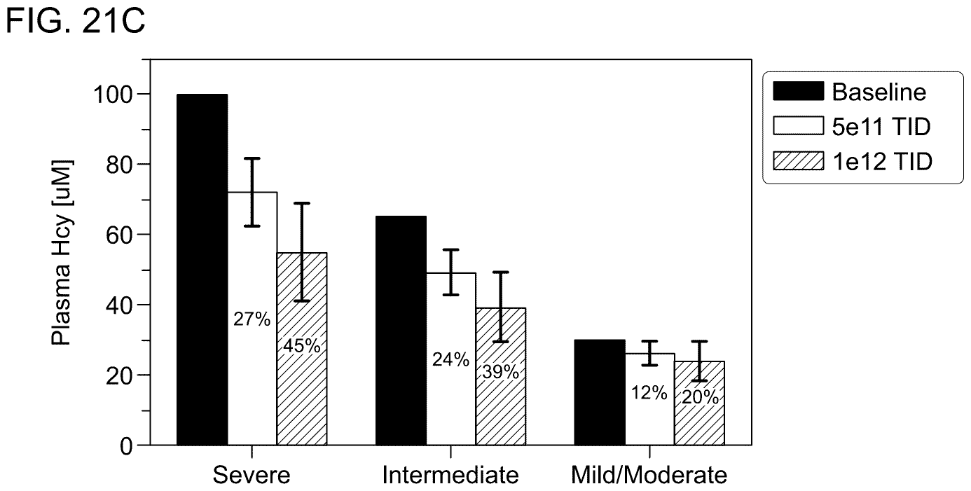

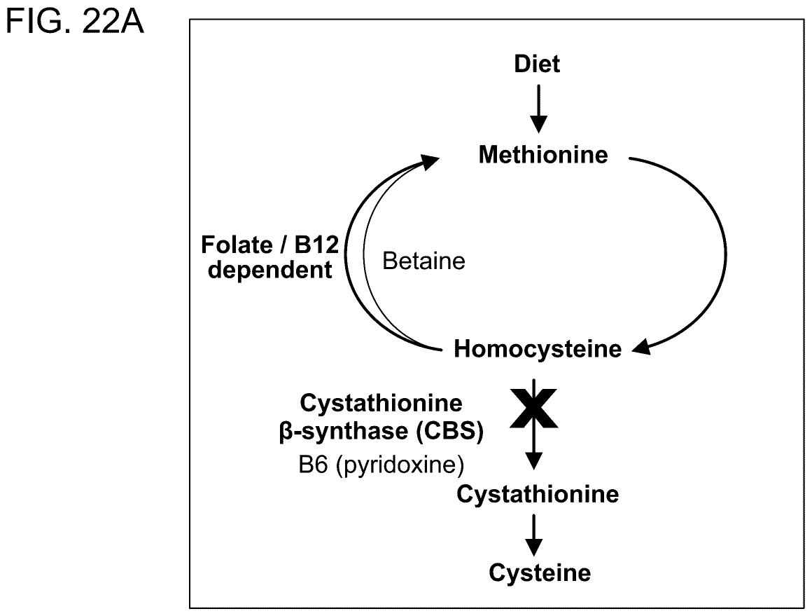

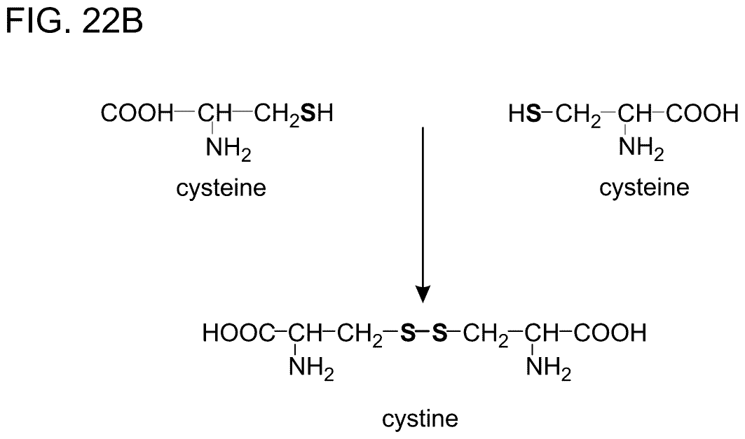

In a second study, healthy male C57BL/6 mice (n=10-18/group) were fasted overnight and received a single IP dose of D4-Met (100 mg/kg) followed by 2 doses of SYNB1353 PO 0.5 and 1.5 hours later. Blood and urine were collected for D4-Met, D4-tHcy and D4-3-MTP measurements. Results are shown in FIGS. 20A-20C and illustrate that SYNB1353 is capable of consuming peripherally-administered labeled methionine and blunts plasma labeled methionine and labeled homocysteine levels.

### Example 14. Effect of Methionine-Restricted Diet in a Mouse Model of Cystinuria

Cystinuria is a genetic disorder of amino acid import in the kidney characterized by excessive excretion of cystine, and dibasic amino acids (ornitihine, lysine, and arginine) in the urine, and cystine stone formation in the urinary tract.

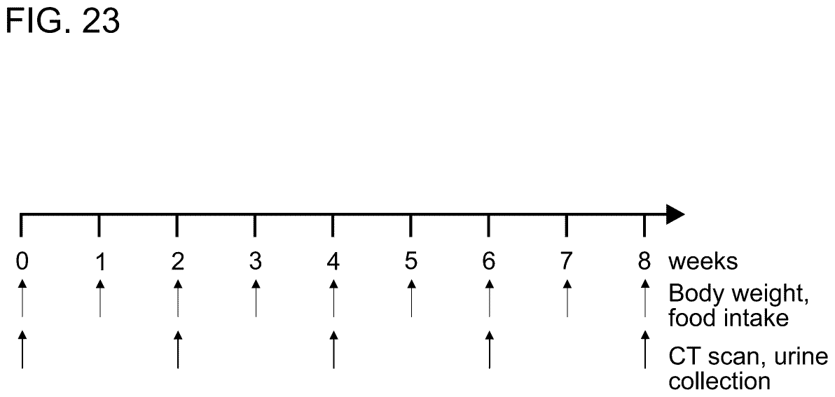

The potential of a methionine consuming strain described herein to treat, prevent, or reduce cystinuria was evaluated by analyzing the effect of a methionine restricted diet in a Slc3a1 knockout (KO) mouse model for cystinuria. Slc3a1 KO mice were subjected to a reduction in the methionine content of diet from the standard 0.62% to 0.12% for eight weeks, and cysteine as well as cystine levels in urine and plasma, and stone formation in the bladder were evaluated according to a scheme shown in FIG. 23.

Cystine stone formation was not observed in any of the twelve mice on the low-methionine diet. In contrast, bladder stones were observed in nine out of twelve mice (75%) on the 0.62% diet. Time of stone formation ranged from 2-8 weeks following diet treatment.

These data suggest that a treatment resulting in a reduction in plasma or urinary methionine, e.g., administration of a methionine-consuming strain described herein, is a promising approach for the treatment of cystinuria.

### Methods

Mouse Breeding and Diet Modification

KO mice were generated using Slc−/−×Slc−/− matings and one-half of the male mice from a given litter were used for the 0.12% methionine study and the other half for the 0.62% study. Mice were identified using ear tags. One group of KO mice (N=12, age six weeks) was placed on a diet containing 0.12% methionine and another group on a diet containing 0.62% methionine. A third group of WT mice was placed on the 0.62% methionine diet. Both diets contained 10 kcal % fat. The 0.62% methionine diet is equivalent to the regular mouse diet with respect to methionine content, whereas the 0.12% diet is approximately 20% of regular methionine content.

Computed Tomography

KO mice were scanned by computed tomography (CT) on the Albira PET/CT system (Bruker Corporation) and only mice showing no evidence of stone at the age of six weeks were used for the diet studies. KO mice were then scanned every two weeks and bladder stone volume determined using the VivoQuant software (from Invicro) installed on the CT scanner. WT mice were not scanned.

Body Weight, Food Consumption, and Urine Collection

Mice were weighed and urine collected at baseline. The amount of food added per cage was also weighed. Mice were maintained on the above diets for eight weeks and the amount of food in each cage weighed weekly. There were three or four mice per cage. From these data, the average food consumption per mouse per day was calculated. Water was provided ad libitum, but water consumption was not measured. Mice were then weighed weekly and urine collected every two weeks. Urine samples were stored at −80° C.

Mouse Sacrifice, Blood Collection, and Plasma Separation

Mice were sacrificed using CO2 exposure at the end of the 8-week treatment period and blood was collected by cardiac puncture into heparin (green top tubes) from each of the low- and high-methionine groups and from nine WT mice. Plasma was separated by centrifugation and then stored at −80° C.

A couple of the KO mice on 0.12% methionine diet had lost 20% of their body weights and were therefore euthanized at the recommendations of veterinary staff.

Tissue Fixation and Storage

Kidneys, bladder, and a small portion of the liver were removed from each mouse and placed in 10% formalin.

Bladder Dissection and Stone Enumeration

After three days in formalin, bladders were dissected, and any stones removed and weighed. The dissected bladders were also weighed, photographed and then returned to formalin. Stone number and size distribution (based on surface area of the stone image) were determined using NIH Image J software.

Data Analysis

Where appropriate, data were analyzed using standard statistical techniques and a p value of <0.05 was considered significant.

### Results

Body Weight

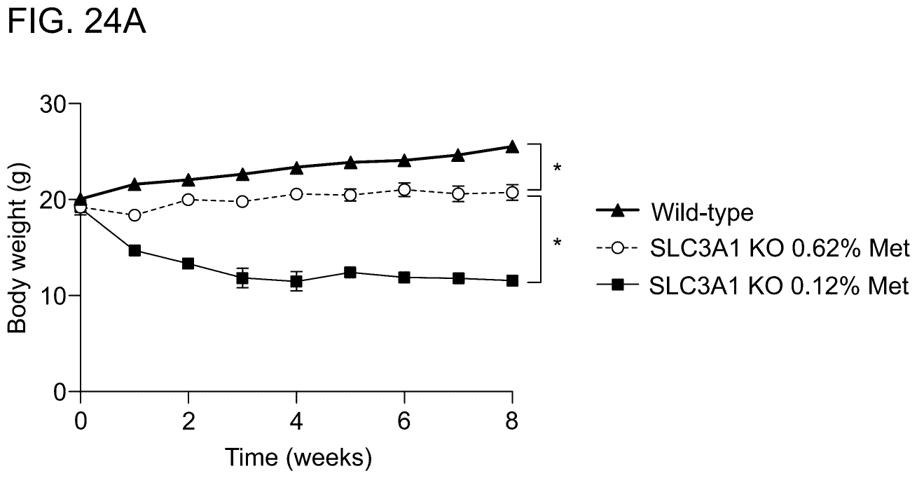

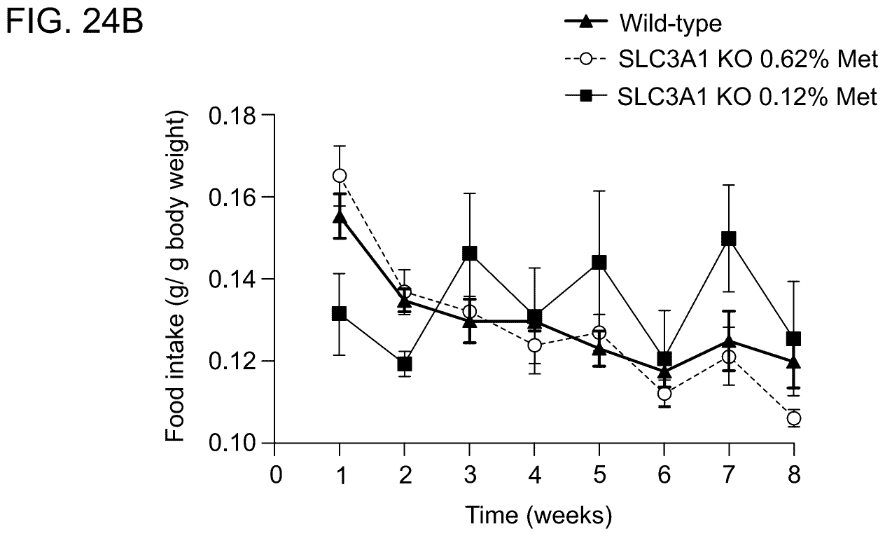

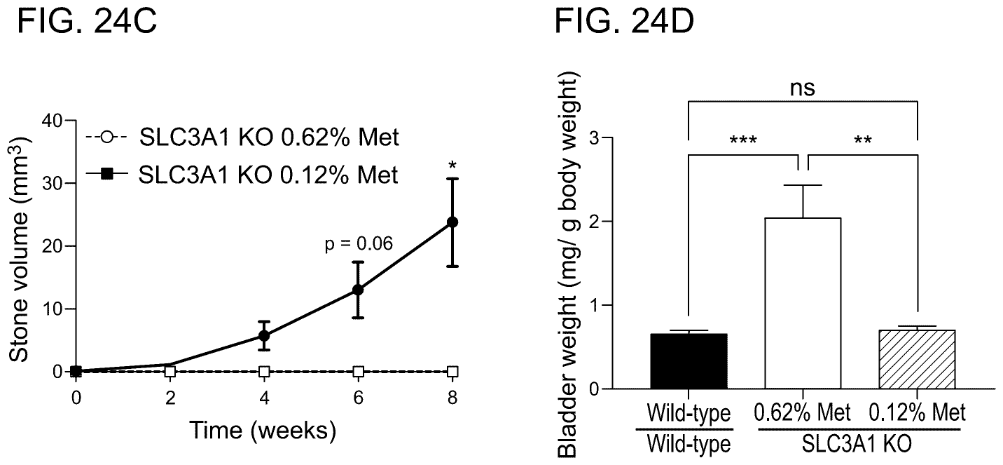

The average body weight in the two KO groups at the start of the study was 19.24 g, but there was a rapid decline in body weight to 14.73 g after one-week on the 0.12% diet (a loss of 23.4% relative to baseline). This was followed by a gradual decline over time, and the weight at eight weeks of treatment was 11.92 g (a loss of 38.0% relative to baseline). As indicated in the Methods section, two mice in this group were sacrificed at 3- and 8-weeks of treatment, respectively. By comparison, body weight of KO mice on the 0.62% diet increased over time, reaching 20.55 g after eight weeks of treatment (an increase of 6.81%). Results are shown in FIG. 24A.

Body weight in the WT mice on the 0.62% diet increased in a linear manner over time (black line), going from 20.48 g at baseline to 25.17 g after eight weeks (an increase of 22.9%).

Food Consumption

Three cages (with four mice per cage) were set-up for each of the 0.12% and 0.62% diets for the KO mice and four cages (with three mice per cage) were set-up for the 0.62% diet for WT mice. The average food consumption in the three groups was 1.68, 2.57, and 3.00 g/day/mouse, respectively. This may be related to differences in body weight among the three groups. Results normalized to body weight are shown in FIG. 24B.

CT Scanning

The KO mice on the 0.12 and 0.62% methionine diets were CT scanned every two weeks. Bladder stones were not detected in any mice on the 0.12% diet. Bladder stones were detected by CT in 7 of the 12 KO mice on the 0.62% diet and the onset of stone detection ranged from two weeks to eight weeks of dietary treatment. In two mice, stones were not detected by CT, but small amounts of stone material were present when the bladders were dissected. Therefore, nine of the 12 KO mice on the 0.62% diet demonstrated evidence of stone presence. Results are showing in FIG. 24C and graphs of stone volume versus treatment period were almost parallel, indicating that, once stone formation has started, the rate of stone volume increase was comparable in all seven mice in which stones were detected by CT.

Bladder and Stone Weight

As indicated above, stones were identified in bladders from nine KO mice on the 0.62% methionine diet (7 by CT and two following bladder dissection). In the absence of stones, bladder weight in these mice was typically 16 mg. In the presence of stones, bladders were enlarged, with bladder and stone weights ranging from 19.6-82.1 and 20.2-83.3 mg, respectively, in the seven mice with CT-verified stones.

Bladder weight in the 11 KO mice on the 0.12% diet was in the range 5.7-11.0 g (mean=8.38, SD=1.45). Bladder weights normalized to body weight are shown in FIG. 24D.

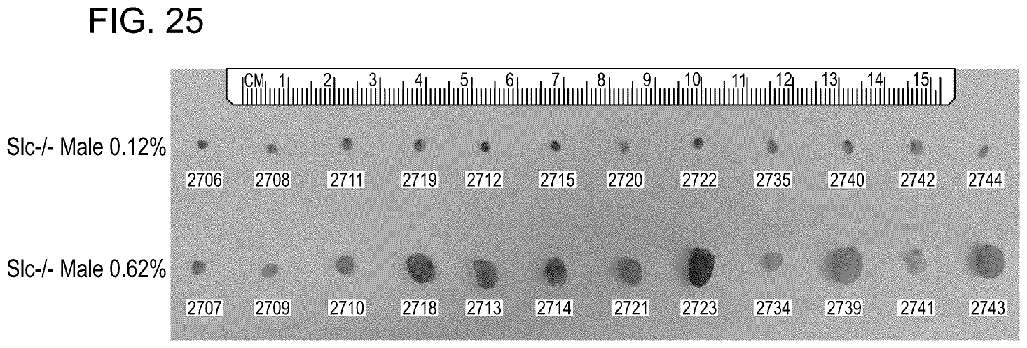

Bladder weight in the 12 WT mice on the 0.62% diet was in the range 13.5-23.5 g, but Grubbs' test using GraphPad software (GraphPad.com/quickcalcs/) indicated that the bladder of mouse #2728 (23.5 g) was an outlier (P<0.05). It was therefore removed from the data, giving an adjusted mean and SD of 16.36 and 1.70, respectively. The difference in bladder weight of this mouse compared with the others is evident in the photographs of the bladders from KO mice on the 012% and 0.62% methionine diets (FIG. 25).

The difference in bladder weight between the KO mice on the 0.12% diet and WT mice on the 0.62% diet was statistically significant using the unpaired t-test (P<0.0001). This is due to the decrease in body weight in the KO mice on the 0.12% diet versus WT mice. As shown in FIG. 24D, when the bladder weight was normalized to total body weight, the difference in bladder weight between KO on 0.12% diet and WT was no longer significant.

Bladder Stone Enumeration

Stones from each of the nine mice were enumerated and the surface area of the stone image determined using NIH Image J software.

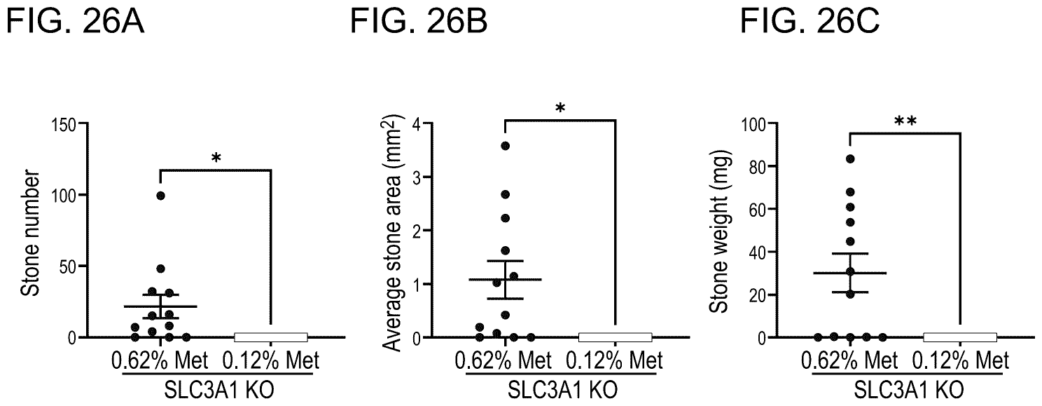

Stone number, average stone area, and stone weight are summarized in FIGS. 26A-26C. The number of stones per mouse was in the range 4-99 and the average surface area was in the range 0.077-3.574 mm². Of the 260 stones identified, 137 occupied a surface area within the range 0.00-0.50 mm2 and one stone was at the extreme end (20.01-25.00 mm²).

### CONCLUSION

A diet containing 0.62% methionine promoted stone formation in nine out of 12 KO mice (75%), whereas stones were not observed in any of the 12 KO mice that were on a diet containing 0.12% methionine for 8 weeks. These results indicate that a reduction in methionine levels as a result of methionine restriction leads to a reduction in urolithiasis and provides a rationale for using a methionine consuming strain described herein as a treatment for the reduction of stone formation in a subject having cystinuria.

### Example 15. Metabolite Levels in a Mouse Model of Homocystinuria

Urine and plasma samples collected from several mice in each of the three groups in the study described in the previous example, and urinary cystine, cysteine, and methionine levels as well as plasma cysteine levels were measured.

In brief, cysteine, cystine, ornithine, lysine, and arginine were quantitated in mouse plasma and urine by LC-MS/MS. Samples were deprotonated with sulfosalicylic acid then diluted with acetonitrile containing heavy-isotope internal standards for each analyte. To measure total cysteine, separate sample aliquots were first reduced with DL-dithiothreitol. Analytes were separated using hydrophilic interaction chromatography and detected using selected reaction monitoring of compound specific ions. Peaks were integrated and analyte/internal standard area ratios were used to calculate unknown concentrations relative to a standard curve.

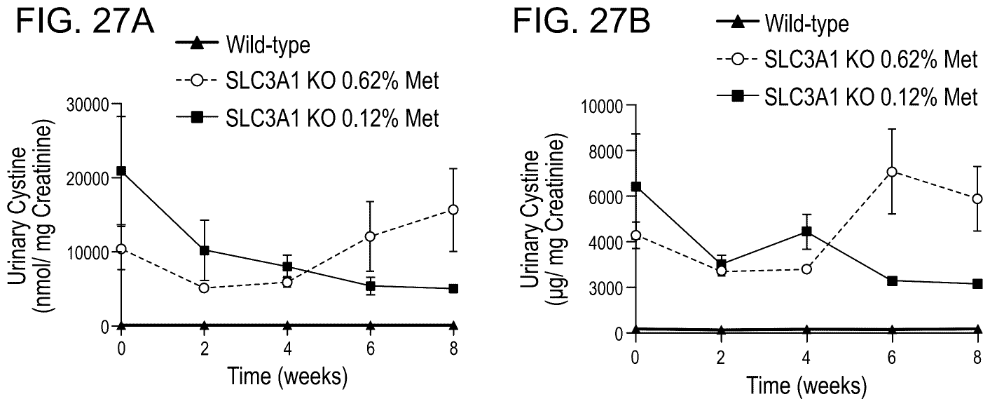

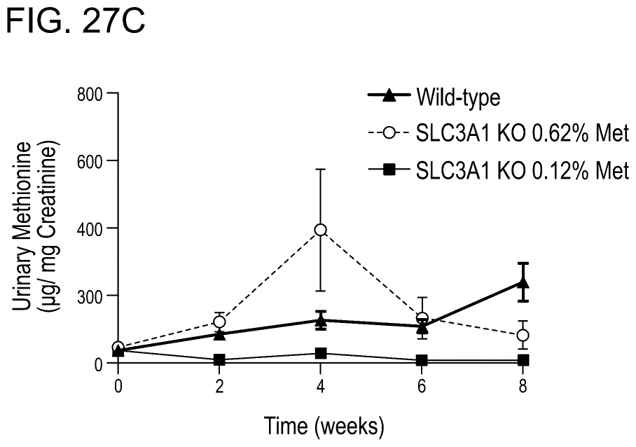

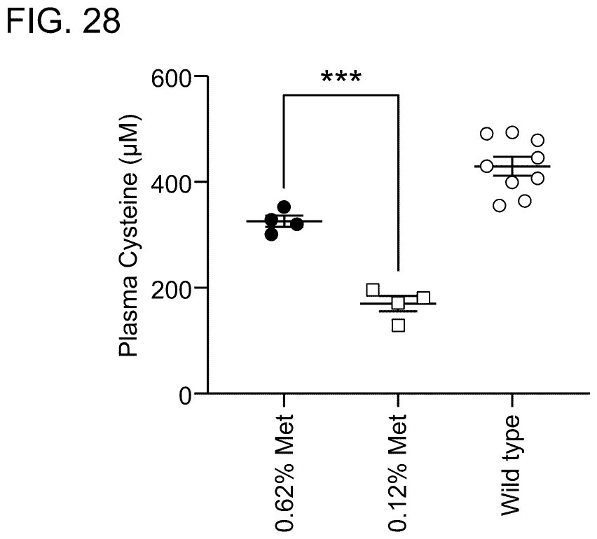

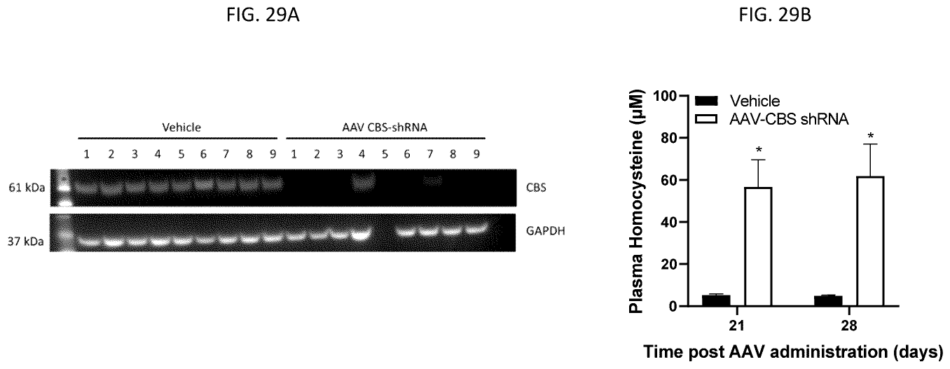

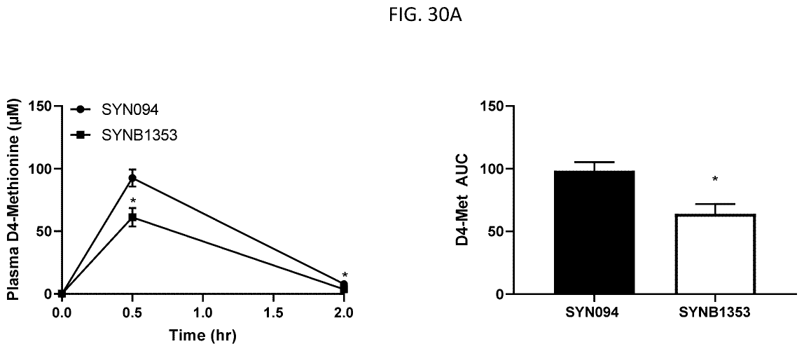

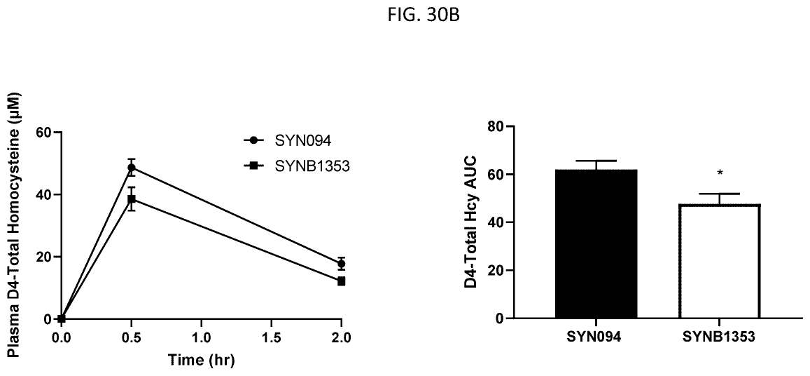

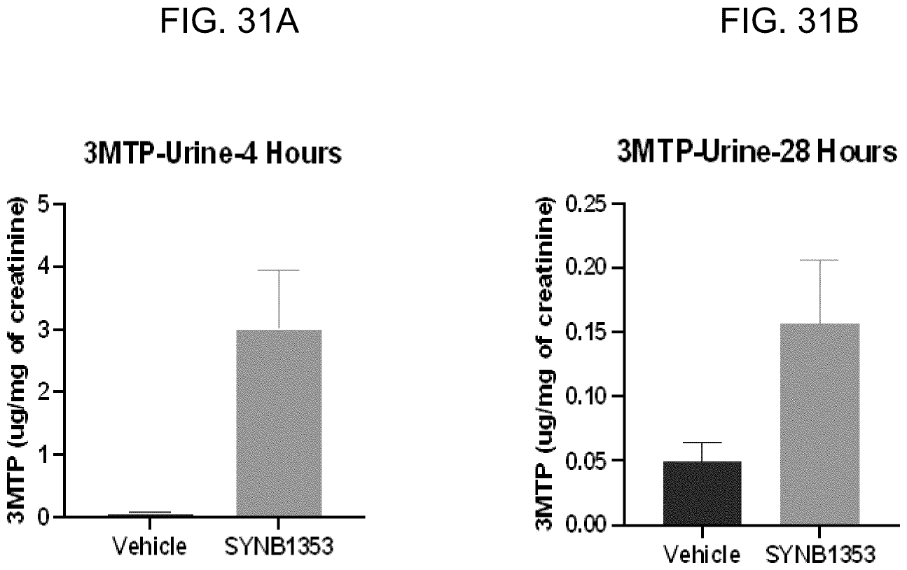

Results of the urine analysis are shown in FIGS. 27A-27C, and results of the plasma analysis is shown in FIG. 28 and demonstrate that methionine restriction in the mouse model of cystinuria lowered urinary cysteine, cystine and methionine levels. Low-Methionine group was also associated with decreased plasma cysteine. These results further support the conclusion that reducing methionine levels in subjects with cystinuria, e.g., by using a methionine consuming strain described herein, can reduce elevated cysteine and cystine in urine and/or plasma and consequently can reduce or prevent cystine stone formation in kidney or bladder in these subjects.

### Example 16. Mouse Model of Homocystinuria

Methods

Generation of an Inducible Mouse Model of Homocystinuria

To develop an inducible model of homocystinuria, short hairpin RNA (shRNA) targeting cystathionine β-synthase (CBS) packaged in adeno-associated virus (AAV) particles were purchased from Vector Biolabs (Malvern, PA) and used to inject 6-week-old C57BL/6J male mice. For this experiment, 18 male C57BL/6J mice were group housed and assigned to groups (n=9/group) based on average cage body weight. Mice received a single intravenous dose (tail vein) of vehicle (PBS) or AAV CBS-shRNA (1×10¹² genomic copies [GC]) and left unmanipulated for 72 hours. Blood was collected 21- or 28-days post IV dosing for tHcy analysis. Animals were euthanized by CO2 asphyxiation on day 35 and livers were snap-frozen in liquid nitrogen for future analysis.

Liver samples were added to bead-bug tubes and homogenized for 30 seconds in 1 mL of lysis buffer (TPER) with a cocktail of protease inhibitors, followed by a 3-minutes centrifugation at 25,200 g. Protein concentration was determined on the supernatants by the Bradford method, 50 pg of liver homogenate was loaded onto a 4-12% Bis-Tris gel and proteins transferred onto PVDF membranes. Membranes were incubated overnight with a primary rabbit monoclonal antibody against CBS (D8F2P, Cell Signaling Technologies Cat. 14782S) at 1:333 dilution. Membranes were then washed and incubated with anti-rabbit IgG HRP-linked secondary antibody (Cell Signaling Technologies Cat. 70745) at 1:1000 dilution for 60 minutes at room temperature. Protein signal was revealed using ECL Reagent and developed using SignalFire™ ECL Reagent #6883 (Cell Signaling Technologies Cat. 6883S).

Assessment of SYNB1353 Activity in AAV-CBS Mice

ShRNA targeting CBS packaged in AAV particles was administered to 8-week-old C57BL/6J male mice by IV injection (1×10¹² genomic copies (GC) of AAV). Six weeks post-AAV injection, mice were orally administered a bolus of labeled methionine (50 mg/kg) with EcN (2.7×10¹⁰ live cells, n=8/group) or SYNB1353 (2.7×10¹⁰ live cells, n=8/group). One hour later, mice received another dose of bacteria (EcN or SYNB1353 at 2.7×10¹⁰ live cells) and blood was collected 0, 0.5, or 2 hours post-labeled methionine.

Results

To assess the activity of SYNB1353 on plasma total homocysteine (tHcy) levels in mice, we developed a new model by delivering short hairpin RNA (shRNA) targeting CBS packaged in adeno-associated virus (AAV) particles by intravenous (IV) injection to 6-week-old C57BL/6J male mice. Mice were administered vehicle or 1×10¹² genomic copies (GC) of AAV by tail vein injection. On days 21 and 28 post AAV injection, blood was collected for total homocysteine determination and livers were harvested on day 35 to assess CBS expression. AAV-targeted delivery of CBS shRNA resulted in substantial lowering of hepatic CBS protein (61 kDa) expression by Western Blot compared to vehicle treated animals. Because of residual CBS protein in one AAV CBS-shRNA, suggesting incomplete CBS knockdown, and lack of housekeeping protein expression (GAPDH) in another, these 2 animals were removed from further analysis. Total homocysteine levels remained low in animals with intact CBS (average 5.1 μM), while downregulation of hepatic CBS expression resulted in a significant 10-12-fold elevation in plasma tHcy.

Short hairpin RNA (shRNA) targeting cystathionine β-synthase (CBS) packaged in adeno-associated virus (AAV) particles was administered to 8-week-old C57BL/6J male mice by intravenous (IV) injection (1×10¹² genomic copies (GC) of AAV). Six weeks post-AAV injection, mice were orally administered a bolus of labeled methionine (50 mg/kg) with EcN (2.7×10¹⁰ live cells, n=8/group) or SYNB1353 (2.7×10¹⁰ live cells, n=8/group). One hour later, mice received another dose of bacteria (EcN or SYNB1353 at 2.7×10¹⁰ live cells) and blood was collected 0, 0.5, or 2 hours post-labeled methionine. The bolus of labeled methionine resulted in significant elevations in plasma labeled methionine and labeled homocysteine in AAV mice, and SYNB1353 significantly blunted the appearance of both amino acids in plasma as demonstrated by a significant reduction in the area under the curve (AUC) with 35% and 23% for methionine and homocysteine, respectively.

TABLE 9
E. coli Strains

| | | Antibiotic |
|---|---|---|
| Strain No. | Background/genotype | resistance |
| SYN094 | wt Nissle, strepR | Strep |
| SYN7349 | Logic2279, which is a medium-copy p15a plasmid (pTET, atc | cam, carb, |
| | induction) expressing a methionine decarboxylase from | kan |
| | Streptomyces sp. 590 (SEQ ID NO: 1003) to convert methionine | |
| | into 3-methylthiopropylamine and Logic2375, which is a low- | |
| | copy pSC101 plasmid (pTET, atc induction) expressing an | |
| | endogenous methionine importer (metNIQ); ΔyjeH | |
| SYN7815 | ΔyjeH, containing Logic2279. Also containing logic2501 with an | Cam, kan, |
| | engineered MetNIQ importer (MetN P281S) (SEQ ID NO: 1047 | spec |
| | (metN(P281S)IQ)); (pLacO, IPTG induction) | |
| SYN7816 | Logic2279, methionine decarboxylase from Streptomyces sp. 590 | Cam, kan, |
| | (SEQ ID NO: 1003). Also containing plasmid logic2502 with an | spec |
| | engineered MetNIQ importer (MetN P281G) (SEQ ID NO: 1045 | |
| | (metN(P281G)IQ)); (pLacO, IPTG induction); ΔyjeH | |
| SYN7817 | Logic2279, methionine decarboxylase from Streptomyces sp. 590 | Cam, kan, |
| | (SEQ ID NO: 1003). Also containing logic2503 with a recoded | spec |
| | MetNIQ importer (SEQ ID NO: 1046); (pLacO, IPTG | |
| | induction); ΔyjeH | |
| SYN7818 | Logic2279, methionine decarboxylase from Streptomyces sp. 590 | Cam, kan, |
| | (SEQ ID NO: 1003). Also containing logic2534 with the MetP | spec |
| | importer (F. frigoris) (SEQ ID NO: 1042); (pLacO I, IPTG | |
| | induction); ΔyjeH | |
| SYN7819 | Logic2279, methionine decarboxylase from Streptomyces sp. 590 | Cam, kan, |
| | (SEQ ID NO: 1003). Also containing logic2535 with the MetP | spec |
| | importer (F. segetis) (SEQ ID NO: 1041); (pLacO, IPTG | |
| | induction); ΔyjeH | |
| SYN7346 | Logic2279, methionine decarboxylase from Streptomyces sp. 590 | cam, kan |
| | (SEQ ID NO: 1003); ΔyjeH | |
| SYN7640 | recoded Streptomyces MetDC (SEQ ID NO: 1003), pSC101 | Cam, kan |
| | (pTET, atc induction); ΔyjeH | |
| SYN7641 | protein engineered MetDC (V491L A500P) (SEQ ID NO: 1035) | Cam, kan |
| | with high 3MTP production, pSC101 (pTET, atc induction); | |
| | ΔyjeH | |
| SYN7642 | protein engineered MetDC (Q70D N82H) (SEQ ID NO: 1034) | Cam, kan |
| | with high 3MTP production, pSC101 (pTET, atc induction); | |
| | ΔyjeH | |
| SYN7643 | protein engineered MetDC (R41Q Q70D) (SEQ ID NO: 1036) | Cam, kan |
| | with high 3MTP production, pSC101 (pTET, atc induction); | |
| | ΔyjeH | |
| SYN7644 | Stanieria sp. NIES-3757 Methionine decarboxylase (SEQ ID | Cam kan |
| | NO: 1037) from MetDC metagenomic library screen with high | |
| | 3MTP production, pSC101 (pTET, atc induction); ΔyjeH, | |
| SYN7689 | SYN7345 containing Logic2491, which expresses an engineered | Cam kan |
| | MetDC from Mus musculus (SEQ ID NO: 1039). pSC101 | |
| | (pTET, atc induction); ΔyjeH | |
| SYN7690 | SYN7345 containing Logic2492, which expresses an engineered | Cam kan |
| | LeuDC from Mus musculus (SEQ ID NO: 1038). pSC101 | |
| | (pTET, atc induction); ΔyjeH | |
| SYN7691 | SYN7345 containing Logic2493, which expresses a MetDC from | Cam kan |
| | Entamoeba histolytica (SEQ ID NO: 1040). pSC101 (pTET, atc | |
| | induction); ΔyjeH | |
| SYN7345 | EcN with endogenous methionine and branched-chain a.a. | cam |
| | exporter (yjeH) knocked out. | |
| SYN7970 | (lacI-Ptac, IPTG) 2× MetDC (Q70D N82H) (SEQ ID NO: 1034); | none |
| | 1× MetP (F. segetis) (SEQ ID NO: 1041); Δdap; ΔyjeH, Δϕ | |
| SYN8002 | (lacI-Ptac, IPTG) 3× MetDC (Q70D N82H) (SEQ ID NO: 1034); | None |
| | 1× MetP (F. segetis) (SEQ ID NO: 1041); Δdap; ΔyjeH; Δϕ | |
| SYN8003 | (lacI-Ptac, IPTG) 3× MetDC (Q70D N82H) (SEQ ID NO: 1034); | none |
| | 1× MetP (F. segetis) (SEQ ID NO: 1041); Δdap; ΔyjeH; Δϕ | |
| | (stronger RBS than SYN8002) | |
| SYN8070 | (lacI-Ptac, IPTG) 3× MetDC (Q70D N82H) (SEQ ID NO: 1034); | None |
| | 1× MetP (F. segetis) (SEQ ID NO: 1041); Δϕ; Δdap; ΔyjeH; | |
| | Δpks (integration: thiC/rsd::attB2-lacI-P_{tac}-metDC; glmS/ | |
| | pstS::attB5-lacI-P_{tac}-metP-P_{tac}-metDC; hypothetical protein/ | |
| | yfjJ::attB7-P_{tac}-metDC) | |
| SYNB1353 | (lacI-Ptac, IPTG) 3× MetDC (Q70D N82H) (SEQ ID NO: 1034); | None |
| | 1× MetP (F. segetis) (SEQ ID NO: 1041); Δϕ; Δdap; ΔyjeH; | |
| | Δpks (integration: thiC/rsd::attB2-lacI-P_{tac}-metDC; | |
| | glmS/pstS::attB5-lacI-P_{tac}-metP-P_{tac}-metDC; hypothetical | |
| | protein/yfjJ::attB7-P_{tac}-metDC) | |

TABLE 10
Exemplary Methionine Decarboxylase Nucleotide Sequences

| Description | |
|---|---|
| SEQ ID NO: | Nucleotide Sequence |
| MetDC Q70D | ATGtccccgacggcgtttccagcggccgaaacagctactgcccctgcaactgccgtcgatcctgggccagaac |
| N82H | tggacggcggagatttcgcccttccagagggcgggctggatgacgatcgtcgcttacgtgcattggacgcagttga |
| SEQ ID NO: | cgagtatttgacccgcaagcgcaagcatttggttgggtaccaagctacccaggatatggacggaacggccttggat |
| 1034 | ttagcccgtttcatgccccacaacatcaacaacctgggagatcctttccagtcgggtgggtataaaccaaatacgaa |
| | agtcgttgagcgtgccgtactggactactatgcaaaattgtggcacgcagaacgtccacacgacccagctgaccca |
| | gaaagctactggggttacatgttatcgatgggctcaactgagggcaacatgtacgccctgtggaatgcacgtgacta |
| | cctgtcgggtaaggctttgattcagcctcccacggcaccatttgacgctgttcgctacgtgaaggctgaccccgatc |
| | gccgcaatcctaacgcacaccacccagtcgcattctactcggaggatacccactattcttttgctaaagccgttgcgg |
| | tgctgggtgtcgaaactttccacgctgtgggtctggagaaatacgctgacgagtgccccttggtggatccagtaacc |
| | ggccttcgtacctggccgaccgaagttccatcgcgcccggggccgtcgggtttaagctgggacggccctggtgag |
| | attgatgttgatgcgcttgcagtactggtcgagttcttcgcagcgaagggtcaccccgtcttcgtcaaccttaacttgg |
| | ggtctacatttaaaggagcacatgatgacgtacgtgcggtatgtgaacgcttattaccaatcttcgagcgccatggctt |
| | agtacaacgtgaagttgtatatgggagctgtccccaaaccggccgccctttagtggatgtacgtcgcggattttggat |
| | ccacgtagatggggcacttggggcggggtatgccccttttctgcgtcttgccgccgaagacccggaaggttatggtt |
| | ggacccctgaggcagaattacctgagttcgacttcggcttacgtttgccgacggcggggcatggagaagttgatat |
| | ggttagcagcatcgccatgagtggacataagtgggcaggcgcgccgtggccatgcggcatctatatgacgaaagt |
| | gaaatatcagattagtccaccgtcacagcccgattatattggtgctcctgacacaacatttgccggttcccgtaacgg |
| | cttttcgccgttaattttgtgggatcatttatcgcgctactcgtaccgcgaccaggtagagcgcatccgcgaagcaca |
| | ggagcttgcagcatatttggaacgccgccttaccgctatggagcgcgagctgggagtggaactttggccagcccg |
| | cacaccgggtgctgtaaccgtacgttttcgcaaaccctctgctgagctggttgcgaagtggtccttgtcgtcgcagg |
| | atgttttaatggtgccgggtgatgaaactacgcgtcgtagttacgttcatgtgttcgtgatgccttctgttgatcgtgcaa |
| | agttagatgcgttgctggcagaattggccgaagatcccgtcatcttgggtgcgccttaa |
| MetDC | ATGTCCCCGACGGCGTTTCCAGCGGCCGAAACAGCTACTGCCCCTGC |
| (Streptomyces) | AACTGCCGTCGATCCTGGGCCAGAACTGGACGGCGGAGATTTCGCCC |
| SEQ ID NO: | TTCCAGAGGGCGGGCTGGATGACGATCGTCGCTTACGTGCATTGGAC |
| 1003 | GCAGTTGACGAGTATTTGACCCGCAAGCGCAAGCATTTGGTTGGGTA |
| | CCAAGCTACCCAGGATATGCAGGGAACGGCCTTGGATTTAGCCCGTT |
| | TCATGCCCAACAACATCAACAACCTGGGAGATCCTTTCCAGTCGGGT |
| | GGGTATAAACCAAATACGAAAGTCGTTGAGCGTGCCGTACTGGACTA |
| | CTATGCAAAATTGTGGCACGCAGAACGTCCACACGACCCAGCTGACC |
| | CAGAAAGCTACTGGGGTTACATGTTATCGATGGGCTCAACTGAGGGC |
| | AACATGTACGCCCTGTGGAATGCACGTGACTACCTGTCGGGTAAGGC |
| | TTTGATTCAGCCTCCCACGGCACCATTTGACGCTGTTCGCTACGTGAA |
| | GGCTGACCCCGATCGCCGCAATCCTAACGCACACCACCCAGTCGCAT |
| | TCTACTCGGAGGATACCCACTATTCTTTTGCTAAAGCCGTTGCGGTGC |
| | TGGGTGTCGAAACTTTCCACGCTGTGGGTCTGGAGAAATACGCTGAC |
| | GAGTGCCCCTTGGTGGATCCAGTAACCGGCCTTCGTACCTGGCCGAC |
| | CGAAGTTCCATCGCGCCCGGGGCCGTCGGGTTTAAGCTGGGACGGCC |
| | CTGGTGAGATTGATGTTGATGCGCTTGCAGTACTGGTCGAGTTCTTCG |
| | CAGCGAAGGGTCACCCCGTCTTCGTCAACCTTAACTTGGGGTCTACAT |
| | TTAAAGGAGCACATGATGACGTACGTGCGGTATGTGAACGCTTATTA |
| | CCAATCTTCGAGCGCCATGGCTTAGTACAACGTGAAGTTGTATATGG |
| | GAGCTGTCCCCAAACCGGCCGCCCTTTAGTGGATGTACGTCGCGGAT |
| | TTTGGATCCACGTAGATGGGGCACTTGGGGCGGGGTATGCCCCTTTTC |
| | TGCGTCTTGCCGCCGAAGACCCGGAAGGTTATGGTTGGACCCCTGAG |
| | GCAGAATTACCTGAGTTCGACTTCGGCTTACGTTTGCCGACGGCGGG |
| | GCATGGAGAAGTTGATATGGTTAGCAGCATCGCCATGAGTGGACATA |
| | AGTGGGCAGGCGCGCCGTGGCCATGCGGCATCTATATGACGAAAGTG |
| | AAATATCAGATTAGTCCACCGTCACAGCCCGATTATATTGGTGCTCCT |
| | GACACAACATTTGCCGGTTCCCGTAACGGCTTTTCGCCGTTAATTTTG |
| | TGGGATCATTTATCGCGCTACTCGTACCGCGACCAGGTAGAGCGCAT |
| | CCGCGAAGCACAGGAGCTTGCAGCATATTTGGAACGCCGCCTTACCG |
| | CTATGGAGCGCGAGCTGGGAGTGGAACTTTGGCCAGCCCGCACACCG |
| | GGTGCTGTAACCGTACGTTTTCGCAAACCCTCTGCTGAGCTGGTTGCG |
| | AAGTGGTCCTTGTCGTCGCAGGATGTTTTAATGGTGCCGGGTGATGA |
| | AACTACGCGTCGTAGTTACGTTCATGTGTTCGTGATGCCTTCTGTTGA |
| | TCGTGCAAAGTTAGATGCGTTGCTGGCAGAATTGGCCGAAGATCCCG |
| | TCATCTTGGGTGCGCCTtaa |
| MetDC | atgagcccgaccgccttccccgccgccgagaccgcgaccgcgcccgcgaccgccgtcgatcccggtccggag |
| (Streptomyces) | ctggacggcggtgacttcgccctccccgagggcggcctggacgacgaccggcggctgcgcgcgctcgacgcc |
| SEQ ID NO: | gtggacgagtacctgacccgcaagcgcaagcacctggtcggctaccaggccacccaggacatgcagggcaccg |
| 1018 | cactggacctcgcccggttcatgccgaacaacatcaacaacctcggcgacccgttccagagcggcggatacaag |
| | cccaacaccaaggtcgtcgagcgggccgtgctcgactactacgcgaagctctggcacgccgagcgcccgcacg |
| | acccggccgacccggagtcgtactggggctacatgctgtccatgggctcgaccgagggcaacatgtacgccctct |
| | ggaacgccagggactacctgagcggcaaggcgctgatccagccgccgaccgcccccttcgacgcggtgcgcta |
| | cgtcaaggccgaccccgaccgacggaacccgaacgcccaccacccggtggccttctactccgaggacacccac |
| | tactccttcgccaaggccgtggccgtcctcggcgtggagaccttccacgccgtcggcctggagaagtacgccgac |
| | gagtgcccgctggtcgacccggtgaccgggctgcgcacctggcccaccgaggtgccctcccgcccgggtccgt |
| | ccggcctgtcctgggacggccccggcgagatagacgtcgacgccctcgccgtactcgtcgagttcttcgccgcca |
| | agggtcacccggtcttcgtcaacctcaacctcggcagcaccttcaagggcgcccacgacgacgtccgcgccgtct |
| | gcgagcgcttgctgccgatcttcgagcggcacgggctcgtccagcgcgaggtggtctacggcagctgcccgcag |
| | accggccggccgctggtggacgtgcgccgcggcttctggatccacgtggacggcgcgctcggcgccggctacg |
| | cgccgttcctgcggctggccgccgaggacccggaagggtacggctggacgcccgaggcggagctgcccgagt |
| | tcgacttcggcctgcggctgcccaccgccgggcacggcgaggtggacatggtctcctcgatcgcgatgagcggc |
| | cacaagtgggccggcgcgccgtggccgtgcggcatctacatgaccaaggtgaagtaccagatctcgccgccgtc |
| | ccagccggactacatcggcgccccggacaccaccttcgccggctcccgcaacggcttctccccgctgatcctctg |
| | ggaccacctgtcccggtactcctaccgggaccaggtggagcggatccgcgaggcccaggagctggccgcctac |
| | ctggagcggcggctgaccgccatggagcgcgaactcggcgtcgagctctggccggcccgtaccccgggcgcc |
| | gtcaccgtacggttccgcaagccgagcgccgagctggtggccaagtggtcgctgtcctcccaggacgtgctgatg |
| | gtcccgggcgacgagaccacccggcgcagctacgtgcacgtcttcgtgatgccctcggtcgaccgggccaagct |
| | ggacgcgctgctcgccgaactcgccgaggacccggtgatcctgggcgcaccgtag |
| MetDC | atgtccccgacggcgtttccagcggccgaaacagctactgcccctgcaactgccgtcgatcctgggccagaactg |
| (V491L | gacggcggagatttcgcccttccagagggcgggctggatgacgatcgtcgcttacgtgcattggacgcagttgac |
| A500P) | gagtatttgacccgcaagcgcaagcatttggttgggtaccaagctacccaggatatgcagggaacggccttggattt |
| SEQ ID NO: | agcccgtttcatgcccaacaacatcaacaacctgggagatcctttccagtcgggtgggtataaaccaaatacgaaa |
| 1035 | gtcgttgagcgtgccgtactggactactatgcaaaattgtggcacgcagaacgtccacacgacccagctgacccag |
| | aaagctactggggttacatgttatcgatgggctcaactgagggcaacatgtacgccctgtggaatgcacgtgactac |
| | ctgtcgggtaaggctttgattcagcctcccacggcaccatttgacgctgttcgctacgtgaaggctgaccccgatcg |
| | ccgcaatcctaacgcacaccacccagtcgcattctactcggaggatacccactattcttttgctaaagccgttgcggt |
| | gctgggtgtcgaaactttccacgctgtgggtctggagaaatacgctgacgagtgccccttggtggatccagtaacc |
| | ggccttcgtacctggccgaccgaagttccatcgcgcccggggccgtcgggtttaagctgggacggccctggtgag |
| | attgatgttgatgcgcttgcagtactggtcgagttcttcgcagcgaagggtcaccccgtcttcgtcaaccttaacttgg |
| | ggtctacatttaaaggagcacatgatgacgtacgtgcggtatgtgaacgcttattaccaatcttcgagcgccatggctt |
| | agtacaacgtgaagttgtatatgggagctgtccccaaaccggccgccctttagtggatgtacgtcgcggattttggat |
| | ccacgtagatggggcacttggggcggggtatgccccttttctgcgtcttgccgccgaagacccggaaggttatggtt |
| | ggacccctgaggcagaattacctgagttcgacttcggcttacgtttgccgacggcggggcatggagaagttgatat |
| | ggttagcagcatcgccatgagtggacataagtgggcaggcgcgccgtggccatgcggcatctatatgacgaaagt |
| | gaaatatcagattagtccaccgtcacagcccgattatattggtgctcctgacacaacatttgccggttcccgtaacgg |
| | cttttcgccgttaattttgtgggatcatttatcgcgctactcgtaccgcgaccaggtagagcgcatccgcgaagcaca |
| | ggagcttgcagcatatttggaacgccgccttaccgctatggagcgcgagctgggagtggaactttggccagcccg |
| | cacaccgggtgctctgaccgtacgttttcgcaaaccctctccggagctggttgcgaagtggtccttgtcgtcgcagg |
| | atgttttaatggtgccgggtgatgaaactacgcgtcgtagttacgttcatgtgttcgtgatgccttctgttgatcgtgcaa |
| | agttagatgcgttgctggcagaattggccgaagatcccgtcatcttgggtgcgccttaa |
| MetDC (R41Q | atgtccccgacggcgtttccagcggccgaaacagctactgcccctgcaactgccgtcgatcctgggccagaactg |
| Q70D) | gacggcggagatttcgcccttccagagggcgggctggatgacgatcagcgcttacgtgcattggacgcagttgac |
| SEQ ID NO: | gagtatttgacccgcaagcgcaagcatttggttgggtaccaagctacccaggatatggacggaacggccttggattt |
| 1036 | agcccgtttcatgcccaacaacatcaacaacctgggagatcctttccagtcgggtgggtataaaccaaatacgaaa |
| | gtcgttgagcgtgccgtactggactactatgcaaaattgtggcacgcagaacgtccacacgacccagctgacccag |
| | aaagctactggggttacatgttatcgatgggctcaactgagggcaacatgtacgccctgtggaatgcacgtgactac |
| | ctgtcgggtaaggctttgattcagcctcccacggcaccatttgacgctgttcgctacgtgaaggctgaccccgatcg |
| | ccgcaatcctaacgcacaccacccagtcgcattctactcggaggatacccactattcttttgctaaagccgttgcggt |
| | gctgggtgtcgaaactttccacgctgtgggtctggagaaatacgctgacgagtgccccttggtggatccagtaacc |
| | ggccttcgtacctggccgaccgaagttccatcgcgcccggggccgtcgggtttaagctgggacggccctggtgag |
| | attgatgttgatgcgcttgcagtactggtcgagttcttcgcagcgaagggtcaccccgtcttcgtcaaccttaacttgg |
| | ggtctacatttaaaggagcacatgatgacgtacgtgcggtatgtgaacgcttattaccaatcttcgagcgccatggctt |
| | agtacaacgtgaagttgtatatgggagctgtccccaaaccggccgccctttagtggatgtacgtcgcggattttggat |
| | ccacgtagatggggcacttggggcggggtatgccccttttctgcgtcttgccgccgaagacccggaaggttatggtt |
| | ggacccctgaggcagaattacctgagttcgacttcggcttacgtttgccgacggcggggcatggagaagttgatat |
| | ggttagcagcatcgccatgagtggacataagtgggcaggcgcgccgtggccatgcggcatctatatgacgaaagt |
| | gaaatatcagattagtccaccgtcacagcccgattatattggtgctcctgacacaacatttgccggttcccgtaacgg |
| | cttttcgccgttaattttgtgggatcatttatcgcgctactcgtaccgcgaccaggtagagcgcatccgcgaagcaca |
| | ggagcttgcagcatatttggaacgccgccttaccgctatggagcgcgagctgggagtggaactttggccagcccg |
| | cacaccgggtgctgtaaccgtacgttttcgcaaaccctctgctgagctggttgcgaagtggtccttgtcgtcgcagg |
| | atgttttaatggtgccgggtgatgaaactacgcgtcgtagttacgttcatgtgttcgtgatgccttctgttgatcgtgcaa |
| | agttagatgcgttgctggcagaattggccgaagatcccgtcatcttgggtgcgccttaa |
| MetDC | atggggttccagttactgtctaaacataagctgtcagccgaggatcaacagaaacttgaccgcttttatcgtgatattc |
| (Stanieria sp. | agacagaagcagaacgattcctgggttacccatgtaacgaactctttgactactcccccttgttccggttcctgcaata |
| NIES-3757) | tccgctgaataacgtcggcgacccgtacctgccgagtaactaccacctgaacacgcacaactttgagtgcgaagta |
| SEQ ID NO: | ctggaaatcttccgtaccctgaccgaggctactgaaggttcgacttggggctacgtgaccaacggcggtacggaa |
| 1037 | ggtaatcattatggtcttttcctggcgagagagctgctgcctgaaggccttgtttactattctcaggatgcgcactactc |
| | gatcgataaaatcctgaggtgcctcaacctccgtagcataatgattcgcagccacgacgacggacgcatggacctg |
| | gatgatctgcgtgaaactctgcgtatccatcgcgacttgccgccgatcgtttgcgctaccattggtactacaatgaag |
| | ggcgctgtagatgacatcgcaggcattaaaaagatcttcaaagatctggcaatacaccgtcactatatccatgctgac |
| | gcggccctaggtggcatgattttaccgttcctggataactccccaccgtggaattttaaagctggaatcgactctatcg |
| | ctatctccggtcacaaaatggtgggcagtcctatcccgtgtggggttgtcctggctaaaaagtcgaacgttgaacgta |
| | ttgcacagagcgtggaatacattggtactctggataccaccctgtctggctcccgtaacgccttgactccgttatttctg |
| | tggtacgcgttccacaccgttggtatcgaaggtttcaaacgtatcatcccggcatgcttaaaaatggcggactatgcc |
| | atcgctcagctgaacaaaattaaccgcaatgcgtggcgctacccttacagcaacacggtagtcttcgatcgcccaa |
| | gccccgaagtgactcgttattggcagctggcttgtcagggcaacctgagccacctaatcaccatgccacacgttaca |
| | tctactcaaattgatcatctggttgctgacatcatcgcttctgagccgataccgccgctgccgaccctgtcagttactcc |
| | ggcatgcgaactgctgacttctaccccggaccaggatattacgctgatcggcaccgctaatcataatctgctctccg |
| | aagtatctaccgccctggctgccgagggtctgtcaattgaaaacctggctgctgtggcggtagaaagcgaggacgt |
| | tgaagttgtaaggctccgcgttaacaaccgtgagcgtgcactgcaaatcctgaaccagaacctggatatcggtcgtt |
| | gctacggtcaggctcgaccctttggcaacgaagaagcgacgcaggtactgtcccagctggaatatcaaagcgtgg |
| | gggaggatgcactactggtccagcttgacgattgccctggcagcctggcggagctgttgaaggattgccgcaacg |
| | aagcggtaaaaatccgtaatatccgactgctttggcgtgggcacggtaagggcgtcgtagcaattgctaccacttct |
| | ccagatgcgctgaaaacgctgctgaaagaccgtattcttttgagctaa |
| LeuDC | ATGtccacacctagtgaagtaaagaaggatttgctgggtgcagcagggtcattatggccgtcggagcccattac |
| (Mus musculus) | gctgggtccaggtgaaagtgcttggcagctggtattgaagaagatccaagagttgagtgacagcggtcatcaagac |
| SEQ ID NO: | ccgttcatggttgcagaccttgatgtccttgtgtctcgtcatcagacgttctgtcaagcactgcctagagtacaaccctt |
| 1038 | ctatgcagtaaagtgcaatagtaacccatgggtgttacgggtgttggcagctcttggcacgggatttgattgtgcttct |
| | cagggagaattggagcaagttttgggcttgggtgtagcgccgtcacggataatcttcgcaaatccctgtaaagcagt |
| | cagccacattcagtttgcagctcggtgcggtgtgcaattgttgacattcgacagcgaagaggagttaatcaaggttg |
| | cgcagtaccatccaggcgcacggttggtgcttcggattcaaacccaggactcacaatcaacgttcccactttccacc |
| | aagttcggtgcttctttagaagcatgtggacaccttctgcaggttgccagagagctgggtcttgccgtggtaggtgct |
| | agctttcatgtaggaagcgactgccacacacctcagagttttcgtcaggccatcgcagattgtcatcgtgtgttcgag |
| | atgggccgtaaggcaggtcatgatatgtcgcttcttgatttgggtggagggttcccaggtgtggaaggttccgaggc |
| | gaagtttgaggagatggcaagagtaatcaatgccgctcttgctcagtactttccggaagagactggcatcgaggtga |
| | tcgcggaacctggtcgtttctacgctgggtcggtgtgcactgcagctgtgaacatcatcgcgaagaagtctgtcttgg |
| | aaccaggtggtcatcgtaagcttatgtactaccttaatgaaggacattacggttctttcagattgttcttgcgtgatccag |
| | tgcctcgtattcccatcgtggtgaaagagttcccatccgaaccaccactgtttccttgcactttgtacggtcccacatgt |
| | gacgcctatgatcggttgttttccgaagaggtacaattgccagagctggatgttggagattggttgatcttcccagata |
| | tgggtgcctatacctcctcaatgtcctcgaccttcaacggatttccaccggccaccgtgtattgcgcaatgtcaccgc |
| | agttacgctccctgttggagactgtaccataa |
| MetDC | atgaacacacctagtgaagtaaagaaggatttgctgggtgttgcagaacatttacgtccgtcggagcccattacgct |
| (Mus musculus) | gggtccaggtgcgagtgcttggcagctggtattgaagaagatcaaggagttgagtattagcggtcgtcaagacgct |
| SEQ ID NO: | ttcatggttgcagaccttgatgtccttgtgtctcgtcatcggacgttcttacaagcactgcctagagtacaacccttctat |
| 1039 | gcagtaaagtgcaatagtaacccatgggtgttacttgtgttggcagctcttggcacgggatttgattgtgcttctcagg |
| | gagaattggagcaagttttgggcttgggtgtagcgccgtcacggataatcttcgcaaatccctgtaaagcagtcagc |
| | cacattcagtttgcagctcggtgcggtgtgcaattgttgacattcgacaatgaagaggagttaatcaagttagcgcgtt |
| | accatccacgtgcacggttggtgcttcggattcaaaccctggactcacaatcaacgttcccacttagcaccaagttcg |
| | gtgctcacttagaagcatgtggacaccttctgcaggttgccagagagctgggtcttgccgtggtaggtgctagctttc |
| | atgtaggaagcgactgccacacacctgagagttaccgtcaggccatcgcagattgtcatcgtgtgttcgagatggg |
| | ctgtaaggcaggtcatcacatgtcgcttcttgatttgggtggagggttcccaggtgtgaaaggttccgaggcgaagtt |
| | tgaggaggttgcaagagtaatcaataccgctcttgctcagtactttccggaagagactggcatcgaggtgatcgcgg |
| | aacctggtcgtttctacgctgggtcggtgtgcactgcagctgtgaacatcatcgccaagaagtctagtttggacccag |
| | gtggtcatcgtaagcttgcttactaccttaatgaaggacattacggtgtattcagattgttcttgcgtgatccagtgcctc |
| | gtattcccatcgtggtgaaagagttcccatccgaaccaccactgtttccttgcactttgtacggtcccacatgtgacgc |
| | ctatgatcggttgttttccaccgaggtacaattgccagagctggatgttggagattggttgatcttcccagatatgggtg |
| | cctattcgtcctcaatgtcctcgaccttcaacggatttccaatagccaccgtgtatgatgcaatgtcaccgcagttacg |
| | ctccctgttggagactgtaccataa |
| MetDC | atgaaacaaacgtcccttgaggtgaaggaatttgccttgaatctcatttctcagttcgaaccagaaaaccagcctctg |
| (Entamoeba | ggtttctggatattcgacaccgaaggcgttgagaaagcggtagaacgctggaaaaagaacatgccgactgtccgtc |
| histolytica) | cctgttttgcagttaaatgcaacccggagccgcacctggtgaaattactgggggaactgggttgcggcttcgattgc |
| SEQ ID NO: | gctagcctgaacgaaatcaaagaggtactggacttgggttttaatccggaagatatcacttatagtcagaccttcaaa |
| 1040 | ccgtacaaccagttaattgaagcttcgcatctgggcatcaaccacacgatcgttgattcaatcgacgaagttcaaaaa |
| | attgctaaatacgcgcctaagatgggtatcatgattcggatcatggaaaatgacacaagcgcaggccacgtctttgg |
| | agagaaattcggtctgcatgatgatgaagttgagatcgtactgaaggaaattaaagacaaaggtctgaacctggac |
| | ggcgttcatttccacgttggctctgattcccacaacagcgaagtgtttactaaggcactgaccaaagctcgtaacact |
| | gtaaccctggccgaacagttcggcatgaaaccgtacctgatcgacattggtggcgggttctctcaggttgcgccgtt |
| | cgaagaatttgctgctaccatcgaaaaaactataaaggaactggaatttccagagcgaactcgtttcattgcagagcc |
| | gggtcgctatatggcatcaaatgcctttcaccttgtctcttcgctgcatggtaaaagggtgcgcatccagaacggtaa |
| | gaaacagatcgaatacaccagcggcgatgggctgcacggctccttcggctgttgcatctggttcgaaaaacagaa |
| | gtcttgcgaatgtataacacaaaaagtaaacgagaacaccaaaatgtatgaaagcatcatctacggcccatcttgca |
| | acggttcggacaaagtggccacgcaggagttgccggaaatggagccgggtaaagattggctgctgttccccaata |
| | tgggtgcttacactatttccatggcgaccaactttaacggcttcgaagaacgtaaccatgtaatctatacgttaccactc |
| | aaaagtactaaaataattcagatccctaaaagcattgaatgcaactccgttccgtctttaaacggaatcccacactacg |
| | cgtaa |
| SpMetDC | atgtccccgacggcgtttccagcggccgaaacagctactgcccctgcaactgccgtcgatcctgggccagaactg |
| T66N | gacggcggagatttcgcccttccagagggcgggctggatgacgatcgtcgcttacgtgcattggacgcagttgac |
| Codon- | gagtatttgacccgcaagcgcaagcatttggttgggtaccaagctaatcaggatatgcagggaacggccttggattt |
| optimized | agcccgtttcatgcccaacaacatcaacaacctgggagatcctttccagtcgggtgggtataaaccaaatacgaaa |
| sequence | gtcgttgagcgtgccgtactggactactatgcaaaattgtggcacgcagaacgtccacacgacccagctgacccag |
| SEQ ID NO: | aaagctactggggttacatgttatcgatgggctcaactgagggcaacatgtacgccctgtggaatgcacgtgactac |
| 1123 | ctgtcgggtaaggctttgattcagcctcccacggcaccatttgacgctgttcgctacgtgaaggctgaccccgatcg |
| | ccgcaatcctaacgcacaccacccagtcgcattctactcggaggatacccactattcttttgctaaagccgttgcggt |
| | gctgggtgtcgaaactttccacgctgtgggtctggagaaatacgctgacgagtgccccttggtggatccagtaacc |
| | ggccttcgtacctggccgaccgaagttccatcgcgcccggggccgtcgggtttaagctgggacggccctggtgag |
| | attgatgttgatgcgcttgcagtactggtcgagttcttcgcagcgaagggtcaccccgtcttcgtcaaccttaacttgg |
| | ggtctacatttaaaggagcacatgatgacgtacgtgcggtatgtgaacgcttattaccaatcttcgagcgccatggctt |
| | agtacaacgtgaagttgtatatgggagctgtccccaaaccggccgccctttagtggatgtacgtcgcggattttggat |
| | ccacgtagatggggcacttggggcggggtatgccccttttctgcgtcttgccgccgaagacccggaaggttatggtt |
| | ggacccctgaggcagaattacctgagttcgacttcggcttacgtttgccgacggcggggcatggagaagttgatat |
| | ggttagcagcatcgccatgagtggacataagtgggcaggcgcgccgtggccatgcggcatctatatgacgaaagt |
| | gaaatatcagattagtccaccgtcacagcccgattatattggtgctcctgacacaacatttgccggttcccgtaacgg |
| | cttttcgccgttaattttgtgggatcatttatcgcgctactcgtaccgcgaccaggtagagcgcatccgcgaagcaca |
| | ggagcttgcagcatatttggaacgccgccttaccgctatggagcgcgagctgggagtggaactttggccagcccg |
| | cacaccgggtgctgtaaccgtacgttttcgcaaaccctctgctgagctggttgcgaagtggtccttgtcgtcgcagg |
| | atgttttaatggtgccgggtgatgaaactacgcgtcgtagttacgttcatgtgttcgtgatgccttctgttgatcgtgcaa |
| | agttagatgcgttgctggcagaattggccgaagatcccgtcatcttgggtgcgccttaa |
| SpMetDC | atgtccccgacggcgtttccagcggccgaaacagctactgcccctgcaactgccgtcgatcctgggccagaactg |
| A203H | gacggcggagatttcgcccttccagagggcgggctggatgacgatcgtcgcttacgtgcattggacgcagttgac |
| Codon- | gagtatttgacccgcaagcgcaagcatttggttgggtaccaagctacccaggatatgcagggaacggccttggattt |
| optimized | agcccgtttcatgcccaacaacatcaacaacctgggagatcctttccagtcgggtgggtataaaccaaatacgaaa |
| sequence | gtcgttgagcgtgccgtactggactactatgcaaaattgtggcacgcagaacgtccacacgacccagctgacccag |
| SEQ ID NO: | aaagctactggggttacatgttatcgatgggctcaactgagggcaacatgtacgccctgtggaatgcacgtgactac |
| 1125 | ctgtcgggtaaggctttgattcagcctcccacggcaccatttgacgctgttcgctacgtgaaggctgaccccgatcg |
| | ccgcaatcctaacgcacaccacccagtcgcattctactcggaggatacccactattcttttgctaaagccgttcatgtg |
| | ctgggtgtcgaaactttccacgctgtgggtctggagaaatacgctgacgagtgccccttggtggatccagtaaccg |
| | gccttcgtacctggccgaccgaagttccatcgcgcccggggccgtcgggtttaagctgggacggccctggtgaga |
| | ttgatgttgatgcgcttgcagtactggtcgagttcttcgcagcgaagggtcaccccgtcttcgtcaaccttaacttggg |
| | gtctacatttaaaggagcacatgatgacgtacgtgcggtatgtgaacgcttattaccaatcttcgagcgccatggctta |
| | gtacaacgtgaagttgtatatgggagctgtccccaaaccggccgccctttagtggatgtacgtcgcggattttggatc |
| | cacgtagatggggcacttggggcggggtatgccccttttctgcgtcttgccgccgaagacccggaaggttatggtt |
| | ggacccctgaggcagaattacctgagttcgacttcggcttacgtttgccgacggcggggcatggagaagttgatat |
| | ggttagcagcatcgccatgagtggacataagtgggcaggcgcgccgtggccatgcggcatctatatgacgaaagt |
| | gaaatatcagattagtccaccgtcacagcccgattatattggtgctcctgacacaacatttgccggttcccgtaacgg |
| | cttttcgccgttaattttgtgggatcatttatcgcgctactcgtaccgcgaccaggtagagcgcatccgcgaagcaca |
| | ggagcttgcagcatatttggaacgccgccttaccgctatggagcgcgagctgggagtggaactttggccagcccg |
| | cacaccgggtgctgtaaccgtacgttttcgcaaaccctctgctgagctggttgcgaagtggtccttgtcgtcgcagg |
| | atgttttaatggtgccgggtgatgaaactacgcgtcgtagttacgttcatgtgttcgtgatgccttctgttgatcgtgcaa |
| | agttagatgcgttgctggcagaattggccgaagatcccgtcatcttgggtgcgccttaa |
| SpMetDC | atgtccccgacggcgtttccagcggccgaaacagctactgcccctgcaactgccgtcgatcctgggccagaactg |
| H379G | gacggcggagatttcgcccttccagagggcgggctggatgacgatcgtcgcttacgtgcattggacgcagttgac |
| Codon- | gagtatttgacccgcaagcgcaagcatttggttgggtaccaagctacccaggatatgcagggaacggccttggattt |
| optimized | agcccgtttcatgcccaacaacatcaacaacctgggagatcctttccagtcgggtgggtataaaccaaatacgaaa |
| sequence | gtcgttgagcgtgccgtactggactactatgcaaaattgtggcacgcagaacgtccacacgacccagctgacccag |
| SEQ ID NO: | aaagctactggggttacatgttatcgatgggctcaactgagggcaacatgtacgccctgtggaatgcacgtgactac |
| 1127 | ctgtcgggtaaggctttgattcagcctcccacggcaccatttgacgctgttcgctacgtgaaggctgaccccgatcg |
| | ccgcaatcctaacgcacaccacccagtcgcattctactcggaggatacccactattcttttgctaaagccgttgcggt |
| | gctgggtgtcgaaactttccacgctgtgggtctggagaaatacgctgacgagtgccccttggtggatccagtaacc |
| | ggccttcgtacctggccgaccgaagttccatcgcgcccggggccgtcgggtttaagctgggacggccctggtgag |
| | attgatgttgatgcgcttgcagtactggtcgagttcttcgcagcgaagggtcaccccgtcttcgtcaaccttaacttgg |
| | ggtctacatttaaaggagcacatgatgacgtacgtgcggtatgtgaacgcttattaccaatcttcgagcgccatggctt |
| | agtacaacgtgaagttgtatatgggagctgtccccaaaccggccgccctttagtggatgtacgtcgcggattttggat |
| | ccacgtagatggggcacttggggcggggtatgccccttttctgcgtcttgccgccgaagacccggaaggttatggtt |
| | ggacccctgaggcagaattacctgagttcgacttcggcttacgtttgccgacggcggggggcggagaagttgatat |
| | ggttagcagcatcgccatgagtggacataagtgggcaggcgcgccgtggccatgcggcatctatatgacgaaagt |
| | gaaatatcagattagtccaccgtcacagcccgattatattggtgctcctgacacaacatttgccggttcccgtaacgg |
| | cttttcgccgttaattttgtgggatcatttatcgcgctactcgtaccgcgaccaggtagagcgcatccgcgaagcaca |
| | ggagcttgcagcatatttggaacgccgccttaccgctatggagcgcgagctgggagtggaactttggccagcccg |
| | cacaccgggtgctgtaaccgtacgttttcgcaaaccctctgctgagctggttgcgaagtggtccttgtcgtcgcagg |
| | atgttttaatggtgccgggtgatgaaactacgcgtcgtagttacgttcatgtgttcgtgatgccttctgttgatcgtgcaa |
| | agttagatgcgttgctggcagaattggccgaagatcccgtcatcttgggtgcgccttaa |

TABLE 11
Exemplary Importer Nucleotide Sequences

| Description | |
|---|---|
| SEQ ID NO: | Nucleotide Sequence |
| MetP | atggggaccattaacacgaagatctataaatacatgagcatctggaaaacaaaacctctgtccgtgctcttgtctgaa |
| (F. segetis) | gcaactgaggatgaaaaaggcctgaagcgcactctgtcggcccgttcacttgttgcgctgggtgtcggtgctattat |
| SEQ ID NO: | cggcgctggtttattctctctgaccggcatagctgcggcagacaatgctggaccggcagtaaccctgagctttatcct |
| 1041 | ggcctccgttggttgcgcgttcgctggcctgtgttacgcagaatttgcttctatgattccagttgcgggtagcgcctac |
| | acttatagttatgctaccatgggcgagttcgtggcgtggatcatcggttgggatctggtactcgaatacgcattgggc |
| | gcagctactgttgccgttagctggtcccagtacgtggacaaattcttgcaaaactacggcatccatattccgaactcta |
| | tcctccacgggccgtgggataccacccccggtattatcaatttaccgtcgatatttatcatctgcctgctgagcgtgct |
| | gctgattcgtggtactaaagaatctgctctgatcaacaacattctggtaatcctgaaagtcacggttgtcatcgtgttca |
| | ttggcctgggctgggggttcatgaactccgcaaaccacacgccctttatcccggttaacgaaggtgaggctctactg |
| | tcttctggtgaaatgagtttcctcaactttttcagcagtgactactttggacactacggatggtccggtattcttcgcggc |
| | gctggtgtagtattcttcgcatttatcggcttcgacgcggtgagcactgcggcacaggaggccaaggatccgcaga |
| | aaggcatgccaatcggtattctgggctcactgatcatttgcaccgttctgtacgtgcttttcgctttcgttctgaccggtc |
| | tggaaaactatctaaacttcaaaggtgacgcttctcctgtcaccactgcatttgccaaaacaggctatactttcctgaat |
| | agcggtctgacgatcgctatcatagcgggctacacatccgttatgctggtaatgttgatgggtcagtcccgtgtctttt |
| | atagtatgtctgtggatggcctgcttccgaagtttttctcgaccctgcataccaaaaacaggactccgtacaaaactaa |
| | tttgctgttcatggttttcgtaagcctgttcgctggctttgttccggtcagcgacctgggccatatggtatccatcggtac |
| | cctcttcgctttctgcctggtgtgtatcggcgttatcgttatgcgaaaaaccaacccagacgccgttcgcggttttcgtg |
| | ttccttttgtaccggttttcccgattatcggtgtagttatttgtctggttctaatggcgggcctgccgattgaatcttggga |
| | acgtctggcgatctggatgattctgggtgtcgtgatctacttcttctactctaaaaagaactctaaactgaataaccccg |
| | aataa |
| MetP | atggggacgatcaatactaagaccaacaaatatatgagcatttggaaaaccaaaccgttgtctgtactgttaaacgag |
| (F. frigoris) | gcctcagaagatgaaaagggcctgaaaaggactctgtcctctcgttccctcgtggctctgggtgtcggtgcgatcat |
| SEQ ID NO: | tggcgcaggtctgtttagcctaacaggcatcgcagctgcggaacatgctggtccagcggttactctgagtttcatact |
| 1042 | ggccgctgttggttgtgctttcgcaggcctgtgctacgcggagtttgcgtcgatgatccctgtggctgggtctgcttac |
| | acctatagctacgcaaccatgggcgaatttatggcgtggatcattggctgggaccttgtactggaatacgctctgggt |
| | gcagcgactgttggtgtatcctggtcccgttacttactggaattgctgaacaaatatggtgttcacctgaacccgaaat |
| | tcatctgctctccgtgggagacacttaccctgggcgacggcactattatcgatggcgggtacatcaatctgccggca |
| | attctgatcgtgagcgccctcagcttgctgctgattagaggtacccaggaatctgcttctattaacaacatcctggttgt |
| | gctgaaagtaatagtcgtgatcatgttcatcgttttaggatgggactatatcgatcccgcaaattactcaccttacatcc |
| | cggaaaacaccggcgtaaagggccaattcggttggtcgggtatcgctgcgggtgctggtacggttttctttgccttc |
| | attggtttcgacgccgtttccactgcggctcaggaggctaaaaacccgcagaaaggcatgccaattggcatcctgg |
| | ggtctttggtaatttgtacgatcctgtacgtcctttttgcccacgttatgacgggcctggtgccgtattataagttcgctg |
| | gagatgctaaacccgctgcgacagcattcgcagtcaccggttacagttttctgcaaactggactgattgttgcgatcc |
| | tggctggctatactagcgttatgctggtcatgctgatggggcagagtcgtgttttctacaccatgagcaaagacggtc |
| | tgctaccaccgctgttcggtcagatccattcgaaatttcgcactccgtacaagactaacctgttctttatggtattcgtttc |
| | tttattcgcgggtttcgtgccggttagcgacctcggccacatggtcagcatcggtaccctcctggcgtttgttcttgtgt |
| | gcataggtgtgctggtgatgcgaaaaaagatgccagatgctccgcgttctttcaaaaccccgttcgttccgtatgtac |
| | ccatcgcaggcgtcctggtgtgcacttacctgatgtactccctcccttacgaatcctggattcgcttagtgctttggatg |
| | gctatcggcgtagccctgtacttcgtgtatggaaaaaagcactcaaaactgaacaatccggataa |
| MetNIQ | atgataaaactttcgaatatcaccaaagtgttccaccagggcacccgcaccatccaggcgttgaacaacgtcagcct |
| SEQ ID NO: | gcatgtgccagctggacaaatttatggcgttatcggtgcctcaggcgcgggtaagagtacgcttatacgttgtgtaaa |
| 1043 | cctgctggagcgcccaaccgagggtagcgtgctggtcgatggccaggaactgaccacgctgtcagaatccgagtt |
| | gaccaaagctcgtcgccagattggtatgattttccagcattttaacctgctctcttcgcgtactgtttttggcaacgtggc |
| | tctgccgctggagctggacaacacaccgaaagacgagatcaaacgtcgcgtgacggaattgctgtcattggttggt |
| | cttggcgataagcatgatagctacccgtcgaatctttccggtgggcagaaacaacgtgtggcgattgcccgtgcatt |
| | agccagcaatcccaaagtattgctgtgtgatgaagccaccagcgcgctggacccggcaacgacacgttctattctc |
| | gaactgctgaaagacatcaaccgccgtctgggtttgacgattctgttgatcactcacgaaatggacgttgtgaagcg |
| | catttgtgattgcgtggcggtcatcagcaatggcgaactgatcgagcaggacacggtaagtgaagtgttctcgcatc |
| | cgaaaacgccgctggcgcagaagtttattcagtcgaccctgcatctggatatcccggaagattaccaggaacgtct |
| | gcaagcggagccatttactgactgcgtgccgatgctgcgtctggagtttaccggtcaatcggtcgatgccccactgc |
| | tttctgaaaccgcgcgtcgtttcaacgtcaacaacaacattattagcgcgcagatggattacgccggtggcgttaagt |
| | tcggcatcatgctgactgaaatgcacggcacacaacaagatacgcaagccgccattgcctggctgcaggaacacc |
| | atgtaaaagtagaggtactgggttatgtctgagccgatgatgtggctgctggttcgtggcgtatgggaaacgctggc |
| | aatgaccttcgtatccggtttttttggctttgtgattggtctgccggttggcgttctgctttatgtcacgcgtccggggca |
| | aattattgctaacgcgaagttgtatcgtaccatttctgcgattgtgaacattttccgttccatcccgttcattatcttgctgg |
| | tatggatgattccgtttacccgcgttattgtcggtacatcgattggattgcaggcagcgattgttccgttaaccgttggt |
| | gcagcaccgtttattgcccgtatggtcgagaacgctctgctggagatcccaaccgggttaattgaagcttcccgcgc |
| | aatgggggccacgccaatgcagatcgtccgtaaagtgctgttaccggaagcgttgccgggtctggtgaatgcggc |
| | aactatcaccctgattaccctggttggttattccgcgatgggtggtgcagtcggtgccggtggtttaggtcagattggc |
| | tatcagtatggctacatcggctacaacgcgacggtgatgaatacggtactggtattgctggtcattctggtttatttaatt |
| | cagttcgcaggcgaccgcatcgtccgggctgtcactcgcaagtaacgttcaacacaacataaataattgaagaagg |
| | aataaggtatggcgttcaaattcaaaacctttgcggcagtgggagccctgattggatcactggcactggtaggctgc |
| | ggtcaggatgaaaaagatccaaaccacattaaagtcggcgtgattgttggtgccgaacagcaggttgcagaagtcg |
| | cgcagaaagttgcgaaagacaaatatggcctggacgttgagctggtaaccttcaacgactatgttctgccaaacga |
| | agcattgagcaaaggcgatatcgacgccaacgccttccagcataaaccgtaccttgatcagcaactgaaagatcgt |
| | ggctacaaactggtcgcagtaggcaacacatttgtttatccgattgctggttactccaagaaaatcaaatcactggatg |
| | aactgcaggatggttcgcaggttgccgtgccaaacgacccaactaaccttggtcgttcactgctgctgctgcaaaaa |
| | gtgggcttgatcaaactgaaagatggcgttggcctgctgccgaccgttcttgatgttgttgagaacccaaaaaatctg |
| | aaaattgttgaactggaagcaccgcagctaccgcgctctctggacgacgcgcaaatcgctctggcagttatcaatac |
| | cacctatgccagccagattggcctgactccagcgaaagacggtatctttgtcgaagataaagagtccccgtacgtaa |
| | acctgatcgtaacgcgtgaagacaacaaagacgccgaaaacgtgaagaaattcgttcaggcttatcagtctgacga |
| | agtttacgaagcagcaaacaaagtgtttaacggcggcgctgttaaaggctggtaa |
| Methionine | atgtttgaga agtattttcc aaatgttgac ttgaccgagt tatggaatgc cacatatgaaactctgtata |
| import system | tgacattgat ttccttactg tttgccttcg taatcggcgt catcctgggattgctgttat tcttaacatc |
| permease | taaggggtct ctttggcaaa ataaagcagt aaattccgttatcgcagccg ttgtcaacat ctttcgttca |
| protein MetP | attcccttcc ttattttaat catcctgcttcttggtttca ctaaattctt agtgggaaca attttgggac |
| (Bacillus | caaatgcggc tcttcccgcgttagtcatcg gtagtgctcc cttttatgct cgtctggtcg aaatcgcact |
| subtilis) | tcgtgaagtggacaaaggag tgattgaggc ggcgaaatcg atgggggcta agacgagcac tattattttt |
| SEQ ID NO: | aaggttctta tccccgagtc catgcccgcg ctgatttccg gaattacagt gactgcgatt gcattgatcg |
| 1044 | ggtcaaccgc catcgcagga gctattggtt ctggtggatt gggaaacttagcatacgttg aaggctatca |
| | atcgaataat gcggatgtga ccttcgtggc cacagttttcatcctgatta ttgttttcat cattcagatc |
| | attggtgacc ttattaccaa catcatcgataaacgc |
| MetNIQ | atgataaaactttcgaatatcaccaaagtgttccaccagggcacccgcaccatccaggcgttgaacaacgtcagcct |
| (P281G) | gcatgtgccagctggacaaatttatggcgttatcggtgcctcaggcgcgggtaagagtacgcttatacgttgtgtaaa |
| SEQ ID NO: | cctgctggagcgcccaaccgagggtagcgtgctggtcgatggccaggaactgaccacgctgtcagaatccgagtt |
| 1045 | gaccaaagctcgtcgccagattggtatgattttccagcattttaacctgctctcttcgcgtactgtttttggcaacgtggc |
| | tctgccgctggagctggacaacacaccgaaagacgagatcaaacgtcgcgtgacggaattgctgtcattggttggt |
| | cttggcgataagcatgatagctacccgtcgaatctttccggtgggcagaaacaacgtgtggcgattgcccgtgcatt |
| | agccagcaatcccaaagtattgctgtgtgatgaagccaccagcgcgctggacccggcaacgacacgttctattctc |
| | gaactgctgaaagacatcaaccgccgtctgggtttgacgattctgttgatcactcacgaaatggacgttgtgaagcg |
| | catttgtgattgcgtggcggtcatcagcaatggcgaactgatcgagcaggacacggtaagtgaagtgttctcgcatc |
| | cgaaaacgccgctggcgcagaagtttattcagtcgaccctgcatctggatatcccggaagattaccaggaacgtct |
| | gcaagcggagccatttactgactgcgtgccgatgctgcgtctggagtttaccggtcaatcggtcgatgccggcctg |
| | ctttctgaaaccgcgcgtcgtttcaacgtcaacaacaacattattagcgcgcagatggattacgccggtggcgttaag |
| | ttcggcatcatgctgactgaaatgcacggcacacaacaagatacgcaagccgccattgcctggctgcaagaacac |
| | catgtaaaagtagaggtactgggttatgtctgagccgatgatgtggctgctggttcgtggcgtatgggaaacgctgg |
| | caatgaccttcgtatccggtttttttggctttgtgattggtctgccggttggcgttctgctttatgtcacgcgtccggggc |
| | aaattattgctaacgcgaagttgtatcgtaccatttctgcgattgtgaacattttccgttccatcccgttcattatcttgctg |
| | gtatggatgattccgtttacccgcgttattgtcggtacatcgattggattgcaggcagcgattgttccgttaaccgttgg |
| | tgcagcaccgtttattgcccgtatggtcgagaacgctctgctggagatcccaaccgggttaattgaagcttcccgcg |
| | caatgggggccacgccaatgcagatcgtccgtaaagtgctgttaccggaagcgttgccgggtctggtgaatgcgg |
| | caactatcaccctgattaccctggttggttattccgcgatgggtggtgcagtcggtgccggtggtttaggtcagattgg |
| | ctatcagtatggctacatcggctacaacgcgacggtgatgaatacggtactggtattgctggtcattctggtttatttaa |
| | ttcagttcgcaggcgaccgcatcgtccgggctgtcactcgcaagtaacgttcaacacaacataaataattgaagaag |
| | gaataaggtatggcgttcaaattcaaaacctttgcggcagtgggagccctgattggatcactggcactggtaggctg |
| | cggtcaggatgaaaaagatccaaaccacattaaagtcggcgtgattgttggtgccgaacagcaggttgcagaagtc |
| | gcgcagaaagttgcgaaagacaaatatggcctggacgttgagctggtaaccttcaacgactatgttctgccaaacg |
| | aagcattgagcaaaggcgatatcgacgccaacgccttccagcataaaccgtaccttgatcagcaactgaaagatcg |
| | tggctacaaactggtcgcagtaggcaacacatttgtttatccgattgctggttactccaagaaaatcaaatcactggat |
| | gaactgcaagatggttcgcaggttgccgtgccaaacgacccaactaaccttggtcgttcactgctgctgctgcaaaa |
| | agtgggcttgatcaaactgaaagatggcgttggcctgctgccgaccgttcttgatgttgttgagaacccaaaaaatct |
| | gaaaattgttgaactggaagcaccgcagctaccgcgctctctggacgacgcgcaaatcgctctggcagttatcaat |
| | accacctatgccagccagattggcctgactccagcgaaagacggtatctttgtcgaagataaagagtccccgtacgt |
| | aaacctgatcgtaacgcgtgaagacaacaaagacgccgaaaacgtgaagaaattcgttcaggcttatcagtctgac |
| | gaagtttacgaagcagcaaacaaagtgtttaacggcggcgctgttaaaggctggtaa |
| Recoded | atgataaaactttcgaatatcaccaaagtgttccaccagggcacccgcaccatccaggcgttgaacaacgtcagcct |
| MetNIQ | gcatgtgccagctggacaaatttatggcgttatcggtgcctcaggcgcgggtaagagtacgcttatacgttgtgtaaa |
| SEQ ID NO: | cctgctggagcgcccaaccgagggtagcgtgctggtcgatggccaggaactgaccacgctgtcagaatccgagtt |
| 1046 | gaccaaagctcgtcgccagattggtatgattttccagcattttaacctgctctcttcgcgtactgtttttggcaacgtggc |
| | tctgccgctggagctggacaacacaccgaaagacgagatcaaacgtcgcgtgacggaattgctgtcattggttggt |
| | cttggcgataagcatgatagctacccgtcgaatctttccggtgggcagaaacaacgtgtggcgattgcccgtgcatt |
| | agccagcaatcccaaagtattgctgtgtgatgaagccaccagcgcgctggacccggcaacgacacgttctattctc |
| | gaactgctgaaagacatcaaccgccgtctgggtttgacgattctgttgatcactcacgaaatggacgttgtgaagcg |
| | catttgtgattgcgtggcggtcatcagcaatggcgaactgatcgagcaggacacggtaagtgaagtgttctcgcatc |
| | cgaaaacgccgctggcgcagaagtttattcagtcgaccctgcatctggatatcccggaagattaccaggaacgtct |
| | gcaagcggagccatttactgactgcgtgccgatgctgcgtctggagtttaccggtcaatcggtcgatgccccactgc |
| | tttctgaaaccgcgcgtcgtttcaacgtcaacaacaacattattagcgcgcagatggattacgccggtggcgttaagt |
| | tcggcatcatgctgactgaaatgcacggcacacaacaagatacgcaagccgccattgcctggctgcaagaacacc |
| | atgtaaaagtagaggtactgggttatgtctgagccgatgatgtggctgctggttcgtggcgtatgggaaacgctggc |
| | aatgaccttcgtatccggtttttttggctttgtgattggtctgccggttggcgttctgctttatgtcacgcgtccggggca |
| | aattattgctaacgcgaagttgtatcgtaccatttctgcgattgtgaacattttccgttccatcccgttcattatcttgctgg |
| | tatggatgattccgtttacccgcgttattgtcggtacatcgattggattgcaggcagcgattgttccgttaaccgttggt |
| | gcagcaccgtttattgcccgtatggtcgagaacgctctgctggagatcccaaccgggttaattgaagcttcccgcgc |
| | aatgggggccacgccaatgcagatcgtccgtaaagtgctgttaccggaagcgttgccgggtctggtgaatgcggc |
| | aactatcaccctgattaccctggttggttattccgcgatgggtggtgcagtcggtgccggtggtttaggtcagattggc |
| | tatcagtatggctacatcggctacaacgcgacggtgatgaatacggtactggtattgctggtcattctggtttatttaatt |
| | cagttcgcaggcgaccgcatcgtccgggctgtcactcgcaagtaacgttcaacacaacataaataattgaagaagg |
| | aataaggtatggcgttcaaattcaaaacctttgcggcagtgggagccctgattggatcactggcactggtaggctgc |
| | ggtcaggatgaaaaagatccaaaccacattaaagtcggcgtgattgttggtgccgaacagcaggttgcagaagtcg |
| | cgcagaaagttgcgaaagacaaatatggcctggacgttgagctggtaaccttcaacgactatgttctgccaaacga |
| | agcattgagcaaaggcgatatcgacgccaacgccttccagcataaaccgtaccttgatcagcaactgaaagatcgt |
| | ggctacaaactggtcgcagtaggcaacacatttgtttatccgattgctggttactccaagaaaatcaaatcactggatg |
| | aactgcaagatggttcgcaggttgccgtgccaaacgacccaactaaccttggtcgttcactgctgctgctgcaaaaa |
| | gtgggcttgatcaaactgaaagatggcgttggcctgctgccgaccgttcttgatgttgttgagaacccaaaaaatctg |
| | aaaattgttgaactggaagcaccgcagctaccgcgctctctggacgacgcgcaaatcgctctggcagttatcaatac |
| | cacctatgccagccagattggcctgactccagcgaaagacggtatctttgtcgaagataaagagtccccgtacgtaa |
| | acctgatcgtaacgcgtgaagacaacaaagacgccgaaaacgtgaagaaattcgttcaggcttatcagtctgacga |
| | agtttacgaagcagcaaacaaagtgtttaacggcggcgctgttaaaggctggtaa |
| MetNIQ | atgataaaactttcgaatatcaccaaagtgttccaccagggcacccgcaccatccaggcgttgaacaacgtcagcct |
| (P281S) | gcatgtgccagctggacaaatttatggcgttatcggtgcctcaggcgcgggtaagagtacgcttatacgttgtgtaaa |
| SEQ ID NO: | cctgctggagcgcccaaccgagggtagcgtgctggtcgatggccaggaactgaccacgctgtcagaatccgagtt |
| 1047 | gaccaaagctcgtcgccagattggtatgattttccagcattttaacctgctctcttcgcgtactgtttttggcaacgtggc |
| | tctgccgctggagctggacaacacaccgaaagacgagatcaaacgtcgcgtgacggaattgctgtcattggttggt |
| | cttggcgataagcatgatagctacccgtcgaatctttccggtgggcagaaacaacgtgtggcgattgcccgtgcatt |
| | agccagcaatcccaaagtattgctgtgtgatgaagccaccagcgcgctggacccggcaacgacacgttctattctc |
| | gaactgctgaaagacatcaaccgccgtctgggtttgacgattctgttgatcactcacgaaatggacgttgtgaagcg |
| | catttgtgattgcgtggcggtcatcagcaatggcgaactgatcgagcaggacacggtaagtgaagtgttctcgcatc |
| | cgaaaacgccgctggcgcagaagtttattcagtcgaccctgcatctggatatcccggaagattaccaggaacgtct |
| | gcaagcggagccatttactgactgcgtgccgatgctgcgtctggagtttaccggtcaatcggtcgatgcctccctgc |
| | tttctgaaaccgcgcgtcgtttcaacgtcaacaacaacattattagcgcgcagatggattacgccggtggcgttaagt |
| | tcggcatcatgctgactgaaatgcacggcacacaacaagatacgcaagccgccattgcctggctgcaagaacacc |
| | atgtaaaagtagaggtactgggttatgtctgagccgatgatgtggctgctggttcgtggcgtatgggaaacgctggc |
| | aatgaccttcgtatccggtttttttggctttgtgattggtctgccggttggcgttctgctttatgtcacgcgtccggggca |
| | aattattgctaacgcgaagttgtatcgtaccatttctgcgattgtgaacattttccgttccatcccgttcattatcttgctgg |
| | tatggatgattccgtttacccgcgttattgtcggtacatcgattggattgcaggcagcgattgttccgttaaccgttggt |
| | gcagcaccgtttattgcccgtatggtcgagaacgctctgctggagatcccaaccgggttaattgaagcttcccgcgc |
| | aatgggggccacgccaatgcagatcgtccgtaaagtgctgttaccggaagcgttgccgggtctggtgaatgcggc |
| | aactatcaccctgattaccctggttggttattccgcgatgggtggtgcagtcggtgccggtggtttaggtcagattggc |
| | tatcagtatggctacatcggctacaacgcgacggtgatgaatacggtactggtattgctggtcattctggtttatttaatt |
| | cagttcgcaggcgaccgcatcgtccgggctgtcactcgcaagtaacgttcaacacaacataaataattgaagaagg |
| | aataaggtatggcgttcaaattcaaaacctttgcggcagtgggagccctgattggatcactggcactggtaggctgc |
| | ggtcaggatgaaaaagatccaaaccacattaaagtcggcgtgattgttggtgccgaacagcaggttgcagaagtcg |
| | cgcagaaagttgcgaaagacaaatatggcctggacgttgagctggtaaccttcaacgactatgttctgccaaacga |
| | agcattgagcaaaggcgatatcgacgccaacgccttccagcataaaccgtaccttgatcagcaactgaaagatcgt |
| | ggctacaaactggtcgcagtaggcaacacatttgtttatccgattgctggttactccaagaaaatcaaatcactggatg |
| | aactgcaagatggttcgcaggttgccgtgccaaacgacccaactaaccttggtcgttcactgctgctgctgcaaaaa |
| | gtgggcttgatcaaactgaaagatggcgttggcctgctgccgaccgttcttgatgttgttgagaacccaaaaaatctg |
| | aaaattgttgaactggaagcaccgcagctaccgcgctctctggacgacgcgcaaatcgctctggcagttatcaatac |
| | cacctatgccagccagattggcctgactccagcgaaagacggtatctttgtcgaagataaagagtccccgtacgtaa |
| | acctgatcgtaacgcgtgaagacaacaaagacgccgaaaacgtgaagaaattcgttcaggcttatcagtctgacga |
| | agtttacgaagcagcaaacaaagtgtttaacggcggcgctgttaaaggctggtaa |
| Sporomusa | atgaactttttccgcactaaatgtattgacaagttaaaagaaggcgcagagcagcaaggtttgaaaaagagtctggg |
| termitida MetP - | ggctaccgatctgatccttctgggtatcggatgcatcattggcacaggcatcttcgttctgacgggcgtcgcggctgc |
| Codon- | aaattatgccggtccgggtataatgctgtccttcgtgatctcgggtctggcgtgcgcttttgcagctctggcctacgcg |
| optimized | gaactagctgctatggttccaattgctggcagcgcgtacacttattcttacgccgcgttaggcgaaatcgtagcatgg |
| sequence | attgtaggttggaacctgatcctcgagtacagcgttgggtcttcagctgtggccgcgggttggtccggctacatggta |
| SEQ ID NO: | ggcctgctgaaaagcggtggtatcgaactgcctaaagctttcaccgcagttccggctgatggtggtttggtgaacttg |
| 1129 | ccggccatgttaattgctctgctgctgtcggttctgctagtccgtggcaccaaagaatctgtgactctgaataaggtcc |
| | tggttgtgattaaactggcagcggttttcatctttctggctctggcgggccccaaagtaaacccggctaactggtccc |
| | ctctgatgccgtatgggttctctggtgtagcggcgggtgcagccattatctttttcgcttacatcggcttcgacgcagta |
| | gctaccgctgctgaagagtgccggaacccgaaacgagacttaccagcaggtatcatcgggtcactggttatctgca |
| | ctatactgtatattgttgttgcgggcgtcctgactggcgtcgttccgtaccagcagctcaacaacgctgaaccggttg |
| | catacgctctgagagcgatcggctacaatttcgggtctgctctcgttggtaccggagctatcgccggcattacgaca |
| | gtgctgcttgtcctgatgtatggtcagacccgcatcttctttgcaatgagccgtgatggcctgatcccggctcgtatct |
| | gtaaagtacatccacgttatggaactcctcacataattaccatggcagcgggtatcgcagttgctctgattgcaggtttt |
| | acacctatcggcattatcgcggaactgactaacatcggcaccttgttcgcgttcgtggtagccgccatcggtgtactg |
| | gtgctcaggtacacccgcccggacatcccgcgtagctttaagtgcccggctgtcaaagtgattgctccgctggctgt |
| | tctgtcctgcggatacctgatggccaatctgccagcagagacttggatccgcttcggtatctggtccgccattggctt |
| | cgtcgtttactttgtttattcttatcaccatagcgttctgaacaaagcggaagtggctgggaaggaataa |
| Bacteroidetes | atgggtatctttgcgaaaaagcagctgaatcaattgattgccgaggcttccgaatctgaaaaaggcttaaaaaagact |
| bacterium 43- | ctgtcagctggagcactcgtgagtctagggatcggtgccataatcggcgcgggcctgttctctcttacgggcatggc |
| 16 MetP - | tgctgccgacaacgcaggtccggcggttgtattcagctttatcctggcagctgtcggttgcgggttcgctggtctgtg |
| Codon- | ttacgcagaatttgcgagcatgattcctgttgctggctccgcatatacatactcttatgctaccatgggcgaactgattg |
| optimized | cttggatcatcggttgggatctggtactggagtacgcgctgggtgccgcaaccgttgctgtgtcgtggagccagtac |
| sequence | gttaacaaattccttcactccgtcggcatcgacctgccacagtatctattgcatggtccgtgggatgaagtgaacggc |
| SEQ ID NO: | gttgcgatgaacggtattattaacctgcccgcgatcatcattgtatgcctgctgtctctgctgctgatacgcggcacta |
| 1131 | aagagagtgcgctattgaataacatcctggtgatcctgaaagtcgttgtagttttggtcttcatctgtattggctggtcttt |
| | catcaacccggctaatcacgaaccgtttattccggttaacgctggtgaagagatggtaaaaagcggtaccatgtcttt |
| | ttggagcttcttcacttccgaaagcttcggatcttacggtatctcgggcatcctgcgtggcgctggtgtggttttctttgc |
| | atttatcggattcgatgccgttagcaccgctgctcaagaagccaagaacccgcagaaaggcatgccaatcgggatc |
| | attggtagtctggttgtgtgcactatcctgtatgtgctcttcgcatacgtactgactggcctggagaactacataaacttt |
| | aaaggtaatgcgtccccggttaccaccgcgttcgcacacaccggttatacgtttttaaactctttccttactatggcgat |
| | tatcgcaggttacacctcagttatgcttgtaatgctgatgggccagtcccgtgtgttctatagtatgtcggtggacgga |
| | ctgctgcctaaaatgttttctgacctgcataagaaaaacaggacaccgtacaagactaacctgatcttcatggtgtttgt |
| | ctcactgttcgcaggcttcgttccggtagcagatctggggcacatggtcagcatcggtacattattcgcattcgctctg |
| | gtgtgcattggcgttatagtgatgcgcaaaactaatcccgatgccgttagagggttccgtactccgttcgtcccagttc |
| | tccctatcctgggtgtcttagtatgtgtagtactgatgctgggcctgccgaaagaatcctgggaacgtttggccatctg |
| | gcttggtttgggcctgattatctactttgcttacagcaagaaaaactctaaaattggaaacaaataa |

TABLE 12
Exemplary Methionine Decarboxylase Amino Acid Sequences

| Description | |
|---|---|
| SEQ ID NO: | Amino Acid Sequence |
| MetDC Q70D | MSPTAFPAAETATAPATAVDPGPELDGGDFALPEGGLDDDRRLRALDAV |
| N82H | DEYLTRKRKHLVGYQATQDMDGTALDLARFMPHNINNLGDPFQSGGY |
| SEQ ID NO: | KPNTKVVERAVLDYYAKLWHAERPHDPADPESYWGYMLSMGSTEGN |
| 1048 | MYALWNARDYLSGKALIQPPTAPFDAVRYVKADPDRRNPNAHHPVAFY |
| | SEDTHYSFAKAVAVLGVETFHAVGLEKYADECPLVDPVTGLRTWPTEV |
| | PSRPGPSGLSWDGPGEIDVDALAVLVEFFAAKGHPVFVNLNLGSTFKGA |
| | HDDVRAVCERLLPIFERHGLVQREVVYGSCPQTGRPLVDVRRGFWIHVD |
| | GALGAGYAPFLRLAAEDPEGYGWTPEAELPEFDFGLRLPTAGHGEVDM |
| | VSSIAMSGHKWAGAPWPCGIYMTKVKYQISPPSQPDYIGAPDTTFAGSR |
| | NGFSPLILWDHLSRYSYRDQVERIREAQELAAYLERRLTAMERELGVEL |
| | WPARTPGAVTVRFRKPSAELVAKWSLSSQDVLMVPGDETTRRSYVHVF |
| | VMPSVDRAKLDALLAELAEDPVILGAP |
| MetDC | MSPTAFPAAETATAPATAVDPGPELDGGDFALPEGGLDDDRRLRALDAV |
| (Streptomyces) | DEYLTRKRKHLVGYQATQDMQGTALDLARFMPNNINNLGDPFQSGGY |
| SEQ ID NO: | KPNTKVVERAVLDYYAKLWHAERPHDPADPESYWGYMLSMGSTEGN |
| 1049 | MYALWNARDYLSGKALIQPPTAPFDAVRYVKADPDRRNPNAHHPVAFY |
| | SEDTHYSFAKAVAVLGVETFHAVGLEKYADECPLVDPVTGLRTWPTEV |
| | PSRPGPSGLSWDGPGEIDVDALAVLVEFFAAKGHPVFVNLNLGSTFKGA |
| | HDDVRAVCERLLPIFERHGLVQREVVYGSCPQTGRPLVDVRRGFWIHVD |
| | GALGAGYAPFLRLAAEDPEGYGWTPEAELPEFDFGLRLPTAGHGEVDM |
| | VSSIAMSGHKWAGAPWPCGIYMTKVKYQISPPSQPDYIGAPDTTFAGSR |
| | NGFSPLILWDHLSRYSYRDQVERIREAQELAAYLERRLTAMERELGVEL |
| | WPARTPGAVTVRFRKPSAELVAKWSLSSQDVLMVPGDETTRRSYVHVF |
| | VMPSVDRAKLDALLAELAEDPVILGAP |
| MetDC | MSPTAFPAAETATAPATAVDPGPELDGGDFALPEGGLDDDRRLRALDAV |
| (V491L | DEYLTRKRKHLVGYQATQDMQGTALDLARFMPNNINNLGDPFQSGGY |
| A500P) | KPNTKVVERAVLDYYAKLWHAERPHDPADPESYWGYMLSMGSTEGN |
| SEQ ID NO: | MYALWNARDYLSGKALIQPPTAPFDAVRYVKADPDRRNPNAHHPVAFY |
| 1050 | SEDTHYSFAKAVAVLGVETFHAVGLEKYADECPLVDPVTGLRTWPTEV |
| | PSRPGPSGLSWDGPGEIDVDALAVLVEFFAAKGHPVFVNLNLGSTFKGA |
| | HDDVRAVCERLLPIFERHGLVQREVVYGSCPQTGRPLVDVRRGFWIHVD |
| | GALGAGYAPFLRLAAEDPEGYGWTPEAELPEFDFGLRLPTAGHGEVDM |
| | VSSIAMSGHKWAGAPWPCGIYMTKVKYQISPPSQPDYIGAPDTTFAGSR |
| | NGFSPLILWDHLSRYSYRDQVERIREAQELAAYLERRLTAMERELGVEL |
| | WPARTPGALTVRFRKPSPELVAKWSLSSQDVLMVPGDETTRRSYVHVF |
| | VMPSVDRAKLDALLAELAEDPVILGAP |
| MetDC (R41Q | MSPTAFPAAETATAPATAVDPGPELDGGDFALPEGGLDDDQRLRALDA |
| Q70D) | VDEYLTRKRKHLVGYQATQDMDGTALDLARFMPNNINNLGDPFQSGG |
| SEQ ID NO: | YKPNTKVVERAVLDYYAKLWHAERPHDPADPESYWGYMLSMGSTEGN |
| 1051 | MYALWNARDYLSGKALIQPPTAPFDAVRYVKADPDRRNPNAHHPVAFY |
| | SEDTHYSFAKAVAVLGVETFHAVGLEKYADECPLVDPVTGLRTWPTEV |
| | PSRPGPSGLSWDGPGEIDVDALAVLVEFFAAKGHPVFVNLNLGSTFKGA |
| | HDDVRAVCERLLPIFERHGLVQREVVYGSCPQTGRPLVDVRRGFWIHVD |
| | GALGAGYAPFLRLAAEDPEGYGWTPEAELPEFDFGLRLPTAGHGEVDM |
| | VSSIAMSGHKWAGAPWPCGIYMTKVKYQISPPSQPDYIGAPDTTFAGSR |
| | NGFSPLILWDHLSRYSYRDQVERIREAQELAAYLERRLTAMERELGVEL |
| | WPARTPGAVTVRFRKPSAELVAKWSLSSQDVLMVPGDETTRRSYVHVF |
| | VMPSVDRAKLDALLAELAEDPVILGAP |
| MetDC | MGFQLLSKHKLSAEDQQKLDRFYRDIQTEAERFLGYPCNELFDYSPLFRF |
| (Stanieria sp. | LQYPLNNVGDPYLPSNYHLNTHNFECEVLEIFRTLTEATEGSTWGYVTN |
| NIES-3757) | GGTEGNHYGLFLARELLPEGLVYYSQDAHYSIDKILRCLNLRSIMIRSHD |
| SEQ ID NO: | DGRMDLDDLRETLRIHRDLPPIVCATIGTTMKGAVDDIAGIKKIFKDLAIH |
| 1052 | RHYIHADAALGGMILPFLDNSPPWNFKAGIDSIAISGHKMVGSPIPCGVV |
| | LAKKSNVERIAQSVEYIGTLDTTLSGSRNALTPLFLWYAFHTVGIEGFKRI |
| | IPACLKMADYAIAQLNKINRNAWRYPYSNTVVFDRPSPEVTRYWQLAC |
| | QGNLSHLITMPHVTSTQIDHLVADIIASEPIPPLPTLSVTPACELLTSTPDQ |
| | DITLIGTANHNLLSEVSTALAAEGLSIENLAAVAVESEDVEVVRLRVNNR |
| | ERALQILNQNLDIGRCYGQARPFGNEEATQVLSQLEYQSVGEDALLVQL |
| | DDCPGSLAELLKDCRNEAVKIRNIRLLWRGHGKGVVAIATTSPDALKTL |
| | LKDRILLS |
| LeuDC | MSTPSEVKKDLLGAAGSLWPSEPITLGPGESAWQLVLKKIQELSDSGHQ |
| (Mus musculus) | DPFMVADLDVLVSRHQTFCQALPRVQPFYAVKCNSNPWVLRVLAALGT |
| SEQ ID NO: | GFDCASQGELEQVLGLGVAPSRIIFANPCKAVSHIQFAARCGVQLLTFDS |
| 1053 | EEELIKVAQYHPGARLVLRIQTQDSQSTFPLSTKFGASLEACGHLLQVAR |
| | ELGLAVVGASFHVGSDCHTPQSFRQAIADCHRVFEMGRKAGHDMSLLD |
| | LGGGFPGVEGSEAKFEEMARVINAALAQYFPEETGIEVIAEPGRFYAGSV |
| | CTAAVNIIAKKSVLEPGGHRKLMYYLNEGHYGSFRLFLRDPVPRIPIVVK |
| | EFPSEPPLFPCTLYGPTCDAYDRLFSEEVQLPELDVGDWLIFPDMGAYTS |
| | SMSSTFNGFPPATVYCAMSPQLRSLLETVP |
| MetDC | MNTPSEVKKDLLGVAEHLRPSEPITLGPGASAWQLVLKKIKELSISGRQD |
| (Mus musculus) | AFMVADLDVLVSRHRTFLQALPRVQPFYAVKCNSNPWVLLVLAALGTG |
| SEQ ID NO: | FDCASQGELEQVLGLGVAPSRIIFANPCKAVSHIQFAARCGVQLLTFDNE |
| 1054 | EELIKLARYHPRARLVLRIQTLDSQSTFPLSTKFGAHLEACGHLLQVARE |
| | LGLAVVGASFHVGSDCHTPESYRQAIADCHRVFEMGCKAGHHMSLLDL |
| | GGGFPGVKGSEAKFEEVARVINTALAQYFPEETGIEVIAEPGRFYAGSVC |
| | TAAVNIIAKKSSLDPGGHRKLAYYLNEGHYGVFRLFLRDPVPRIPIVVKE |
| | FPSEPPLFPCTLYGPTCDAYDRLFSTEVQLPELDVGDWLIFPDMGAYSSS |
| | MSSTFNGFPIATVYDAMSPQLRSLLETVP |
| MetDC | MKQTSLEVKEFALNLISQFEPENQPLGFWIFDTEGVEKAVERWKKNMPT |
| (Entamoeba | VRPCFAVKCNPEPHLVKLLGELGCGFDCASLNEIKEVLDLGFNPEDITYS |
| histolytica) | QTFKPYNQLIEASHLGINHTIVDSIDEVQKIAKYAPKMGIMIRIMENDTSA |
| SEQ ID NO: | GHVFGEKFGLHDDEVEIVLKEIKDKGLNLDGVHFHVGSDSHNSEVFTKA |
| 1055 | LTKARNTVTLAEQFGMKPYLIDIGGGFSQVAPFEEFAATIEKTIKELEFPE |
| | RTRFIAEPGRYMASNAFHLVSSLHGKRVRIQNGKKQIEYTSGDGLHGSFG |
| | CCIWFEKQKSCECITQKVNENTKMYESIIYGPSCNGSDKVATQELPEMEP |
| | GKDWLLFPNMGAYTISMATNFNGFEERNHVIYTLPLKSTKIIQIPKSIECN |
| | SVPSLNGIPHYA |
| SpMetDC | MSPTAFPAAETATAPATAVDPGPELDGGDFALPEGGLDDDRRLRALDAV |
| T66N | DEYLTRKRKHLVGYQANQDMQGTALDLARFMPNNINNLGDPFQSGGY |
| SEQ ID NO: | KPNTKVVERAVLDYYAKLWHAERPHDPADPESYWGYMLSMGSTEGN |
| 1124 | MYALWNARDYLSGKALIQPPTAPFDAVRYVKADPDRRNPNAHHPVAFY |
| | SEDTHYSFAKAVAVLGVETFHAVGLEKYADECPLVDPVTGLRTWPTEV |
| | PSRPGPSGLSWDGPGEIDVDALAVLVEFFAAKGHPVFVNLNLGSTFKGA |
| | HDDVRAVCERLLPIFERHGLVQREVVYGSCPQTGRPLVDVRRGFWIHVD |
| | GALGAGYAPFLRLAAEDPEGYGWTPEAELPEFDFGLRLPTAGHGEVDM |
| | VSSIAMSGHKWAGAPWPCGIYMTKVKYQISPPSQPDYIGAPDTTFAGSR |
| | NGFSPLILWDHLSRYSYRDQVERIREAQELAAYLERRLTAMERELGVEL |
| | WPARTPGAVTVRFRKPSAELVAKWSLSSQDVLMVPGDETTRRSYVHVF |
| | VMPSVDRAKLDALLAELAEDPVILGAP |
| SpMetDC | MSPTAFPAAETATAPATAVDPGPELDGGDFALPEGGLDDDRRLRALDAV |
| A203H | DEYLTRKRKHLVGYQATQDMQGTALDLARFMPNNINNLGDPFQSGGY |
| SEQ ID NO: | KPNTKVVERAVLDYYAKLWHAERPHDPADPESYWGYMLSMGSTEGN |
| 1126 | MYALWNARDYLSGKALIQPPTAPFDAVRYVKADPDRRNPNAHHPVAFY |
| | SEDTHYSFAKAVHVLGVETFHAVGLEKYADECPLVDPVTGLRTWPTEV |
| | PSRPGPSGLSWDGPGEIDVDALAVLVEFFAAKGHPVFVNLNLGSTFKGA |
| | HDDVRAVCERLLPIFERHGLVQREVVYGSCPQTGRPLVDVRRGFWIHVD |
| | GALGAGYAPFLRLAAEDPEGYGWTPEAELPEFDFGLRLPTAGHGEVDM |
| | VSSIAMSGHKWAGAPWPCGIYMTKVKYQISPPSQPDYIGAPDTTFAGSR |
| | NGFSPLILWDHLSRYSYRDQVERIREAQELAAYLERRLTAMERELGVEL |
| | WPARTPGAVTVRFRKPSAELVAKWSLSSQDVLMVPGDETTRRSYVHVF |
| | VMPSVDRAKLDALLAELAEDPVILGAP |
| SpMetDC | MSPTAFPAAETATAPATAVDPGPELDGGDFALPEGGLDDDRRLRALDAV |
| H379G | DEYLTRKRKHLVGYQATQDMQGTALDLARFMPNNINNLGDPFQSGGY |
| SEQ ID NO: | KPNTKVVERAVLDYYAKLWHAERPHDPADPESYWGYMLSMGSTEGN |
| 1128 | MYALWNARDYLSGKALIQPPTAPFDAVRYVKADPDRRNPNAHHPVAFY |
| | SEDTHYSFAKAVAVLGVETFHAVGLEKYADECPLVDPVTGLRTWPTEV |
| | PSRPGPSGLSWDGPGEIDVDALAVLVEFFAAKGHPVFVNLNLGSTFKGA |
| | HDDVRAVCERLLPIFERHGLVQREVVYGSCPQTGRPLVDVRRGFWIHVD |
| | GALGAGYAPFLRLAAEDPEGYGWTPEAELPEFDFGLRLPTAGGGEVDM |
| | VSSIAMSGHKWAGAPWPCGIYMTKVKYQISPPSQPDYIGAPDTTFAGSR |
| | NGFSPLILWDHLSRYSYRDQVERIREAQELAAYLERRLTAMERELGVEL |
| | WPARTPGAVTVRFRKPSAELVAKWSLSSQDVLMVPGDETTRRSYVHVF |
| | VMPSVDRAKLDALLAELAEDPVILGAP |

TABLE 13
Exemplary Importer Amino Acid Sequences

| Description | |
|---|---|
| SEQ ID NO: | Amino Acid Sequence |
| MetP | MGTINTKIYKYMSIWKTKPLSVLLSEATEDEKGLKRTLSARSLVALGVG |
| (F. segetis) | AIIGAGLFSLTGIAAADNAGPAVTLSFILASVGCAFAGLCYAEFASMIPVA |
| SEQ ID NO: | GSAYTYSYATMGEFVAWIIGWDLVLEYALGAATVAVSWSQYVDKFLQ |
| 1056 | NYGIHIPNSILHGPWDTTPGIINLPSIFIICLLSVLLIRGTKESALINNILVILK |
| | VTVVIVFIGLGWGFMNSANHTPFIPVNEGEALLSSGEMSFLNFFSSDYFG |
| | HYGWSGILRGAGVVFFAFIGFDAVSTAAQEAKDPQKGMPIGILGSLIICT |
| | VLYVLFAFVLTGLENYLNFKGDASPVTTAFAKTGYTFLNSGLTIAIIAGY |
| | TSVMLVMLMGQSRVFYSMSVDGLLPKFFSTLHTKNRTPYKTNLLFMVF |
| | VSLFAGFVPVSDLGHMVSIGTLFAFCLVCIGVIVMRKTNPDAVRGFRVPF |
| | VPVFPIIGVVICLVLMAGLPIESWERLAIWMILGVVIYFFYSKKNSKLNNP |
| | E |
| MetP | MGTINTKTNKYMSIWKTKPLSVLLNEASEDEKGLKRTLSSRSLVALGVG |
| (F. frigoris) | AIIGAGLFSLTGIAAAEHAGPAVTLSFILAAVGCAFAGLCYAEFASMIPVA |
| SEQ ID NO: | GSAYTYSYATMGEFMAWIIGWDLVLEYALGAATVGVSWSRYLLELLNK |
| 1057 | YGVHLNPKFICSPWETLTLGDGTIIDGGYINLPAILIVSALSLLLIRGTQES |
| | ASINNILVVLKVIVVIMFIVLGWDYIDPANYSPYIPENTGVKGQFGWSGIA |
| | AGAGTVFFAFIGFDAVSTAAQEAKNPQKGMPIGILGSLVICTILYVLFAH |
| | VMTGLVPYYKFAGDAKPAATAFAVTGYSFLQTGLIVAILAGYTSVMLV |
| | MLMGQSRVFYTMSKDGLLPPLFGQIHSKFRTPYKTNLFFMVFVSLFAGF |
| | VPVSDLGHMVSIGTLLAFVLVCIGVLVMRKKMPDAPRSFKTPFVPYVPIA |
| | GVLVCTYLMYSLPYESWIRLVLWMAIGVALYFVYGKKHSKLNNPD |
| MetN | MIKLSNITKVFHQGTRTIQALNNVSLHVPAGQIYGVIGASGAGKSTLIRC |
| SEQ ID NO: | VNLLERPTEGSVLVDGQELTTLSESELTKARRQIGMIFQHFNLLSSRTVFG |
| 1058 | NVALPLELDNTPKDEIKRRVTELLSLVGLGDKHDSYPSNLSGGQKQRVAI |
| | ARALASNPKVLLCDEATSALDPATTRSILELLKDINRRLGLTILLITHEMD |
| | VVKRICDCVAVISNGELIEQDTVSEVFSHPKTPLAQKFIQSTLHLDIPEDY |
| | QERLQAEPFTDCVPMLRLEFTGQSVDAPLLSETARRFNVNNNIISAQMDY |
| | AGGVKFGIMLTEMHGTQQDTQAAIAWLQEHHVKVEVLGYV |
| MetI | MSEPMMWLLVRGVWETLAMTFVSGFFGFVIGLPVGVLLYVTRPGQIIAN |
| SEQ ID NO: | AKLYRTISAIVNIFRSIPFIILLVWMIPFTRVIVGTSIGLQAAIVPLTVGAAPF |
| 1059 | IARMVENALLEIPTGLIEASRAMGATPMQIVRKVLLPEALPGLVNAATIT |
| | LITLVGYSAMGGAVGAGGLGQIGYQYGYIGYNATVMNTVLVLLVILVY |
| | LIQFAGDRIVRAVTRK |
| MetQ | MAFKFKTFAAVGALIGSLALVGCGQDEKDPNHIKVGVIVGAEQQVAEV |
| SEQ ID NO: | AQKVAKDKYGLDVELVTFNDYVLPNEALSKGDIDANAFQHKPYLDQQL |
| 1060 | KDRGYKLVAVGNTFVYPIAGYSKKIKSLDELQDGSQVAVPNDPTNLGRS |
| | LLLLQKVGLIKLKDGVGLLPTVLDVVENPKNLKIVELEAPQLPRSLDDAQ |
| | IALAVINTTYASQIGLTPAKDGIFVEDKESPYVNLIVTREDNKDAENVKK |
| | FVQAYQSDEVYEAANKVFNGGAVKGW |
| Methionine | MFEKYFPNVDLTELWNATYETLYMTLISLLFAFVIGVILGLLLFLTSKGSL |
| import system | WQNKAVNSVIAAVVNIFRSIPFLILIILLLGFTKFLVGTILGPNAALPALVI |
| permease | GSAPFYARLVEIALREVDKGVIEAAKSMGAKTSTIIFKVLIPESMPALISGI |
| protein MetP | TVTAIALIGSTAIAGAIGSGGLGNLAYVEGYQSNNADVTFVATVFILIIVFI |
| (Bacillus | IQIIGDLITNIIDKR |
| subtilis) | |
| SEQ ID NO: | |
| 1061 | |
| MetN (P281G) | MIKLSNITKVFHQGTRTIQALNNVSLHVPAGQIYGVIGASGAGKSTLIRC |
| SEQ ID NO: | VNLLERPTEGSVLVDGQELTTLSESELTKARRQIGMIFQHFNLLSSRTVFG |
| 1062 | NVALPLELDNTPKDEIKRRVTELLSLVGLGDKHDSYPSNLSGGQKQRVAI |
| | ARALASNPKVLLCDEATSALDPATTRSILELLKDINRRLGLTILLITHEMD |
| | VVKRICDCVAVISNGELIEQDTVSEVFSHPKTPLAQKFIQSTLHLDIPEDY |
| | QERLQAEPFTDCVPMLRLEFTGQSVDAGLLSETARRFNVNNNIISAQMD |
| | YAGGVKFGIMLTEMHGTQQDTQAAIAWLQEHHVKVEVLGYV |
| MetN (P281S) | MIKLSNITKVFHQGTRTIQALNNVSLHVPAGQIYGVIGASGAGKSTLIRC |
| SEQ ID NO: | VNLLERPTEGSVLVDGQELTTLSESELTKARRQIGMIFQHFNLLSSRTVFG |
| 1063 | NVALPLELDNTPKDEIKRRVTELLSLVGLGDKHDSYPSNLSGGQKQRVAI |
| | ARALASNPKVLLCDEATSALDPATTRSILELLKDINRRLGLTILLITHEMD |
| | VVKRICDCVAVISNGELIEQDTVSEVFSHPKTPLAQKFIQSTLHLDIPEDY |
| | QERLQAEPFTDCVPMLRLEFTGQSVDASLLSETARRFNVNNNIISAQMDY |
| | AGGVKFGIMLTEMHGTQQDTQAAIAWLQEHHVKVEVLGYV |
| Sporomusa | MNFFRTKCIDKLKEGAEQQGLKKSLGATDLILLGIGCIIGTGIFVLTGVAA |
| termitida MetP | ANYAGPGIMLSFVISGLACAFAALAYAELAAMVPIAGSAYTYSYAALGE |
| SEQ ID NO: | IVAWIVGWNLILEYSVGSSAVAAGWSGYMVGLLKSGGIELPKAFTAVPA |
| 1130 | DGGLVNLPAMLIALLLSVLLVRGTKESVTLNKVLVVIKLAAVFIFLALAG |
| | PKVNPANWSPLMPYGFSGVAAGAAIIFFAYIGFDAVATAAEECRNPKRD |
| | LPAGIIGSLVICTILYIVVAGVLTGVVPYQQLNNAEPVAYALRAIGYNFGS |
| | ALVGTGAIAGITTVLLVLMYGQTRIFFAMSRDGLIPARICKVHPRYGTPHI |
| | ITMAAGIAVALIAGFTPIGIIAELTNIGTLFAFVVAAIGVLVLRYTRPDIPRS |
| | FKCPAVKVIAPLAVLSCGYLMANLPAETWIRFGIWSAIGFVVYFVYSYH |
| | HSVLNKAEVAGKE |
| Bacteroidetes | MGIFAKKQLNQLIAEASESEKGLKKTLSAGALVSLGIGAIIGAGLFSLTG |
| bacterium 43- | MAAADNAGPAVVFSFILAAVGCGFAGLCYAEFASMIPVAGSAYTYSYA |
| 16 MetP | TMGELIAWIIGWDLVLEYALGAATVAVSWSQYVNKFLHSVGIDLPQYLL |
| SEQ ID NO: | HGPWDEVNGVAMNGIINLPAIIIVCLLSLLLIRGTKESALLNNILVILKVV |
| 1132 | VVLVFICIGWSFINPANHEPFIPVNAGEEMVKSGTMSFWSFFTSESFGSYG |
| | ISGILRGAGVVFFAFIGFDAVSTAAQEAKNPQKGMPIGIIGSLVVCTILYV |
| | LFAYVLTGLENYINFKGNASPVTTAFAHTGYTFLNSFLTMAIIAGYTSVM |
| | LVMLMGQSRVFYSMSVDGLLPKMFSDLHKKNRTPYKTNLIFMVFVSLF |
| | AGFVPVADLGHMVSIGTLFAFALVCIGVIVMRKTNPDAVRGFRTPFVPV |
| | LPILGVLVCVVLMLGLPKESWERLAIWLGLGLIIYFAYSKKNSKIGNK |

TABLE 14
Phage Nucleotide Sequence

| Description | SEQ ID NO: |
|---|---|
| Phage 3 | SEQ ID NO: 1064 |

TABLE 15
Colibactin Nucleotide Sequences

| Description | SEQ ID NO: |
|---|---|
| clbA | SEQ ID NO: 1065 |
| clbB | SEQ ID NO: 1066 |
| clbC | SEQ ID NO: 1067 |
| clbD | SEQ ID NO: 1068 |
| clbE | SEQ ID NO: 1069 |
| clbF | SEQ ID NO: 1070 |
| clbG | SEQ ID NO: 1071 |
| clbH | SEQ ID NO: 1072 |
| clbI | SEQ ID NO: 1073 |
| clbJ | SEQ ID NO: 1074 |
| clbK | SEQ ID NO: 1075 |
| clbL | SEQ ID NO: 1076 |
| clbM | SEQ ID NO: 1077 |
| clbN | SEQ ID NO: 1078 |
| clbO | SEQ ID NO: 1079 |
| clbP | SEQ ID NO: 1080 |
| clbQ | SEQ ID NO: 1081 |
| clbR | SEQ ID NO: 1082 |
| clbS | SEQ ID NO: 1083 |

TABLE 16
Colibactin Amino Acid Sequences

| Description | |
|---|---|
| SEQ ID NO: | Amino Acid Sequence |
| clbA | SEQ ID NO: 1084 |
| clbB | SEQ ID NO: 1085 |
| clbC | SEQ ID NO: 1086 |
| clbD | SEQ ID NO: 1087 |
| clbE | SEQ ID NO: 1088 |
| clbF | SEQ ID NO: 1089 |
| clbG | SEQ ID NO: 1090 |
| clbH | SEQ ID NO: 1091 |
| clbI | SEQ ID NO: 1092 |
| clbJ | SEQ ID NO: 1093 |
| clbK | SEQ ID NO: 1094 |
| clbL | SEQ ID NO: 1095 |
| clbM | SEQ ID NO: 1096 |
| clbN | SEQ ID NO: 1097 |
| clbO | SEQ ID NO: 1098 |
| clbP | SEQ ID NO: 1099 |
| clbQ | SEQ ID NO: 1100 |
| clbR | SEQ ID NO: 1101 |
| clbS | SEQ ID NO: 1102 |

TABLE 17
Other Exemplary Sequences

| Description | Sequence |
|---|---|
| Ptac-metP | ttgacaattaatcatcggctcgtataatgtgtggaattgtgagcgctcacaattagctgtgaccagaggtaaggaggtaac |
| SEQ ID NO: | aaccatgcgagtgttgaagaaacatcttaatcatgctgcggagggtttctaatggggaccattaacacgaagatctataaa |
| 1118 | tacatgagcatctggaaaacaaaacctctgtccgtgctcttgtctgaagcaactgaggatgaaaaaggcctgaagcgca |
| | ctctgtcggcccgttcacttgttgcgctgggtgtcggtgctattatcggcgctggtttattctctctgaccggcatagctgcg |
| | gcagacaatgctggaccggcagtaaccctgagctttatcctggcctccgttggttgcgcgttcgctggcctgtgttacgc |
| | agaatttgcttctatgattccagttgcgggtagcgcctacacttatagttatgctaccatgggcgagttcgtggcgtggatc |
| | atcggttgggatctggtactcgaatacgcattgggcgcagctactgttgccgttagctggtcccagtacgtggacaaattc |
| | ttgcaaaactacggcatccatattccgaactctatcctccacgggccgtgggataccacccccggtattatcaatttaccgt |
| | cgatatttatcatctgcctgctgagcgtgctgctgattcgtggtactaaagaatctgctctgatcaacaacattctggtaatcc |
| | tgaaagtcacggttgtcatcgtgttcattggcctgggctgggggttcatgaactccgcaaaccacacgccctttatcccgg |
| | ttaacgaaggtgaggctctactgtcttctggtgaaatgagtttcctcaactttttcagcagtgactactttggacactacggat |
| | ggtccggtattcttcgcggcgctggtgtagtattcttcgcatttatcggcttcgacgcggtgagcactgcggcacaggag |
| | gccaaggatccgcagaaaggcatgccaatcggtattctgggctcactgatcatttgcaccgttctgtacgtgcttttcgctt |
| | tcgttctgaccggtctggaaaactatctaaacttcaaaggtgacgcttctcctgtcaccactgcatttgccaaaacaggcta |
| | tactttcctgaatagcggtctgacgatcgctatcatagcgggctacacatccgttatgctggtaatgttgatgggtcagtcc |
| | cgtgtcttttatagtatgtctgtggatggcctgcttccgaagtttttctcgaccctgcataccaaaaacaggactccgtacaa |
| | aactaatttgctgttcatggttttcgtaagcctgttcgctggctttgttccggtcagcgacctgggccatatggtatccatcgg |
| | taccctcttcgctttctgcctggtgtgtatcggcgttatcgttatgcgaaaaaccaacccagacgccgttcgcggttttcgtg |
| | ttccttttgtaccggttttcccgattatcggtgtagttatttgtctggttctaatggcgggcctgccgattgaatcttgggaacg |
| | tctggcgatctggatgattctgggtgtcgtgatctacttcttctactctaaaaagaactctaaactgaataaccccgaataa |
| Ptac-metDC | ttgacaattaatcatcggctcgtataatgtgtggaattgtgagcgctcacaattaagtgaATTGCCAATAACA |
| SEQ ID NO: | ATTACTAAGGAGGTTTTTTATGtccccgacggcgtttccagcggccgaaacagctactgcccctgc |
| 1119 | aactgccgtcgatcctgggccagaactggacggcggagatttcgcccttccagagggcgggctggatgacgatcgtc |
| | gcttacgtgcattggacgcagttgacgagtatttgacccgcaagcgcaagcatttggttgggtaccaagctacccaggat |
| | atggacggaacggccttggatttagcccgtttcatgccccacaacatcaacaacctgggagatcctttccagtcgggtgg |
| | gtataaaccaaatacgaaagtcgttgagcgtgccgtactggactactatgcaaaattgtggcacgcagaacgtccacac |
| | gacccagctgacccagaaagctactggggttacatgttatcgatgggctcaactgagggcaacatgtacgccctgtgga |
| | atgcacgtgactacctgtcgggtaaggctttgattcagcctcccacggcaccatttgacgctgttcgctacgtgaaggctg |
| | accccgatcgccgcaatcctaacgcacaccacccagtcgcattctactcggaggatacccactattcttttgctaaagcc |
| | gttgcggtgctgggtgtcgaaactttccacgctgtgggtctggagaaatacgctgacgagtgccccttggtggatccagt |
| | aaccggccttcgtacctggccgaccgaagttccatcgcgcccggggccgtcgggtttaagctgggacggccctggtg |
| | agattgatgttgatgcgcttgcagtactggtcgagttcttcgcagcgaagggtcaccccgtcttcgtcaaccttaacttggg |
| | gtctacatttaaaggagcacatgatgacgtacgtgcggtatgtgaacgcttattaccaatcttcgagcgccatggcttagta |
| | caacgtgaagttgtatatgggagctgtccccaaaccggccgccctttagtggatgtacgtcgcggattttggatccacgta |
| | gatggggcacttggggcggggtatgccccttttctgcgtcttgccgccgaagacccggaaggttatggttggacccctg |
| | aggcagaattacctgagttcgacttcggcttacgtttgccgacggcggggcatggagaagttgatatggttagcagcatc |
| | gccatgagtggacataagtgggcaggcgcgccgtggccatgcggcatctatatgacgaaagtgaaatatcagattagt |
| | ccaccgtcacagcccgattatattggtgctcctgacacaacatttgccggttcccgtaacggcttttcgccgttaattttgtg |
| | ggatcatttatcgcgctactcgtaccgcgaccaggtagagcgcatccgcgaagcacaggagcttgcagcatatttggaa |
| | cgccgccttaccgctatggagcgcgagctgggagtggaactttggccagcccgcacaccgggtgctgtaaccgtacgt |
| | tttcgcaaaccctctgctgagctggttgcgaagtggtccttgtcgtcgcaggatgttttaatggtgccgggtgatgaaacta |
| | cgcgtcgtagttacgttcatgtgttcgtgatgccttctgttgatcgtgcaaagttagatgcgttgctggcagaattggccgaa |
| | gatcccgtcatcttgggtgcgccttaa |
| yjeH | atgagtggactcaaacaagaactggggctggcccagggcatcggcctactatcgacgtcattattaggcactggcgtgt |
| SEQ ID NO: | ttgccgttcctgcgttagctgcgctagtagcaggcaataacagcctgtgggcgtggcccgttttgattatcttagtgttccc |
| 1014 | gattgcgattgtgtttgcgattctgggtcgccactatcccagcgcaggcggcgtcgcacacttcgtcggtatggcgtttgg |
| | ttcgcggcttgagcgagtcaccggctggttgtttttatcggtcattcccgtgggtttgcctgccgcgctacaaattgctgcc |
| | ggattcggccaggcaatgtttggctggcatagcgggcaactgttgttggcagaactcggtacgctggcgctggtgtggt |
| | atatcggtactcgaggtgccagttccagtgctaatctacaaacagttattgccgggcttatcgtcgcactgattgtcgctatc |
| | tggtgggcgggcgatatcaaacctgcgaatatccccttccctgcgccaggaaatatcgaacttaccgggttattcgctgc |
| | gttatcagtgatgttctggtgttttgtcggtctggaagcatttgcccatcttgcctcggaatttaaaaatccagagcgtgatttt |
| | cctcgtgctttgatgattggcctgctgctggcaggattagtctattggggctgtacggtagtcgtcttacacttcgacgccta |
| | tggtgaacaaatggcggcggcagcatcgcttcccaaaattgtagtgcagttattcggtgtaggagcgttatggattgcctg |
| | cgtaattggctatctggcctgctttgccagtctcaacatttatatacagagcttcgcccgcctggtctggtcgcaggcgcaa |
| | cataatcctgaccattacctggcacgcctctcttctcgccatattccgaataatgccctcaatgcggtgctcggctgctgcg |
| | tggtgagcacgttggtgattcatgctttagagatcaatctggacgctcttattatttatgccaatggcatctttattatgatttat |
| | ctgttatgcatgctggcaggctgtaaattattgcaaggacgttatcgactactggcagtggttggcgggctattatgcgttct |
| | gttactggcaatggtcggctggaaaagtctctacgcgctgatcatgctggcggggttatggctgtttctgccaaaacgaa |
| | aaacgccggaaaatggcataaccacataa |
| yjeH K/O | aatgtgaatggcacgattatgcgggatacttacaccaccgacggaatatgaaaatcaatattatcgacggctcagaagtg |
| (100 bp up | tctagattatccgtggcgatCTGACATGGGAATTAGCCATGGTCCATATGAATATCCT |
| and | CCttAGTTCCTATTCCGAAGTTCCTATTCTCTAGAAAGTATAGGAACTTCG |
| downstream | AAGCAGCTCCAGCCTACACAATCGCTCAAGACGTGTAATtccggcgtttcgacatt |
| and the scar | aatcctggcgatcgtctttatgatcaaggcggtcgcggtcatcatcctttcgctggtactcaccatcaaaagtattaccgcc |
| site in | a |
| between) | |
| SEQ ID NO: | |
| 1120 | |
| YjeH Amino | MSGLKQELGLAQGIGLLSTSLLGTGVFAVPALAALVAGNNSLWAWPVLIIL |
| acid | VFPIAIVFAILGRHYPSAGGVAHFVGMAFGSRLERVTGWLFLSVIPVGLPAA |
| sequence | LQIAAGFGQAMFGWHSGQLLLAELGTLALVWYIGTRGASSSANLQTVIAGL |
| SEQ ID NO: | IVALIVAIWWAGDIKPANIPFPAPGNIELTGLFAALSVMFWCFVGLEAFAHL |
| 1121 | ASEFKNPERDFPRALMIGLLLAGLVYWGCTVVVLHFDAYGEQMAAAASLP |
| | KIVVQLFGVGALWIACVIGYLACFASLNIYIQSFARLVWSQAQHNPDHYLA |
| | RLSSRHIPNNALNAVLGCCVVSTLVIHALEINLDALIIYANGIFIMIYLLCMLA |
| | GCKLLQGRYRLLAVVGGLLCVLLLAMVGWKSLYALIMLAGLWLFLPKRK |
| | TPENGITT |
| Exemplary | gaccagaggtaaggaggtaacaaccatgcgagtgttgaagaaacatcttaatcatgctgcggagggtttcta |
| RBS for | |
| MetP | |
| SEQ ID NO: | |
| 1109 | |
| Exemplary | ATTGCCAATAACAATTACTAAGGAGGTTTTTT |
| RBS for | |
| MetDC | |
| SEQ ID NO: | |
| 1122 | |

1.-28. (canceled)

29. A recombinant bacterial cell comprising:
three copies of a methionine decarboxylase (metDC) gene, wherein each copy of the metDC gene is operably linked to a separate inducible promoter, and wherein the metDC gene encodes a polypeptide having at least 90% identity to SEQ ID NO: 1048;
a metP gene operably linked to an inducible promoter.

30. The recombinant bacterial cell of claim 29, wherein the metDC gene encodes a polypeptide having at least 95% identity to SEQ ID NO: 1048.

31. The recombinant bacterial cell of claim 30, wherein the metDC gene encodes a polypeptide comprising SEQ ID NO: 1048.

32. The recombinant bacterial cell of claim 31, wherein the metP gene encodes a polypeptide having at least 90% identity to SEQ ID NO: 1056.

33. The recombinant bacterial cell of claim 32, wherein the metP gene encodes a polypeptide having at least 95% identity to SEQ ID NO: 1056.

34. The recombinant bacterial cell of claim 33, wherein the metP gene encodes a polypeptide comprising SEQ ID NO: 1056.

35. The recombinant bacterial cell of claim 34, further comprising a knock-out of an endogenous methionine efflux pump, wherein the endogenous methionine efflux pump is encoded by a yjeH gene.

36. The recombinant bacterial cell of claim 35, wherein the yjeH gene comprises a sequence having at least 90% identity to SEQ ID NO: 1014.

37. The recombinant bacterial cell of claim 36, wherein the yjeH gene comprises a sequence having at least 95% identity to SEQ ID NO: 1014.

38. The recombinant bacterial cell of claim 37, wherein the yjeH gene comprises a sequence of SEQ ID NO: 1014.

39. The recombinant bacterial cell of claim 38, comprising a ΔdapA auxotrophy.

40. The recombinant bacterial cell of claim 39, comprising a modified endogenous colibactin island, wherein the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and/or clbR (SEQ ID NO: 1082).

41. The recombinant bacterial cell of claim 40, comprising a deletion of an endogenous phage gene, wherein the endogenous phage gene comprises a sequence having at least 90% identity to SEQ ID NO: 1064.

42. The recombinant bacterial cell of claim 41, wherein the endogenous phage gene comprises a sequence having at least 95% identity to SEQ ID NO: 1064.

43. The recombinant bacterial cell of claim 42, wherein the endogenous phage gene comprises a sequence of SEQ ID NO: 1064.

44. The recombinant bacterial cell of claim 43, wherein the recombinant bacterial cell is a recombinant probiotic bacterial cell.

45. The recombinant bacterial cell of claim 44, wherein the recombinant bacterial cell is of the species Escherichia coli strain Nissle.

46. The recombinant bacterial cell of claim 45, wherein each copy of the metDC gene is operably linked to a separate IPTG inducible promoter.

47. The recombinant bacterial cell of claim 46, wherein the metP gene is operably linked to an IPTG inducible promoter.

48. A recombinant bacterial cell comprising:
three copies of a methionine decarboxylase (metDC) gene, wherein each copy of the metDC gene is operably linked to a separate IPTG inducible promoter, and wherein the metDC gene encodes a polypeptide comprising SEQ ID NO: 1048;
a metP gene operably linked to an IPTG inducible promoter, wherein the metP gene encodes a polypeptide comprising SEQ ID NO: 1056;
a knock-out of an endogenous methionine efflux pump, wherein the endogenous methionine efflux pump is encoded by a yjeH gene, and wherein the yjeH gene comprises a sequence comprising SEQ ID NO: 1014;
a ΔdapA auxotrophy;
a modified endogenous colibactin island, wherein the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and/or clbR (SEQ ID NO: 1082); and
a deletion of an endogenous phage gene, wherein the endogenous phage gene comprises a sequence comprising SEQ ID NO: 1064.

49. A pharmaceutical composition comprising the recombinant bacterial cell of claim 47 and a pharmaceutically acceptable carrier.

50. A pharmaceutical composition comprising the recombinant bacterial cell of claim 48 and a pharmaceutically acceptable carrier.

51. A method for reducing the levels of methionine, cysteine and/or homocysteine in a subject, the method comprising administering to the subject the pharmaceutical composition of claim 49, thereby reducing the levels of methionine, cysteine, and/or homocysteine in the subject.

52. The method of claim 51, wherein the subject has homocystinuria, cystinuria, or a metabolic disease.

53. The method of claim 51, wherein the pharmaceutical composition comprises about 1×10¹¹ to about 6×10¹¹ live recombinant bacterial cells/mL.

54. A method for reducing the levels of methionine, cysteine and/or homocysteine in a subject, the method comprising administering to the subject the pharmaceutical composition of claim 50, thereby reducing the levels of methionine, cysteine, and/or homocysteine in the subject.

55. A method for treating a disease associated with methionine metabolism in a subject, the method comprising administering the pharmaceutical composition of claim 49 to the subject, thereby treating the disease.

56. A method for treating a disease associated with methionine metabolism in a subject, the method comprising administering the pharmaceutical composition of claim 50 to the subject, thereby treating the disease.

57. A method for monitoring the effectiveness of a treatment of a subject, the method comprising administering to the subject the pharmaceutical composition of claim 49, and measuring a level of 3-MTP in urine of the subject.

58. A method for monitoring the effectiveness of a treatment of a subject, the method comprising administering to the subject the pharmaceutical composition of claim 50, and measuring a level of 3-MTP in urine of the subject.
